(19) United States
(12) Patent Application Publication
(10) Pub. No.: US 2024/0271122 A1
(43) Pub. Date: Aug. 15, 2024

(54) METHODS FOR CODON OPTIMIZATION AND USES THEREOF

(71) Applicant: Opentrons Labworks Inc., Brooklyn, NY (US)

(72) Inventors: Joel S. BADER, Bronx, NY (US); Leslie MITCHELL, New York, NY (US)

(21) Appl. No.: 18/286,611

(22) PCT Filed: Apr. 14, 2022
(86) PCT No.: PCT/US2022/024888

§ 371 Date: Oct. 12, 2023

Related U.S. Application Data
(60) Provisional application No. 63/174,823, filed on Apr. 14, 2021.

(51) Int. Cl. C12N 15/10

(52) U.S. Cl. CPC C12N 15/1058; C12N 15/1082; C12N 15/1089

(57) ABSTRACT

Provided herein are methods and systems for codon rewriting and replacement. In some aspects, provided herein, is a method comprising: analyzing at least a portion of a genome of an organism to identify a first plurality of codons based on at least in part on a first local context of a codon-of-interest in the genome of the organism to be rewritten; and rewriting the first plurality of codons in the genome of the organism to a second codon. Also provided herein are methods and systems for producing a synthetic genome.

### CROSS REFERENCE

This application claims the benefit of U.S. Provisional Application No. 63/174,823, filed on Apr. 14, 2021, which is incorporated herein by reference in its entirety.

### SEQUENCE LISTING

This instant application contains a Sequence Listing which has been submitted electronically in ASCII format and is hereby incorporated by reference in its entirety. Said ASCII copy, created on Apr. 14, 2022, is named 59725703601_SL.txt and is 23,977,365 bytes in size.

### BACKGROUND

Codon rewriting and repurposing translational machinery may be important tools to expand the genetic code artificially and ultimately to custom-design a synthetic genome.

These may also be important tools to enable incorporation of non-canonical amino acids (ncAAs) into proteins. However, approaches for determining codon replacement remain limited, and there is a need for improved approaches for selecting a codon/s for rewriting and replacement.

### SUMMARY

In some aspects, provided herein, is a method comprising: a) analyzing at least a portion of a genome of an organism to identify a first plurality of codons based on at least in part on a first local context of a codon-of-interest in the genome of the organism to be rewritten; b) rewriting the first plurality of codons in the genome of the organism to a second codon, wherein the first plurality of codons and the second codon encode a first amino acid, and wherein the rewriting of the first plurality of codons modulates an occurrence of the first plurality of codons; and c) synthesizing a nucleic acid construct comprising the portion of the genome, wherein the first plurality of codons is rewritten to the second codon.

Another aspect of the present disclosure provides a method of producing a polypeptide comprising a non-canonical amino acid (ncAA) or a population of polypeptide molecules comprising the ncAA in an organism, the method comprising: rewriting a first codon encoding a first amino acid to a second codon encoding the first amino acid in a genome of the organism, wherein the rewriting comprises identifying the first codon based at least in part on a first local context of a codon-of-interest in the genome of the organism; reassigning the first codon to encode the ncAA in the genome of the organism; and introducing into the organism an aminoacyl-tRNA synthetase (aaRS)/tRNA pair engineered to recognize the first codon and incorporate the ncAA into an amino acid sequence of the polypeptide or the population of the polypeptide molecules.

Another aspect of the present disclosure provides a method of producing a peptide, the method comprising editing a genome of an organism, wherein the editing comprises revising a codon of the genome to encode a non-canonical amino acid, wherein the peptide comprises the non-canonical amino acid.

Another aspect of the present disclosure provides a cell or a population of cells comprising a genome, wherein a first plurality of codons in the genome of the organism is rewritten to a second codon, wherein the first plurality of codons and the second codon encode a first amino acid, and wherein an occurrence of the first plurality of codons is modulated responsive to being rewritten to the second codon. Another aspect of the present disclosure provides an organism comprising the cell or the population of cells described herein.

Another aspect of the present disclosure provides a computer system for editing a genome of an organism, comprising: a database that is configured to store at least a portion of the genome of the organism; and one or more computer processors operatively coupled to said database, wherein said one or more computer processors are individually or collectively programmed to: a) analyze the at least the portion of the genome of the organism to identify a first plurality of codons in the genome of the organism to be rewritten; and b) rewrite the first plurality of codons in the genome of the organism to a second codon, wherein the first plurality of codons and the second codon encode a first amino acid, and wherein the rewriting of the first plurality of codons modulates an occurrence of the first plurality of codons, thereby editing the genome of the organism.

Another aspect of the present disclosure provides a non-transitory computer-readable storage medium comprising machine-executable code that, upon execution by one or more computer processors, implements a method for editing a genome of an organism, the method comprising: a) analyzing at least a portion of the genome of the organism to identify a first plurality of codons in the genome of the organism to be rewritten; and b) rewriting the first plurality of codons in the genome of the organism to a second codon, wherein the first plurality of codons and the second codon encode a first amino acid, and wherein the rewriting of the first plurality of codons modulates an occurrence of the first plurality of codons, thereby editing the genome of the organism.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

Each patent, publication, and non-patent literature cited in the application is hereby incorporated by reference in its entirety as if each was incorporated by reference individually. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 depicts deviations from overall relative synonymous codon usage for codons in specific contexts. The context is determined as the codons on either side of a central codon. Given the amino acid of the central codon, the codon usage for the central codon is compared to the overall relative synonymous codon usage (RSCU) and a p-value is determined. Labels indicate central codons with significant deviations from the null, and the dashed line represents the significance threshold corrected for the number of tests.

FIG. 2 illustrates genome features that may be impacted by genome writing.

FIG. 3 illustrates exemplary genome features that may be impacted by genome writing. As seen in FIG. 3, lncRNA refers to long non-coding RNA.

FIG. 4 is an exemplary schematic for optimizing recoding one or more codons in a synthetic strain. As seen in FIG. 4, aaRS refers to a aminoacyl-tRNA synthetase, and ncAA refers to a non-canonical amino acid.

FIGS. 5A-5C show an exemplary quantitative report platform to evaluate non-canonical amino acid (ncAA) incorporation (FIG. 5A), including a dual reporter system for surface display (FIG. 5B) and for intracellular fluorescence (FIG. 5C).

FIG. 6 depicts an exemplary codon replacement design for leucine (Leu). See Example 1 for details. Anticodon TAG recognizes CTG, and a 3-gene family must be deleted to rewrite CTG. tRNA tL(GAG) is a single-copy gene and cells with deletion of this gene are viable. tL(UAG) is known to recognize all 6 Leu codons. Fitness of cells with tL(UAG)J/L1/L2 deletion likely requires supplementation with additional copies of tL(GAG). In some example embodiments, Candida and other yeasts where CTG encodes Ser may have tL(AAG) genes. Adenine (A) can be modified to inosine (I) and I recognizes uridine (U)/cytosine (C)/adenine(A) but not guanine (G) in the 3rd position. RSCU refers to Relative Synonymous Codon Usage; KO refers to knock out; an exemplary codon block for removal comprises CAG and TAG; in some example embodiments, codons that may be better to retain comprise AAG and GAG.

FIG. 7 depicts an exemplary codon replacement design for serine (Ser). See Example 1 for details. tS(CGA)C/SUP61 is a single-copy essential tRNA that recognizes TCG. By normal rules, tS(UGA) should recognize UCG by wobble. For robustness, 3 copies of tS(UGA) may need to be deleted in addition to single-copy tS(CGA). Recognition of AGT/AGC is standard, 4-copy tS(GCU) family, single deletions have slow growth. Ser TCG/TCA rewrite: 78K codons, 4 tRNAs (one single gene, one triple gene). Ser AGT/AGC rewrite: 70K codons, 4 tRNAs. RSCU refers to Relative Synonymous Codon Usage; KO refers to knock out; in some example embodiments, a codon block for removal comprises CGA and TGA; in some example embodiments, an alternative codon block for removal comprises ACT and GCT.

FIG. 8 depicts an exemplary codon replacement design for arginine (Arg). See Example 1 for details. In some example embodiments, a yeast mitochondrial genome is devoid of rare codons comprising CGG, CGA codons (vs. E. coli where the 2-codon box is rare). TRR4/tR(CCG) is a single-copy essential tRNA. According to the standard rules, TRR4 should have no wobble. CGA is likely recognized by tR(ACG), a 6-gene family which may recognize CGU/C/A through wobble, not CGG. CGA is low copy. Cross-talk risk can be reduced by rewriting CGG and CGA. Arg CGG/CGA rewrite: 14K codons, 1 tRNA. RSCU refers to Relative Synonymous Codon Usage; KO refers to knock out; in some example embodiments, a codon block for removal comprises CCG and TCG in some example embodiments, codons that may be better to retain comprise CCT and TCT.

FIG. 9 depicts an exemplary codon replacement using Goldilocks method.

FIG. 10 depicts an illustrative example for constructing a yeast strain with in silico designed synthetic genome.

FIG. 11 depicts an example of how a codon is selected for replacement and reassignment.

FIG. 12 is a table depicting pilot regions to select in yeast genome for best derisk design based on number of essential genes, number of codons to rewrite in essential genes, and/or additional genes and codons. Some of these regions may be extended to capture additional essential genes.

FIG. 13 is a table depicting a yeast codon usage.

FIG. 14 depicts a computer system comprising a program configured to implement methods provided herein. In some cases, the program comprises an algorithm. The computer system may be a machine learning-based computer system that determines codon frequency.

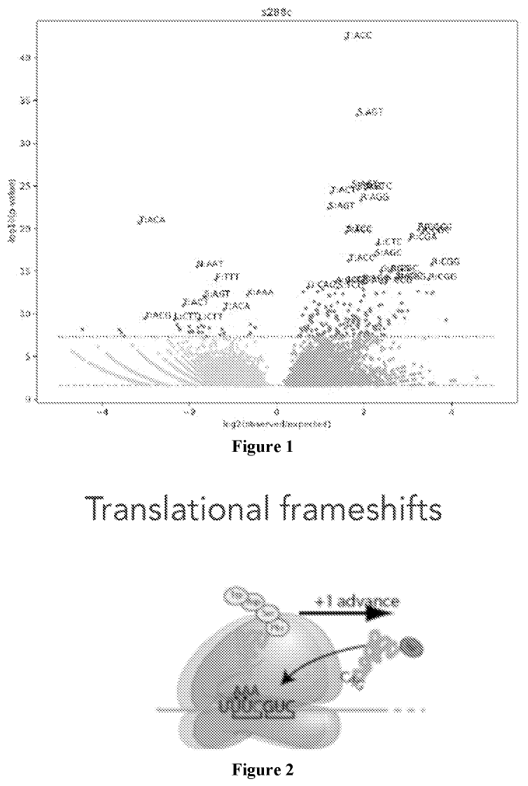

In some cases, the computer system comprises a computer processing unit and a sequence processing unit, wherein the computer processing unit and the sequence processing unit are bilaterally communicatively coupled. In some embodiments, the sequence processing unit and the computer processing unit comprise a storage component. 1410: Computer system. 1420: Central processing unit of computer system. 1430: Data storage with files containing the translation tables representing the genetic code of the organism whose genome is being rewritten. 1440: Instructions describing which translation table to use, the codons to be eliminated, and the locations of input and output files. 1450: Computer program implementing the methods to perform the codon rewriting. 1460: Input file, possibly on the same computer system or accessible from a different computer system, providing the sequence of protein-coding regions in the original genome. 1470, 1460: Output file, possibly on the same computer system or writeable on a different computer system, with the gene sequences rewritten to eliminate specified codons, and possible additional files with diagnostics, statistical analyses providing context-specific codon usage, and other reports. 1480: The computer system may also be attached to cloud resources for data import and export.

### DETAILED DESCRIPTION

Provided herein are methods for designing a genome of an organism by rewriting one or more codons. In some aspects, methods described herein may comprise replacing one or more codons with another codon encoding the same amino acid. In some aspects, the one or more codons being replaced may be used to encode another amino acid, for example, a non-canonical amino acid (ncAA). Provided herein are methods for reducing or minimizing an occurrence of one or more synonymous codons used to encode an amino acid. Also provided herein are methods for efficient translation of a protein or a portion thereof with one or more ncAAs. The present specification also describes how to identify one or more codons for rewriting and/or replacement.

### Definitions

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and Cₙ” The term “or” can be used conjunctively or disjunctively, unless the context specifically refers to a disjunctive use.

The term “about” or “approximately” can mean within an acceptable error range for the particular value, which may depend in part on how the value is measured or determined, e.g., the limitations of the measurement system. For example, “about” can mean within 1 or more than 1 standard deviation. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the present disclosure, subject to any specifically excluded limit in the stated range. Where the stated range includes one or both of the limits, ranges excluding either or both of those included limits are also included in the present disclosure, unless the context clearly dictates otherwise.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

Certain specific details of this description are set forth in order to provide a thorough understanding of various embodiments. However, one skilled in the art will understand that the present disclosure may be practiced without these details. In other instances, well-known techniques or methods have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure.

The nomenclature used to describe polypeptides or proteins follows the conventional practice wherein the amino group is presented to the left (the amino- or N-terminus) and the carboxyl group to the right (the carboxy- or C-terminus) of each amino acid residue. When amino acid residue positions are referred to in a polypeptide or a protein, they are numbered in an amino to carboxyl direction with position one being the residue located at the amino terminal end of the polypeptide or the protein of which it can be a part. The amino acid sequences of peptides set forth herein are generally designated using the standard single letter or three letter symbol. (A or Ala for Alanine; C or Cys for Cysteine; D or Asp for Aspartic Acid; E or Glu for Glutamic Acid; F or Phe for Phenylalanine; G or Gly for Glycine; H or His for Histidine; I or Ile for Isoleucine; K or Lys for Lysine; L or Leu for Leucine; M or Met for Methionine; N or Asn for Asparagine; P or Pro for Proline; Q or Gln for Glutamine; R or Arg for Arginine; S or Ser for Serine; T or Thr for Threonine; V or Val for Valine; W or Trp for Tryptophan; and Y or Tyr for Tyrosine).

The term “non-canonical amino acid” or “ncAA” refers to any amino acid other than the 20 standard amino acids (alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine). There are over 700 known ncAA any of which may be used in the methods described herein. In some embodiments, examples of ncAA include, but are not limited to, L-Tryptazan, 5-Fluoro-L-tryptophan, L-Ethionine, L-Selenomethionine, Trifluoro-L-methionine, L-Norleucine, L-Homopropargylglycine, (2S)-2-amino-5-(methylsulfanyl) pentanoic acid, (2S)-2-amino-6-(methylsulfanyl) hexanoic acid, Para-fluoro-L-phenylalanine, Para-iodo-L-phenylalanine, Para-azido-L-phenylalanine, Para-acetyl-L-phenylalanine, Para-benzoyl-L-phenylalanine, Meta-fluoro-L-tyrosine, O-methyl-L-tyrosine, Para-propargyloxy-L-phenylalanine, (2S)-2-aminooctanoic acid, (2S)-2-aminononanoic acid, (2S)-2-aminodecanoic acid, (2S)-2-aminohept-6-enoic acid, (2S)-2-aminooct-7-enoic acid, L-Homocysteine, (2S)-2-amino-5-sulfanylpentanoic acid, (2S)-2-amino-6-sulfanylhexanoic acid, L-S-(2-nitrobenzyl) cysteine, L-S-ferrocenyl-cysteine, L-O-crotylserine, L-O-(pent-4-en-1-yl)serine, L-O-(4,5-dimethoxy-2-nitrobenzyl)serine, (2S)-2-amino-3-({[5-(dimethylamino)naphthaleₙ₋1-yl]sulfonyl}amino)propanoic acid, (2S)-3-[(6-acetyl-naphthaleₙ₋1-yl)amino]-2-aminopropanoic acid, L-Pyrrolysine, N⁶-[(propargyloxy)carbonyl]-L-lysine, L-N⁶-acetyllysine, N⁶-trifluoroacetyl-L-lysine, N⁶—{[1-(6-nitro-1,3-benzodioxol-5-yl)ethoxy]carbonyl}-L-lysine, N⁶—{[2-(3-methyl-3H-diaziren-3-yl)ethoxy]carbonyl}-L-lysine, p-azidophenylalanine, and 2-aminoisobutyric acid. In some embodiments, examples of ncAA include, but are not limited to, AbK (unnatural amino acid for Photo-crosslinking probe), 3-Aminotyrosine (unnatural amino acid for inducing red shift in fluorescent proteins and fluorescent protein-based biosensors), L-Azidohomoalanine hydrochloride (unnatural amino acid for bio-orthogonal labeling of newly synthesized proteins), L-Azidonorleucine hydrochloride (unnatural amino acid for bio-orthogonal or fluorescent labeling of newly synthesized proteins), BzF (photoreactive unnatural amino acid; photo-crosslinker), DMNB-caged-Serine (caged serine; excited by visible blue light), HADA (blue fluorescent D-amino acid for labeling peptidoglycans in live bacteria), NADA-green (fluorescent D-amino acid for labeling peptidoglycans in live bacteria), NB-caged Tyrosine hydrochloride (ortho-nitrobenzyl caged L-tyrosine), RADA (orange-red TAMRA-based fluorescent D-amino acid for labeling peptidoglycans in live bacteria), Rf470DL (blue rotor-fluorogenic fluorescent D-amino acid for labeling peptidoglycans in live bacteria), sBADA (green fluorescent D-amino acid for labeling peptidoglycans in bacteria), and YADA (green-yellow lucifer yellow-based fluorescent D-amino acid for labeling peptidoglycans in live bacteria). In some embodiments, examples of ncAA include, but are not limited to, β-alanine, D-alanine, 4-hydroxyproline, desmosine, D-glutamic acid, γ-aminobutyric acid, β-cyanoalanine, norvaline, 4-(E)-butenyl-4(R)-methyl-N-methyl-L-threonine, N-methyl-L-leucine, selenocysteine, and statine. In some embodiments, a ncAA comprises p-azidophenylalanine or 2-aminoisobutyric acid (also known as α-aminoisobutyric acid, AIB, α-methylalanine, or 2-methylalanine).

The terms “codon” and “anticodon” as used herein may refer to DNA or RNA. In some embodiments, DNA comprises nucleotide bases adenine (A), guanine (G), cytosine (C), or thymine (T). In some embodiments, RNA comprises nucleotide bases adenine (A), guanine (G), cytosine (C), or uracil (U). In some embodiments, DNA or RNA may comprise inosine (I). in some embodiments, inosine (I) may pair with adenine (A), cytosine (C), or uracil (U). In some embodiments, DNA or RNA may comprise queuosine (Q). In some embodiments, queuosine (Q) may pair with cytosine (C) or uracil (U).

Although methods and materials similar or equivalent to those described herein can be used in the practice or testing of the present disclosure, suitable methods, and materials are described below.

### Design Derisking for Genome Editing Design

### RNA Notation

In some aspects, provided herein are methods for selecting a codon for rewriting or replacement. In some embodiments, a codon may be selected based on an analysis of the genetic code. In some embodiments, the analysis may depend on messenger RNA (mRNA) codon recognition by a tRNA anticodon. In some embodiments, ribonucleotides (e.g., A, C, G, U, or I) may be used. In some embodiments, deoxyribonucleotides (e.g., A, C, G, or T) may be used.

### Wobble Minimization

In some aspects, a codon may be selected for replacement to minimize wobble. In some embodiments, more than one codon ending in different nucleotides can encode the same amino acid. For example, this may happen because a single transfer RNA (tRNA) anticodon can recognize multiple mRNA codons through wobble. The third nucleotide position of a codon is the wobble position, corresponding to the first nucleotide position of a corresponding anticodon.

For example, the wobble rule may be that an anticodon starting with the nucleotide C (e.g., CXX from 5′ to 3′ direction of an anticodon, wherein X can be any nucleotide) can only recognize the nucleotide G in the third nucleotide position of a corresponding codon (e.g., XXG from 5′ to 3′ direction of a codon, wherein X can be any nucleotide). In some embodiments, an anticodon starting with the nucleotide C may only recognize G in the third nucleotide position of a codon. Thus, in some embodiments, ATG codon may only encode methionine (Met). In some embodiments, UGG codon may only encode tryptophan (Trp). In some embodiments, CUA anticodon may suppress the amber stop codon UAG. In some embodiments, CUA anticodon may not suppress the ochre stop codon UAA.

In some embodiments, an anticodon may start with nucleotide G and G may be converted to queuosine (Q) that can recognize nucleotide C or U in a codon. In some embodiments, an anticodon may start with nucleotide A, and A may be converted to I (inosine) that can recognize nucleotide A, C, or U in a codon. In some embodiments, an anticodon may start with U and may be modified to recognize nucleotide A or G, or in some cases C or U. Thus, in an embodiment, a codon starting with G may be used in the wobble position as a target for rewriting.

TABLE 1
Codon-Anticodon Pairing under Wobble Rules

| 3′ end of a codon (or third | 5′ end of an anticodon (or first |
|---|---|
| nucleotide position of a codon) | nucleotide position of an anticodon) |
| C or U | G or Q (queosine) |
| G only | C (no wobble) |
| U only | A |
| A or G (or A, G, C, U in bacteria) | U |
| U, C, or A | A edited to I (inosine) |

In some embodiments, an amino acid may be encoded by one codon (e.g., out of 64 possible permutations of codons, having one of 4 different nucleotides at each of 3 different positions). For example, Methionine (Met) can be encoded by a single codon AUG. In some embodiments, an amino acid may be encoded by one or more codons. In some embodiments, an amino acid may be encoded by one or two codons (e.g., out of 64 possible permutations of codons). For example, Lysine (Lys) can be encoded by either of the two codons AAA or AAG. For example, Glutamic acid (Glu) can be encoded by either of the two codons GAA or GAG. In these embodiments, an anticodon starting with U may recognize AAA or GAA, and in addition, AAG or GAG, due to cross-talk (see Table 1). Thus, in some embodiments, a codon encoding an amino acid encoded by one or two codons may not be used for genome rewriting or replacement.

In some embodiments, an amino acid may be encoded by any of one, two, three, four, five, or six codons. For example, arginine (Arg) can be encoded by any of the six codons CGU, CGC, CGA, CGG, AGA, or AGG. For example, serine (Ser) can be encoded by any of the six codons AGU, AGC, UCU, UCC, UCA, or UCG. For examples, leucine (Leu) can be encoded by any of the six codons UUA, UUG, CUU, CUC, CUA, or CUG. In some embodiments, a codon of the set of one, two, three, four, five, or six codons that encode the same amino acid may be selected for rewriting or replacement.

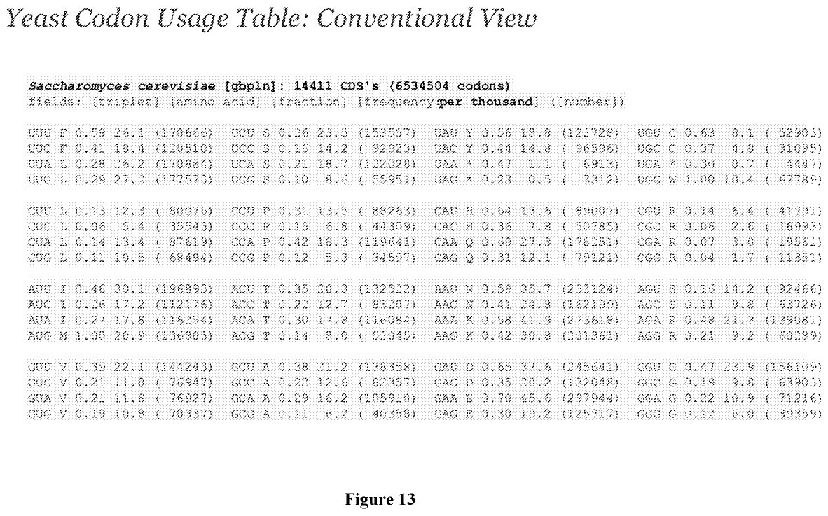

Table 2 below shows standard rules for anticodon-codon pairing in a model organism, yeast. FIG. 13 shows codon usage in yeast.

TABLE 2
Standard Rules for Anticodon-Codon Pairing in Yeast

| tDNA | Number | | | |
|---|---|---|---|---|
| anticodon | of genes | Anticodon | Codon | Amino acid |
| AGC | 11 | IGC | gcu, gcc | Ala |
| TGC | 5 | UGC | gca, gcg | |
| ACG | 6 | ICG | cgu, cgc, cga | Arg |
| CCG | 1 | CCG | ccg | |
| TCT | 11 | UCU | aga | |
| CCT | 1 | CCU | agg | |
| GTT | 10 | GUU | aau, aac | Asn |
| GTC | 15 | GUC | gau, gac | Asp |
| GCA | 4 | GCA | ugu, ugc | Cys |
| TTG | 9 | UUG | caa | Gln |
| CTG | 1 | CUG | cag | |
| TTC | 14 | UUC | gaa | Glu |
| CTC | 2 | CUC | gag | |
| GCC | 16 | GCC | ggu, gge | Gly |
| TCC | 3 | UCC | gga | |
| CCC | 2 | CCC | ggg | |
| GTG | 7 | GUG | cau, cac | His |
| AAT | 13 | IAU | auu, auc | Ile |
| TAT | 2 | UAU | aua | |
| TAA | 7 | UAA | uua | Leu |
| CAA | 10 | CAA | uug | |
| GAG | 1 | GAG | cuu, cuc | |
| TAG | 3 | UAG | cua, cug | |
| TTT | 7 | UUU | aaa | Lys: |
| CTT | 14 | CUU | aag | |
| CAT | 5 | CAU | aug | Met |
| CAT | 5 | CAU | aug | Met |
| GAA | 10 | GAA | uuu, uuc | Phe |
| AGG | 2 | IGG | ccu, ccc | Pro |
| TGG | 10 | UGG | cca, ccg | |
| AGA | 11 | IGA | ucu, ucc | Ser |
| TGA | 3 | UGA | uca | |
| CGA | 1 | CGA | uga | |
| GCT | 4 | GCU | agu, agc | |
| AGT | 11 | IGU | acu, acc | Thr |
| TGT | 4 | UGU | aca | |
| CGT | 1 | CGU | acg | |
| CCA | 6 | CCA | ugg | Trp |
| GTA | 8 | GUA | uau, uac | Tyr |
| AAC | 14 | IAC | guu, guc | Val |
| TAC | 2 | UAC | gua | |
| CAC | 2 | CAC | gug | |

Gene copy number and predicted decoding specificities of yeast tRNAs

In some embodiments, a class of codons for which a corresponding anticodon is not a part of the tRNA identity element recognized by a corresponding aminoacyl-tRNA synthetase (aaRS) may be considered. In some embodiments, this class of codons comprises, but is not limited to, leucine (Leu), serine (Ser), or alanine (Ala).

### Codon Reassignment (Codon Capture)

In some aspects, provided herein are methods for codon rewriting and replacement that allow high fitness of an organism. In some embodiments, at the amino acid-to-tRNA level, aminoacyl-tRNA synthetase (aaRS) that may not interact with an anticodon for clean codon reassignment downstream may be considered. In some embodiments, yeast genetic code evolution may be considered. In some embodiments, at the codon-to-anticodon level, codon removal may allow for deletion of all tRNAs used for decoding. In some embodiments, deletion of tRNAs may not disable decoding of synonymous codons through wobble. In some embodiments, no remaining natural tRNAs can decode rewritten, replaced, or eliminated codon(s), if reinserted.

In some embodiments, methods for codon rewriting and/or replacement disclosed herein can use a context-sensitive design (e.g., learned from a host organism) for unbiased discovery of problematic motifs based on positive evolutionary selection and/or negative evolutionary selection. In some embodiments, each codon may be considered in the local context (e.g., based on the codons on either side of a given codon of interest), and codons may be selected for re-writing at least in part by normalizing for the observed frequency of the codon in the context of its surrounding codons relative to the null hypothesis of overall relative synonymous codon usage.

In some embodiments, genes such as Saccharomyces cerevisiae genes can be examined for context-sensitive codon usage. In some embodiments, S. cerevisiae genes may have statistically significant evolutionary signals, such as negative selection leading to predictable de-enriched sequences, such as “slippery sites” (e.g., homopolymer runs), and/or positive selection for functional regulatory motifs, such as Rap1 binding sites. In some embodiments, methods for selecting a replacement codon may comprise a statistical optimization or outlier avoidance approach (e.g., a “Goldilocks” approach) to avoid selection of a replacement codon with a positive evolutionary signal (e.g., a codon that is too “hot” having a usage that is significantly higher than the overall RSCU for that given codon) or a negative evolutionary signal (e.g., a codon that is too “cold” having a usage that is significantly lower than the overall RSCU for that given codon), and instead to select a replacement codon based at least in part on consideration of the codon's local context (e.g., by considering replacement codons whose relative synonymous usage in the given context most closely matches its relative synonymous usage overall). In some embodiments, such selection of replacement codons may comprise determining context-sensitive relative synonymous codon usage (RSCU) value for each of a plurality of codons (e.g., representing a local context of a given codon of interest), and identifying a codon from among the plurality of codons having a maximum or largest RSCU value. For example, the plurality of codons may comprise a codon of interest, a second codon that is upstream of the codon of interest, and a third codon that is downstream of the codon of interest. For example, the plurality of codons may comprise a set of at least three consecutive codons: a codon of interest, a second codon that is upstream of and adjacent to the codon of interest, and a third codon that is downstream of and adjacent to the codon of interest. For example, the maximal RSCU value may be at least about 0.01, at least about 0.05, at least about 0.10, at least about 0.11, at least about 0.12, at least about 0.13, at least about 0.14, at least about 0.15, at least about 0.16, at least about 0.17, at least about 0.18, at least about 0.19, at least about 0.20, at least about 0.21, at least about 0.22, at least about 0.23, at least about 0.24, at least about 0.25, at least about 0.26, at least about 0.27, at least about 0.28, at least about 0.29, at least about 0.30, at least about 0.31, at least about 0.32, at least about 0.33, at least about 0.34, at least about 0.35, at least about 0.36, at least about 0.37, at least about 0.38, at least about 0.39, at least about 0.40, at least about 0.41, at least about 0.42, at least about 0.43, at least about 0.44, at least about 0.45, at least about 0.46, at least about 0.47, at least about 0.48, at least about 0.49, at least about 0.50, at least about 0.51, at least about 0.52, at least about 0.53, at least about 0.54, at least about 0.55, at least about 0.56, at least about 0.57, at least about 0.58, at least about 0.59, at least about 0.60, at least about 0.61, at least about 0.62, at least about 0.63, at least about 0.64, at least about 0.65, at least about 0.66, at least about 0.67, at least about 0.68, at least about 0.69, at least about 0.70, at least about 0.71, at least about 0.72, at least about 0.73, at least about 0.74, at least about 0.75, at least about 0.76, at least about 0.77, at least about 0.78, at least about 0.79, at least about 0.80, at least about 0.81, at least about 0.82, at least about 0.83, at least about 0.84, at least about 0.85, at least about 0.86, at least about 0.87, at least about 0.88, at least about 0.89, or at least about 0.90, at least about 0.91, at least about 0.92, at least about 0.93, at least about 0.94, at least about 0.95, at least about 0.96, at least about 0.97, at least about 0.98, at least about 0.99, or about 1.00. This approach may advantageously select the replacement codon having the maximum context-sensitive codon usage. In some embodiments, motifs identified as associated with positive evolutionary signals or negative evolutionary signals that include codons that are to be replaced by a rewriting design may be highlighted as requiring greater scrutiny to avoid introducing fitness defects by rewriting. In this embodiment, methods using an approach to use a replacement codon that shares the same evolutionary signal as the re-written codon may be used. In some embodiments, rewriting designs may be selected to minimize the number of evolutionary motifs affected. In some embodiments, nonsynonymous codons may be introduced instead of introducing a motif with an evolutionary signal through replacement with a synonymous codon.

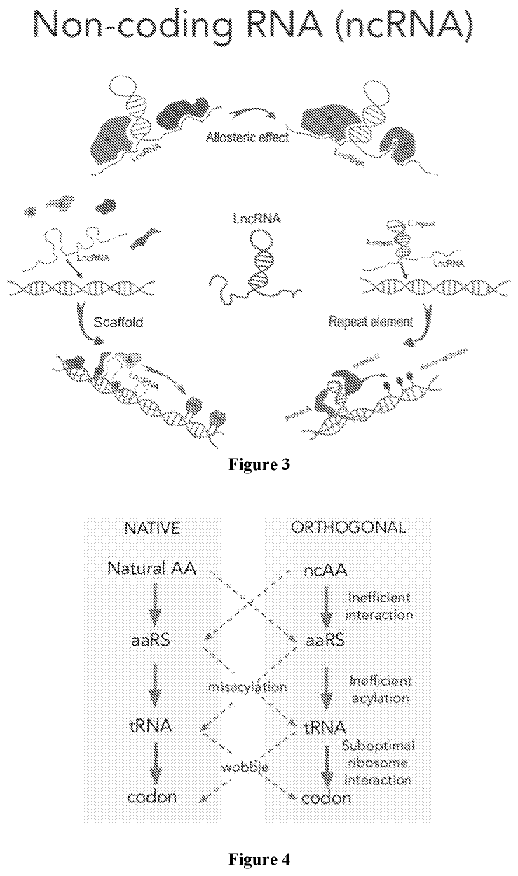

In some embodiments, codon and/or genome rewriting may comprise a risk. In some embodiments, the risk may comprise translational frameshifts (FIG. 2) or non-coding RNA (ncRNA, FIG. 3). In some embodiments, translational frameshifts may be used for gene regulation by a Ty repeat, killer virus elements, or yeast genes comprising OAZ1, ABP140, EST3, or YFS1. In some embodiments, ncRNA may comprise tRNA, small nuclear (snRNA), or small nucleolar RNA (snoRNA). In some embodiments, an ncRNA may be functional. In some embodiments, an ncRNA may not be functional. In some embodiments, the risk described herein can be addressed computationally during genome design through genome-wide alignment of designed CDSs to annotated ncRNAs to identify antisense binding.

In some embodiments, the risk may be related to orthogonal translation system. In some embodiments, the risk may comprise low uptake of ncAA from media into an organism (e.g., yeast), low expression levels of aaRS, or mislocalization of aaRS. In some embodiments, the risk may comprise inefficient interaction between an ncAA and the corresponding aaRS, inefficient acylation of a tRNA, or suboptimal ribosome interaction of tRNA or codon (FIG. 4). In some embodiments, the risk described herein can be obviated by, for example, rapid yeast pathway engineering, codon optimization, CDS copy number, tRNA copy number, promoter/terminator shuffling, transplant aaRS orthologs, CDS molecular breeding, or titratable gene expression systems. In some embodiments, the risk described herein can be obviated by, for example, two to four week cycle time for design-build-deliver-test-learn. In some embodiments, the risk described herein can be mitigated or obviated by, for example, performing parallelizable strain construction and screening.

In some embodiments, each aaRS may recognize all of the tRNAs for an amino acid for amino acid targeting. In some embodiments, recognition may involve amino acid and depending on the aaRS, regions of the tRNA, for example, attachment region, variable loops and stems, and/or an anticodon loop. In some embodiments, the anticodon loop recognition may pose an issue for a method disclosed herein. For example, if an anticodon that is part of aaRS recognition is used, then the native aaRS may still recognize the anticodon and give a mixture of canonical and non-canonical amino acid incorporation. Serine, leucine, and alanine are special in this regard as aaRS generally does not recognize the anticodon. In some embodiments, it may be because serine and leucine have 6 codon blocks, which can provide more diversity in the anticodon. In some embodiments, it may be because in yeast, a part of the anticodon loop is recognized for leucine.

### Derisked by Evolution: Leu, Arg, Ser, Stop

In some aspects, the genetic code may have variations depending on organism. This may be because of evolutionary reassignment of codons (see Table 3). For example, leucine codons are captured by serine in Candida (e.g., CTG). For example, leucine codons are captured by alanine in a fungal clade including Pachysolen. In another example, arginine codons have been lost in yeast mitochondria. In another example, serine-aaRS does not recognize serine anticodon.

In some embodiments, stop codons deleted for codon reassignment/replacement may be captured by nearby amino acids (eRFI in ciliates evolved for UGA vs UAA/UAG recognition). In some embodiments, alanine is not captured by evolution. In some embodiments, alanine's 4-codon block (i.e., there are 4 synonymous codons encoding alanine) in yeast is covered by two larger tRNA families, so it may be difficult to completely eliminate one of the families. In some embodiments, tRNA-aaRS interaction with amino acid works by excluding large sidechains.

TABLE 3
Codons Derisked by evolution: Leu, Arg, Ser and Stop codons

| | Standard | |
|---|---|---|
| Codon | Code | Alternative Code |
| UUY | Phe | |
| UUR | Leu | |
| CUY | Leu | Thr (mitoch) |
| CUA | Leu | Thr (mitoch) |
| CUG | Leu | Ser (Candida), Ala (Pachysolen), Thr/Ser (mitochi) |
| AUY | Ile | |
| AUA | Ile | Met (mitoch) |
| AUG | Met | |
| GUN | Val | |
| UCY | Ser | |
| UCR | Ser | Absent (Ec61) |
| CCN | Pro | |
| ACN | Thr | |
| GCN | Ala | |
| UAY | Tyr | |
| UAA | Stop | Gln/glu/Tyr (ciliate, mitoch) |
| UAG | Stop | Absent (Sc2O), Pyl *archae, eubact, Gln/Leu/Tyr |
| | | (ciliate, mitoch) |
| CAY | His | |
| CAR | Gln | |
| AAY | Asn | |
| AAA | Lys | Asn (mitoch) |
| AAG | Lys | |
| GAY | Asp | |
| GAR | Glu | |
| UGY | Cys | |
| UGA | Stop | Sec (Fungal ancestors), Trp/Gly/Cys (ciliate, |
| | | mitoch) |
| UGG | Trp | |
| CGY | Arg | |
| CGR | Arg | Absent (yeast mitoch) |
| AGY | Ser | |
| AGA | Arg | Ser (mitoch) |
| AGG | Arg | Set/Lys (mitoch) |
| GGN | Gly | |

Codon Capture across ~3B years of evolution
Calculated from S. cerevisiae S288C reference genome

In some embodiments, the following codons may be removed for rewriting and/or replacement.

TABLE 4
Possible Codon Replacement

| Amino | | Total number | |
|---|---|---|---|
| acid | Codons | of codons | Total number of tRNAs |
| Leucine | CTG/CTA | 69K codons | 3 tRNAs |
| Arginine | CGG/CGA | 14K codons | 1 tRNA |
| Serine | AGT/AGC | 70K codons | 4 tRNAs (choose one pair) |
| | TCG/TCA | 78K codons | 4 tRNAs (choose one pair) |
| Total | Over 6 | 153-161K codons | 8 tRNAs |
| | codons | | |

In some embodiments, a host genome may be divided into multiple regions for codon replacement design. In some embodiments, a host genome may be divided into at least 2, 3, 4,5,6,7,8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or at least 50 regions for codon design. In some embodiments, a host genome may be divided into approximately 2, 3,4,5,6,7,8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or approximately 50 regions for codon design. In some embodiments, a host genome may be divided into 5 regions for codon design.

In some embodiments, each region may be at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or at least about 50 kilobases (kb). In some embodiments, each region may be approximately 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or approximately 50 kb. In some embodiments, each region may have at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or at least 50 designs. In some embodiments, each region may have approximately 1,2,3,4,5,6,7,8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or approximately 50 designs.

In some embodiments, the total region of codon removal design may comprise at least 1,2,3,4,5,6,7,8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or at least 1000 kb. In some embodiments, the total region of codon removal design may comprise approximately 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or approximately 1000 kb.

In some embodiments, each region may have at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or at least 50 codons removed. In some embodiments, each region may have approximately 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or approximately 50 codons removed. In some embodiments, each region may have 2 codons removed (e.g., “Individual” design). In some embodiments, the “Individual” design may comprise removing one or more codons encoding leucine, arginine, or serine. In some embodiments, each region may have 3 codons removed (e.g., “Paired” design). In some embodiments, the “Paired” design may comprise removing one or more codons encoding leucine/arginine, leucine/serine, or arginine/serine. In some embodiments, each region may have 6 codons removed (e.g., “All” design). In some embodiments, the “All” design may comprise removing one or more codons encoding leucine, arginine, and serine.

In some embodiments, the total number of codons removed, rewritten, or replaced may comprise at least 1, 10, 50, 100, 150, 200, 250, 300, 350, 400, 450, 500, 550, 600, 650, 700, 750, 800, 850, 900, 950, or at least 1000 codons. In some embodiments, the total number of codons removed, rewritten, or replaced may comprise approximately 1, 10, 50, 100, 150, 200, 250, 300, 350, 400, 450, 500, 550, 600, 650, 700, 750, 800, 850, 900, 950, or approximately 1000 codons. In some embodiments, the total number of codons removed, rewritten, or replaced may comprise at least 1K, 2K, 3K, 4K, 5K, 6K, 7K, 8K, 9K, 10K, 20K, 30K, 40K, 50K, 60K, 70K, 80K, 90K, 100K, 110K, 120K, 130K, 140K, 150K, 160K, 170K, 180K, 190K, 200K, 250K, 300K, 350K, 400K, 450K, 500K, 550K, 600K, 650K, 700K, 750K, 800K, 850K, 900K, 950K, or at least 1000K codons. In some embodiments, the total number of codons removed, rewritten, or replaced may comprise approximately 1K, 2K, 3K, 4K, 5K, 6K, 7K, 8K, 9K, 10K, 20K, 30K, 40K, 50K, 60K, 70K, 80K, 90K, 100K, 110K, 120K, 130K, 140K, 150K, 160K, 170K, 180K, 190K, 200K, 250K, 300K, 350K, 400K, 450K, 500K, 550K, 600K, 650K, 700K, 750K, 800K, 850K, 900K, 950K, or approximately 1000K codons.

### Codon Replacement: Synonymous Rewriting & Observed Bug Rate

In some aspects, provided herein are methods for synonymous codon rewriting and design rules for synonymous codon rewriting and observed bug rate. A bug or bugs, as used here, may refer to unanticipated fitness defect(s) caused by designed DNA sequence. In some embodiments, a bug may also be referred to a risk. Methods for synonymous codon rewriting may follow design rules that provide technical improvements in decreasing or minimizing a bug rate (e.g., by avoiding the selection of codons for use in re-writing that may introduce unanticipated fitness defects in the designed DNA sequence). In some embodiments, methods disclosed herein may comprise utilizing encoded watermarks (e.g., PCRTags or any other DNA barcodes) in the genome. For example, watermarks may be encoded in non-protein-coding regions. In some embodiments, watermarks may be encoded in ORFs. In some embodiments, methods described herein may synonymously rewrite 1 out of approximately every 20 codons globally. In some embodiments, methods disclosed herein may comprise performing a PCRTag algorithm. In some embodiments, the PCRTag algorithm may specify a ‘most-different’ design. In some embodiments, the “most-different” design may ignore the relative synonymous codon usage (RSCU), codon adaptation, or translation efficiency matching to maximize base pair changes. In some embodiments, the “most-different” design may yield about 1 bug per 10K codons removed, rewritten, or replaced. In some embodiments, the “most-different” design may yield about 3 bugs per 20K codons removed, rewritten, or replaced (details described in Richardson, et al., Science (2017) 355, 1040-1044, which is incorporated by reference herein in its entirety). In some embodiments, methods disclosed herein may decrease the number of bugs. In some embodiments, methods disclosed herein may eliminate one or more bugs. In some embodiments, methods disclosed herein may avoid a bug or a risk. In some embodiments, the risk may comprise a known regulatory site in ORFs that can impede transcription. In some embodiments, the known regulatory site may comprise a binding site of Repressor Activator Protein 1 (Rap1p, essential DNA-binding transcription regulator) in ORFs. Details are described in Yarrington, et al. Genetics (2012) 190(2):523-35 and Wu, et al., Science (2017) 355, 1048, each of which is incorporated by reference herein in its entirety. In some embodiments, a Rap1p binding site consensus sequence may comprise ACACCCRYACAYM (SEQ ID NO: 11,813), wherein R may be G or A, Y may be C or T, and M may be A or Cₙ

### Codon Replacement: Simple/Conventional Method

In some aspects, provided herein are methods for codon rewriting and/or replacement. In some embodiments, methods described herein may comprise rewriting and/or replacing a codon while retaining GC content. In some embodiments, a nucleotide in the wobble position of a codon (third position of a codon) is changed in a way that retains GC content. For example, a codon ending in G or A in a 4-codon block may be changed to C or T, respectively, to retain GC content. In some embodiments, these changes may also replace codons with other codons having the same frequency. Alternatively, in some embodiments, methods for codon rewriting and/or replacing described herein, may comprise changing one or more codons encoding an amino acid to the most frequently used codon for that specific amino acid in the genome. For example, one or more synonymous codons can be replaced with a synonymous codon with the highest number of occurrences for that specific amino acid in the genome. In some embodiments, methods that have the smallest effect on tRNA pools may be used.

### Codon Replacement Via Statistical Analysis: Goldilocks Method

Many synonymous codon rewriting methods are based on matching single-codon properties such as, for example, relative synonymous codon usage (RSCU) over all genes, codon adaptation index (CAI) over highly-expressed or stress-response genes, and translational efficiency (TE) incorporating tRNA pool. Some methods optimize over 2-codon windows or mRNA secondary structure using a hidden Markov model (HMM). Another new approach for codon rewriting and/or replacement is a Goldilocks method which utilizes machine learning analysis (e.g., statistical analysis) of a host genome.

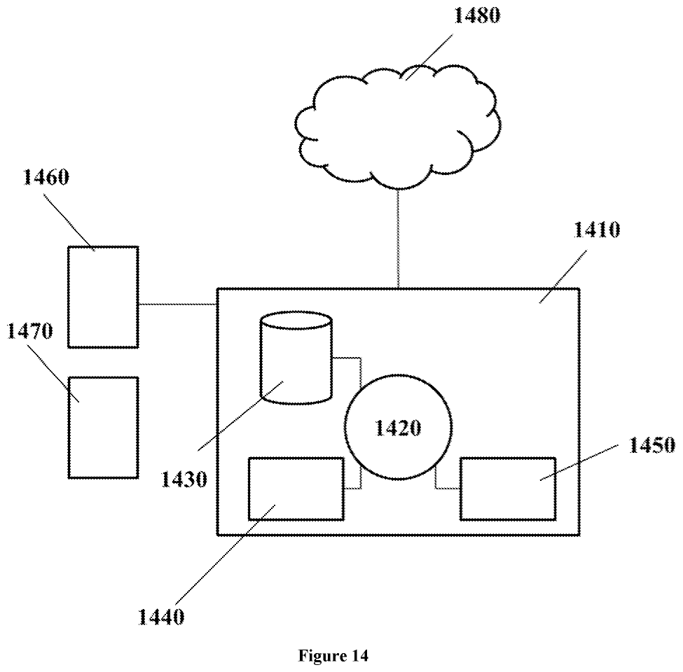

The present disclosure provides computer systems that are programmed to implement methods of the disclosure. FIG. 14 depicts a computer system that is programmed or otherwise configured to implement methods provided herein. The computer system 1410 may be programmed or otherwise configured to, for example, analyze at least a portion of the genome of the organism to identify a first plurality of codons in the genome of the organism to be rewritten, rewrite the first plurality of codons in the genome of the organism to a second codon, and analyze a local context of a codon-of-interest in the genome of the organism.

The computer system 1410 can regulate various aspects of analysis, calculation, and generation of the present disclosure, such as, for example, analyzing at least a portion of the genome of the organism to identify a first plurality of codons in the genome of the organism to be rewritten, rewriting the first plurality of codons in the genome of the organism to a second codon, and analyzing a local context of a codon-of-interest in the genome of the organism. The computer system 1410 can be an electronic device of a user or a computer system that is remotely located with respect to the electronic device. The electronic device can be a mobile electronic device.

The computer system 1410 includes a central processing unit (CPU, also “processor” and “computer processor” herein) 1420, which can be a single core or multi core processor, or a plurality of processors for parallel processing. The computer system 1410 also includes memory or memory location 1440 (e.g., random-access memory, read-only memory, flash memory), electronic storage unit 1430 (e.g., hard disk), communication interface 1420 (e.g., network adapter) for communicating with one or more other systems, and peripheral devices 1450, such as cache, other memory, data storage and/or electronic display adapters. The memory 1440, storage unit 1430, interface 1420 and peripheral devices 1450 are in communication with the CPU 1420 through a communication bus (solid lines), such as a motherboard. The storage unit 1430 can be a data storage unit (or data repository) for storing data. The computer system 1410 can be operatively coupled to a computer network (“network”) 1480 with the aid of the communication interface 1420. The network 1480 can be the Internet, an internet and/or extranet, or an intranet and/or extranet that is in communication with the Internet.

The network 1480 in some cases is a telecommunication and/or data network. The network 1480 can include one or more computer servers, which can enable distributed computing, such as cloud computing. For example, one or more computer servers may enable cloud computing over the network 1480 (“the cloud”) to perform various aspects of analysis, calculation, and generation of the present disclosure, such as, for example, analyzing at least a portion of the genome of the organism to identify a first plurality of codons in the genome of the organism to be rewritten, rewriting the first plurality of codons in the genome of the organism to a second codon, and analyzing a local context of a codon-of-interest in the genome of the organism. Such cloud computing may be provided by cloud computing platforms such as, for example, Amazon Web Services (AWS), Microsoft Azure, Google Cloud Platform, and IBM cloud. The network 1480, in some cases with the aid of the computer system 1410, can implement a peer-to-peer network, which may enable devices coupled to the computer system 1410 to behave as a client or a server.

The CPU 1420 may comprise one or more computer processors and/or one or more graphics processing units (GPUs). The CPU 1420 can execute a sequence of machine-readable instructions, which can be embodied in a program or software. The instructions may be stored in a memory location, such as the memory 1440. The instructions can be directed to the CPU 1420, which can subsequently program or otherwise configure the CPU 1420 to implement methods of the present disclosure. Examples of operations performed by the CPU 1420 can include fetch, decode, execute, and writeback.

The CPU 1420 can be part of a circuit, such as an integrated circuit. One or more other components of the system 1410 can be included in the circuit. In some cases, the circuit is an application specific integrated circuit (ASIC).

The storage unit 1430 can store files, such as drivers, libraries and saved programs. The storage unit 1430 can store user data, e.g., user preferences and user programs. The computer system 1410 in some cases can include one or more additional data storage units that are external to the computer system 1410, such as located on a remote server that is in communication with the computer system 1410 through an intranet or the Internet.

The computer system 1410 can communicate with one or more remote computer systems through the network 1480. For instance, the computer system 1410 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1410 via the network 1480.

Methods as described herein can be implemented by way of machine (e.g., computer processor) executable code stored on an electronic storage location of the computer system 1410, such as, for example, on the memory 1440 or electronic storage unit 1430. The machine executable or machine readable code can be provided in the form of software. During use, the code can be executed by the processor 1420. In some cases, the code can be retrieved from the storage unit 1430 and stored on the memory 1440 for ready access by the processor 1420. In some situations, the electronic storage unit 1430 can be precluded, and machine-executable instructions are stored on memory 1440.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1410, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1410 can include or be in communication with an electronic display 1460 that comprises a user interface (UI) 1470 for providing, for example, a visual display indicative of training and testing of a trained algorithm. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 1420. The algorithm can, for example, analyze at least a portion of the genome of the organism to identify a first plurality of codons in the genome of the organism to be rewritten, rewrite the first plurality of codons in the genome of the organism to a second codon, and analyze a local context of a codon-of-interest in the genome of the organism.

In some embodiments, the computer system may be a machine learning-based computer system comprising a computer processing unit communicatively coupled to a sequence processing unit via a first controller and to a storage unit via a second controller. In some embodiments, the machine learning-based computer system optionally comprises a sequence analyzer that sequences at least a portion of a genome of an organism (e.g., at least in part by assaying nucleic acid molecules obtained or derived from the organism to determine genetic sequences of the at least the portion of the genome of the organism). In some embodiments, the sequence processing unit comprises a storage component that retains genome sequence data generated by the sequence processing unit. The sequence processing unit may receive input data from the computer processing unit. For example, the input data may comprise translation tables obtained from the National Center for Biotechnology Information (NCBI), a sequence read of at least a portion of a genome of an organism contained in a sample, or a combination thereof. In some embodiments, the at least the portion of the genome comprises a nucleus-derived DNA. In some embodiments, the at least the portion of the genome comprises protein-coding genes. In some embodiments, mitochondrial genes, transposable element genes, pseudogenes, and blocked reading frames are excluded from the method disclosed herein. The sequence processing unit determines the codon count for each of a plurality of codons in the genome (e.g., including stop codons). In some embodiments, a translation table is used to map codons to amino acids. In some embodiments, the sequence processing unit determines an RSCU for each codon (e.g., as the number of counts for the codon divided by the number of counts for all codons for the same amino acid).

In some embodiments, the sequence processing unit determines the frequency of 9 mers in coding domains of a genome of an organism. In some embodiments, the 9 mers are converted to contexts. Contexts, as disclosed herein, may comprise a codon-amino acid-codon pattern.

In some embodiments, the sequence processing unit comprises an algorithm that determines a value for each coding sequence by identifying positions of one or more codons to eliminate; analyzing each codon, in turn; and rewriting the codon with the most frequently used codon as the central codon in a 3-codon (9 mer) context. In some embodiments, the first codon is unique because there is no preceding context. In standard genetic codes, however, the first codon is always ATG. In some cases, the last codon (e.g., stop codon) has no following context. In some embodiments, if stop codons are rewritten, a favored design comprises changing TAA and TAG to TGA. TGA has only one single choice. Alternatively, in some embodiments, a 6nt (6-nucleotide) context or 9nt (9-nucleotide) context with the stop codon as the final 3nt may be used.

In some embodiments, the sequence processing unit performs dynamical programming for treatment of neighboring codons. In some embodiments, the sequencing processing unit uses a different codon selection criterion, such as maintaining GC content, codon adaptation index, or translational efficiency, as the main codon replacement rule. In some embodiments, the sequence processing unit employs a Goldilocks codon with the greatest fold-enrichment, rather than a Goldilocks codon that is most often used, in the context. In some embodiments, the sequence processing unit uses random codons selected using the Goldilocks context-dependent probabilities as the probability distribution.

In some embodiments, the final codon is a stop codon and a special case. Most designs may be a single choice for the stop codon, TGA, or a pair of choices, TGA and TAA. For the stop codon, a 9 mer pattern or a 5 mer pattern ending with the stop codon may be used instead of the 9 mer pattern with the codon of interest in the middle position. Some example embodiments avoid significantly enriched codons as possible regulatory signals (e.g., too hot), thereby choosing codons whose usage matches the overall RSCU. Some example embodiments avoid codons that are used significantly less (e.g., too cold), thereby choosing codons whose usage matches the overall RSCU. Some example embodiments may consider the RSCU value for the specific codon. In some embodiments, a codon with an RSCU value of at least about 0.01, at least about 0.05, at least about 0.10, at least about 0.11, at least about 0.12, at least about 0.13, at least about 0.14, at least about 0.15, at least about 0.16, at least about 0.17, at least about 0.18, at least about 0.19, at least about 0.20, at least about 0.21, at least about 0.22, at least about 0.23, at least about 0.24, at least about 0.25, at least about 0.26, at least about 0.27, at least about 0.28, at least about 0.29, at least about 0.30, at least about 0.31, at least about 0.32, at least about 0.33, at least about 0.34, at least about 0.35, at least about 0.36, at least about 0.37, at least about 0.38, at least about 0.39, at least about 0.40, at least about 0.41, at least about 0.42, at least about 0.43, at least about 0.44, at least about 0.45, at least about 0.46, at least about 0.47, at least about 0.48, at least about 0.49, at least about 0.50, at least about 0.51, at least about 0.52, at least about 0.53, at least about 0.54, at least about 0.55, at least about 0.56, at least about 0.57, at least about 0.58, at least about 0.59, at least about 0.60, at least about 0.61, at least about 0.62, at least about 0.63, at least about 0.64, at least about 0.65, at least about 0.66, at least about 0.67, at least about 0.68, at least about 0.69, at least about 0.70, at least about 0.71, at least about 0.72, at least about 0.73, at least about 0.74, at least about 0.75, at least about 0.76, at least about 0.77, at least about 0.78, at least about 0.79, at least about 0.80, at least about 0.81, at least about 0.82, at least about 0.83, at least about 0.84, at least about 0.85, at least about 0.86, at least about 0.87, at least about 0.88, at least about 0.89, or at least about 0.90, at least about 0.91, at least about 0.92, at least about 0.93, at least about 0.94, at least about 0.95, at least about 0.96, at least about 0.97, at least about 0.98, at least about 0.99, or about 1.00 may be selected. In some embodiments, a codon with the highest RSCU value for a local context may be selected.

Codons are under evolutionary selection pressure such as positive selection or negative selection. For example, positive selection can include, but is not limited to, within-ORF regulatory elements. For example, negative selection can include, but is not limited to, frameshifts, ribosome stalls, and secondary structure interfering with transcription/translation. Codon choice can depend on context of surrounding codons.

For example, a Goldilocks method may be performed based on a principle that 1) most open reading frame (ORF) regions are not regulatory, 2) a replacement codon that is not too “hot” (e.g., a codon with usage that is significantly higher than the overall RSCU for that specific codon; positive selection) and not too “cold” (e.g., a codon with usage that is significantly lower than the overall RSCU for that specific codon; negative selection) is chosen, and 3) a replacement codon depends on context of upstream and downstream codons. In some embodiments, a replacement codon that is “too hot” may comprise a codon that may have been evolutionarily positively selected.

In some embodiments, methods for selecting a replacement codon may comprise an optimization or outlier avoidance approach (e.g., a “Goldilocks”) approach to avoid selection of a replacement codon with a positive evolutionary signal (e.g., a codon that is too “hot” having a usage that is significantly higher than the overall RSCU for that given codon) or a negative evolutionary signal (e.g., a codon that is too “cold” having a usage that is significantly lower than the overall RSCU for that given codon), and instead to select a replacement codon based at least in part on consideration of the codon's local context (e.g., by considering replacement codons whose relative synonymous usage in the given context most closely matches its relative synonymous usage overall). In some embodiments, such selection of replacement codons may comprise determining context-sensitive relative synonymous codon usage (RSCU) value for each of a plurality of codons (e.g., representing a local context of a given codon of interest), and identifying a codon from among the plurality of codons having a maximum or largest RSCU value. For example, the plurality of codons may comprise a codon of interest, a second codon that is upstream of the codon of interest, and a third codon that is downstream of the codon of interest. For example, the plurality of codons may comprise a set of at least three consecutive codons: a codon of interest, a second codon that is upstream of and adjacent to the codon of interest, and a third codon that is downstream of and adjacent to the codon of interest. For example, the maximal RSCU value may be at least about 0.01, at least about 0.05, at least about 0.10, at least about 0.11, at least about 0.12, at least about 0.13, at least about 0.14, at least about 0.15, at least about 0.16, at least about 0.17, at least about 0.18, at least about 0.19, at least about 0.20, at least about 0.21, at least about 0.22, at least about 0.23, at least about 0.24, at least about 0.25, at least about 0.26, at least about 0.27, at least about 0.28, at least about 0.29, at least about 0.30, at least about 0.31, at least about 0.32, at least about 0.33, at least about 0.34, at least about 0.35, at least about 0.36, at least about 0.37, at least about 0.38, at least about 0.39, at least about 0.40, at least about 0.41, at least about 0.42, at least about 0.43, at least about 0.44, at least about 0.45, at least about 0.46, at least about 0.47, at least about 0.48, at least about 0.49, at least about 0.50, at least about 0.51, at least about 0.52, at least about 0.53, at least about 0.54, at least about 0.55, at least about 0.56, at least about 0.57, at least about 0.58, at least about 0.59, at least about 0.60, at least about 0.61, at least about 0.62, at least about 0.63, at least about 0.64, at least about 0.65, at least about 0.66, at least about 0.67, at least about 0.68, at least about 0.69, at least about 0.70, at least about 0.71, at least about 0.72, at least about 0.73, at least about 0.74, at least about 0.75, at least about 0.76, at least about 0.77, at least about 0.78, at least about 0.79, at least about 0.80, at least about 0.81, at least about 0.82, at least about 0.83, at least about 0.84, at least about 0.85, at least about 0.86, at least about 0.87, at least about 0.88, at least about 0.89, or at least about 0.90, at least about 0.91, at least about 0.92, at least about 0.93, at least about 0.94, at least about 0.95, at least about 0.96, at least about 0.97, at least about 0.98, at least about 0.99, or about 1.00. This approach may advantageously select the replacement codon having the maximum context-sensitive codon usage. In some embodiments, motifs identified as associated with positive evolutionary signals or negative evolutionary signals that include codons that are to be replaced by a rewriting design may be highlighted as requiring greater scrutiny to avoid introducing fitness defects by rewriting. In this embodiment, methods using an approach to use a replacement codon that shares the same evolutionary signal as the re-written codon may be used. In some embodiments, rewriting designs may be selected to minimize the number of evolutionary motifs affected. In some embodiments, nonsynonymous codons may be introduced instead of introducing a motif with an evolutionary signal through replacement with a synonymous codon.

In some embodiments, a replacement codon that is “too hot” may comprise a codon that may be a regulatory element, e.g., an within-ORF regulatory element. In some embodiments, a replacement codon that is not “too hot” may comprise a codon that may not be an regulatory element, e.g., an within-ORF regulatory element. In some embodiments, a replacement codon that is “too cold” may comprise a codon that may have been evolutionarily negatively selected. In some embodiments, a replacement codon that is “too cold” may comprise a codon that may cause frameshifts, ribosome stalls, or secondary structure interfering with transcription and/or translation. In some embodiments, a replacement codon that is not “too cold” may comprise a codon that may not cause frameshifts, ribosome stalls, or secondary structure interfering with transcription and/or translation. In some embodiments, machine learning approaches (e.g., statistical analysis approaches) can be performed to determine the rules for Goldilocks methods for codon replacement from the host genome. Details of examples of Goldilocks methods are provided in, for example, Example 3 and Example 4. In some embodiments, sequences of original yeast ORFs (Saccharomyces cerevisiae S288C strain) and rewritten yeast ORFs using methods described herein are shown as SEQ ID NOs: 1-11,812.

In some aspects, provided herein are methods for codon rewriting and/or replacement, wherein a codon may be selected by examining a local context of the codon. In some embodiments, a codon may be selected by examining a local context of a codon-of-interest within an ORF or a gene. In some embodiments, a local context of a codon-of-interest may comprise the codon-of-interest and a codon on each side of the codon-of-interest. In some embodiments, a local context of a codon-of-interest may comprise the codon-of-interest and codons on both 5′ and 3′ side of the codon-of-interest. In some embodiments, a local context of a codon-of-interest may comprise a preceding codon, the codon-of-interest, and the subsequent codon. In some embodiments, a local context of a codon-of-interest may comprise a codon upstream of the codon-of-interest, the codon-of-interest, and a codon downstream of the codon-of-interest. In some embodiments, a local context of a codon-of-interest may comprise a codon 5′ to the codon-of-interest, the codon-of-interest, and a codon 3′ to the codon-of-interest.

In some embodiments, a local context of a codon-of-interest may comprise at least 1, 2, 3, 4, 5, 6, 7, 8, 9, 10, 11, 12, 13, 14, 15, 16, 17, 18, 19, 20, or at least 21 codons. In some embodiments, a local context of a codon-of-interest may comprise 3 codons, i.e., a preceding codon, the codon-of-interest, and the subsequent codon. In some embodiments, a local context of a codon-of-interest may comprise 3 codons, i.e., a codon upstream of (or 5′ to) the codon-of-interest, the codon-of-interest, and a codon downstream of (or 3′ to) the codon-of-interest. In some embodiments, a local context of a codon-of-interest may comprise 5 codons, i.e., two preceding codons, the codon-of-interest, and the two subsequent codons. In some embodiments, a local context of a codon-of-interest may comprise 5 codons, i.e., two codons upstream of (or 5′ to) the codon-of-interest, the codon-of-interest, and two codons downstream of (or 3′ to) the codon-of-interest.

In some embodiments, a local context of a codon-of-interest may comprise at least 3, 4,5,6,7,8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, or at least 63 nucleotides or base pairs. In some embodiments, a local context of a codon-of-interest may comprise a total of 9 nucleotides. For example, a local context of a codon-of-interest may comprise a 3 nucleotide preceding codon, the 3 nucleotide codon-of-interest, and a 3 nucleotide subsequent codon. For example, a local context of a codon-of-interest may comprise a 3 nucleotide codon upstream of (or 5′ to) the codon-of-interest, the 3 nucleotide codon-of-interest, and a 3 nucleotide codon downstream of (or 3′ to) the codon-of-interest. In some embodiments, a local context of a codon-of-interest may comprise a total of 11 nucleotides. For example, a local context of a codon-of-interest may comprise 4 nucleotides upstream of (or 5′ to) the codon-of-interest, the 3 nucleotide codon-of-interest, and 4 nucleotides downstream of (or 3′ to) the codon-of-interest. In some embodiments, a local context of a codon-of-interest may comprise a total of 15 nucleotides. For example, a local context of a codon-of-interest may comprise two preceding codons, each having 3 nucleotides, the 3 nucleotide codon-of-interest, and two subsequent codons, each having 3 nucleotides. For example, a local context of a codon-of-interest may comprise two codons, each having 3 nucleotides, upstream of (or 5′ to) the codon-of-interest, the 3 nucleotide codon-of-interest, and two codons, each having 3 nucleotides, downstream of (or 3′ to) the codon-of-interest.

In some embodiments, a local context of a codon-of-interest may comprise

C₍ₙ₋₁₎−Cₙ−C₍ₙ₊₁₎, wherein

C₍ₙ₋₁₎ denotes a codon downstream of the codon-of-interest;
Cₙ denotes the codon-of-interest; and
C₍ₙ₊₁₎ denotes a codon upstream of the codon-of-interest.

In some embodiments, a local context of a codon-of-interest may comprise

C₍ₙ₋1)−AAₙ−C₍ₙ₊₁₎, wherein

C₍ₙ₋₁₎ denotes a codon downstream of the codon-of-interest;
AAₙ is an amino acid encoded by the codon-of-interest; and
C₍ₙ₊₁₎ denotes a codon upstream of the codon-of-interest.

In some embodiments, methods described herein may comprise determining a number of occurrences of the local context of the codon-of-interest. In some embodiments, methods described herein may comprise determining a relative synonymous codon usage (RSCU) of the codon-of-interest (Cₙ). In some embodiments, the RSCU may be determined as the frequency of a codon divided by the frequency of all codons encoding the same amino acid.

In some embodiments, a codon may be selected based on the RSCU value of the codon for a local context. In some embodiments, a codon with an RSCU value of at least about 0.01, at least about 0.05, at least about 0.10, at least about 0.11, at least about 0.12, at least about 0.13, at least about 0.14, at least about 0.15, at least about 0.16, at least about 0.17, at least about 0.18, at least about 0.19, at least about 0.20, at least about 0.21, at least about 0.22, at least about 0.23, at least about 0.24, at least about 0.25, at least about 0.26, at least about 0.27, at least about 0.28, at least about 0.29, at least about 0.30, at least about 0.31, at least about 0.32, at least about 0.33, at least about 0.34, at least about 0.35, at least about 0.36, at least about 0.37, at least about 0.38, at least about 0.39, at least about 0.40, at least about 0.41, at least about 0.42, at least about 0.43, at least about 0.44, at least about 0.45, at least about 0.46, at least about 0.47, at least about 0.48, at least about 0.49, at least about 0.50, at least about 0.51, at least about 0.52, at least about 0.53, at least about 0.54, at least about 0.55, at least about 0.56, at least about 0.57, at least about 0.58, at least about 0.59, at least about 0.60, at least about 0.61, at least about 0.62, at least about 0.63, at least about 0.64, at least about 0.65, at least about 0.66, at least about 0.67, at least about 0.68, at least about 0.69, at least about 0.70, at least about 0.71, at least about 0.72, at least about 0.73, at least about 0.74, at least about 0.75, at least about 0.76, at least about 0.77, at least about 0.78, at least about 0.79, at least about 0.80, at least about 0.81, at least about 0.82, at least about 0.83, at least about 0.84, at least about 0.85, at least about 0.86, at least about 0.87, at least about 0.88, at least about 0.89, or at least about 0.90, at least about 0.91, at least about 0.92, at least about 0.93, at least about 0.94, at least about 0.95, at least about 0.96, at least about 0.97, at least about 0.98, at least about 0.99, or about 1.00 may be selected. In some embodiments, a codon with the highest RSCU value for a local context may be selected.

In some embodiments, methods described herein may comprise determining an expected number of occurrences of the local context of the codon-of-interest. In some embodiments, the expected number of occurrences of the first local context of the codon-of-interest is determined as a product of: a number of occurrences of the second local context of the codon-of-interest, and the determined RCSU of the codon-of-interest. In some embodiments, the expected number of occurrences of C₍ₙ₋₁₎−Cₙ−C₍ₙ₊₁₎ is determined as:

(a number of occurrences of C₍ₙ₋₁₎−AAₙ−C₍ₙ₊₁₎)X(RCSU of theCₙ).

In some embodiments, methods described herein may comprise identifying a statistically significant evolutionary signal. In some embodiments, statistically significant evolutionary signals may comprise a negative evolutionary selection signal, a positive evolutionary selection signal, or a combination thereof. For example, the negative selection signal may include, but is not limited to, a frameshift, a ribosome stall, or a secondary RNA structure interfering with transcription and/or translation. For example, the positive selection signal may include, but is not limited to, a regulatory element within an open reading frame (ORF).

tRNA Removal & Supplementation

In some embodiments, methods described herein may comprise removing or supplementing one or more tRNAs with corresponding codons to one or more codons to be rewritten or replaced. In some embodiments, methods described herein may comprise supplementing the ones that may be oversubscribed as a function of replacement strategy

In some embodiments, performing genome design may comprise removing codons and corresponding tRNAs for rewriting and/or replacement. For example, codons may be rewritten synonymously and tRNAs with complementary anticodons may be deleted as part of the genome design (e.g., deleting tRNA genes). In this embodiment, deleting one or more tRNA genes prior to rewriting the entire genome may cause slow growth or lethality of an organism. In some embodiments, tRNA genes may be provided on a plasmid or chromosomal region that may be removed at the final step of genome rewriting or strain construction.

In some embodiments, additional tRNAs with anticodons recognizing the newly assigned codons (i.e., codons encoding a newly assigned amino acid or an ncAA) may be provided. In some embodiments, the total number of tRNA genes deleted can be determined, and the copy number of the remaining tRNA genes for an amino acid can be increased by the same amount. In some embodiments, wobble rules can be used to identify the tRNA genes responsible for decoding the replacement codons, and copy number increases can be allocated proportionally. In some embodiments, one or more non-native tRNA genes may be introduced. For example, for leucine, tL(AAG) from Candida species may be introduced.

### Nucleic Acid Construction and Replacing Genome

In some aspects, methods described herein may comprise synthesizing a nucleic acid construct comprising one or more codons rewritten based on codon rewriting/replacement methods described herein. In some embodiments, any known methods in the art can be used to synthesize the nucleic acid construct comprising one or more codons rewritten based on codon rewriting/replacement methods described herein. In some embodiments, a chromosome can be computationally divided into 30-60 kilobase long constructs, each comprising a set of segments that is less than about 10 kilobase in length. Each segment can be synthesized using any known methods in the art, e.g., a polymerase chain reaction (PCR), and/or restriction enzyme digestion/ligation. In some embodiments, these segments can be assembled into a construct by restriction enzyme cutting and ligation in vitro, or any other methods known in the art. In some embodiments, the construct can be sequenced to confirm the sequence of the nucleic acid construct and subsequently integrated into the host genome, e.g., an yeast genome, using any known methods in the art to replace the corresponding portion, region, or segment of the wile-type.

In some aspects, methods described herein may further comprise replacing a portion of a genome with a nucleic acid construct comprising one or more codons rewritten based on codon rewriting/replacement methods described herein. In some embodiments, site-specific nucleases (SSNs) or homology-directed recombination (HR) can be used to replace a portion of a genome. In some embodiments, HR can be used utilizing an endogenous homologous recombination machinery. In some embodiments, a yeast homologous recombination machinery can be used as detailed in Example 6.

In some embodiments, SSN may comprise meganucleases, zinc-finger nucleases (ZFN), TAL effector nucleases (TALEN), and clustered regularly interspaced short palindromic repeats (CRISPR)/CRISPR-associated (Cas) system. These four major classes of gene-editing techniques, namely, meganucleases, ZFNs, TALENs, CRISPR/Cas systems share a common mode of action in binding a user-defined sequence of DNA and mediating a double-stranded DNA break (DSB). DSB may then be repaired by HR, an event that introduces the homologous sequence from a donor DNA fragment, or by non-homologous end joining (NHEJ), when there is no donor DNA present.

CRISPR-Cas system may be used with a guide target sequence for genetic screening, targeted transcriptional regulation, targeted knock-in, and targeted genome editing, including base editing, epigenetic editing, and introducing double strand breaks (DSBs) for homologous recombination-mediated insertion of a nucleotide sequence. CRISPR-Cas system comprises an endonuclease protein whose DNA-targeting specificity and cutting activity can be programmed by a short guide RNA or a duplex crRNA/TracrRNA. A CRISPR endonuclease comprises a caspase effector nuclease, typically microbial Cas9 and a short guide RNA (gRNA) or a RNA duplex comprising a 18 to 20 nucleotide targeting sequence that directs the nuclease to a location of interest in the genome. Genome editing can refer to the targeted modification of a DNA sequence, including but not limited to, adding, removing, replacing, or modifying existing DNA sequences, and inducing chromosomal rearrangements or modifying transcription regulation elements (e.g., methylation/demethylation of a promoter sequence of a gene) to alter gene expression. As described above CRISPR-Cas system requires a guide system that can locate Cas protein to the target DNA site in the genome. In some instances, the guide system comprises a crispr RNA (crRNA) with a 17-20 nucleotide sequence that is complementary to a target DNA site and a trans-activating crRNA (tracrRNA) scaffold recognized by the Cas protein (e.g., Cas9). The 17-20 nucleotide sequence complementary to a target DNA site is referred to as a spacer while the 17-20 nucleotide target DNA sequence is referred to a protospacer. While crRNAs and tracrRNAs exist as two separate RNA molecules in nature, single guide RNA (sgRNA or gRNA) can be engineered to combine and fuse crRNA and tracrRNA elements into one single RNA molecule. Thus, in one embodiment, the gRNA comprises two or more RNAs, e.g., crRNA and tracrRNA. In another embodiment, the gRNA comprises a sgRNA comprising a spacer sequence for genomic targeting and a scaffold sequence for Cas protein binding. In some instances, the guide system naturally comprises a sgRNA. For example, Cas12a/Cpf1 utilizes a guide system lacking tracrRNA and comprising only a crRNA containing a spacer sequence and a scaffold for Cas12a/Cpf1 binding. While the spacer sequence can be varied depending on a target site in the genome, the scaffold sequence for Cas protein binding can be identical for all gRNAs.

CRISPR-Cas systems described herein can comprise different CRISPR enzymes. For example, the CRISPR-Cas system can comprise Cas9, Cas12a/Cpf1, Cas12b/C2cl, Cas12c/C2c3, Cas12d/CasY, Cas12e/CasX, Cas12g, Cas12h, or Cas12i. Non-limiting examples of Cas enzymes include, but are not limited to, Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (also known as Csn1 or Csx12), Cas10, Cas10d, Cas12a/Cpf1, Cas12b/C2cl, Cas12c/C2c3, Cas12d/CasY, Cas12e/CasX, Cas12f/Cas14/C2c10, Cas12g, Cas12h, Cas12i, Cas12k/C2c5, Cas13a/C2c2, Cas13b, Cas13c, Cas13d, C2c4, C2c8, C2c9, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, GSU0054, Type II Cas effector proteins, Type V Cas effector proteins, Type VI Cas effector proteins, CARF, DinG, homologues thereof, or modified or engineered versions thereof such as dCas9 (endonuclease-dead Cas9) and nCas9 (Cas9 nickase that has inactive DNA cleavage domain). In some cases, the compositions, methods, devices, and systems, described herein, may use the Cas9 nuclease from Streptococcus pyogenes, of which amino acid sequences and structures are well known to those skilled in the art.

In some aspects, described herein, are methods for contacting a genome from a sample with one or more agents configured to cleave the genome at a locus. In some embodiments, the contacting may occur in vitro. In some embodiments, the contacting may occur in vivo, e.g., in a cell. In some embodiments, the one or more agents comprise a polypeptide, a polynucleotide, or a combination thereof. In some embodiments, the polypeptide comprises an enzyme, e.g., a site-specific nuclease. Examples of a site-specific nuclease are shown above. In some embodiments, a site-specific nuclease comprises an engineered homing endonuclease or meganuclease, a zinc-finger nuclease (ZFN), a transcription activator-like effector nuclease (TALEN), a clustered regularly interspaced short palindromic repeat (CRISPR/Cas), or a combination thereof. In some embodiments, the polynucleotide comprises a guide RNA (gRNA). In some embodiments, the one or more agents comprise a site-specific nuclease and a gRNA (e.g., CRISPR/Cas system).

Agents described herein can be delivered into cells in vitro or in vivo by art-known methods or as described herein. Delivery methods such as physical, chemical, and viral methods are also known in the art. In some instances, physical delivery methods can be selected from the methods but not limited to electroporation, microinjection, or use of ballistic particles. On the other hand, chemical delivery methods require use of complex molecules such calcium phosphate, lipid, or protein. In some embodiments, viral delivery methods are applied for gene editing techniques using viruses such as but not limited to adenovirus, lentivirus, and retrovirus. In some embodiments, agents described herein can be delivered via a carrier. In some embodiments, agents described herein can be delivered by, e.g., vectors (e.g., viral or non-viral vectors), non-vector based methods (e.g., using naked DNA, DNA complexes, lipid nanoparticles, RNA such as mRNA), or a combination thereof. In some embodiments, a carrier can comprise comprises a vector, a messenger RNA (mRNA), double stranded DNA (dsDNA), single stranded DNA (ssDNA), or a plasmid. In some embodiments, agents can be delivered directly to cells as naked DNA or RNA, for instance by means of transfection or electroporation, or can be conjugated to molecules (e.g., N-acetylgalactosamine) promoting uptake by cells.

In some embodiments, vectors can comprise one or more sequences encoding one or more agents described herein. Vectors can also comprise a sequence encoding a signal peptide (e.g., for nuclear localization, nucleolar localization, or mitochondrial localization), associated with (e.g., inserted into or fused to) a sequence coding for a protein. As one example, vectors can include a Cas9 coding sequence that includes one or more nuclear localization sequences (e.g., a nuclear localization sequence from SV40). Vectors described herein can also include any suitable number of regulatory/control elements, e.g., promoters, enhancers, introns, polyadenylation signals, Kozak consensus sequences, or internal ribosome entry sites (IRES). These elements are well known in the art. Vectors described herein may include recombinant viral vectors. Any viral vectors known in the art can be used. Examples of viral vectors include, but are not limited to lentivirus (e.g., HIV and FIV-based vectors), Adenovirus (e.g., AD100), Retrovirus (e.g., Maloney murine leukemia virus, MML-V), herpesvirus vectors (e.g., HSV-2), and Adeno-associated viruses (AAVs), or other plasmid or viral vector types. In some embodiments, agents described herein may be delivered in one carrier (e.g., one vector). In some embodiments, agents described herein may be delivered in in multiple carriers (e.g., multiple vectors).

In addition, viral particles can be used to deliver agents in nucleic acid and/or peptide form. For example, “empty” viral particles can be assembled to contain any suitable cargo. Viral vectors and viral particles can also be engineered to incorporate targeting ligands to alter target tissue specificity. Non-viral vectors can be also used to deliver agents according to the present disclosure. One example of non-viral nucleic acid vectors is an nanoparticle, which can be organic or inorganic. Nanoparticles are well known in the art. Any suitable nanoparticle design can be used to deliver agents described herein (e.g., nucleic acids encoding such agents).

In some embodiments, agents described herein can be delivered as a ribonucleoprotein (RNP) to cells. An RNP may comprise a nucleic acid binding protein, e.g., Cas9, in a complex with a gRNA targeting a genome/locus/sequence of interest. RNPs can be delivered to cells using known methods in the art, including, but not limited to electroporation, nucleofection, or cationic lipid-mediated methods, for example, as reported by Zuris, J. A. et al., 2015, Nat. Biotechnology, 33(1):73-80.

### Machine Learning-Based Computer Systems

In some aspects, methods described herein may comprise utilizing a machine learning-based computer system. In some embodiments, machine learning-based computer systems described herein may comprise one or more storage units comprising, respectively, one or more storage devices included within respective storage arrays controlled by a respective one or more storage controllers; and one or more computer processing units, wherein the one or more computer processing units are configured to communicate with the one or more storage units over a communication interface.

In some embodiments, the machine learning-based computer system provides the plurality of intermediate scores to a machine learning algorithm that processes the plurality of intermediate scores to generate the rewritten codons (e.g., the first plurality of codons that are selected to be rewritten into a second codon). The machine learning algorithm may comprise a function that determines how intermediate scores are combined and weighted. The machine learning algorithm may comprise a supervised machine learning algorithm. The supervised machine learning algorithm may be trained on prior data from a reference genome, or on prior data from multiple genomes. The prior data may include observed fitness values for genomes, including growth rates on different media. The machine learning-based computer system can train the supervised machine learning algorithm by providing examples of fitness values to an untrained or partially trained version of the algorithm to generate replacement codons for one or more of the input genomes or of a different genome. The system can compare the predicted fitness to the measured fitness (i.e., whether the cell growth rate was maintained), and if there is a difference, the system can perform training at least in part by updating the parameters of the supervised machine learning algorithm. The supervised machine learning algorithm may comprise a regression algorithm, a support vector machine, a decision tree, a neural network, or the like. In cases in which the machine learning algorithm comprises a regression algorithm, the weights may be regression parameters. The supervised machine learning algorithm may comprise a classifier or a predictor that determines a prediction of which replacement codons (e.g., selected from among a plurality of possible replacement codons) are least likely to result in a fitness deficit. The predictor may generate a fitness risk score that is indicative of a likelihood of being indicative of a fitness risk (e.g., probabilistic fitness risk score between 0 and 1). In some cases, the machine learning-based computer system may map the probabilistic risk score to a qualitative risk category (e.g., selected from among a plurality of risk categories). For example, a fitness risk score that is at least 0.5 may be considered a high risk, while a fitness risk score that is less than 0.5 may be considered a low risk. Alternatively, the supervised machine learning algorithm may be a multi-class classifier (e.g., binary classifier) that predicts a qualitative risk category directly.

The machine learning algorithm may be comprise unsupervised machine learning algorithm. The unsupervised machine learning algorithm may identify patterns in a genome or multiple genomes of interest. For example, it may identify a set of codon usage contexts that are an outlier as compared to other sets of codon usage for the same amino acid. If the unsupervised machine learning algorithm determines that a particular context-dependent codon usage is an outlier, the machine learning-based computer system may determine that relying on genome-wide codon usage for codon selection may lead to a fitness deficit. On the other hand, a set of codon usage scores that is consistent with overall codon usage for the genome may indicate that codon replacement has lower risk of generating a fitness defect. The unsupervised machine learning algorithm may comprise a clustering algorithm, an isolation forest, an autoencoder, or the like.

### Trained Algorithms

In some aspects, methods and systems described herein may employ one or more trained algorithms. The trained algorithm(s) may process or operate on one or more datasets comprising information about a codon-of-interest, a codon upstream of (or 5′ to) the codon-of-interest, a codon downstream of (or 3′ to) the codon-of-interest, or any combination thereof. In some embodiments, the datasets comprise structural or sequence information about codons. In some embodiments, the datasets comprise one or more datasets of codons. The one or more datasets may be observed empirically, derived from computational studies, be derived from or retrieved from one or more databases, be artificially generated (e.g., as in silico variants of empirically observed datasets), or any combination thereof.

The trained algorithm may comprise an unsupervised machine learning algorithm. The trained algorithm may comprise a supervised machine learning algorithm. The trained algorithm may comprise a classification and regression tree (CART) algorithm. The supervised machine learning algorithm may comprise, for example, a Random Forest, a support vector machine (SVM), a neural network, or a deep learning algorithm. The trained algorithm may comprise a self-supervised machine learning algorithm. The trained algorithm may comprise a statistical model, statistical analysis, or statistical learning.

In some embodiments, a machine learning algorithm (or software module) of a platform as described herein utilizes one or more neural networks. In some embodiments, a neural network is a type of computational system that can learn the relationships between an input dataset and a target dataset. A neural network may be a software representation of a human neural system (e.g., cognitive system), intended to capture “learning” and “generalization” abilities as used by a human. In some embodiments, the machine learning algorithm (or software module) comprises a neural network comprising a convolutional neural network (CNN). Non-limiting examples of structural components of embodiments of the machine learning software described herein include: CNNs, recurrent neural networks, dilated CNNs, fully-connected neural networks, deep generative models, and Boltzmann machines.

In some embodiments, a neural network comprises a series of layers termed “neurons.” In some embodiments, a neural network comprises an input layer, to which data is presented; one or more internal, and/or “hidden”, layers; and an output layer. A neuron may be connected to neurons in other layers via connections that have weights, which are parameters that control the strength of the connection. The number of neurons in each layer may be related to the complexity of the problem to be solved. The minimum number of neurons required in a layer may be determined by the problem complexity, and the maximum number may be limited by the ability of the neural network to generalize. The input neurons may receive data being presented and then transmit that data to the first hidden layer through connections' weights, which are modified during training. The first hidden layer may process the data and transmit its result to the next layer through a second set of weighted connections. Each subsequent layer may “pool” the results from a set of the previous layers into more complex relationships. In addition, whereas some software programs require writing specific instructions to perform a task, neural networks are programmed by training them with a known sample set and allowing them to modify themselves during (and after) training so as to provide a desired output such as an output value (e.g., predicted value). After training, when a neural network is presented with new input data, it generalizes what was “learned” during training and applies what was learned from training to the new, previously unseen, input data in order to generate an output associated with that input (e.g., a predicted value). The output may be generated in order to minimize an expected error or loss function between the output value and an expected value.

In some embodiments, the neural network comprises artificial neural networks (ANNs). ANNs may be machine learning algorithms that may be trained to map an input dataset to an output dataset, where the ANN comprises an interconnected group of nodes organized into multiple layers of nodes. For example, the ANN architecture may comprise at least an input layer, one or more hidden layers, and an output layer. The ANN may comprise any total number of layers, and any number of hidden layers, where the hidden layers function as trainable feature extractors that allow mapping of a set of input data to an output value or set of output values. As used herein, a deep learning algorithm (such as a deep neural network, or DNN) is an ANN comprising a plurality of hidden layers, e.g., two or more hidden layers. Each layer of the neural network may comprise a number of nodes (or “neurons”). A node receives a set of inputs that are retrieved from either directly from the input data or the output of nodes in previous layers, and performs a specific operation, e.g., a summation operation, on the set of inputs. A connection from an input to a node is associated with a weight (or weighting factor). The node may determine a sum of the products of all pairs of inputs and their associated weights. The weighted sum may be offset with a bias. The output of a node or neuron may be gated using a threshold or activation function. The activation function may be a linear or non-linear function. The activation function may be, for example, a rectified linear unit (ReLU) activation function, a Leaky ReLU activation function, or other function such as a saturating hyperbolic tangent, identity, binary step, logistic, arctan, softsign, parametric rectified linear unit, exponential linear unit, softplus, bent identity, softexponential, sinusoid, sine, Gaussian, or sigmoid function, or any combination thereof.

The weighting factors, bias values, and threshold values, or other computational parameters of the neural network, may be “taught” or “learned” in a training phase using one or more sets of training data. For example, the parameters may be trained using the input data from a training dataset and a gradient descent or backward propagation method so that the output value(s) that the ANN determines are consistent with the examples included in the training dataset.

The number of nodes used in the input layer of the ANN or DNN may be at least about 10, 50, 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, 100,000, or greater. In other instances, the number of node used in the input layer may be at most about 100,000, 90,000, 80,000, 70,000, 60,000, 50,000, 40,000, 30,000, 20,000, 10,000, 9,000, 8,000, 7,000, 6,000, 5,000, 4,000, 3,000, 2,000, 1,000, 900, 800, 700, 600, 500, 400, 300, 200, 100, 50, 10, or fewer. In some instances, the total number of layers used in the ANN or DNN (including input and output layers) may be at least about 3, 4, 5, 10, 15, 20, or greater. In other instances, the total number of layers may be at most about 20, 15, 10, 5, 4, 3, or fewer.

In some instances, the total number of learnable or trainable parameters, e.g., weighting factors, biases, or threshold values, used in the ANN or DNN may be at least about 10, 50, 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, 100,000, or greater. In other instances, the number of learnable parameters may be at most about 100,000, 90,000, 80,000, 70,000, 60,000, 50,000, 40,000, 30,000, 20,000, 10,000, 9,000, 8,000, 7,000, 6,000, 5,000, 4,000, 3,000, 2,000, 1,000, 900, 800, 700, 600, 500, 400, 300, 200, 100, 50, 10, or fewer.

In some embodiments of a machine learning software module as described herein, a machine learning software module comprises a neural network such as a deep CNN. In some embodiments in which a CNN is used, the network is constructed with any number of convolutional layers, dilated layers, or fully-connected layers. In some embodiments, the number of convolutional layers is between 1-10, and the number of dilated layers is between 0-10. The total number of convolutional layers (including input and output layers) may be at least about 1, 2, 3, 4, 5, 10, 15, 20, or greater, and the total number of dilated layers may be at least about 1, 2, 3, 4, 5, 10, 15, 20, or greater. The total number of convolutional layers may be at most about 20, 15, 10, 5, 4, 3, or fewer, and the total number of dilated layers may be at most about 20, 15, 10, 5, 4, 3, or fewer. In some embodiments, the number of convolutional layers is between 1-10 and the fully-connected layers between 0-10. The total number of convolutional layers (including input and output layers) may be at least about 1, 2, 3, 4, 5, 10, 15, 20, or greater, and the total number of fully-connected layers may be at least about 1, 2, 3, 4, 5, 10, 15, 20, or greater. The total number of convolutional layers may be at most about 20, 15, 10, 5, 4, 3, 2, 1, or less, and the total number of fully-connected layers may be at most about 20, 15, 10, 5, 4, 3, 2, 1, or fewer.

In some embodiments, the input data for training of the ANN may comprise a variety of input values depending whether the machine learning algorithm is used for processing sequence or structural data. In some embodiments, the ANN or deep learning algorithm may be trained using one or more training datasets comprising the same or different sets of input and paired output data.

In some embodiments, a machine learning software module comprises a neural network comprising a CNN, recurrent neural network (RNN), dilated CNN, fully-connected neural networks, deep generative models, and deep restricted Boltzmann machines.

In some embodiments, a machine learning algorithm comprises CNNs. The CNN may be deep and feedforward ANNs. The CNN may be applicable to analyzing visual imagery. The CNN may comprise an input, an output layer, and multiple hidden layers. The hidden layers of a CNN may comprise convolutional layers, pooling layers, fully-connected layers, and normalization layers. The layers may be organized in 3 dimensions: width, height, and depth.

The convolutional layers may apply a convolution operation to the input and pass results of the convolution operation to the next layer. For processing sequence data, the convolution operation may reduce the number of free parameters, allowing the network to be deeper with fewer parameters. In neural networks, each neuron may receive input from some number of locations in the previous layer. In a convolutional layer, neurons may receive input from only a restricted subarea of the previous layer. The convolutional layer's parameters may comprise a set of learnable filters (or kernels). The learnable filters may have a small receptive field and extend through the full depth of the input volume. During the forward pass, each filter may be convolved across the length of the input sequence, determine the dot product between the entries of the filter and the input, and produce a two-dimensional activation map of that filter. As a result, the network may learn filters that activate when it detects some specific type of feature at some spatial position in the input.

In some embodiments, the pooling layers comprise global pooling layers. The global pooling layers may combine the outputs of neuron clusters at one layer into a single neuron in the next layer. For example, max pooling layers may use the maximum value from each of a cluster of neurons in the prior layer; and average pooling layers may use the average value from each of a cluster of neurons at the prior layer.

In some embodiments, the fully-connected layers connect every neuron in one layer to every neuron in another layer. In neural networks, each neuron may receive input from some number locations in the previous layer. In a fully-connected layer, each neuron may receive input from every element of the previous layer.

In some embodiments, the normalization layer is a batch normalization layer. The batch normalization layer may improve the performance and stability of neural networks. The batch normalization layer may provide any layer in a neural network with inputs that are zero mean/unit variance. The advantages of using batch normalization layer may include faster trained networks, higher learning rates, easier to initialize weights, more activation functions viable, and simpler process of creating deep networks.

In some embodiments, a machine learning software module comprises a recurrent neural network software module. A recurrent neural network software module may receive sequential data as an input, such as consecutive data inputs, and the recurrent neural network software module updates an internal state at every time step. A recurrent neural network can use internal state (memory) to process sequences of inputs. The recurrent neural network may be applicable to tasks such as codon selection. The recurrent neural network may also be applicable to next codon prediction, and codon usage anomaly detection. A recurrent neural network may comprise fully recurrent neural network, independently recurrent neural network, Elman networks, Jordan networks, Echo state, neural history compressor, long short-term memory, gated recurrent unit, multiple timescales model, neural Turing machines, differentiable neural computer, and neural network pushdown automata.

In some embodiments, a machine learning software module comprises a supervised or unsupervised learning method such as, for example, support vector machines (“SVMs”), random forests, clustering algorithm (or software module), gradient boosting, linear regression, logistic regression, and/or decision trees. The supervised learning algorithms may be algorithms that rely on the use of a set of labeled, paired training data examples to infer the relationship between an input data and output data. The unsupervised learning algorithms may be algorithms used to draw inferences from training datasets to the output data. The unsupervised learning algorithm may comprise cluster analysis, which may be used for exploratory data analysis to find hidden patterns or groupings in process data. One example of unsupervised learning method may comprise principal component analysis. The principal component analysis may comprise reducing the dimensionality of one or more variables. The dimensionality of a given variable may be at least 1, 5, 10, 50, 100, 200, 300, 400, 500, 600, 700, 800, 900, 1,000, 1,100, 1,200, 1,300, 1,400, 1,500, 1,600, 1,700, 1,800, or greater. The dimensionality of a given variables may be at most 1,800, 1,700, 1,600, 1,500, 1,400, 1,300, 1,200, 1,100, 1,000, 900, 800, 700, 600, 500, 400, 300, 200, 100, 50, 10, or fewer.

In some embodiments, the machine learning algorithm may comprise reinforcement learning algorithms. The reinforcement learning algorithm may be used for optimizing Markov decision processes (i.e., mathematical models used for studying a wide range of optimization problems where future behavior cannot be accurately predicted from past behavior alone, but rather also depends on random chance or probability). One example of reinforcement learning may be Q-learning. Reinforcement learning algorithms may differ from supervised learning algorithms in that correct training data input/output pairs are not presented, nor are sub-optimal actions explicitly corrected. The reinforcement learning algorithms may be implemented with a focus on real-time performance through finding a balance between exploration of possible outcomes (e.g., correct compound identification) based on updated input data and exploitation of past training.

In some embodiments, training data resides in a cloud-based database that is accessible from local and/or remote computer systems on which the machine learning-based sensor signal processing algorithms are running. The cloud-based database and associated software may be used for archiving electronic data, sharing electronic data, and analyzing electronic data. In some embodiments, training data generated locally may be uploaded to a cloud-based database, from which it may be accessed and used to train other machine learning-based detection systems at the same site or a different site.

The trained algorithm may accept a plurality of input variables and produce one or more output variables based on the plurality of input variables. The input variables may comprise one or more datasets of codons. For example, the input variables may comprise information about a codon-of-interest, a codon upstream of (or 5′ to) the codon-of-interest, a codon downstream of (or 3′ to) the codon-of-interest, or any combination thereof.

The trained algorithm may be trained with a plurality of independent training samples. Each of the independent training samples may comprise information about a codon-of-interest, a codon upstream of (or 5′ to) the codon-of-interest, a codon downstream of (or 3′ to) the codon-of-interest, or a combination thereof. The trained algorithm may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, at least about 500, at least about 1,000, at least about 1,500, at least about 2,000, at least about 2,500, at least about 3,000, at least about 3,500, at least about 4,000, at least about 4,500, at least about 5,000, at least about, 5,500, at least about 6,000, at least about 6,500, at least about 7,000, at least about 7,500, at least about 8,000, at least about 8,500, at least about 9,000, at least about 9,500, at least about 10,000, or more independent training samples.

The trained algorithm may associate information about a codon-of-interest, a codon upstream of (or 5′ to) the codon-of-interest, a codon downstream of (or 3′ to) the codon-of-interest, or a combination thereof for the best selection of codons for rewriting/replacement at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The trained algorithm may be adjusted or tuned to improve a performance or accuracy of determining the prediction or classification. The trained algorithm may be adjusted or tuned by adjusting parameters of the trained algorithm. The trained algorithm may be adjusted or tuned continuously during the training process or after the training process has completed.

After the trained algorithm is initially trained, a subset of the inputs may be identified as most influential or most important to be included for making high-quality predictions. For example, a subset of the data may be identified as most influential or most important to be included for making high-quality choice for selecting codons for rewriting and/or replacement. The data or a subset thereof may be ranked based on classification metrics indicative of each parameter's influence or importance toward making high-quality selection of codons for rewriting and/or replacement. Such metrics may be used to reduce, in some embodiments significantly, the number of input variables (e.g., predictor variables) that may be used to train the trained algorithm to a desired performance level (e.g., based on a desired minimum accuracy). For example, if training the trained algorithm with a plurality comprising several dozen or hundreds of input variables in the trained algorithm results in an accuracy of classification of more than 99%, then training the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The subset may be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best association metrics.

Systems and methods as described herein may use more than one trained algorithm to determine an output. Systems and methods may comprise 1, 2, 3, 4, 5, 6, 7, 8, 9, 10, 11, 12, 13, 14, 15, 16, 17, 18, 19, 20, or more trained algorithms. A trained algorithm of the plurality of trained algorithms may be trained on a particular type of data (e.g., sequence data, structural data). Alternatively, a trained algorithm may be trained on more than one type of data. The inputs of one trained algorithm may comprise the outputs of one or more other trained algorithms. Additionally, a trained algorithm may receive as its input the output of one or more trained algorithms. A set of outputs generated using one or more trained algorithms may be combined into a single output (e.g., by determining a sum, an average, a minimum, a maximum, or any other function applied to the set of outputs).

### New Assignment of Rewritten/Replaced Codons

In some aspects, provided herein, are methods for codon rewriting and replacement. In some embodiments, codons rewritten or replaced can be used to encode a new amino acid. In some embodiments, the new amino acid can be any canonical amino acids. For example, the new amino acid can be alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine. In some embodiments, the new amino acid can be a non-canonical amino acid (ncAA).

In some aspects, provided herein, are methods for genetic code expansion using codon rewriting and replacement. In some embodiments, methods described herein, may enable site-specific, co-translational incorporation of one or more ncAAs into a polypeptide or a protein. In some embodiments, methods described herein can provide transformational approaches to understand and control one or more biological functions. For example, codon rewriting/replacement can allow genetically encoding amino acids corresponding to post-translationally modified versions of natural amino acids. For example, codon rewriting/replacement to allow genetically encoding photocaged amino acids can enable the rapid activation of protein function with light to dissect dynamic processes in cells. For example, codon rewriting/replacement to allow genetically encoding crosslinkers can provide a way to map protein interactions. For example, ncAAs containing fluorophores or other biophysical probes can be used to follow changes in protein structure and/or activity. In some embodiments, ncAAs may be used to alter enzyme function. In some embodiments, ncAAs may be used to trap labile enzyme-substrate intermediates for structural studies and substrate identification. In some embodiments, ncAAs bearing bio-orthogonal and chemically reactive groups may provide strategies for rapidly attaching a wide range of functionalities to proteins to precisely control and image protein function in cells and to create protein conjugates, including defined therapeutic conjugates. In some embodiments, genetic code expansion using codon rewriting and replacement methods described herein may form the basis of strategies for the reversible control of gene expression in animals and strategies for determining cell type-specific proteomes in animals. In some embodiments, genetic code expansion using codon rewriting and replacement methods described herein may allow incorporating multiple distinct ncAAs into polypeptides or proteins.

Non-Canonical Amino Acid (ncAA)

As used herein, a non-canonical amino acid (ncAA) can refer to any amino acid other than the 20 genetically encoded alpha-amino acids comprising alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine. In some aspects, described herein are non-canonical amino acids (ncAAs) that may comprise side chain chemistries and/or structures that are not available from canonical amino acids (cAAs). In some embodiments, ncAAs may comprise fluorinated amino acids or amino acids comprising a reactive group (e.g., carbonyl, alkene, or alkyne moieties), or photoactivatable group (e.g., azide, benzophenone, or fluorophores). Translation of ncAAs into proteins may allow chemical modification and accordingly, ncAAs may be useful for in vivo structure-function studies, protein-protein interaction studies, protein localization studies, protein activity regulation studies or studies to generate new protein function. ncAA can be incorporated in different cells, including, but not limited to bacterial cells (e.g., Escherichia coli), yeast cells (e.g., Saccharomyces cerevisiae, Pichia pastoris, or Candida albicans), mammalian cells and plant cells or in organisms, including, but not limited to Drosophila melanogaster, Caenorhabditis elegans, Bombyx mori, rabbit and cow.

In some embodiments, a ncAA may comprise Para-fluoro-L-phenylalanine, Para-iodo-L-phenylalanine, Para-azido-L-phenylalanine, Para-acetyl-L-phenylalanine, Para-benzoyl-L-phenylalanine, Meta-fluoro-L-tyrosine, O-methyl-L-tyrosine, Para-propargyloxy-L-phenylalanine, (2S)-2-aminooctanoic acid, (2S)-2-aminononanoic acid, (2S)-2-aminodecanoic acid, (2S)-2-aminohept-6-enoic acid, (2S)-2-aminooct-7-enoic acid, L-Homocysteine, (2S)-2-amino-5-sulfanylpentanoic acid, (2S)-2-amino-6-sulfanylhexanoic acid, L-S-(2-nitrobenzyl) cysteine, L-S-ferrocenyl-cysteine, L-O-crotylserine, L-O-(pent-4-en-1-yl)serine, L-O—(4,5-dimethoxy-2-nitrobenzyl)serine, (2S)-2-amino-3-({[5-(dimethylamino)naphthalen-1-yl]sulfonyl}amino)propanoic acid, (2S)-3-[(6-acetyl-naphthalen-1-yl)amino]-2-aminopropanoic acid, L-Pyrrolysine, N6-[(propargyloxy)carbonyl]-L-lysine, L-N6-acetyllysine, N6-trifluoroacetyl-L-lysine, N6-{[1-(6-nitro-1,3-benzodioxol-5-yl)ethoxy]carbonyl}-L-lysine, N6-{[2-(3-methyl-3H-diaziren-3-yl)ethoxy]carbonyl}-L-lysine, p-azidophenylalanine or 2-aminoisobutyric acid (also known as α-aminoisobutyric acid, AIB, α-methylalanine, or 2-methylalanine).

In some embodiments, a ncAA may comprise AbK (unnatural amino acid for Photo-crosslinking probe), 3-Aminotyrosine (unnatural amino acid for inducing red shift in fluorescent proteins and fluorescent protein-based biosensors), L-Azidohomoalanine hydrochloride (unnatural amino acid for bio-orthogonal labeling of newly synthesized proteins), L-Azidonorleucine hydrochloride (unnatural amino acid for bio-orthogonal or fluorescent labeling of newly synthesized proteins), BzF (photoreactive unnatural amino acid; photo-crosslinker), DMNB-caged-Serine (caged serine; excited by visible blue light), HADA (blue fluorescent D-amino acid for labeling peptidoglycans in live bacteria), NADA-green (fluorescent D-amino acid for labeling peptidoglycans in live bacteria), NB-caged Tyrosine hydrochloride (ortho-nitrobenzyl caged L-tyrosine), RADA (orange-red TAMRA-based fluorescent D-amino acid for labeling peptidoglycans in live bacteria), Rf470DL (blue rotor-fluorogenic fluorescent D-amino acid for labeling peptidoglycans in live bacteria), sBADA (green fluorescent D-amino acid for labeling peptidoglycans in bacteria), or YADA (green-yellow lucifer yellow-based fluorescent D-amino acid for labeling peptidoglycans in live bacteria).

In some embodiments, a ncAA may comprise an O-methyl-L-tyrosine, an L-3-(2-naphthyl)alanine, a 3-methyl-phenylalanine, an O—4-allyl-L-tyrosine, a 4-propyl-L-tyrosine, a tri-O-acetyl-GlcNAcβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, or an isopropyl-L-phenylalanine.

In some embodiments, a ncAA may comprise an unnatural analogue of a canonical amino acid. For example, a ncAA may comprise an unnatural analogue of a tyrosine amino acid, an unnatural analogue of a glutamine amino acid, an unnatural analogue of a phenylalanine amino acid, an unnatural analogue of a serine amino acid, an unnatural analogue of a threonine amino acid. In some embodiments, a ncAA may comprise an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, or any combination thereof.

In some embodiments, a ncAA may comprise an amino acid with a photoactivatable cross-linker, a spin-labeled amino acid, a fluorescent amino acid, an amino acid with a novel functional group, an amino acid that covalently or noncovalently interacts with another molecule, a metal binding amino acid, a metal-containing amino acid, a radioactive amino acid, a photocaged amino acid, a photoisomerizable amino acid, a biotin or biotin-analogue containing amino acid, a glycosylated or carbohydrate modified amino acid, a keto containing amino acid, an amino acid comprising polyethylene glycol, an amino acid comprising polyether, a heavy atom substituted amino acid, a chemically cleavable or photocleavable amino acid, an amino acid with an elongated side chain, an amino acid containing a toxic group, or a sugar substituted amino acid. In some embodiments, a sugar substituted amino acid may comprise a sugar substituted serine. In some embodiments, a ncAA may comprise a carbon-linked sugar-containing amino acid, a redox-active amino acid, an α-hydroxy containing amino acid, an amino thio acid containing amino acid, an α,α disubstituted amino acid, a β-amino acid, or a cyclic amino acid other than proline.

In some embodiments, a ncAA may comprise p-azidophenylalanine or 2-aminoisobutyric acid (also known as α-aminoisobutyric acid, AIB, α-methylalanine, or 2-methylalanine).

### Orthogonal Translation System

The ribosome uses tRNA adaptors, aminoacylated with their cognate amino acids by specific aminoacyl-tRNA synthetases (aaRSs), to progressively decode the triplet codons in a coding sequence and polymerize the corresponding sequence of amino acids into a protein. 64 triplet codons are used to encode the 20 canonical amino acids, and the initiation and termination of protein synthesis. In some aspects, codon rewriting and replacement methods described herein may allow reassigning those rewritten codons to encode a new amino acid (referred to as orthogonal codons). In some embodiments, orthogonal codons can be assigned to ncAAs. In some embodiments, each new orthogonal codon must be decoded by an additional aminoacyl-tRNA synthetase (aaRS)/tRNA pair. In some embodiments, these aaRS/tRNA pairs may uniquely decode distinct codons and recognize distinct ncAAs.

In some aspects, methods described herein may require an orthogonal aaRS/tRNA pairs. In some embodiments, each orthogonal aaRS may aminoacylate its cognate orthogonal tRNA, and/or minimally aminoacylate the other tRNAs in an organism. In some embodiments, the orthogonal tRNA may be aminoacylated by its cognate synthetase and/or minimally be aminoacylated by the aaRSs of the organism. In some embodiments, the orthogonal tRNA may be engineered to recognize an orthogonal codon that is not assigned to a canonical amino acid (i.e., rewritten/replaced codons), while maintaining selective aminoacylation by the orthogonal synthetase. In some embodiments, an active site of the orthogonal synthetase may be engineered.

In some aspects, provided herein are methods for reassigning a codon to encode an amino acid that the codon does not naturally encode. For example, a codon may be reassigned to a ncAA, i.e., the codon encodes a ncAA instead of an amino acid naturally encoded by the codon. Over 100 ncAAs with diverse chemistries may be synthesized and co-translationally incorporated into polypeptides and proteins using evolved orthogonal aminoacyl-tRNA synthetase (aaRSs)/tRNA pairs. Various aaRS/tRNA pairs can be used for methods described herein. In some embodiments, an ncAA may be designed based on tyrosine or pyrrolysine. In some embodiments, an aaRS/tRNA pair may be provided on a plasmid or into the genome of a cell or an organism comprising one or more reassigned codons. In some embodiments, an orthogonal aaRS/tRNA pair can be used to bioorthogonally incorporate ncAAs into polypeptides or proteins.

In some embodiments, vector-based over-expression systems may be used. In some embodiments, vector-based over-expression systems may outcompete natural codon function with its reassigned function. In some embodiments where natural aaRS and/or tRNAs for the rewritten codon are completely abolished or removed, lower amount of aaRS/tRNA for the newly assigned ncAA may be sufficient to achieve efficient ncAA incorporation. In some embodiments, genome-based aaRS/tRNA pairs (i.e., aaRS/tRNA pairs incorporated into the genome of the cell or organism) may be used to reduce the mis-incorporation of canonical amino acids in the absence of available ncAAs. In some embodiments, ncAA incorporation into polypeptides or proteins may involve supplementing the growth media with the ncAA described herein and an inducer for the aaRS expression. Alternatively, the aaRS may be expressed constitutively.

In some embodiments, aaRS/tRNA pairs may be imported from evolutionarily divergent organisms, wherein the sequence has diverged from that of the aaRS/tRNA pairs in the host organism or cell of interest (e.g., archaeal and eukaryotic pairs in an E. coli host). In some embodiments, derivatives of the Methanocaldococcus janaschii tyrosyl-tRNA synthetase (MjTyrRS)/MjtRNA^{Tyr} pair may be used to incorporate a wide variety of ncAAs into polypeptides or proteins. In some embodiments, derivatives of the E. coli leucyl-tRNA synthetase (EcLeuRS)/EctRNA^{Leu}, E. coli tryptophanyl-tRNA synthetase (EcTrpRS)/EctRNA^{Trp}, or EcTyrRS/EctRNA^{Tyr} pairs may be used to incorporate one or more ncAAs into polypeptides or proteins. In some embodiments, EcTyrRS/EctRNA^{Tyr} pair or EcTrpRS/EctRNA^{Trp}pair may be directly evolved for a new ncAA specificity. In some embodiments, endogenous copies of aaRS/tRNA pairs maybe replaced with pairs that are orthogonal in another host organism.

In some embodiments, evolved derivatives of a Methanococcus maripaludis phosphoseryl-tRNA synthetase (MmpSepRS)/MjtRNA^{Sep} pair may be used to incorporate phosphoserine, its non-hydrolysable analogue, or phosphothreonine. In some embodiments, Methanosarcina mazei pyrrolysyl-tRNA synthetase (MmPylRS)/MmtRNA^{Pyl}CUA pair, Methanosarcina barkeri PylRS (MbPylRS)/MbtRNA^{PYl}_{CUA} pair, or derivatives thereof, may be used to incorporate one or more ncAAs. In some embodiments, Archaeoglobus fulgidus (Af)TyrRS/AffRNA^{Tyr}CUA may be used to incorporate one or more ncAAs. In some embodiments, engineered aaRS/tRNA pairs may be used to incorporate one or more ncAAs.

An organism or a host organism described herein can be an animal. In some embodiments, the animal may be a mammal. In some embodiments, the mammal comprises a human, non-human primate, rodent, caprine, bovine, ovine, equine, canine, feline, mouse, rat, rabbit, horse or goat. In some embodiments, an organism or a host organism may comprise E. coli, Salmonella enterica subsp. enterica serovar Typhimurium, Saccharomyces cerevisiae, cultured mammalian cells, Arabidopsis thaliana, Caenorhabditis elegans, Drosophila melanogaster or Mus musculus.

A cell or a host cell described herein can be a bacterial cell, a yeast cell, a fungal cell, an insect cell, or a mammalian cell. In some embodiments, a cell may comprise a mammalian cell. Mammalian cells can be derived or isolated from a tissue of a mammal. In some embodiments, mammalian cells may comprise COS cells, BHK cells, 293 cells, 3T3 cells, NSO hybridoma cells, baby hamster kidney (BHK) cells, PER.C6™ human cells, HEK293 cells or Cricetulus griseus (CHO) cells. In some embodiments, a mammalian cell may comprise a human cell, a rodent cell, or a mouse cell. Examples of mammalian cells can also include but are not limited to cells from humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In some embodiments, a mammalian cell is a human cell. In some embodiments, a mammalian cell is a mouse cell. In some embodiments, a mammalian cell comprises an embryonic stem cell (ESC), a pluripotent stem cell (PSC), or an induced pluripotent stem cell (iPSC). In some embodiments, a cell or a host cell may comprise an eukaryotic cell or a prokaryotic cell. In some embodiments, the prokaryotic cell comprises an archaebacteria cell, a bacterial cell, or a combination thereof. In some embodiments, the eukaryotic cell comprises an yeast cell, a fungal cell, a plant cell, an animal cell, an insect cell, a mammalian cell, or a combination thereof. In some embodiments, the mammalian cell comprises a rodent cell, a mouse cell, or a human cell, or a combination thereof.

Methods for incorporating non-canonical amino acids in yeast are described in, for example, Stieglitz J. T., Van Deventer J. A. (2022) Incorporating, Quantifying, and Leveraging Noncanonical Amino Acids in Yeast. In: Rasooly A., Baker H., Ossandon M. R. (eds) Biomedical Engineering Technologies. Methods in Molecular Biology, vol 2394. Humana, New York, NY (doi.org/10.1007/978-1-0716-1811-0_21), which is incorporated by reference herein in its entirety.

Applications of proteins with non-canonical amino acids are described in, for example, Jeremiah A Johnson, Ying Y Lu, James A Van Deventer, David A Tirrell, Residue-specific incorporation of non-canonical amino acids into proteins: recent developments and applications,

Current Opinion in Chemical Biology, Volume 14, Issue 6, 2010, Pages 774-780, ISSN 1367-5931, doi.org/10.1016/j.cbpa.2010.09.013 (www.sciencedirect.com/science/article/pii/S1367593110001390), which is incorporated by reference herein in its entirety.

Examples of orthogonal translation in E. coli with a genome rewritten to exclude a subset of sense codons are described in, for example, Robertson W E, Funke LFH, de la Torre D, Fredens J, Elliott T S, Spinck M, Christova Y, Cervettini D, Böge FL, Liu K C, Buse S, Maslen S, Salmond GPC, Chin JW. Sense codon reassignment enables viral resistance and encoded polymer synthesis. Science. 2021 Jun. 4; 372(6546):1057-1062. doi: 10.1126/science.abg3029. PMID: 34083482; PMCID: PMC7611380, which is incorporated by reference herein in its entirety.

Additional examples of orthogonal translation are described in, for example, de la Torre, D., Chin, J. W. Reprogramming the genetic code. Nat Rev Genet 22, 169-184 (2021) (doi.org/10.1038/s41576-020-00307-7), which is incorporated by reference herein in its entirety.

Quantitative Reporter Platform to Evaluate ncAA Incorporation

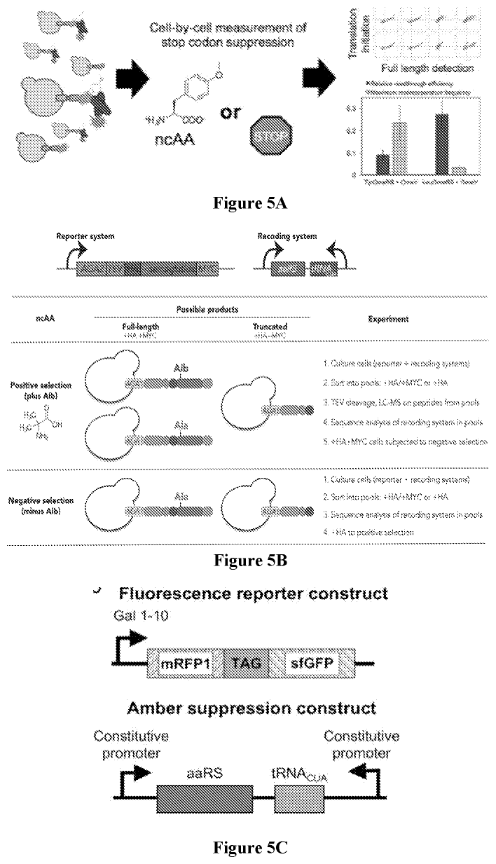

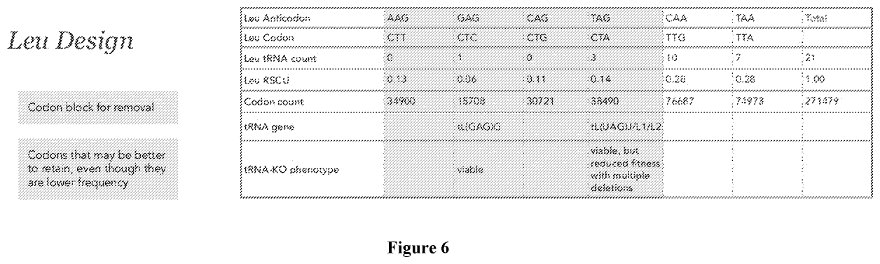

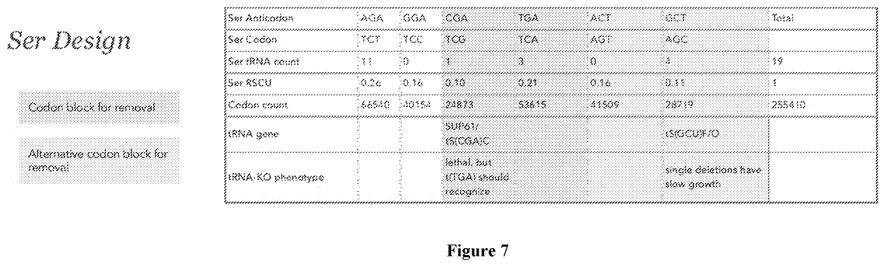

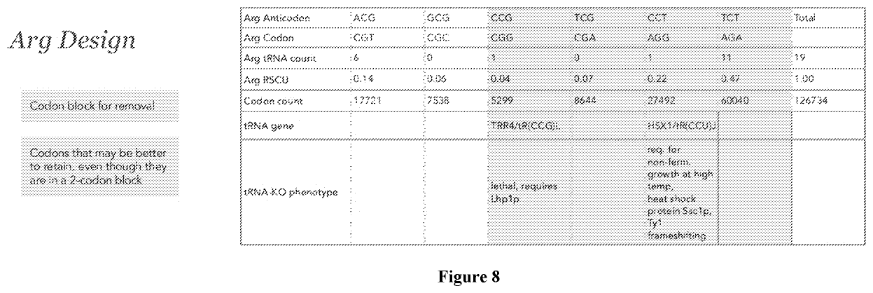

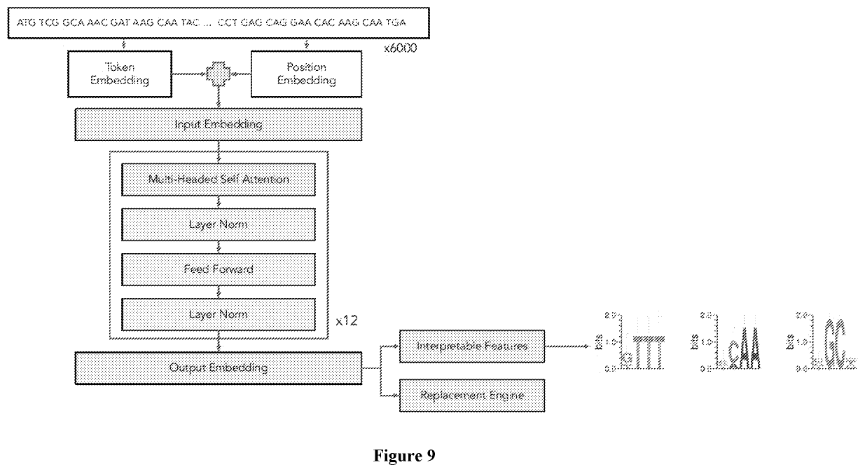

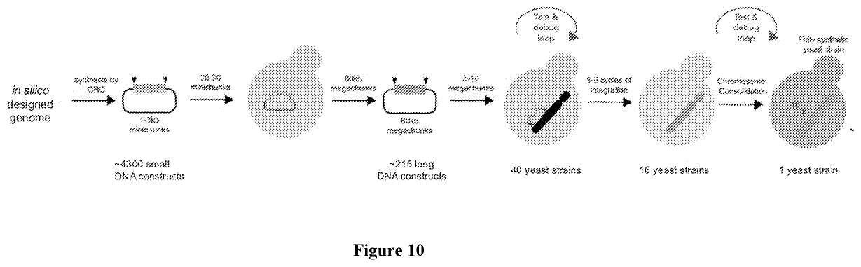

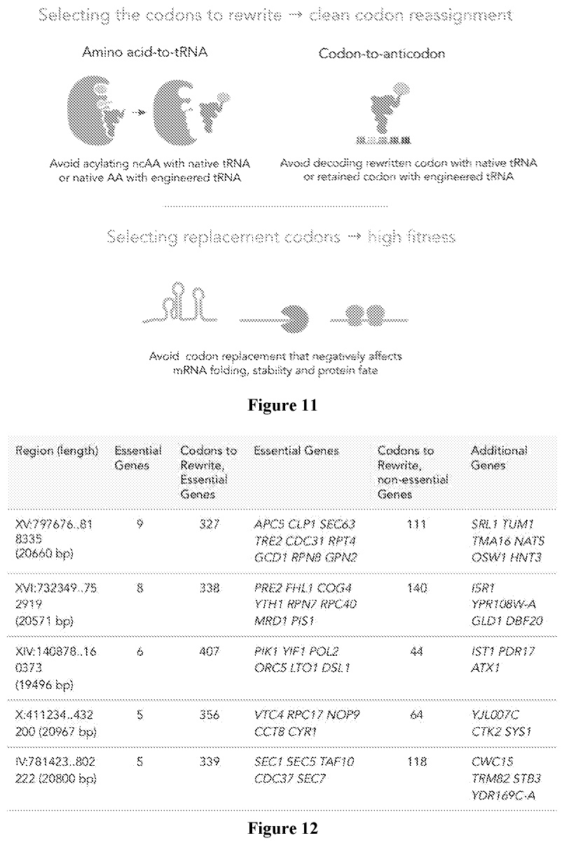

In some embodiments, a precise plate-based assay using flow cytometry-based endpoint readouts can be used to measure efficiency and fidelity of an orthogonal translation system (as shown in FIG. 5). In some embodiments, a high throughput assay can be used for ncAA incorporation with additional mass spectrometry assays. In some embodiments, a dual reporter system is used for surface display. In some embodiments, a dual reporter system using two fluorescent tags can be employed to evaluate orthogonal evaluation. Details of assays provided herein are described in, for example, Stieglitz, et al. ACS Synth Biol. 2018 Sep. 21; 7(9): 2256-2269 A robust and quantitative report system to evaluate noncanonical amino acid incorporation in yeast, which is incorporated by reference herein in its entirety.

### Other Embodiments

In some aspects, provided herein, is a method comprising: a) analyzing at least a portion of a genome of an organism to identify a first plurality of codons based on at least in part on a first local context of a codon-of-interest in the genome of the organism to be rewritten; b) rewriting the first plurality of codons in the genome of the organism to a second codon, wherein the first plurality of codons and the second codon encode a first amino acid, and wherein the rewriting of the first plurality of codons modulates an occurrence of the first plurality of codons; and c) synthesizing a nucleic acid construct comprising the portion of the genome, wherein the first plurality of codons is rewritten to the second codon.

In some embodiments, the method further comprises introducing the nucleic acid construct into a cell of the organism to replace the portion of the genome of the organism. In some embodiments, the modulating of the occurrence of the first plurality of codons comprises eliminating the occurrence of the first plurality of codons. In some embodiments, the analyzing comprises identifying one or more synonymous codons with a least number of occurrences in the genome of the organism. In some embodiments, the first plurality of codons comprises the one or more synonymous codons with the least number of occurrences.

In some embodiments, the first local context of the codon-of-interest comprises C₍ₙ₋₁₎ Cₙ−C₍ₙ₊₁₎, wherein C₍ₙ₋₁₎ denotes a codon downstream of the codon-of-interest; Cₙ denotes the codon-of-interest; and C₍ₙ₊₁₎ denotes a codon upstream of the codon-of-interest. In some embodiments, the analyzing further comprises determining a number of occurrences of the first local context of the codon-of-interest. In some embodiments, the analyzing further comprises determining a relative synonymous codon usage (RSCU) of the codon-of-interest.

In some embodiments, the analyzing further comprises identifying the first plurality of codons based at least in part on a second local context of the codon-of-interest in the genome of the organism. In some embodiments, the second local context of the codon-of-interest comprises C₍ₙ₋₁₎−AAₙ−C₍ₙ₊₁₎, wherein C₍ₙ₋₁₎ denotes a codon downstream of the codon-of-interest; AAₙ denotes an amino acid encoded by the codon-of-interest; and C₍ₙ₊₁₎ denotes a codon upstream of the codon-of-interest. In some embodiments, the analyzing further comprises determining a number of occurrences of the second local context of the codon-of-interest. In some embodiments, the analyzing further comprises determining an expected number of occurrences of the first local context of the codon-of-interest. In some embodiments, the expected number of occurrences of the first local context of the codon-of-interest is determined as a product of: a number of occurrences of the second local context of the codon-of-interest, and the determined RCSU of the codon-of-interest.

In some embodiments, the analyzing comprises processing the at least the portion of the genome of the organism using a machine learning-based computer system. In some embodiments, the machine learning-based computer system comprises one or more storage units comprising, respectively, one or more storage devices included within respective storage arrays controlled by a respective one or more storage controllers; and one or more computer processing units, wherein the one or more computer processing units communicate with the one or more storage units over a communication interface.

In some embodiments, the analyzing further comprises identifying one or more statistically significant evolutionary signals. In some embodiments, the one or more statistically significant evolutionary signals comprise a negative evolutionary selection signal, a positive evolutionary selection signal, or a combination thereof. In some embodiments, the negative selection signal comprises a frameshift, a ribosome stall, or a secondary RNA structure interfering with transcription or translation. In some embodiments, the positive selection signal comprises a regulatory element within an open reading frame (ORF).

In some embodiments, the method further comprises reassigning the first plurality of codons to a second amino acid. In some embodiments, the first amino acid or the second amino acid comprises alanine, cysteine, aspartic acid, glutamic acid, phenylalanine, glycine, histidine, isoleucine, lysine, leucine, methionine, asparagine, proline, glutamine, arginine, serine, threonine, valine, tryptophan, or tyrosine. In some embodiments, the first amino acid comprises arginine, leucine, or serine. In some embodiments, the first plurality of codons comprises CGT, CGC, CGA, CGG, AGA, AGG, or a combination thereof. In some embodiments, the first plurality of codons comprises CGA, CGG, or a combination thereof. In some embodiments, the first plurality of codons comprises TTA, TTG, CTT, CTC, CTA, CTG, or a combination thereof. In some embodiments, the first plurality of codons comprises CTA, CTG, or a combination thereof. In some embodiments, the first plurality of codons comprises TCT, TCC, TCA, TCG, AGT, AGC, or a combination thereof. In some embodiments, the first plurality of codons comprises AGT, AGC, TCG, TCA, or a combination thereof.

In some embodiments, the rewriting further comprises removing a plurality of tRNA molecules with anticodons that recognize the first plurality of codons. In some embodiments, the removing comprises deleting one or more genes that encode the plurality of tRNA molecules that recognize the first plurality of codons. In some embodiments, the method further comprises providing additional tRNA molecules that recognize the first plurality of codons and aminoacyl-tRNA synthetases (aaRSs) for charging the additional tRNA molecules with the second amino acid. In some embodiments, the method further comprises providing a tRNA pre-charged with the second amino acid.

In some embodiments, the second amino acid comprises a non-canonical amino acid. In some embodiments, the non-canonical amino acid comprises p-azidophenylalanine, 2-aminoisobutyric acid (Aib), or a combination thereof.

In some embodiments, the rewriting of the first plurality of codons comprises modulating one or more codons in the first plurality of codons, wherein the one or more codons are within 4 codons of each other. In some embodiments, the rewriting of the first plurality of codons comprises modulating a codon fragment of one or more codons in the first plurality of codons. In some embodiments, the codon fragment comprises a trimer, a hexamer, a 9 mer, or a combination thereof.

In some aspects, provided herein, is a method of producing a polypeptide comprising a non-canonical amino acid (ncAA) or a population of polypeptide molecules comprising the ncAA in an organism, the method comprising: rewriting a first codon encoding a first amino acid to a second codon encoding the first amino acid in a genome of the organism, wherein the rewriting comprises identifying the first codon based at least in part on a first local context of a codon-of-interest in the genome of the organism; reassigning the first codon to encode the ncAA in the genome of the organism; and introducing into the organism an aminoacyl-tRNA synthetase (aaRS)/tRNA pair engineered to recognize the first codon and incorporate the ncAA into an amino acid sequence of the polypeptide or the population of the polypeptide molecules.

In some embodiments, the first codon has a least number of occurrences for the first amino acid in the genome of the organism. In some embodiments, the first local context of the codon-of-interest comprises C₍ₙ₋₁₎−Cₙ−C₍ₙ₊₁₎, wherein C₍ₙ₋₁₎ denotes a codon downstream of the codon-of-interest; Cₙ denotes the codon-of-interest; and C₍ₙ₊₁₎ denotes a codon upstream of the codon-of-interest. In some embodiments, the rewriting comprises determining a number of occurrences of the first local context of the codon-of-interest. In some embodiments, the rewriting further comprises determining a relative synonymous codon usage (RSCU) of the codon-of-interest.

In some embodiments, the rewriting further comprises identifying the first codon based at least in part on a second local context of the codon-of-interest in the genome of the organism. In some embodiments, the second local context of the codon-of-interest comprises C₍ₙ₋₁₎−AAₙ−C₍ₙ₊₁₎, wherein C₍ₙ₋₁₎ denotes a codon downstream of the codon-of-interest; AAₙ denotes an amino acid encoded by the codon-of-interest; and C₍ₙ₊₁₎ denotes a codon upstream of the codon-of-interest. In some embodiments, the rewriting further comprises determining a number of occurrences of the second local context of the codon-of-interest. In some embodiments, the rewriting further comprises determining an expected number of occurrences of the first local context of the codon-of-interest. In some embodiments, the expected number of occurrences of the first local context of the codon-of-interest is determined as a product of: a number of occurrences of the second local context of the codon-of-interest, and the determined RCSU of the codon-of-interest.

In some embodiments, the rewriting comprises analyzing at least a portion of the genome of the organism using a machine learning-based computer system. In some embodiments, the machine learning-based computer system comprises one or more storage units comprising, respectively, one or more storage devices included within respective storage arrays controlled by a respective one or more storage controllers; and one or more computer processing units, wherein the one or more computer processing units communicate with the one or more storage units over a communication interface.

In some embodiments, the method further comprises identifying one or more statistically significant evolutionary signals. In some embodiments, the one or more statistically significant evolutionary signals comprises a negative evolutionary selection signal, a positive evolutionary selection signal, or a combination thereof. In some embodiments, the negative selection signal comprises a frameshift, a ribosome stall, or a secondary RNA structure interfering with transcription or translation. In some embodiments, the positive selection signal comprises a regulatory element within an open reading frame (ORF).

In some embodiments, the first amino acid comprises alanine, cysteine, aspartic acid, glutamic acid, phenylalanine, glycine, histidine, isoleucine, lysine, leucine, methionine, asparagine, proline, glutamine, arginine, serine, threonine, valine, tryptophan, or tyrosine. In some embodiments, the first amino acid comprises arginine, leucine, or serine. In some embodiments, the first codon or the second codon comprises CGT, CGC, CGA, CGG, AGA, AGG, or a combination thereof. In some embodiments, the first codon comprises CGA, CGG, or a combination thereof. In some embodiments, the first codon or the second codon comprises TTA, TTG, CTT, CTC, CTA, CTG, or a combination thereof. In some embodiments, the first codon comprises CTA, CTG, or a combination thereof. In some embodiments, the first codon or the second codon comprises TCT, TCC, TCA, TCG, AGT, AGC, or a combination thereof. In some embodiments, the first codon comprises AGT, AGC, TCG, TCA, or a combination thereof.

In some embodiments, the first codon comprises a plurality of codons. In some embodiments, the rewriting further comprises removing a plurality of tRNA molecules that recognize the first codon. In some embodiments, the removing comprises deleting one or more genes that encode the plurality of tRNA molecules that recognize the first codon. In some embodiments, the introducing further comprises providing a tRNA pre-charged with the ncAA. In some embodiments, the ncAA comprises p-azidophenylalanine, 2-aminoisobutyric acid (Aib), or a combination thereof.

In some aspects, provided herein, is a method of producing a peptide, the method comprising editing a genome of an organism, wherein the editing comprises revising a codon of the genome to encode a non-canonical amino acid, wherein the peptide comprises the non-canonical amino acid.

In some aspects, provided herein, is a cell or a population of cells comprising a genome, wherein a first plurality of codons in the genome of the organism is rewritten to a second codon, wherein the first plurality of codons and the second codon encode a first amino acid, and wherein an occurrence of the first plurality of codons is modulated responsive to being rewritten to the second codon.

In some embodiments, the occurrence of the first plurality of codons is eliminated. In some embodiments, the first plurality of codons is reassigned to a second amino acid. In some embodiments, the first plurality of codons is identified based on a first plurality of codons based on at least in part on a first local context of a codon-of-interest.

In some embodiments, the first local context of the codon-of-interest comprises C₍ₙ₋₁₎ Cₙ−C₍ₙ₊₁₎, wherein C₍ₙ₋₁₎ denotes a codon downstream of the codon-of-interest; Cₙ denotes the codon-of-interest; and C₍ₙ₊₁₎ denotes a codon upstream of the codon-of-interest. In some embodiments, the identifying comprises determining a number of occurrences of the first local context of the codon-of-interest. In some embodiments, the identifying further comprises determining a relative synonymous codon usage (RSCU) of the codon-of-interest.

In some embodiments, the first plurality of codons is further identified based at least in part on a second local context of the codon-of-interest in the genome of the organism. In some embodiments, the second local context of the codon-of-interest comprises C₍ₙ₋₁₎−AAₙ C₍ₙ₊₁₎, wherein C₍ₙ₋₁₎ denotes a codon downstream of the codon-of-interest; AAₙ denotes an amino acid encoded by the codon-of-interest; and C₍ₙ₊₁₎ denotes a codon upstream of the codon-of-interest.

In some embodiments, the identifying further comprises determining a number of occurrences of the second local context of the codon-of-interest. In some embodiments, the identifying further comprises determining an expected number of occurrences of the first local context of the codon-of-interest. In some embodiments, the expected number of occurrences of the first local context of the codon-of-interest is determined as a product of: a number of occurrences of the second local context of the codon-of-interest, and the determined RCSU of the codon-of-interest.

In some embodiments, the identifying comprises analyzing at least a portion of the genome of the organism using a machine learning-based computer system. In some embodiments, the machine learning-based computer system comprises one or more storage units comprising, respectively, one or more storage devices included within respective storage arrays controlled by a respective one or more storage controllers; and one or more computer processing units, wherein the one or more computer processing units communicate with the one or more storage units over a communication interface.

In some embodiments, the identifying further comprises identifying one or more statistically significant evolutionary signals. In some embodiments, the one or more statistically significant evolutionary signals comprises a negative evolutionary selection signal, a positive evolutionary selection signal, or a combination thereof. In some embodiments, the negative selection signal comprises a frameshift, a ribosome stall, or a secondary RNA structure interfering with transcription or translation. In some embodiments, the positive selection signal comprises a regulatory element within an open reading frame (ORF). In some embodiments, the cell or the population of cells comprises an eukaryotic cell or a prokaryotic cell. In some embodiments, the prokaryotic cell comprises an archaebacteria cell, a bacterial cell, or a combination thereof. In some embodiments, the eukaryotic cell comprises an yeast cell, a fungal cell, a plant cell, an animal cell, an insect cell, a mammalian cell, or a combination thereof. In some embodiments, the mammalian cell comprises a rodent cell, a mouse cell, or a human cell, or a combination thereof.

In some embodiments, the first amino acid comprises alanine, cysteine, aspartic acid, glutamic acid, phenylalanine, glycine, histidine, isoleucine, lysine, leucine, methionine, asparagine, proline, glutamine, arginine, serine, threonine, valine, tryptophan, or tyrosine. In some embodiments, the first amino acid comprises arginine, leucine, or serine. In some embodiments, the first plurality of codons comprises CGT, CGC, CGA, CGG, AGA, AGG, or a combination thereof. In some embodiments, the first plurality of codons comprises CGA, CGG, or a combination thereof. In some embodiments, the first plurality of codons comprises TTA, TTG, CTT, CTC, CTA, CTG, or a combination thereof. In some embodiments, the first plurality of codons comprises CTA, CTG, or a combination thereof. In some embodiments, the first plurality of codons comprises TCT, TCC, TCA, TCG, AGT, AGC, or a combination thereof. In some embodiments, the first plurality of codons comprises AGT, AGC, TCG, TCA, or a combination thereof.

In some embodiments, the second amino acid comprises alanine, cysteine, aspartic acid, glutamic acid, phenylalanine, glycine, histidine, isoleucine, lysine, leucine, methionine, asparagine, proline, glutamine, arginine, serine, threonine, valine, tryptophan, or tyrosine. In some embodiments, the second amino acid comprises a non-canonical amino acid (ncAA). In some embodiments, the ncAA comprises p-azidophenylalanine, 2-aminoisobutyric acid (Aib), or a combination thereof.

In some aspects, provided herein, is an organism comprising the cell or the population of cells described herein.

In some aspects, provided herein, is a computer system for editing a genome of an organism, comprising: a database that is configured to store at least a portion of the genome of the organism; and one or more computer processors operatively coupled to said database, wherein said one or more computer processors are individually or collectively programmed to: a) analyze the at least the portion of the genome of the organism to identify a first plurality of codons in the genome of the organism to be rewritten; and b) rewrite the first plurality of codons in the genome of the organism to a second codon, wherein the first plurality of codons and the second codon encode a first amino acid, and wherein the rewriting of the first plurality of codons modulates an occurrence of the first plurality of codons, thereby editing the genome of the organism.

In some aspects, provided herein, is a non-transitory computer-readable storage medium comprising machine-executable code that, upon execution by one or more computer processors, implements a method for editing a genome of an organism, the method comprising: a) analyzing at least a portion of the genome of the organism to identify a first plurality of codons in the genome of the organism to be rewritten; and b) rewriting the first plurality of codons in the genome of the organism to a second codon, wherein the first plurality of codons and the second codon encode a first amino acid, and wherein the rewriting of the first plurality of codons modulates an occurrence of the first plurality of codons, thereby editing the genome of the organism.

### Examples

These examples are provided for illustrative purposes only and not to limit the scope of the claims provided herein.

### Example 1: Codon Selection for Rewriting/Replacement

For maximum flexibility in selecting replacement codons, amino acids encoded by 6 different codons are used for this example using Saccharomyces cerevisiae as the model organism. As this example focuses on DNA genes, DNA nomenclature, e.g., A, C, G, or T, is used.

Leucine: Leucine may be encoded by a set of 6 codons, which include CTT, CTC, CTG, CTA, TTG, and TTA. The choices are to rewrite CTG/CTA (1.42% of all Leucine codons) or TTG/TTA (5.2% of all Leucine codons). To reduce the number of rewritten codons, CTG/CTA is chosen to be rewritten. It's noteworthy that the Candida genus of yeast has lineages in which CTG has been reassigned from leucine (the ancestral state) to serine.

This demonstrates the ability to reassign this codon. The leucine anticodons for the 4-block are GAG (1 copy) and TAG (3 copies). It is most likely the TAG anticodon that decodes CTG. The GAG anticodon may decode CTC and CTT. Deleting the GAG anticodon tRNA (YNCG0028 W) causes no fitness defect, which means that the 3-copy TAG anticodon supplies it. Candida species have additional tRNAs with the AAG anticodon for the 4-block. If the TAG tRNAs are deleted, then these additional tRNAs may have to be supplied.

Leucine design summary: rewrite CTG/CTA codons, or possibly just the CTG codons. Delete the tL(TAG) genes, 3 copies. Possibly supplement with tL(AAG) tRNA genes from a related yeast species.

Serine: Serine may be encoded by a set of 6 codons, which include TCT, TCC, TCG, TCA, AGT, and AGC. The candidates for rewriting are TCG/TCA (2.78% of all serine codons) or AGT/AGC (2.47% of all serine codons). For the TCG/TCA choice, the anticodons are tS(CGA) 1 copy and tS(TGA) 3 copies. For the AGT/AGC choice, the anticodons are tS(GCT) 4 copies. Although in some embodiments it is favored to rewrite codons ending in G, in this case it may be reasonable to rewrite the AGT/AGC pair, because the GCT anticodon may not give cross-talk outside of the AGT/AGC 2-block.

Serine design summary, design 1: rewrite TCG/TCA codons, delete tS(CGA) 1 copy, tS(TGA) 3 copies. Increase copy numbers of other tS tRNA genes.

Serine design summary, design 2: rewrite AGT, AGC codons, delete tS(GCT) 4 copies. Increase copy numbers of other tS tRNA genes.

Arginine: Arginine may be encoded by a set of 6 codons, which include CGT, CGC, CGG, CGA, AGG, and AGA. The choices are to rewrite CGG/CGA (0.56% of all arginine codons) or AGG/AGA (3.110% of all arginine codons). To reduce the number of rewritten codons, CGG/CGA is chosen to be rewritten. The anticodons in the 4-block are ACG (6 copies) and CCG (1 copy). The single-copy CCG anticodon tRNA is TRR4. It is an essential tRNA gene, suggesting that no other tRNA recognizes CGG. Rewriting CGG and deleting TRR4 may permit use of CGG for orthogonal translation. In this case it may not be necessary to rewrite CGA because it is decoded by the ACG tRNA that may not recognize CGG.

Arginine design summary: rewrite CGG/CGA codons, delete tR(CCG) single-copy tRNA. Possibly increase copy number of remaining Arg tRNA genes to account for rewritten codons.

### Codon Removal Strategy

Leu CTG/CTA rewrite: 69K codons, 3 tRNAs.

Arg CGG/CGA rewrite: 14K codons, 1 tRNA.

Ser AGT/AGC rewrite: 70K codons, 4 tRNAs.

Ser TCG/TCA rewrite: 78K codons, 4 tRNAs.

Total over 6 codons: ˜160K codons to rewrite.

### Designs

5 regions of 20 kb each, 7 designs per region, 700 kb total.

‘Individual’ designs: 2 codons removed: Leu, Arg, Ser.

‘Paired’ designs: 3 codons removed: Leu/Arg, Leu/Ser, Arg/Ser.

‘All’ design: 6 codons removed: Leu/Arg/Ser.

### Example 2: Codon Replacement-Other Methods

A simple method for rewriting a codon is to change a nucleotide in the wobble position (third position of a codon) in a way that retains GC content. For example, a codon that ends with G or A in a 4-codon block (4 codons encoding a same amino acid) may be to change C or T, respectively. Alternatively, a codon may be changed to another codon having the highest frequency for that specific amino acid.

### Example 3: Codon Replacement-Goldilocks Design

The Goldilocks method for codon replacement can start with examining the local context of a codon. First, the frequency of each single codon is determined, and the relative synonymous codon usage (RSCU) may be determined (e.g., as the frequency of a codon divided by the frequency of all codons encoding the same amino acid). Second, the context of a codon is determined considering the preceding codon, the codon under consideration, and the subsequent codon. A protein-coding gene of a host species is examined, and the number of times each codon-codon-codon 9 mer occurs is determined. For example, in yeast, there are 4\hat{ }9 (=262,144) different 9 mers and approximately 3 million different codons. On average, each 9 mer occurs 11 times. The observed number of occurrences of the 9 mer may be defined as O(9 mer). The 9 mer contexts are then converted to patterns of codon-amino acid (aa)-codon, wherein aa is the amino acid encoded by the central codon. There are 4\hat{ }3×20×4\hat{ }3( =8,190) different patterns.

Next, the number of times that the central codon is expected to be observed under the null hypothesis is the number of times that the codon-aa-codon pattern occurs times the RCSU for the central codon. This is denoted as E(9 mer) for the expected number of occurrences of the 9 mer.

The p-value is then determined for a two-sided Poisson test for enrichment or depletion of the 9 mer relative to the null distribution. Standard significance at the 0.05 level, corrected for 262,144 9 mer tests, requires a single-test p-value of 1.9E-7 for significance.

The 9 mers that are over-represented or under-represented suggest selective pressure. Over-represented 9 mers may include regulatory motifs. Under-represented 9 mers may have undesired functions, such as frameshifts. The Goldilocks approach may have a goal to avoid creating 9 mers that have a significant deviation from the null.

One implementation is to use a simple codon replacement (maintaining GC content as described in Example 3) unless the result creates a 9 mer that deviates from the null, in which case an alternative is selected. An alternative implementation is to choose the new codon as the 9 mer whose observed frequency is closest to the expected frequency, excluding 9 mers whose central codon is in the set to be replaced. For repeated occurrences of codons that are to be replaced, the Goldilocks method may be applied in overlapping 9 mer windows across the region.

### Example 4: Using the Goldilocks Method to Rewrite Yeast Protein-Coding Genes

This example uses the Goldilocks method to rewrite yeast protein-coding genes. This example uses computer files with the following directory structure (Table 5).

TABLE 5
Directory Structure

| | |
|---|---|
| goldilocks/ | top-level directory |
| ../data/ | external data directory |
| ../../ncbi_translation_table_01.txt | NCBI translation table 1 (the standard genetic code) |
| ../../aa_info.txt | Amino acids, 3-letter codes, 1-letter codes |
| ../../orf_coding.fasta | SGD CDS from ATG through Ter, including verified, |
| | uncharacterized, transposable, excluding dubious |
| | and pseudogenes |
| ../../orf_trans.fasta | SGD translated ORFs, including verified, |
| | uncharacterized, transposable, excluding dubious |
| | and pseudogenes |
| ../src/ | source codes and scripts for running |
| ../../run_goldilocks.sh | script to run |
| ../../goldilocks.py | program implementing Goldilocks design |
| ../results/ | results directory |

### Input Data

Translation tables were retrieved from NCBI from:

www.ncbi.nlm.nih.gov/Taxonomy/Utils/wprintgc.cgi

Yeast ORFs were retrieved from NCBI from: sgd-archive.yeastgenome.org/?prefix=sequence/S288C_reference/

This release is Genome Release 64-3-1.

The ORF files have the following counts:

| | |
|---|---|
| Total records: | 6034 |
| . . . excluding mitochondrial genes | 6015 (excludes 19 mitochondrial) |
| . . . excluding transposable_element_gene | 5924 (excludes 91 transposable elements) |
| . . . excluding pseudogenes | 5912 (excludes 12 pseudogenes) |
| . . . excluding blocked_reading_frames | 5906 (excludes 6 blocked reading frames) |

Mitochondrial genes are excluded because the application is to the nuclear genome, not the mitochondrial genome. Codon usage in the nuclear and mitochondrial genome are different, and in some organisms the genetic codes are different.

The transposable element genes are excluded for two reasons. First, transposable elements are parasitic DNA that may be better to be removed. Therefore, they may not be retained in a rewritten genome. Second, transposable elements have very similar DNA sequences because of recent common ancestors. Their codon usage does not necessarily match the codon usage of the rest of the yeast genome. This can create a spurious statistical signal.

Pseudogenes are excluded because mutations are free to occur in non-functional DNA.

Codon counts, amino acids counts, and relative synonymous codon usage (RSCU)

The codon count for each codon, including stop codons is then determined. For simplicity, when writing “for each amino acid”, the stop symbols and their codons UAA, UAG, and UGA are included as among the amino acids. The translation table for the organism is used—see Tables 6A and 6B (translation table 1 for yeast or the standard table from the website provided above)-to map codons to amino acids. The number of codons for each amino acid is determined. Then for each codon, the RSCU is determined (e.g., as the number of counts for the codon divided by the number of counts for all codons for the same amino acid).

Results for yeast are based on 2,832,327 codons and are in the Table 6C (amino acid counts), Table 6D (codon counts and RSCU for the original yeast genome), and Table 6E (codon counts and RSCU for the yeast genome after rewriting).

TABLE 6A
The Standard Code-format 1 (transl_table = 1)

| | | |
|---|---|---|
| AAs | = | FFLLSSSSYY**CC*WLLLLPPPPHHQQRRRRIIIMTTTTNNKKSSRRVVVVAAAADDEEGGGG |
| Starts | = | ---M------**--*----M---------------M---------------------------- |
| Base1 | = | TTTTTTTTTTTTTTTTCCCCCCCCCCCCCCCCAAAAAAAAAAAAAAAAGGGGGGGGGGGGGGGG |
| Base2 | = | TTTTCCCCAAAAGGGGTTTTCCCCAAAAGGGGTTTTCCCCAAAAGGGGTTTTCCCCAAAAGGGG |
| Base3 | = | TCAGTCAGTCAGTCAGTCAGTCAGTCAGTCAGTCAGTCAGTCAGTCAGTCAGTCAGTCAGTCAG |

TABLE 6B
The Standard Code - format 2 (transl_table = 1)
Codon/Amino Acid (1 letter code)/Amino Acid (3 letter code)

| | | | |
|---|---|---|---|
| TTT F Phe | TCT S Ser | TAT Y Tyr | TGT C Cys |
| TTC F Phe | TCC S Ser | TAC Y Tyr | TGC C Cys |
| TTA L Leu | TCA S Ser | TAA * Ter | TGA * Ter |
| TTG L Leu i | TCG S Ser | TAG * Ter | TGG W Trp |
| CTT L Leu | CCT P Pro | CAT H His | CGT R Arg |
| CTC L Leu | CCC P Pro | CAC H His | CGC R Arg |
| CTA L Leu | CCA P Pro | CAA Q Gln | CGA R Arg |
| CTG L Leu i | CCG P Pro | CAG Q Gln | CGG R Arg |
| ATT I Ile | ACT T Thr | AAT N Asn | AGT S Ser |
| ATC I Ile | ACC T Thr | AAC N Asn | AGC S Ser |
| ATA I Ile | ACA T Thr | AAA K Lys | AGA R Arg |
| ATG M Met i | ACG T Thr | AAG K Lys | AGG R Arg |
| GTT V Val | GCT A Ala | GAT D Asp | GGT G Gly |
| GTC V Val | GCC A Ala | GAC D Asp | GGC G Gly |
| GTA V Val | GCA A Ala | GAA E Glu | GGA G Gly |
| GTG V Val | GCG A Ala | GAG E Glu | GGG G Gly |

i: initiation,
* and ter: termination

TABLE 6C
Results (Amino Acid Count)

| | Amino acid | Amino acid |
|---|---|---|
| Amino acid (aa) | count (aa_cnt) | frequency (aa_freq) |
| * | 5906 | 0.0020852112061919403 |
| A | 156235 | 0.055161356721875686 |
| C | 36213 | 0.012785599967800328 |
| D | 165319 | 0.05836861351108117 |
| E | 186296 | 0.06577489110544087 |
| F | 126645 | 0.044714116696271296 |
| G | 141776 | 0.05005636707908374 |
| H | 60133 | 0.021230952499481873 |
| I | 184781 | 0.06523999524066254 |
| K | 207688 | 0.07332769132942629 |
| L | 270338 | 0.09544731240425276 |
| M | 58747 | 0.020741602223189624 |
| N | 172355 | 0.060852789949748035 |
| P | 121763 | 0.04299044566534867 |
| Q | 110962 | 0.03917697356272775 |
| R | 126042 | 0.044501217550092204 |
| S | 253263 | 0.0894187005949525 |
| T | 165332 | 0.05837320337658752 |
| V | 158480 | 0.05595399118816436 |
| W | 29606 | 0.010452889090842972 |
| Y | 94447 | 0.03334607903677789 |

TABLE 6D
Codon counts and RSCU for the original yeast genome

| Amino acid (aa) | Codon | Count (cnt) | RSCU |
|---|---|---|---|
| * | TAA | 2831 | 0.47934304097527936 |
| * | TAG | 1337 | 0.22637995259058585 |
| * | TGA | 1738 | 0.2942770064341348 |
| A | GCA | 46042 | 0.2946970909207284 |
| A | GCC | 34904 | 0.22340704707651934 |
| A | GCG | 17863 | 0.11433417608090377 |
| A | GCT | 57426 | 0.3675616859218485 |
| C | TGC | 13632 | 0.37643940021539224 |
| C | TGT | 22581 | 0.6235605997846078 |
| D | GAC | 57173 | 0.3458344170966435 |
| D | GAT | 108146 | 0.6541655829033566 |
| E | GAA | 130199 | 0.6988824236698588 |
| E | GAG | 56097 | 0.3011175763301413 |
| F | TTC | 51434 | 0.4061273638911919 |
| F | TTT | 75211 | 0.5938726361088081 |
| G | GGA | 31715 | 0.2236979460557499 |
| G | GGC | 28033 | 0.1977274009705451 |
| G | GGG | 17610 | 0.12421002144227514 |
| G | GGT | 64418 | 0.45436463153142986 |
| H | CAC | 21452 | 0.35674255400528826 |
| H | CAT | 38681 | 0.6432574459947117 |
| I | ATA | 51494 | 0.2786758378837651 |
| I | ATC | 47709 | 0.25819213014325065 |
| I | ATT | 85578 | 0.46313203197298425 |
| K | AAA | 120304 | 0.5792534956280575 |
| K | AAG | 87384 | 0.42074650437194255 |
| L | CTA | 38282 | 0.14160791305698792 |
| L | CTC | 15611 | 0.057746228795063956 |
| L | CTG | 30580 | 0.11311765271622931 |
| L | CTT | 34723 | 0.12844291220620113 |
| L | TTA | 74606 | 0.27597304115588633 |
| L | TTG | 76536 | 0.28311225206963136 |
| M | ATG | 58747 | 1.0 |
| N | AAC | 69568 | 0.40363203852513707 |
| N | AAT | 102787 | 0.5963679614748629 |
| P | CCA | 49607 | 0.40740619071474915 |
| P | CCC | 19542 | 0.1604921035125613 |
| P | CCG | 14967 | 0.12291911335955914 |
| P | CCT | 37647 | 0.30918259241313045 |
| Q | CAA | 75790 | 0.6830266217263568 |
| Q | CAG | 35172 | 0.31697337827364325 |
| R | AGA | 59762 | 0.4741435394551023 |
| R | AGG | 27339 | 0.21690388917979722 |
| R | CGA | 8607 | 0.06828676155567191 |
| R | CGC | 7460 | 0.05918662033290491 |
| R | CGG | 5261 | 0.041740054902334144 |
| R | CGT | 17613 | 0.13973913457418954 |
| S | AGC | 28536 | 0.11267338695348314 |
| S | AGT | 41333 | 0.16320188894548354 |
| S | TCA | 52989 | 0.209225192783786 |
| S | TCC | 39767 | 0.15701859331998752 |
| S | TCG | 24681 | 0.09745205576811457 |
| S | TCT | 65957 | 0.2604288822291452 |
| T | ACA | 50246 | 0.3039097089492657 |
| T | ACC | 35028 | 0.21186461181138558 |
| T | ACG | 23190 | 0.1402632279292575 |
| T | ACT | 56868 | 0.34396245131009123 |
| V | GTA | 34101 | 0.21517541645633517 |
| V | GTC | 31930 | 0.20147652700656235 |
| V | GTG | 31087 | 0.1961572438162544 |
| V | GTT | 61362 | 0.38719081272084804 |
| W | TGG | 29606 | 1.0 |
| Y | TAC | 41031 | 0.4344341270765615 |
| Y | TAT | 53416 | 0.5655658729234385 |

TABLE 6E
Codon counts and RSCU for the yeast genome after rewriting
(0 indicates that the codon has been eliminated)

| Amino acid (aa) | Codon | Count (cnt) | RSCU |
|---|---|---|---|
| * | TAA | 0 | 0.0 |
| * | TAG | 0 | 0.0 |
| * | TGA | 5906 | 1.0 |
| A | GCA | 46042 | 0.2946970909207284 |
| A | GCC | 34904 | 0.22340704707651934 |
| A | GCG | 17863 | 0.11433417608090377 |
| A | GCT | 57426 | 0.3675616859218485 |
| C | TGC | 13632 | 0.37643940021539224 |
| C | TGT | 22581 | 0.6235605997846078 |
| D | GAC | 57173 | 0.3458344170966435 |
| D | GAT | 108146 | 0.6541655829033566 |
| E | GAA | 130199 | 0.6988824236698588 |
| E | GAG | 56097 | 0.3011175763301413 |
| F | TTC | 51434 | 0.4061273638911919 |
| F | TTT | 75211 | 0.5938726361088081 |
| G | GGA | 31715 | 0.2236979460557499 |
| G | GGC | 28033 | 0.1977274009705451 |
| G | GGG | 17610 | 0.12421002144227514 |
| G | GGT | 64418 | 0.45436463153142986 |
| H | CAC | 21452 | 0.35674255400528826 |
| H | CAT | 38681 | 0.6432574459947117 |
| I | ATA | 51494 | 0.2786758378837651 |
| I | ATC | 47709 | 0.25819213014325065 |
| I | ATT | 85578 | 0.46313203197298425 |
| K | AAA | 120304 | 0.5792534956280575 |
| K | AAG | 87384 | 0.42074650437194255 |
| L | CTA | 0 | 0.0 |
| L | CTC | 15718 | 0.058142029607380394 |
| L | CTG | 0 | 0.0 |
| L | CTT | 37985 | 0.1405092883723339 |
| L | TTA | 104383 | 0.38612033824323627 |
| L | TTG | 112252 | 0.4152283437770495 |
| M | ATG | 58747 | 1.0 |
| N | AAC | 69568 | 0.40363203852513707 |
| N | AAT | 102787 | 0.5963679614748629 |
| P | CCA | 49607 | 0.40740619071474915 |
| P | CCC | 19542 | 0.1604921035125613 |
| P | CCG | 14967 | 0.12291911335955914 |
| P | CCT | 37647 | 0.30918259241313045 |
| Q | CAA | 75790 | 0.6830266217263568 |
| Q | CAG | 35172 | 0.31697337827364325 |
| R | AGA | 71852 | 0.5700639469383222 |
| R | AGG | 28218 | 0.22387775503403629 |
| R | CGA | 0 | 0.0 |
| R | CGC | 7545 | 0.05986099871471414 |
| R | CGG | 0 | 0.0 |
| R | CGT | 18427 | 0.14619729931292744 |
| S | AGC | 30587 | 0.12077168792914875 |
| S | AGT | 51674 | 0.20403296178281075 |
| S | TCA | 0 | 0.0 |
| S | TCC | 50208 | 0.19824451262126722 |
| S | TCG | 0 | 0.0 |
| S | TCT | 120794 | 0.47695083766677326 |
| T | ACA | 50246 | 0.3039097089492657 |
| T | ACC | 35028 | 0.21186461181138558 |
| T | ACG | 23190 | 0.1402632279292575 |
| T | ACT | 56868 | 0.34396245131009123 |
| V | GTA | 34101 | 0.21517541645633517 |
| V | GTC | 31930 | 0.20147652700656235 |
| V | GTG | 31087 | 0.1961572438162544 |
| V | GTT | 61362 | 0.38719081272084804 |
| W | TGG | 29606 | 1.0 |
| Y | TAC | 41031 | 0.4344341270765615 |
| Y | TAT | 53416 | 0.5655658729234385 |

### Ninemers (9Mers) and Codon-Aa-Codon Contexts

Next, the frequency of 9 mers in coding domains is determined. The 9 mers are in-frame sliding windows across the coding sequence (CDS). A CDS with n amino acids (including the stop codon) may have (n−2) different 9 mers. The total number of 9 mers determined is 2,820,515 and the number of unique 9 mers is 215,766. The maximum number of unique 9 mers is not 64*64*64=262,144, but rather 61*61*64=238,144, because stop codons can only occur in the third position. The actual number observed is smaller because some codon patterns are too rare to be observed.

Codon-codon-codon patterns are then converted to contexts, which may be determined as a codon-aa-codon patterns. There are 61*20*64=78,080 possible contexts, of which 75,918 are observed in the yeast genome.

Next for each context, a test of the null hypothesis is performed that the frequency of the central codon, conditioned on the context of the surrounding codons, follows the same distribution as the RSCU. This is performed as a single statistical test for all the possible central codons given the central amino acid.

The test is motivated by considering a likelihood ratio test with test statistic

$$Q = 2 ln [ Pr ( D \vert ML ) / Pr ( D \vert null ) ] ,$$

where Pr(D|null) is the probability of central codon counts under the null distribution given by the genome-wide RSCU, and Pr(D|ML) is the probability of the central codon counts under an alternative distribution in which the codon usage depends on the context defined by the outer codons, using the maximum likelihood estimator for the model parameters. Under the null, Q follows a chi-square distribution with a number of degrees of freedom (df) equal to the number of possible codons minus 1. Thus, for amino acids with a single amino acid, the test has 0 df (only a single choice), amino acids with 2 codons have 1 df, amino acids with 4 codons have 3 df, and amino acids with 6 codons have 5 df. The stop signal has 3 codons and 2 df.

For a given context, let c be one of the possible codons, r(c) be the RSCU for that codon, and n(c) be the number of times that codon occurs in the central position of that context. Under the null,

$$Pr ( D \vert null ) = Product\_c {r ( c )}^{∧} n ( c )$$

$$ln Pr ( D \vert null ) = Sum\_c n ( c ) ln r ( c )$$

For the ML distribution, the standard result is that the maximum likelihood probabilities are the observed probabilities. Let N=sum_c n(c) be the number of examples of the context. The maximum likelihood estimate for the frequency of codon c is determined as:

$$r ’ ( c ) = n ( c ) / N , and ln Pr ( D \vert ML ) = Sum\_c n ( c ) ln [ n ( c ) / N ] .$$

Putting this together,

$$Q = 2 Sum\_c n ( c ) ln [ n ( c ) / N r ( c ) ] .$$

Note that the argument of the logarithm is the ratio of the number of codons observed to the number expected under the null.

In the case that a particular codon is not observed,

$$n ( c ) ln [ n ( c ) ] = 0.$$

There are no problems with divergences. Other statistical tests are possible, including using pseudocounts to smooth out the distributions.

The single-tailed p-value is then determined for the chi-square values to identify contexts whose codon usage differs from the null. For a stringent family-wise error of 0.05, an individual test p-value is required to be smaller than 0.05/78,080=6.4E-7.

The likelihood ratio test is asymptotic to a chi-square distribution, but for small values of observations there are standard corrections. Therefore, a chisquare test is also performed as implemented by scipy.stats.chisquare, which takes as arguments the same lists of observed and expected counts, including the zero counts. The test statistics and p-values may be very similar.

A small p-value can result from many observations with a small difference between observed and expected counts, or from fewer observations with a larger difference between observed and effected counts. The difference is quantified as a weighted geometric mean of the observed-to-expected ratio magnitudes as follows.

Let n(c) be the number of occurrences of codon c as before, and N r(c) be the null expectation as before. The weighted log-ratio w is determined as:

$$w = ( 1 / N ) Sum\_c n ( c ) \vert ln [ n ( c ) / N r ( c ) ] \vert$$

where the vertical bars indicate absolute value. The absolute value is taken to count both enrichment, n(c) higher than expected, and depletion, n(c) lower than expected, as contributing their magnitudes rather than cancelling each other out.

The ratio magnitude R is then determined as:

$$R = exp ( w ) .$$

For a context with a small p-value and large ratio magnitude, it is instructive to examine the under-represented codon choices and over-represented codon-choices. For a codon c, the regularized log-ratio is determined as:

$$LR ( c ) = ln [ max ( n ( c ) , 0.5 ) / N r ( c ) ] ,$$

which is just the log ratio, but with n(c) changed from 0 to 0.5 for codons that are never observed. Then, within each context, the 9 mer patterns with the most negative LR and the most positive LR are provided.

Contexts, their observed and null hypothesis counts of central codons, p-values, and ratios are provided in Table 6F (context_cnt.txt as tab-delimited text). Amino acids with a single codon are included in the results. For these amino acids, observed and expected counts are identical, and all p-values are set to 1.

The number of contexts with p-value below 6.4E-7 is 584. The rows of the context_cnt.txt belonging to this subset are provided in Table 9. A few of the patterns observed are discussed.

### Depletion of Ribosomal Frameshifting Slippery Sites

One pattern of depleted codon use is to avoid creating codon patterns that are slippery sites for ribosomal frameshifting. An exemplary pattern for a slippery site is:

nnX XXY YYZ

where spaces indicate codon boundaries, X and Y may be A or T, YYZ may be AAC or TTA, and the small n's at the beginning of the pattern may be any nucleotides. This site promotes a −1 frameshift in which the new codon boundaries are:

nn XXX YYY X.

Note that in both the original reading frame and in the −1 frameshift, the first two codon position are XX in the second codon and YY in the third codon. The only changes in base pairing are to the wobble position codon.

See, for example, these references:
T Jacks, HD Madhani, FR Masiars, HE Varmus 1988 Cell 55: 447, which is incorporated by reference herein in its entirety.
M Chamorro, N Parkin, HE Varmus 1992 PNAS 89: 713, which is incorporated by reference herein in its entirety.
JN Dinman 1995 Yeast 11: 1115, which is incorporated by reference herein in its entirety

An example is the context GAA_K_AAA encoding the three amino acids E_K_K. There are two possible choices for the lysine codon, AAA (195 observed, 312 expected) and AAG (343 observed, 226 expected). The 1.5-fold change from the expected distribution is highly significant, p=2.eE-24.

A second example is the context GGT_G_GGT encoding the three amino acids G_G_G. The most depleted central codon is GGG (5 observed, 28 expected), and the most enriched is GGT (172 observed, 102 expected). The mean ratio magnitude is 1.8, p=1.8E-19.

A third example is the context CTC_P_TTG encoding the three amino acids L_P_L. The most depleted central codon is CCT (0 observed, 3 expected). This creates a possible slippery site with a −1 frameshift:

CTC CCT TTG−>CT CCC TTT C

The most enriched is CCC (22 observed, 4 expected), which eliminates the slippery site.

TABLE 6F
Contexts, their observed and null hypothesis counts of central codons,
p-values, and ratios

| | con- | | | codon_ | | | | | | | | | | |
|---|---|---|---|---|---|---|---|---|---|---|---|---|---|---|
| | text_ | | codon_ | cnt_ | | | | | | context_ | | most_ | most_ | codon_ |
| context | aa | cnt | cnt | null | df | q | spq | pval | sppval | cnt | ratio | depleted | | order |
| GAA_ | E | A | 1 | 31 | 1 | 102. | 103.7 | 4.893 | 2.288 | 53 | 1.544 | 1.6 | 1.5 | AA |
| K_ | K̅ | A | 9 | 1. | | 2501 | 55590 | 48489 | 78321 | 8 | 80286 | :GA | :GA | G |
| AAA | K̅ | A | 5 | 63 | | 6669 | 75673 | 08515 | 97377 | | 32158 | AAA | AAA | AA |
| | | A | 3 | 8 | | 5166 | 644 | 474e- | 122e- | | 135 | AAA | GAA | A |
| | | A | 4 | 22 | | 8 | | 24 | 24 | | | A | A | |
| | | G | 3 | 6. | | | | | | | | | | |
| | | | | 36 | | | | | | | | | | |
| | | | | 2 | | | | | | | | | | |
| GG | G | G | 2 | 50 | 3 | 98.0 | 90.42 | 4.027 | 1.776 | 22 | 1.781 | 5.6 | 1.7 | GG |
| T_ | G̅ | G | 1 | .3 | | 7647 | 34499 | 62466 | 65687 | 5 | 49997 | :GG | :GG | T |
| G_ | G̅ | A | 2 | 32 | | 4297 | 99975 | 99139 | 96921 | | 38840 | TGG | TGG | GG |
| GG | | G | 7 | 44 | | 3268 | 99 | 65e- | 948e- | | 842 | GGG | TGG | C |
| T | | G | 5 | .3 | | | | 21 | 19 | | | T | T | GG |
| | | C | 1 | 89 | | | | | | | | | | A |
| | | G | 7 | 27 | | | | | | | | | | GG |
| | | G | 2 | .9 | | | | | | | | | | G |
| | | G | | 47 | | | | | | | | | | |
| | | G | | 10 | | | | | | | | | | |
| | | G | | 2. | | | | | | | | | | |
| | | T | | 23 | | | | | | | | | | |
| | | | | 2 | | | | | | | | | | |
| TT | L | C | 5 | 27 | 3 | 34.0 | 32.74 | 1.890 | 3.647 | 67 | 1.913 | 5.4 | 1.8 | CC |
| A_ | P̅ | C | 0 | .3 | | 9657 | 36822 | 33174 | 59611 | | 51802 | :TT | :TT | A |
| P_ | R̅ | A | 2 | 96 | | 1086 | 53844 | 73552 | 90134 | | 95466 | ACC | ACC | CC |
| AG | | C | 4 | 10 | | 0602 | 6 | 026e- | 545e- | | 208 | CAG | AAG | T |
| A | | C | 1 | .7 | | 5 | | -7 | 07 | | | A | A | CC |
| | | C | 1 | 53 | | | | | | | | | | G |
| | | C | | 8. | | | | | | | | | | CC |
| | | C | | 23 | | | | | | | | | | C |
| | | G | | 6 | | | | | | | | | | |
| | | C | | 20 | | | | | | | | | | |
| | | C | | .7 | | | | | | | | | | |
| | | T | | 15 | | | | | | | | | | |

### Regulatory Signals

Some patterns of context-dependent codon usage match regulatory signal sequences. An example is the ACCCA sequence recognized by the Raplp binding protein:
D Shore 1994 Trends in Genetics 10: 408, which is incorporated by reference herein in its entirety.

This sequence can cause transcriptional silences, and inadvertent creation of a Raplp binding site created a fitness defect in Sc2.0 synthetic chromosome synX:
Y Wu et al 2017 Science 355: 1048, which is incorporated by reference herein in its entirety.

The context TTA_P_AGA, with amino acids L_P_R, has a depleted central codon CCC (2 observed, 11 expected) that creates the ACCCA Rap1p binding motif. The most enriched central codon is CCA (50 observed, 27 expected), with a mean ratio magnitude 1.9 and p=3.7E-7.

### Implementation

The inspiration for Goldilocks is codon usage that is not too hot, not too cold, but just right for the context. Given a set of codons to avoid throughout the genome, the codon is mapped to the amino acid, and then a replacement codon is determined based at least in part on statistical analysis of a local context of the replacement codon.

A one-pass Goldilocks algorithm is performed as follows, processing each CDS in turn:
1. Identify the positions of codons to eliminate.
2. Consider each codon in turn, replacing the codon with the most frequently used codon as the central codon in a 3-codon context.
3. The first codon is a special case because there is no preceding context. The first codon is always ATG, however, in standard genetic codes.
4. The last codon (stop codon) is a special case because there is no following context. If stop codons are rewritten, however, an example design is to change TAA and TAG to TGA, which has only a single choice. Alternatively, a 6nt context or 9nt context with the stop codon as the final 3nt may be used.

An implementation of a one-pass Goldilocks algorithm is provided, along with sample input and output for the entire yeast genome. The codons removed are as follows (Table 7):

TABLE 7
Codons for removal

| Amino acid | Codon |
|---|---|
| * | TAA |
| * | TAG |
| R | CGA |
| R | CGG |
| L | CTA |
| L | CTG |
| S | TCA |
| S | TCG |

The method rewrites 164,568 out of 2,832,327 codons=5.8% of the total codons.

The output CDS records are validated to lack any instances of the codons, and the translation of the CDS is validated to be identical to the original translation.

### Dynamic Programming Approach for Evaluation of Codons to Rewrite

The one-pass method described above is appropriate for separated instances of codons to rewrite. If adjacent codons are in the rewrite set, however, then rewriting one changes the context for the other. There are many instances of this in the yeast genome. For each CDS, the maximum run length of codons to rewrite was determined. These are the rewrite lengths and numbers of genes (Table 8):

TABLE 8
Rewrite Length and number of genes

| | maxrunlen count |
|---|---|
| 0 | 13 |
| 1 | 1914 |
| 2 | 3176 |
| 3 | 707 |
| 4 | 68 |
| 5 | 16 |
| 6 | 5 |
| 7 | 3 |
| 8 | 1 |
| 9 | 2 |
| 13 | 1 |

The gene with the longest run length of 13 codons in a row is YGR130C SGDID:S000003362, Chr VII from 753844-751394, Genome Release 64-3-1, reverse complement, Verified ORF, “Component of the eisosome with unknown function; GFP-fusion protein localizes to the cytoplasm; specifically phosphorylated in vitro by mammalian diphosphoinositol pentakisphosphate (IP7)”, which is incorporated by reference herein in its entirety.

This is the protein sequence with a run of 16 serine residues highlighted in bold, with many encoded by TCA and TCG codons in the set to be rewritten.

>YGR130C
(SEQ ID NO: 11,814)
MLFNINRQEDDPFTQLINQSSANTQNQQAHQQESPYQFLQKVVSNEPK
GKEEWVSPFRQDALANRQNNRAYGEDAKNRKFPTVSATSAYSKQQPKD
LGYKNIPKNAKRAKDIRFPTYLTQNEERQYQLLTELELKEKHLKYLKK
CQKITDLTKDEKDDTDTTTSSSTSTSSSSSSSSSSSSSSSSDEGDVTS
TTTSEATEATADTATTTTTTTSTSTTSTSTTNAVENSADEATSVEEEH
EDKVSESTSIGKGTADSAQINVAEPISSENGVLEPRTTDQSGGSKSGV
VPTDEQKEEKSDVKKVNPPSGEEKKEVEAEGDAEEETEQSSAEESAER
TSTPETSEPESEEDESPIDPSKAPKVPFQEPSRKERTGIFALWKSPTS
SSTQKSKTAAPSNPVATPENPELIVKTKEHGYLSKAVYDKINYDEKIH
QAWLADLRAKEKDKYDAKNKEYKEKLQDLQNQIDEIENSMKAMREETS
EKIEVSKNRLVKKIIDVNAEHNNKKLMILKDTENMKNQKLQEKNEVLD
KQTNVKSEIDDLNNEKTNVQKEFNDWTTNLSNLSQQLDAQIFKINQIN
LKQGKVQNEIDNLEKKKEDLVTQTEENKKLHEKNVQVLESVENKEYLP
QINDIDNQISSLLNEVTIIKQENANEKTQLSAITKRLEDERRAHEEQL
KLEAEERKRKEENLLEKQRQELEEQAHQAQLDHEQQITQVKQTYNDQL
TELQDKLATEEKELEAVKRERTRLQAEKAIEEQTRQKNADEALKQEIL
SRQHKQAEGIHAAENHKIPNDRSQKNTSVLPKDDSLYEYHTEEDVMYA*

A dynamic programming optimization proceeds as follows. Suppose a sequence of n codons, numbered 1 through n, must be rewritten. Denote c(1) as a permitted codon for position 1, which means that it encodes the same amino acid as the original codon and it is not in the set of codons to remove. Similarly c(2) is a permitted codon for position 2, and so on. Codons c0 and c(ₙ₊₁) are fixed by the pre-existing codons, which by definition are outside the set to be removed. As described above, the boundary case that c(1) is the start codon should not occur because ATG is the only start codon. The boundary case that c(n) is the stop codon is a special case in which our favored design uses only a single stop codon, TGA.

Denote the score for a codon as a value that increases monotonically with our preference for the context with that codon in the middle. Scores should be additive. A suitable value for the score of a codon given its context is In [n(c)], the number of times the codon is observed to occur in that context.

Denote Context[x, y, z] as this type of additive score for the choice of codon y given the amino acid required and the flanking codons x and z.

Denote S[c(1), c(2)] as the best score for codons through position 1 that have position 1 set to c(1) and position 2 set to c(2). This can be determined by enumeration.

Then S[c(2), c(3)]=max_c(1) S[c(1), c(2)]+Context[c(1), c(2), c(3)], which is the best score for having position c(2) and c(3) as specified.

This process continues,

S[c(n), c(ₙ₊₁)]max_c(ₙ₋1) S[c(ₙ₋1), c(n)]+Context[c(ₙ₋1), c(n), c(ₙ₊₁)], which is the best score for having position c(n) and c(ₙ₊₁) as specified.

The search ends here because the codon c(ₙ₊₁) is not in the set to be removed. The traceback of the maximum values leading to this last step provides the codons that together optimize an objective function corresponding to context-dependent codon usage.

### Other Extensions

Alternatively or in combination, one or more of the following algorithm choices may be used:

Use dynamical programming for a more sophisticated treatment of neighboring codons.

Use a different codon selection strategy, for example maintaining GC content, codon adaptation index, or translational efficiency, as the main codon replacement rule, but if this may result in the creation of a pattern that is depleted with statistical significance or other relevant criterion, use the Goldilocks-selected codon instead.

Use the Goldilocks codon with the greatest fold-enrichment over the null hypothesis, rather than the Goldilocks codon that is most often used in the context.

Use a random codon selected using the Goldilocks context-dependent probabilities as the probability distribution.

The final codon is a stop codon and a special case. Some designs may be a single choice for the stop codon, TGA, or a pair of choices, TGA and TAAₙ For the stop codon, a 9 mer pattern or 6 mer pattern ending with the stop codon may be used instead of the 9 mer pattern with the codon of interest in the middle position.

Avoid significantly enriched codons as possible regulatory signals, choosing a codons whose usage matches the overall RSCU and is not too hot, not too cold, but just right.

These and other methods that determine context-dependent codon usage values and use them as the basis for codon selection may be used.

The sequences of original yeast ORFs (Saccharomyces cerevisiae S288C strain) and rewritten yeast ORFs using methods described herein are shown as SEQ ID NOs: 1-11,812.

### Example 5: Orthogonal Translation System

This example shows site-specific incorporation of ncAAs in proteins in Yeast using generic orthogonal translation system with both displayed and intracellular proteins in the yeast display strain RJY100. ncAA incorporation systems comprise a protein construct containing a TAG codon, an orthogonal translation system, and a ncAA added during expression of the protein construct. This method can be adapted for use in other yeast strains, and plasmids encoding the protein of interest and plasmids encoding the orthogonal translation systems need to contain unique selection markers that must be compatible with the genotype of the yeast strain.

### Materials

1. One or more yeast display vectors containing a protein of interest (POI) with and without a TAG stop codon at a permissible site under a galactose-inducible promoter are prepared. The vectors can be named pPOIVector-POI-TAG (with a TAG stop codon) and pPOIVector-POI (without a TAG stop codon), respectively. The vectors also contain an autotrophic marker, e.g., tryptophan marker, for use in yeast and an antibiotic marker, e.g., ampicillin marker, for propagation in E. coli.

2. One or more galactose-inducible vectors for a dual-fluorescent protein construct consisting of a fluorescent protein, e.g., blue fluorescent protein and superfolder green fluorescent protein connected by a linker sequence, with or without a TAG codon (BXG and BYG, respectively) are prepared. These vectors can be named pPOIVector-BXG and pPOIVector-BYG, respectively. The vectors also contain an autotrophic marker, e.g., tryptophan marker, for use in yeast and an antibiotic marker, e.g., ampicillin marker, for propagation in E. coli.

3. One or more galactose-inducible vector for a single-fluorescent protein construct consisting of a fluorescent protein, e.g., superfolder green fluorescent protein containing a TAG codon in place of tyrosine at position 151 are prepared. These vectors can be named pPOIVector-GFP-TAG and pPOIVector-GFP, respectively. The vectors also contain an autotrophic marker, e.g., tryptophan marker, for use in yeast and an antibiotic marker, e.g., ampicillin marker, for propagation in E. coli.

4. One or more constitutive expression vector for orthogonal translation system comprised of an aminoacyl-tRNA synthetase and cognate tRNA is prepared (pOTSVector-OTS). The vectors also contain an autotrophic marker, e.g., leucine marker, for use in yeast and an antibiotic marker, e.g., ampicillin marker, for propagation in E. coli.

5. Saccharomyces cerevisiae yeast display strain RJY100 is prepared for use with conventional yeast display and intracellular fluorescent protein expression.

6. Media preparation:

### Media Preparation

A) SD-SCAA-TRP-LEU-URA and SD-SCAA-TRP-URA media, pH 4.5: Dissolve 20 g glucose, 6.7 g yeast nitrogen base without amino acids, 2 g synthetic casamino acids (-TRP-LEU-URA or -TRP-URA), and citrate buffer salts (10.4 g sodium citrate, 7.4 g citric acid monohydrate) in 1 L ddH2O. Filter sterilize using a 0.2 m filter and store at room temperature.

B) SD-SCAA-TRP-LEU-URA and SD-SCAA-TRP-URA plates, pH 6.0: Mix phosphate buffer salts (5.4 g sodium phosphate dibasic, anhydrous, and 8.56 g sodium phosphate monobasic monohydrate), 15 g agar, and 182 g sorbitol in a final volume of 900 mL with ddH2O in a 1 L bottle with a magnetic stir bar. Autoclave the mixture and cool with stirring at room temperature. At the same time, dissolve 20 g glucose, 6.7 g yeast nitrogen base without amino acids, and 2 g synthetic casamino acids (-TRP-LEU-URA or -TRP-URA) in a final volume of 100 mL using vigorous stirring. Once the autoclaved solution has cooled to approximately 60° C., filter sterilize the glucose/yeast nitrogen base/synthetic casamino acid mixture directly into the autoclaved solution, mix briefly, and pour plates. This recipe is expected to produce approximately 80-100, 100 mm plates. Store at room temperature or at 4° C.

C) SG-SCAA-TRP-LEU-URA and SG-SCAA-TRP-URA media, pH 6.0: Dissolve 20 g galactose, 2 g glucose, 6.7 g yeast nitrogen base without amino acids, 2 g synthetic casamino acids (-TRP-LEU-URA or -TRP-URA), and phosphate buffer salts (5.4 g sodium phosphate dibasic, anhydrous, and 8.56 g sodium phosphate monobasic monohydrate) in 1 L ddH2O. Filter sterilize using a 0.2 m filter and store at room temperature.

D) Yeast Extract-Peptone-Dextrose (YPD) media: Mix 20 g peptone and 10 g yeast extract in 900 mL ddH2O. Separately, prepare a solution of 100 mL 20% glucose (20 g glucose in 100 mL ddH2O). Autoclave both solutions, let them cool, and combine the two to make the final product (see Note 11). Store at room temperature.

E) Yeast Extract Peptone-Glycerol (YPG) media: Mix 20 g peptone and 10 g yeast extract in 900 mL ddH2O. Separately, prepare a solution of 100 mL 20% galactose (20 g galactose in 100 mL ddH2O). Autoclave both solutions, let them cool, and combine the two to make the final product. Store at room temperature.

F) YPD plates: Mix 10 g peptone, 5 g yeast extract, and 7.5 g agar in 450 mL ddH2O in a 1 L bottle with a magnetic stir bar. Separately, make a solution of 50 mL 20% glucose (10 g in 50 mL). Autoclave both solutions, cool both solutions to 55° C. with stirring, mix them together, and pour plates. This recipe is expected to produce approximately 40-50, 100 mm plates. The 20% glucose solution can be made ahead of time. Store at room temperature or at 4° C.

7. Other reagents to be prepared:

A) Penicillin-streptomycin: 10,000 IU/mL and 10,000 μg/mL, respectively, in 100×solution

B) 50 mM noncanonical amino acid (ncAA): Prepare a 50 mM liquid stock of the L-isomer of the ncAAs by dissolving the ncAA in 90% of the final volume ddH2O and vortexing thoroughly. The addition of NaOH may be required to fully dissolve the ncAA. Add ddH2O to a final volume and sterile filter using a 0.2 m filter before use. Use immediately or store at 4° C.

8. Kits, containers and instruments needed:

A) Zymo Research Frozen-EZ Yeast Transformation II Kit (Zymo Research).

B) Cryoprotectant isopropanol containers to slow-freeze competent yeast cells. An example of a suitable isopropanol container is the Thermo Scientific™ Mr. Frosty™ (Thermo Fisher catalog number 5100-0001).

C) Sterile 1.7 mL microcentrifuge tubes.

D) Sterile polyethylene culture tubes.

E) Sterile 15 mL polypropylene conical tubes.

F) Benchtop vortexer.

G) Benchtop centrifuge for spinning culture tubes.

H) Stationary incubator at 30° C. (for yeast plate incubation).

I) Shaking incubator at 30° C., 300 rpm (for yeast liquid culture growth).

J) Shaking incubator at 20° C., 300 rpm (for induction of liquid cultures).

K) NanoDrop or other spectrophotometer for measuring yeast culture density.

9. Flow Cytometry system for Flow Cytometry- and Microplate Reader-based evaluation of ncAA Incorporation events.

A) Refrigerated benchtop centrifuge for spinning microcentrifuge tubes.

B) Rotary wheel at room temperature.

C) Flow cytometer.

D) Flow cytometry data analysis software.

E) Spectrophotometric microplate reader.

F) Flow cytometry tubes compatible with available flow cytometer.

G) 96-well microplates compatible with available flow cytometer for large-scale experiments (provided that the flow cytometer has an autosampler).

H) Adhesive foil for covering 96-well microplates.

I) Primary antibodies: Chicken anti-c-Myc (Gallus Immunotech) and Mouse anti-HA antibody (BioLegend).

J) Secondary antibodies: Goat anti-chicken Alexa Fluor 647 (Invitrogen); Goat anti-chicken Alexa Fluor 488 (Invitrogen); Goat anti-mouse Alexa Fluor 488 (Invitrogen).

K) 96-well clear bottom black-walled microplates.

10. Bioorthogonal Reactions with ncAAs on the yeast surface.

A) Rotary wheel at 4° C.

B) 1×PBS, pH 7.4: Mix 8 g sodium chloride, 0.2 g potassium chloride, 1.44 g sodium phosphate dibasic (anhydrous), and 0.24 g potassium phosphate monobasic (anhydrous) in 1 L ddH2O. Use hydrochloric acid or sodium hydroxide to adjust the pH to 7.4. Sterile filter using a 0.2 m filter and store at room temperature.

C) Sterile PBS+0.1% bovine serum albumin (BSA), pH 7.4 (PBSA): Add 1 g BSA to 1 L1×PBS, pH 7.4, dissolve, and sterile filter using a 0.2 m filter. Store at room temperature.

D) 20 mM copper sulfide (CuSO4): Dissolve 0.0050 g of CuSO4 powder (MW 249.68 g/mol) in 1 mL ddH2O by vortexing. Store at 4° C.

E) 50 mM Tris(benzyltriazolylmethyl)amine (THPTA): Dissolve 0.0217 g THPTA powder (MW 434.50 g/mol) in 1 mL ddH2O by vortexing. Store at 4° C.

F) 1:2 solution of 20 mM CuSO4: 50 mM THPTA: Combine 20 mM CuSO4 and 50 mM THPTA at a 1:2 volume ratio. Prepare immediately prior to use.

G) 20 mM biotin-(PEG)4-alkyne or biotin-(PEG)4-azide: Dissolve biotin-(PEG)4-alkyne or biotin-(PEG)4-azide in dimethyl sulfoxide (DMSO). Store at −20° C. in a desiccant jar.

H) 200 mM cargo-alkyne or cargo-azide: Dissolve the cargo-alkyne or cargo-azide in ddH2O or DMSO for long-term storage at −20° C.

I) 100 mM aminoguanidine: Dissolve 0.011 g aminoguanidine HCl (MW 110.55 g/mol) in 1 mL ddH2O immediately prior to use.

J) 100 mM sodium ascorbate: Dissolve 0.020 g sodium ascorbate (MW 198.11 g/mol) in 1 mL ddH2O immediately prior to use.

K) 20 mM dibenzocyclooctyne-amine (DBCO)-biotin: Dissolve DBCO-biotin (MW=749.91 g/mol) in DMSO and store at −20° C. Dilute to 2 mM in DMSO prior to use.

L) 200 mM dibenzocyclooctyne-amine (DBCO)-cargo: Dissolve DBCO-cargo in DMSO.

11. Click Chemistry Analysis

A) Secondary antibody: Streptavidin, Alexa Fluor 488 conjugate (Invitrogen).

12. Preparation of Libraries Involving the Use of Orthogonal Translation Systems

A) A yeast display vector pCTCON2 that contains tryptophan marker for use in yeast and ampicillin marker for propagation in E. coli.

B) A constitutive expression vector pRS315-LeuOmeRS for orthogonal translation system comprising an E. coli leucyl-tRNA synthetase mutant and cognate tRNA. This vector contains leucine marker for use in yeast and ampicillin marker for propagation in E. coli.

C) Restriction enzymes NcoI and NdeI for preparing libraries of OTSs in pRS315-LeuOmeRS.

D) Restriction enzymes SalI, NheI, and BamHI for preparing libraries of POIs in pCTCON2.

E) DNA polymerase and corresponding buffers for PCR.

F) 10 mM dNTPs.

G) Thin-walled PCR tubes.

H) Template DNA for library amplification.

I) Primers for template amplification with homologous recombination flanking regions. Each protein library will contain different 5′ and 3′ ends and will need to be designed to accommodate the specific library design.

J) Additional primers needed to construct the library of interest.

K) Forward and reverse pCTCON2 sequencing primers.

L) Forward and reverse pRS315 sequencing primers.

M) Molecular biology-grade agarose.

N) Tris-acetate-EDTA (TAE) buffer (50×): Dissolve 242 g Tris base in ddH2O, then add 57.1 mL glacial acetic acid and 100 mL 500 mM EDTA, pH 8.0, and add ddH2O to 1 L. Store at room temperature.

O) Nucleic acid gel stain, DNA gel loading dye (1×), DNA molecular weight size marker.

P) DNA gel electrophoresis equipment: gel mold and extraction combs, gel box, voltage box, gel imager.

Q) Heat block set to 55° C. for melting agarose containing DNA fragments.

R) Gel extraction kit (Gel extraction buffer for melting agarose gel, DNA purification columns and wash buffers).

S) NanoDrop or other spectrophotometer for measuring DNA concentrations.

T) Sterile ddH2O chilled to 4° C.

U) Pellet Paint co-precipitant (EMD Millipore).

V) 70% ethanol in ddH2O and 100% ethanol.

W) SD-SCAA-LEU-URA media, pH 4.5:

Dissolve 20 g glucose, 6.7 g yeast nitrogen base without amino acids, 2 g synthetic casamino acids [25](-LEU-URA), and citrate buffer salts (10.4 g sodium citrate, 7.4 g citric acid monohydrate) in 1 L ddH2O. Filter sterilize using a 0.2 m filter and store at room temperature.

X) 100 mM lithium acetate (sterile) and 1 M dithiothreitol (DTT)

Y) 50 mL conical tubes and 2 mm electroporation cuvettes chilled on ice prior to use in electroporations

Z) Refrigerated benchtop centrifuge for spinning 50 mL conical tubes and for pelleting large volumes (1 L or greater)

AA) Bio-Rad Gene Pulser XCell Total System (Bio-Rad) or other electroporator with square wave protocol capability.

BB) Sterile 250 mL and 2 L flasks for liquid culture growth.

CC) Autoclavable centrifuge bottles (500 mL or greater capacity).

DD) Sterile 60% glycerol: Prepare a solution of 60% v/v glycerol in ddH2O and autoclave to sterilize. Store at room temperature.

EE) 2 mL cryogenic screw-cap vials.

FF) Zymoprep Yeast Plasmid Miniprep II kit (Zymo Research).

GG) Chemically competent E. coli.

HH) SOC medium: Mix 2 g bactotryptone, 0.5 g yeast extract, 0.2 mL 5 M NaCl, and 0.2 mL 1.25 M KCl in ddH2O to approximately 97 mL and autoclave to sterilize. Under sterile conditions, add 1 mL sterile 1 M MgCl2 and 1.8 mL sterile 20% glucose. Store at room temperature.

II) Luria-Bertani (LB) medium (available as premixed powder or use the following recipe: for 1 L, mix 10 g tryptone, 5 g yeast extract, and 10 g sodium chloride in 1 L ddH2O and autoclave to sterilize). Store at room temperature.

JJ) 2000× ampicillin stock: Dissolve ampicillin in ddH2O at 100 mg/mL and sterile filter using a 0.2 m filter. Store at −20° C. for up to 1 year or at 4° C. for up to 1 month. The working concentration of ampicillin in liquid or solid media is 50 μg/mL.

KK) Luria-Bertani (LB) plates with antibiotics: Mix 5 g tryptone, 2.5 g yeast extract, 5 g sodium chloride, and 7.5 g agar in 500 mL ddH2O with a stir bar in a 1 L bottle.

Autoclave to sterilize, allow media to cool with stirring to 55° C., add ampicillin, and pour plates. This recipe is expected to produce approximately 40-50, 100 mm plates. Store at 4° C.

LL) E. coli plasmid DNA miniprep kit such as those sold by Qiagen, Epoch Life Science, or Zymo Research.

### Methods

1. Site-specific Incorporation of ncAAs in Proteins in Yeast

(a) Prepare chemically competent yeast by first streaking out cells from a glycerol or other stock on a YPD plate. Grow at 30° C. in a stationary incubator for 1-2 days, then inoculate a single, isolated colony from the YPD plate into a 5 mL YPD culture supplemented with penicillin-streptomycin. Grow the culture at 30° C. in a shaking incubator overnight or until the culture is saturated, then dilute 500 μL into 4.5 mL YPD supplemented with penicillin-streptomycin and grow for another 4-6 h at 30° C. in a shaking incubator.

Continue to prepare cells using a kit such as the Zymo Research Frozen-EZ Yeast Transformation II Kit. Chemically competent yeast can be used immediately or frozen in a cryoprotectant container at −80° C.

(b) Using the same yeast chemical competence preparation and transformation kit, transform the plasmid DNA of interest into the cells. For yeast-displayed proteins, prepare the following separate transformations: pPOIVector-TAG and pOTSVector, pPOIVector-WT and pOTSVector, and the pPOIVector-WT only (this serves as a control for yeast display). For intracellular proteins, only the pPOIVector-TAG/pOTSVector and pPOIVector-WT/pOTSVector combinations are necessary. Plate on selective media for retention of the specific combinations of plasmids. Grow at 30° C. in a stationary incubator for 2-3 days.

(c) For each non-control plasmid combination, inoculate three single, isolated colonies from the selective media plate into three 5 mL selective media cultures supplemented with penicillin-streptomycin. For yeast-displayed protein controls, only one culture is needed. Note that separate cultures of yeast that do not contain any plasmid DNA are necessary for microplate reader-based data collection. Grow the cultures at 30° C. in a shaking incubator until the culture is saturated, then dilute each culture to OD600 of 1 in 5 mL of the identical growth media supplemented with penicillin-streptomycin until the OD600 is between 2 and 5 (this should take 4-6 h). Induce each culture at OD600 of 1 in 2 mL galactose-containing selective media supplemented with penicillin-streptomycin. For each POI, prepare a culture with no ncAA, and one tube each for the ncAAs of interest. Incubate cultures at 20° C. in a shaking incubator for 16 h.

2. Flow Cytometry- and Microplate Reader-Based Evaluation of ncAA Incorporation Events in Yeast

(a) To prepare cells with yeast-displayed POIs for flow cytometry, begin by removing two million cells to microcentrifuge tubes. Centrifuge to pellet, aspirate supernatant, and resuspend each pellet in 1 mL PBSA to wash. Repeat the wash twice more and then resuspend each sample in 50 μL PBSA with the necessary primary label(s), then incubate on a rotary wheel for 30 min at room temperature. Following this step, all steps should be performed on ice or in a refrigerated centrifuge at 4° C. to reduce label dissociation. Dilute each sample with 950 μL ice-cold PBSA, centrifuge to pellet, and aspirate supernatant. Wash twice more with ice-cold PBSA, then resuspend each sample in 50 μL PBSA with the necessary secondary label(s). Incubate on ice in the dark for 15 min. Cells can be immediately resuspended and evaluated on the flow cytometer or kept as wet pellets on ice or at 4° C. in the dark for short periods before evaluation.

(b) To prepare cells with intracellular POIs for flow cytometry, begin by removing two million cells to microcentrifuge tubes. Centrifuge to pellet, aspirate supernatant, and resuspend each pellet in 1 mL PBSA to wash. Repeat the wash twice more for a total of three washes. Cells can be immediately resuspended and evaluated on the flow cytometer or kept as wet pellets on ice or at 4° C. for short periods before evaluation.

(c) To prepare cells with intracellular POIs for microplate reader assays, begin by removing two million cells to microcentrifuge tubes. Centrifuge to pellet, aspirate supernatant, and resuspend each pellet in 1 mL PBSA to wash. Repeat the wash twice more for a total of three washes. Cells can be immediately resuspended and evaluated on the microplate reader or kept as wet pellets on ice or at 4° C. for short periods before evaluation. Samples should be resuspended and transferred to 96-well black wall microplates, taking care not to introduce any air bubbles, prior to being evaluated on the microplate reader.

### 3. Flow Cytometry Data Analysis for Relative Readthrough Efficiency (RRE) and Maximum Misincorporation Frequency (MMF)

(a) To begin isolating single cells, draw a polygon gate on the unlabeled yeast sample on a log plot of side scatter (SSC) area versus forward scatter (FSC) area. This population is now called Gate 1 and contains cells that are morphologically similar and are likely to be alive based on size and scatter.

(b) Within Gate 1, draw a polygon gate on a log plot of FSC height versus FSC width. This population is now called Gate 2 and contains single cells while excluding doublets, triplets, or other groups of cells. Further isolation of the single-cell populations may be possible on some flow cytometers (such as with SSC height versus SSC width).

(c) Within Gate 2, prepare a dot plot with axes set to the fluorescence heights corresponding to detection of the C-terminus and N-terminus. For samples with only C-terminus detection ability (e.g., GFP-only samples), the second axis should be set to another fluorescence detection channel that is not expected to have crosstalk with the C-terminus detection channel.

(d) For samples with dual-terminus detection capability, gate the population of cells with above-background levels of N-terminus detection on the Gate 2 histogram plot of N-terminus detection.

4. Bioorthogonal Reactions with ncAAs on the Yeast Surface

(a) One-step click chemistry is used as a control for reacting available azide or alkyne functional groups that have been genetically encoded in the protein of interest on the yeast surface with a probe that can be labeled and detected on a flow cytometer, such as biotin. Step 1: react the surface-displayed protein with an encoded ncAA containing an azide or alkyne functional group with an alkyne- or azide-biotin, or cyclooctyne-biotin for use with azide functional groups only (strain-promoted click chemistry).

(b) Two-step click chemistry. Step 1: react the surface-displayed protein with an encoded ncAA containing an azide or alkyne functional group with an alkyne- or azide-cargo, or cyclooctyne-cargo for use with azide functional groups only (strain-promoted click chemistry). The outcome of the first step may include a mixture of unreacted proteins and cargo-modified proteins. Step 2: react the population of yeast from the first step with an alkyne- or azide-biotin, or cyclooctyne-biotin (for use with azide functional groups only; strain-promoted click chemistry). The products of the second step are expected to be a mixture of cargo-modified proteins and biotin-modified proteins (reactions with biotin probes should be performed under conditions known to lead to complete reactions to avoid unreacted functional groups, shown in brackets).

(c) The level of chemical modification with the cargo of interest can be evaluated by determining the extent of reaction. The background-subtracted one-step biotin detection and background-subtracted two-step biotin detection are required for this calculation. CuAAC: copper-catalyzed azide-alkyne cycloaddition. SPAAC: strain-promoted azide-alkyne cycloaddition.

### 5. Click Chemistry Analysis: Flow Cytometry and Extent of Reaction Calculations

Details of click chemistry analysis are shown in for example, Stieglitz and Deventer 2022 Biomedical Engineering Technologies. Methods in Molecular Biology, vol 2394.

Humana, New York, NY.

6. Preparation of Libraries Involving the Use of Orthogonal Translation Systems

(a) To prepare a library of OTSs, begin by performing a double restriction enzyme digest on the pRS315-LeuOmeRS plasmid. Note that other OTS expression vectors can be used with corresponding restriction enzymes specific to that vector. Evaluate on a DNA gel and extract the band corresponding to the vector with no OTS insert. Amplify the OTS library insert(s) via PCR with primers containing the desired degenerate codon(s) or mutation(s), then evaluate and extract from a DNA gel. Follow Pellet Paint manufacturing protocols to concentrate the pooled OTS and vector DNA. Separately, prepare yeast cells that only contain a ncAA incorporation reporter.

(b) To prepare a library of POIs, begin by performing a triple restriction enzyme digest on pCTCON2. Note that other yeast display vectors can be used with corresponding restriction enzymes specific to that vector. Evaluate on a DNA gel and extract the band corresponding to the vector with no POI insert. Amplify the POI library insert(s) via PCR with primers containing the desired degenerate codon(s) or mutation(s), then evaluate and extract from a DNA gel. Follow Pellet Paint manufacturing protocols to concentrate the pooled POI and vector DNA. Separately, prepare yeast cells that only contain the pOTSVector.

(c) Prepare electrocompetent cells then combine with the concentrated library and vector DNA and electroporate. Recover each electroporated sample with 2 mL YPD at 30° C. for 1 h with no shaking. Also, pre-warm one selective media plate for each sample at this time. To determine the transformation efficiency, prepare four serial dilutions of each sample and plate on quadrants of the selective media plates. Grow at 30° C. for 3-4 days and determine a number of the colonies in each quadrant to determine the approximate number of transformants. Centrifuge the remainder of the recovered samples and aspirate the YPD, then resuspend each pellet in 100 mL selective media supplemented with penicillin-streptomycin and grow at 30° C. with shaking for 1-2 days until saturated. Centrifuge the culture to pellet, decant supernatant, and resuspend in 1 L selective media supplemented with penicillin-streptomycin. At this point, remove 200 μL of the 1 L cultures and set aside for additional characterization steps. Grow at 30° C. for 1-2 days until saturated, then centrifuge and resuspend the entire pellet in 5 mL 60% glycerol. Freeze library at −80° C. Take the 200 μL removed after passaging to 1 L and propagate for flow cytometry characterization. Also, use a yeast DNA purification “miniprep” kit such as the Zymoprep Yeast Plasmid Miniprep II kit to isolate the plasmid DNA and characterize the constructed library or libraries.

### Example 6: Yeast Strain with Synthetic Genome

This example uses an assembly strategy to generate an yeast strain with synthetic genome. Yeast has 16 chromosomes (ChrI to ChrXVI). In some embodiments, an assembly strategy may comprise endogenous homologous recombination machinery to replace one or more of 30- to 60-kilobase segments of each wild-type chromosome with the corresponding synthetic sequence. A chromosome can be computationally divided into 30-60 kilobase long “megachunks,” each comprising a set of “chunks” of segments that is less than about 10 kilobase in length. These “chunks” can be assembled into “megachunks” by restriction enzyme cutting and ligation in vitro, or any other methods known in the art. The “megachunks” can be subsequently integrated into the host genome, e.g., an yeast genome, replacing the corresponding wile-type segment.

In some embodiments, “megachunks” can be introduced sequentially from left to right (i.e., from 5′ to 3′ direction) using the endogenous homologous recombination machinery and termini. In some embodiments, the termini may comprise a terminal universal telomere cap (UTC) sequences, for the first and last “megachunk” extremities. In some embodiments, the termini may comprise terminal sequences of up to 500 bp that can facilitate integration into a partially synthetic, partially native chromosome. In some embodiments, “chunks” and/or “megachunks” may comprise a selectable marker. In some embodiments, the right most “chunk” in each “megachunk” (i.e., a “chunk” in the most 3′ side of a “megachunk”) may comprise a selectable marker. For example, the selectable marker can be any auxotrophic marker. In some embodiments, an auxotrophic marker may comprise URA3, LYS2, LEU2, TRP1, HIS3, MET15, or ADE2. In some embodiments, the selectable marker may be LEU2 or URA3. In some embodiments, as each “megachunk” is introduced, the previously used marker is overwritten as a consequence of homologous recombination with the incoming “megachunk.” In some embodiments, if the first “megachunk” is tagged with LEU2, the second “megachunk” is tagged with another marker, such as URA3. In some embodiments, two markers can be alternated. For example if the first “megachunk” is tagged with LEU2, the second “megachunk” is tagged with URA3, and the third “megachunk” is tagged with LEU2.

In other embodiments, “chunks” can be provided as a series of “minichunks” that overlap with each other and can be recombined with each other. In this embodiment, the series of “minichunks” can be integrated into the genome simultaneously by using a selective marker (e.g., auxotrophic marker) switching. In some embodiments, the first (5′) “megachunk” of a synthetic chromosome may be provided with a telomere seed sequence (TeSS) within the larger UTC fragment. In some embodiments, the last (3′) “megachunk” of a synthetic chromosome may be provided with a terminal sequence homology targeting the wild type chromosome. In some embodiments, the TeSS end may be designed to grow a new telomere. In some embodiments, the TeSS may not participate in homologous recombination. In some embodiments, the last or the rightmost “megachunk” of a synthetic chromosome (i.e., the“megachunk” of the 5′ end of a synthetic chromosome) may comprise a selectable marker. In some embodiments, the last or the rightmost “megachunk” of a synthetic chromosome (i.e., the“megachunk” of the 5′ end of a synthetic chromosome) may not comprise a selectable marker. In this embodiments, the second-to-last “megachunk” may comprise a URA3 marker. In this embodiment, selection for the last “megachunk” can be provided by 5-fluoroorotic acid (5′FOA) resistance phenotype conferred by the last “megachunk” as it overwrites the URA3 marker from the second-to-last “megachunk.”

In some embodiments, integration may comprise utilizing an inducible genome rearrangement system. In some embodiments, the inducible genome arrangement system may be based on a chemically inducible Cre recombinase. In some embodiments, a palindromic recombination site loxPsym may be inserted in the genome. In some embodiments, the palindromic recombination site loxPsym may be inserted 3 bp downstream of the stop codon of an nonessential gene/ORF.

Next, the assembled synthetic chromosomes are sequenced to verify and quantify the synthetic content of the genome. A “PCRTagging” watermark system can be used by introducing slight nucleotide sequence alterations through synonymous recoding within ORFs to specify pairs of primers specific to either the wild type or synthetic version of that gene/ORFs. In addition synthetic chromosomes are validated by whole-genome sequencing. In some embodiments, “semisynthetic” strains may be sequenced at major intervals during assembly (e.g., 300 to 500 kb integrated) in order to identify major structural variants that occur at about that frequency and to eliminate them early in assembly.

In addition, the fitness of the resulting recombinant semi-synthetic yeast strains is assessed, and any substitution that proves lethal or leads to a measurable fitness defect can be corrected. The correction can be done by reverting the sequence to wild type (“debugging”). The hierarchical nature of the assembly scheme can facilitate debugging, as specific designer features for codon rewriting can be corrected and fixed once bugs are identified. In some embodiments, this can facilitate a “design-build-assemble-test-learn” cycle used in the final stage of production of synthetic chromosomes.

Once assembly of the various synthetic chromosomes is completed, an efficient meiotic strategy can be used to combine all synthetic chromosomes. In one embodiment, synthetic chromosomes can be consolidated into a single strain by mating and sporulation. In another embodiment, a conditional chromosome destabilization can used (e.g., endoreduplication intercross). In this embodiment, a centromere function of two specified native chromosomes may be simultaneously disrupted in a doubly heterozygous diploid synthetic strain (e.g., synIII/III VI/synVI). In some embodiments, this can be performed by using the GAL1 promoter in cis to generate a “2n-2” strain. In some embodiments, each chromosome can be individually lost, in diploids, yielding hemizygotes for the destabilized chromosome. In some embodiments, most such “2n−1” strains may endoreduplicate the remaining single chromosomes to regenerate a 2n state. In some embodiments, conditional chromosome destabilization can be used to backcross synthetic strains to wild type, called an “endoreduplication backcross,” to revert the sequence to wild type or to debug. Diploid strains can be sporulated to produce haploid strains. Karyotypic analysis by pulsed-field gel electrophoresis in the haploid strains can be used to visualize mobility shifts of synthetic chromosomes in resulting haploid strains to compare with wild type chromosomes.

TABLE 9

| | context_ | | codon_ | codon_ | | | | | | context_ | | most_ | most_ | codon_ |
|---|---|---|---|---|---|---|---|---|---|---|---|---|---|---|
| context | aa | cnt | cnt | cnt_null | df | q | spq | pval | sppval | cnt | ratio | depleted | enriched | order |
| AAA_E_ | K_E_H | GAA | 48 | 71.985 | 1 | 24.1099 | 26.5398216 | 9.09888239 | 2.581612945 | 103 | 1.64009178 | 1.5:AAAG | 1.8:AAAG | GAG |
| CAT | | GAG | 55 | 31.015 | | 5241714 | 25614287 | 4258296e- | 690416e-07 | | 54724906 | AACAT | AGCAT | GAA |
| | | | | | | 5713 | | 07 | | | | | | |
| AAA_F_ | K_F_K | TTC | 147 | 104.375 | 1 | 28.6276 | 29.3120062 | 8.77206294 | 6.161276024 | 257 | 1.39940978 | 1.4:AAATT | 1.4:AAATT | TTC |
| AAA | | TTT | 110 | 152.625 | | 4987340 | 28969352 | 336009e-08 | 059292e-08 | | 38973951 | TAAA | CAAA | TTT |
| | | | | | | 03 | | | | | | | | |
| AAA_F_ | K_F_K | TTC | 99 | 60.919 | 1 | 39.2560 | 40.0836699 | 3.71706546 | 2.433145213 | 150 | 1.66546229 | 1.7:AAATT | 1.6:AAATT | TTC |
| AAG | | TTT | 51 | 89.081 | | 9338219 | 30238244 | 89789557e- | 2630965e-10 | | 97232904 | TAAG | CAAG | TTT |
| | | | | | | 992 | | 10 | | | | | | |
| AAA_F_ | K_F_N | TTC | 118 | 77.570 | 1 | 34.6562 | 35.4822812 | 3.93368270 | 2.573812437 | 191 | 1.53359004 | 1.6:AAATT | 1.5:AAATT | TTC |
| AAT | | TTT | 73 | 113.430 | | 7625902 | 4451543 | 19891175e- | 876598e-09 | | 76184237 | TAAT | CAAT | TTT |
| | | | | | | 542 | | 09 | | | | | | |
| AAA_G_ | K_G_V | GGA | 10 | 24.383 | 3 | 48.3785 | 46.6134562 | 1.76893247 | 4.199972094 | 109 | 1.85991217 | 2.7:AAAG | 1.7:AAAG | GGT |
| GTT | | GGC | 8 | 21.552 | | 5225584 | 1201848 | 21373004e- | 283413e-10 | | 01214172 | GCGTT | GTGTT | GGA |
| | | GGG | 6 | 13.539 | | 942 | | 10 | | | | | | GGC |
| | | GGT | 85 | 49.526 | | | | | | | | | | GGG |
| AAA_G_ | K_G_F | GGA | 49 | 32.884 | 3 | 47.2663 | 52.6559926 | 3.05044196 | 2.170626706 | 147 | 1.74431562 | 1.9:AAAG | 2.2:AAAG | GGA |
| TTT | | GGC | 21 | 29.066 | | 9131215 | 89022305 | 5528308e- | 764458e-11 | | 65060125 | GTTTT | GGTTT | GGG |
| | | GGG | 41 | 18.259 | | 286 | | 10 | | | | | | GGT |
| | | GGT | 36 | 66.792 | | | | | | | | | | GGC |
| AAA_I_ | K_I_K | ATA | 129 | 110.913 | 2 | 32.1661 | 31.7414226 | 1.03564841 | 1.280671187 | 398 | 1.30801881 | 1.4:AAAA | 1.4:AAAA | ATC |
| AAA | | ATC | 139 | 102.760 | | 3587033 | 9036615 | 22176173e- | 5521908e-07 | | 05713606 | TTAAA | TCAAA | ATT |
| | | ATT | 130 | 184.327 | | 305 | | 07 | | | | | | ATA |
| AAA_I_ | K_I_N | ATA | 89 | 83.881 | 2 | 31.8469 | 32.6851395 | 1.21486853 | 7.989362683 | 301 | 1.33755172 | 1.5:AAAA | 1.5:AAAA | ATC |
| AAT | | ATC | 116 | 77.716 | | 1956554 | 0322449 | 36582085e- | 58876e-08 | | 64842502 | TTAAT | TCAAT | ATT |
| | | ATT | 96 | 139.403 | | 1185 | | 07 | | | | | | ATA |
| AAA_K_ | K_K_K | AAA | 198 | 277.462 | 1 | 53.1791 | 54.0877517 | 3.04468837 | 1.917327143 | 479 | 1.39718853 | 1.4:AAAA | 1.4:AAAA | AAG |
| AAA | | AAG | 281 | 201.538 | | 7981993 | 1829723 | 8386057e- | 2196194e-13 | | 13842118 | AAAAA | AGAAA | AAA |
| | | | | | | 921 | | 13 | | | | | | |
| AAA_K_ | K_K_K | AAA | 135 | 209.111 | 1 | 61.4677 | 62.4256746 | 4.50046808 | 2.766924028 | 361 | 1.51046476 | 1.5:AAAA | 1.5:AAAA | AAG |
| AAG | | AAG | 226 | 151.889 | | 8461002 | 6430538 | 5192229e- | 664213e-15 | | 28183967 | AAAAG | AGAAG | AAA |
| | | | | | | 323 | | 15 | | | | | | |
| AAA_K_ | K_K_M | AAA | 141 | 107.162 | 1 | 27.1873 | 25.3951694 | 1.84662713 | 4.670862469 | 185 | 1.41174206 | 1.8:AAAA | 1.3:AAAA | AAA |
| ATG | | AAG | 44 | 77.838 | | 5277235 | 9664933 | 16092957e- | 136582e-07 | | 35658593 | AGATG | AAATG | AAG |
| | | | | | | 0522 | | 07 | | | | | | |
| AAA_K_ | K_K_Q | AAA | 82 | 127.436 | 1 | 37.9143 | 38.5020546 | 7.39192005 | 5.469607231 | 220 | 1.51412066 | 1.6:AAAA | 1.5:AAAA | AAG |
| CAA | | AAG | 138 | 92.564 | | 5864707 | 29316895 | 8302951e- | 202917e-10 | | 37027087 | AACAA | AGCAA | AAA |
| | | | | | | 641 | | 10 | | | | | | |
| AAA_K_ | K_K_E | AAA | 176 | 235.756 | 1 | 35.3955 | 35.9982972 | 2.69099564 | 1.974900328 | 407 | 1.34486776 | 1.3:AAAA | 1.3:AAAA | AAG |
| GAA | | AAG | 231 | 171.244 | | 6041850 | 50677425 | 21538124e- | 9252396e-09 | | 50038756 | AAGAA | AGGAA | AAA |
| | | | | | | 71 | | 09 | | | | | | |
| AAA_K_ | K_K_W | AAA | 90 | 63.718 | 1 | 28.6010 | 25.7655117 | 8.89336765 | 3.855160622 | 110 | 1.54512404 | 2.3:AAAA | 1.4:AAAA | AAA |
| TGG | | AAG | 20 | 46.282 | | 5585771 | 92210464 | 8020597e- | 4489134e-07 | | 48630073 | AGTGG | AATGG | AAG |
| | | | | | | 646 | | 08 | | | | | | |
| AAA_L_ | K_L_K | CTA | 97 | 89.779 | 5 | 71.7277 | 72.9263650 | 4.47621052 | 2.518412585 | 634 | 1.29866112 | 2.0:AAACT | 1.7:AAAC | TTG |
| AAA | | CTC | 37 | 36.611 | | 0464090 | 2089923 | 62420036e- | 77527e-14 | | 86451626 | TAAA | TGAAA | TTA |
| | | CTG | 124 | 71.717 | | 058 | | 14 | | | | | | CTG |
| | | CTT | 40 | 81.433 | | | | | | | | | | CTA |
| | | TTA | 133 | 174.967 | | | | | | | | | | CTT |
| | | TTG | 203 | 179.493 | | | | | | | | | | CTC |
| AAA_L_ | K_L_Y | CTA | 44 | 28.322 | 5 | 62.7377 | 68.4375542 | 3.29891567 | 2.165854189 | 200 | 1.69101289 | 2.0:AAATT | 2.2:AAAC | CTT |
| TAT | | CTC | 14 | 11.549 | | 9136561 | 7182992 | 90336927e- | 129951e-13 | | 78555763 | GTAT | TTTAT | CTA |
| | | CTG | 25 | 22.624 | | 3695 | | 12 | | | | | | TTA |
| | | CTT | 56 | 25.689 | | | | | | | | | | TTG |
| | | TTA | 32 | 55.195 | | | | | | | | | | CTG |
| | | TTG | 29 | 56.622 | | | | | | | | | | CTC |
| AAA_L_ | K_L_S | CTA | 44 | 24.073 | 5 | 71.6024 | 79.1646955 | 4.75328871 | 1.254793964 | 170 | 1.93381903 | 2.0:AAATT | 2.2:AAAC | CTT |
| TCC | | CTC | 16 | 9.817 | | 8719991 | 8928256 | 8920618e- | 396139e-15 | | 6190981 | GTCC | TTTCC | CTA |
| | | CTG | 10 | 19.230 | | 46 | | 14 | | | | | | TTA |
| | | CTT | 49 | 21.835 | | | | | | | | | | TTG |
| | | TTA | 27 | 46.915 | | | | | | | | | | CTC |
| | | TTG | 24 | 48.129 | | | | | | | | | | CTG |
| AAA_L_ | K_L_S | CTA | 52 | 37.101 | 5 | 49.7559 | 47.2536667 | 1.55464310 | 5.043573378 | 262 | 1.38398276 | 2.2:AAATT | 1.7:AAAC | TTA |
| TCT | | CTC | 22 | 15.130 | | 5708313 | 5023515 | 37338621e- | 994853e-09 | | 82959014 | GTCT | TTTCT | CTT |
| | | CTG | 26 | 29.637 | | 0426 | | 09 | | | | | | CTA |
| | | CTT | 56 | 33.652 | | | | | | | | | | TTG |
| | | TTA | 73 | 72.305 | | | | | | | | | | CTG |
| | | TTG | 33 | 74.175 | | | | | | | | | | CTC |
| AAA_L_ | K_L_L | CTA | 73 | 47.439 | 5 | 46.6967 | 45.7297796 | 6.55037912 | 1.030836046 | 335 | 1.39347755 | 1.8:AAATT | 1.5:AAAC | TTA |
| TTA | | CTC | 12 | 19.345 | | 6479627 | 4832812 | 4659852e- | 3917905e-08 | | 1864934 | GTTA | TATTA | CTA |
| | | CTG | 29 | 37.894 | | 845 | | 09 | | | | | | CTT |
| | | CTT | 61 | 43.028 | | | | | | | | | | TTG |
| | | TTA | 106 | 92.451 | | | | | | | | | | CTG |
| | | TTG | 54 | 94.843 | | | | | | | | | | CTC |
| AAA_L_ | K_L_L | CTA | 69 | 48.288 | 5 | 57.2982 | 63.8667660 | 4.38937556 | 1.925196034 | 341 | 1.46412063 | 1.8:AAACT | 1.9:AAAC | CTT |
| TTG | | CTC | 11 | 19.691 | | 7538833 | 8224829 | 177538e-11 | 7471175e-12 | | 65451591 | CTTG | TTTTG | TTA |
| | | CTG | 37 | 38.573 | | 7096 | | | | | | | | CTA |
| | | CTT | 84 | 43.799 | | | | | | | | | | TTG |
| | | TTA | 75 | 94.107 | | | | | | | | | | CTG |
| | | TTG | 65 | 96.541 | | | | | | | | | | CTC |
| AAA_R_ | K_R_K | AGA | 218 | 213.365 | 5 | 40.8801 | 39.8500024 | 9.92050700 | 1.601069743 | 450 | 1.23613893 | 1.8:AAAC | 1.5:AAAA | AGA |
| AAA | | AGG | 142 | 97.607 | | 8285596 | 70568754 | 4552373e- | 608348e-07 | | 61892405 | GTAAA | GGAAA | AGG |
| | | CGA | 23 | 30.729 | | 159 | | 08 | | | | | | CGT |
| | | CGC | 15 | 26.634 | | | | | | | | | | CGA |
| | | CGG | 17 | 18.783 | | | | | | | | | | CGG |
| | | CGT | 35 | 62.883 | | | | | | | | | | CGC |
| AAA_R_ | K_R_S | AGA | 24 | 32.716 | 5 | 49.1250 | 97.7784824 | 2.09240917 | 1.552785481 | 69 | 2.04215371 | 4.7:AAAC | 6.6:AAAC | AGA |
| TCG | | AGG | 16 | 14.966 | | 4927067 | 6844405 | 89529815e- | 6557929e-19 | | 3169908 | GATCG | GGTCG | CGG |
| | | CGA | 1 | 4.712 | | 867 | | 09 | | | | | | AGG |
| | | CGC | 4 | 4.084 | | | | | | | | | | CGT |
| | | CGG | 19 | 2.880 | | | | | | | | | | CGC |
| | | CGT | 5 | 9.642 | | | | | | | | | | CGA |
| AAA_S_ | K_S_D | AGC | 13 | 18.366 | 5 | 56.1105 | 57.3949013 | 7.71168255 | 4.192574029 | 163 | 1.59936744 | 4.3:AAATC | 2.1:AAAA | AGT |
| GAC | | AGT | 57 | 26.602 | | 1018113 | 5966844 | 5190532e- | 1932686e-11 | | 53277928 | CGAC | GTGAC | TCA |
| | | TCA | 42 | 34.104 | | 1204 | | 11 | | | | | | TCT |
| | | TCC | 6 | 25.594 | | | | | | | | | | TCG |
| | | TCG | 16 | 15.885 | | | | | | | | | | AGC |
| | | TCT | 29 | 42.450 | | | | | | | | | | TCC |
| AAA_V_ | K_V_S | GTA | 35 | 15.277 | 3 | 26.8593 | 32.6236085 | 6.30095857 | 3.866584480 | 71 | 1.87412893 | 1.8:AAAG | 2.3:AAAG | GTA |
| TCG | | GTC | 8 | 14.305 | | 3740594 | 3139053 | 4118555e- | 582239e-07 | | 21688612 | TCTCG | TATCG | GTT |
| | | GTG | 10 | 13.927 | | 439 | | 06 | | | | | | GTG |
| | | GTT | 18 | 27.491 | | | | | | | | | | GTC |
| AAC_E_ | N_E_L | GAA | 12 | 27.256 | 1 | 25.2674 | 28.3596018 | 4.99065335 | 1.007458670 | 39 | 2.29055011 | 2.3:AACG | 2.3:AACG | GAG |
| CTC | | GAG | 27 | 11.744 | | 2959736 | 06212172 | 5512901e- | 1243565e-07 | | 518358 | AACTC | AGCTC | GAA |
| | | | | | | 574 | | 07 | | | | | | |
| AAC_I_ | N_I_N | ATA | 40 | 37.900 | 2 | 28.3270 | 29.3153013 | 7.06078509 | 4.307875910 | 136 | 1.49226160 | 1.7:AACAT | 1.7:AACA | ATC |
| AAC | | ATC | 60 | 35.114 | | 7880451 | 93202116 | 5838089e- | 1191305e-07 | | 7992085 | TAAC | TCAAC | ATA |
| | | ATT | 36 | 62.986 | | 8614 | | 07 | | | | | | ATT |
| AAC_L_ | N_L_D | CTA | 21 | 28.038 | 5 | 39.9333 | 47.9939578 | 1.54028852 | 3.562024212 | 198 | 1.52629138 | 1.5:AACTT | 2.1:AACCT | CTT |
| GAT | | CTC | 20 | 11.434 | | 7056443 | 10554946 | 7229374e- | 229175e-09 | | 8410202 | AGAT | TGAT | TTG |
| | | CTG | 18 | 22.397 | | 664 | | 07 | | | | | | TTA |
| | | CTT | 54 | 25.432 | | | | | | | | | | CTA |
| | | TTA | 37 | 54.643 | | | | | | | | | | CTC |
| | | TTG | 48 | 56.056 | | | | | | | | | | CTG |
| AAC_N_ | N_N_N | AAC | 272 | 180.827 | 1 | 75.1647 | 77.0820189 | 4.33035805 | 1.640036839 | 448 | 1.50961647 | 1.5:AACA | 1.5:AACA | AAC |
| AAC | | AAT | 176 | 267.173 | | 2180471 | 4417269 | 8874999e- | 5057556e-18 | | 48918668 | ATAAC | ACAAC | AAT |
| | | | | | | 928 | | 18 | | | | | | |
| AAC_S_ | N_S_N | AGC | 69 | 32.337 | 5 | 96.8687 | 100.701442 | 2.41371629 | 3.760145627 | 287 | 1.73275796 | 2.3:AACTC | 2.1:AACA | TCC |
| AAC | | AGT | 64 | 46.839 | | 7638631 | 15739441 | 6834796e- | 0588833e-20 | | 31481 | TAAC | GCAAC | AGC |
| | | TCA | 33 | 60.048 | | 486 | | 19 | | | | | | AGT |
| | | TCC | 70 | 45.064 | | | | | | | | | | TCT |
| | | TCG | 18 | 27.969 | | | | | | | | | | TCA |
| | | TCT | 33 | 74.743 | | | | | | | | | | TCG |
| AAC_S_ | N_S_N | AGC | 91 | 40.788 | 5 | 96.6011 | 110.340910 | 2.74814486 | 3.470985636 | 362 | 1.64075225 | 1.8:AACTC | 2.2:AACA | AGT |
| AAT | | AGT | 92 | 59.079 | | 2872740 | 20471736 | 907625e-19 | 0757543e-22 | | 27464475 | TAAT | GCAAT | AGC |
| | | TCA | 52 | 75.740 | | 838 | | | | | | | | TCA |
| | | TCC | 48 | 56.841 | | | | | | | | | | TCT |
| | | TCG | 28 | 35.278 | | | | | | | | | | TCC |
| | | TCT | 51 | 94.275 | | | | | | | | | | TCG |
| AAC_S_ | N_S_S | AGC | 36 | 22.985 | 5 | 152.648 | 185.428995 | 3.64457549 | 3.704228326 | 204 | 2.31146482 | 3.3:AACTC | 3.0:AACTC | TCC |
| AGC | | AGT | 27 | 33.293 | | 5795373 | 21088634 | 4890808e- | 554208e-38 | | 562041 | TAGC | CAGC | AGC |
| | | TCA | 20 | 42.682 | | 5782 | | 31 | | | | | | AGT |
| | | TCC | 97 | 32.032 | | | | | | | | | | TCA |
| | | TCG | 8 | 19.880 | | | | | | | | | | TCT |
| | | TCT | 16 | 53.127 | | | | | | | | | | TCG |
| AAC_S_ | N_S_S | AGC | 39 | 18.366 | 5 | 55.3667 | 58.1325289 | 1.09716972 | 2.953540758 | 163 | 1.67758333 | 3.2:AACTC | 2.1:AACA | AGT |
| AGT | | AGT | 46 | 26.602 | | 2127933 | 5082524 | 30289936e- | 048283e-11 | | 00398977 | GAGT | GCAGT | AGC |
| | | TCA | 25 | 34.104 | | 188 | | 10 | | | | | | TCC |
| | | TCC | 27 | 25.594 | | | | | | | | | | TCA |
| | | TCG | 5 | 15.885 | | | | | | | | | | TCT |
| | | TCT | 21 | 42.450 | | | | | | | | | | TCG |
| AAC_S_ | N_S_D | AGC | 32 | 23.774 | 5 | 42.7806 | 40.8151210 | 4.09324678 | 1.022524545 | 211 | 1.43280703 | 2.8:AACTC | 1.7:AACA | AGT |
| GAT | | AGT | 59 | 34.436 | | 8251433 | 1630292 | 5897924e- | 1154318e-07 | | 99829893 | CGAT | GTGAT | TCA |
| | | TCA | 51 | 44.147 | | 714 | | 08 | | | | | | TCT |
| | | TCC | 12 | 33.131 | | | | | | | | | | AGC |
| | | TCG | 11 | 20.562 | | | | | | | | | | TCC |
| | | TCT | 46 | 54.950 | | | | | | | | | | TCG |
| AAC_V_ | N_V_N | GTA | 13 | 20.226 | 3 | 31.2258 | 38.3688038 | 7.61868764 | 2.361324374 | 94 | 1.79338785 | 1.6:AACGT | 2.3:AACG | GTC |
| AAC | | GTC | 43 | 18.939 | | 6991263 | 50243985 | 1115787e- | 739755e-08 | | 35491918 | AAAC | TCAAC | GTT |
| | | GTG | 12 | 18.439 | | 798 | | 07 | | | | | | GTA |
| | | GTT | 26 | 36.396 | | | | | | | | | | GTG |
| AAC_V_ | N_V_R | GTA | 6 | 21.733 | 3 | 179.729 | 241.640935 | 1.00915779 | 4.203868260 | 101 | 4.14034303 | 5.0:AACGT | 4.1:AACG | GTC |
| AGG | | GTC | 83 | 20.349 | | 1395128 | 58932362 | 17773843e- | 79788e-52 | | 7836222 | GAGG | TCAGG | GTT |
| | | GTG | 4 | 19.812 | | 5776 | | 38 | | | | | | GTA |
| | | GTT | 8 | 39.106 | | | | | | | | | | GTG |
| AAG_D_ | K_D_R | GAC | 23 | 10.029 | 1 | 24.3660 | 25.6436447 | 7.96621213 | 4.106456522 | 29 | 2.45085755 | 3.2:AAGG | 2.3:AAGG | GAC |
| CGA | | GAT | 6 | 18.971 | | 0508243 | 92995147 | 6234996e- | 4077895e-07 | | 38828326 | ATCGA | ACCGA | GAT |
| | | | | | | 7987 | | 07 | | | | | | |
| AAG_G_ | K_G_Q | GGA | 14 | 13.646 | 3 | 30.7075 | 36.2415115 | 9.79517155 | 6.657660303 | 61 | 1.92768701 | 2.5:AAGG | 2.5:AAGG | GGC |
| CAG | | GGC | 30 | 12.061 | | 4937623 | 245682 | 2486418e- | 837117e-08 | | 2664023 | GGCAG | GCCAG | GGT |
| | | GGG | 3 | 7.577 | | 7427 | | 07 | | | | | | GGA |
| | | GGT | 14 | 27.716 | | | | | | | | | | GGG |
| AAG_G_ | K_G_C | GGA | 9 | 13.646 | 3 | 62.7018 | 81.7268075 | 1.55530805 | 1.307950391 | 61 | 2.88158660 | 3.8:AAGG | 3.3:AAGG | GGC |
| TGT | | GGC | 40 | 12.061 | | 4000983 | 5441239 | 03723135e- | 4922733e-17 | | 9671602 | GGTGT | GCTGT | GGT |
| | | GGG | 2 | 7.577 | | 122 | | 13 | | | | | | GGA |
| | | GGT | 10 | 27.716 | | | | | | | | | | GGG |
| AAG_K_ | K_K_K | AAA | 265 | 341.180 | 1 | 39.7701 | 40.4278815 | 2.85671402 | 2.040089321 | 589 | 1.29839777 | 1.3:AAGA | 1.3:AAGA | AAG |
| AAG | | AAG | 324 | 247.820 | | 9517633 | 2948292 | 20205875e- | 2701188e-10 | | 80215326 | AAAAG | AGAAG | AAA |
| | | | | | | 735 | | 10 | | | | | | |
| AAG_K_ | K_K_E | AAA | 190 | 253.134 | 1 | 36.7991 | 37.4242353 | 1.30948508 | 9.503385179 | 437 | 1.33853716 | 1.3:AAGA | 1.3:AAGA | AAG |
| GAA | | AAG | 247 | 183.866 | | 3245132 | 1303803 | 4926872e- | 2649e-10 | | 60529113 | AAGAA | AGGAA | AAA |
| | | | | | | 5406 | | 09 | | | | | | |
| AAG_L_ | K_L_N | CTA | 40 | 37.809 | 5 | 58.7240 | 69.9500687 | 2.22985205 | 1.049605014 | 267 | 1.53777811 | 1.7:AAGCT | 2.3:AAGC | TTG |
| AAT | | CTC | 35 | 15.418 | | 1709631 | 604593 | 36983545e- | 3700203e-13 | | 86848625 | TAAT | TCAAT | CTG |
| | | CTG | 60 | 30.202 | | 444 | | 11 | | | | | | TTA |
| | | CTT | 20 | 34.294 | | | | | | | | | | CTA |
| | | TTA | 52 | 73.685 | | | | | | | | | | CTC |
| | | TTG | 60 | 75.591 | | | | | | | | | | CTT |
| AAG_L_ | K_L_L | CTA | 18 | 16.851 | 5 | 35.5465 | 44.0275815 | 1.17026379 | 2.286553729 | 119 | 1.64658458 | 1.7:AAGCT | 2.5:AAGC | CTT |
| CTT | | CTC | 4 | 6.872 | | 9578337 | 6630434 | 65352895e- | 1411365e-08 | | 16122573 | CCTT | TTCTT | TTA |
| | | CTG | 16 | 13.461 | | 9196 | | 06 | | | | | | TTG |
| | | CTT | 38 | 15.285 | | | | | | | | | | CTA |
| | | TTA | 23 | 32.841 | | | | | | | | | | CTG |
| | | TTG | 20 | 33.690 | | | | | | | | | | CTC |
| AAG_L_ | K_L_Y | CTA | 26 | 22.940 | 5 | 35.4853 | 41.8188151 | 1.20370954 | 6.409199244 | 162 | 1.51666329 | 1.7:AAGTT | 2.2:AAGC | CTT |
| TAT | | CTC | 8 | 9.355 | | 4698258 | 2542987 | 31984842e- | 415855e-08 | | 39821239 | GTAT | TTTAT | TTA |
| | | CTG | 21 | 18.325 | | 467 | | 06 | | | | | | TTG |
| | | CTT | 46 | 20.808 | | | | | | | | | | CTA |
| | | TTA | 34 | 44.708 | | | | | | | | | | CTG |
| | | TTG | 27 | 45.864 | | | | | | | | | | CTC |
| AAG_R_ | K_R_K | AGA | 111 | 125.648 | 5 | 32.6465 | 41.3480501 | 4.42321811 | 7.979941975 | 265 | 1.33703672 | 1.5:AAGC | 2.6:AAGC | AGA |
| AAA | | AGG | 74 | 57.480 | | 2483053 | 9812926 | 87449936e- | 512946e-08 | | 85586775 | GAAAA | GGAAA | AGG |
| | | CGA | 12 | 18.096 | | 877 | | 06 | | | | | | CGG |
| | | CGC | 13 | 15.684 | | | | | | | | | | CGT |
| | | CGG | 29 | 11.061 | | | | | | | | | | CGC |
| | | CGT | 26 | 37.031 | | | | | | | | | | CGA |
| AAG_T_ | K_T_L | ACA | 12 | 21.881 | 3 | 32.0574 | 42.7103379 | 5.08960496 | 2.835356495 | 72 | 1.87223062 | 1.8:AAGA | 2.9:AAGA | ACG |
| CTT | | ACC | 9 | 15.254 | | 3791264 | 81897965 | 7488929e- | 7541917e-09 | | 25029537 | CACTT | CGCTT | ACT |
| | | ACG | 29 | 10.099 | | 941 | | 07 | | | | | | ACA |
| | | ACT | 22 | 24.765 | | | | | | | | | | ACC |
| AAG_T_ | K_T_E | ACA | 46 | 31.911 | 3 | 31.7478 | 31.7462016 | 5.91446681 | 5.919314406 | 105 | 1.66846267 | 2.3:AAGA | 1.9:AAGA | ACA |
| GAG | | ACC | 15 | 22.246 | | 9036182 | 81660303 | 8320419e- | 19068e-07 | | 03521799 | CTGAG | CGGAG | ACG |
| | | ACG | 28 | 14.728 | | 9832 | | 07 | | | | | | ACT |
| | | ACT | 16 | 36.116 | | | | | | | | | | ACC |
| AAG_V_ | K_V_K | GTA | 17 | 32.707 | 3 | 30.1050 | 33.5503764 | 1.31158147 | 2.465057221 | 152 | 1.47455620 | 1.9:AAGG | 1.9:AAGG | GTC |
| AAG | | GTC | 58 | 30.624 | | 6620041 | 74424574 | 83957287e- | 5609197e-07 | | 00132517 | TAAAG | TCAAG | GTT |
| | | GTG | 26 | 29.816 | | 734 | | 06 | | | | | | GTG |
| | | GTT | 51 | 58.853 | | | | | | | | | | GTA |
| AAT_A_ | N_A_T | GCA | 33 | 76.327 | 3 | 43.3385 | 37.9353018 | 2.08560045 | 2.917038933 | 259 | 1.34627335 | 2.3:AATGC | 1.5:AATG | GCT |
| ACT | | GCC | 62 | 57.862 | | 2508124 | 8383374 | 26447976e- | 036e-08 | | 13408847 | AACT | CGACT | GCC |
| | | GCG | 43 | 29.613 | | 553 | | 09 | | | | | | GCG |
| | | GCT | 121 | 95.198 | | | | | | | | | | GCA |
| AAT_F_ | N_F_K | TTC | 117 | 80.413 | 1 | 27.3670 | 28.0302949 | 1.68272872 | 1.194310395 | 198 | 1.45363540 | 1.5:AATTT | 1.5:AATTT | TTC |
| AAA | | TTT | 81 | 117.587 | | 6510599 | 0488739 | 96116312e- | 6670442e-07 | | 46598348 | TAAA | CAAA | TTT |
| | | | | | | 7185 | | 07 | | | | | | |
| AAT_F_ | N_F_K | TTC | 101 | 64.168 | 1 | 34.8063 | 35.5987736 | 3.64186707 | 2.424401144 | 158 | 1.59965853 | 1.6:AATTT | 1.6:AATTT | TTC |
| AAG | | TTT | 57 | 93.832 | | 3390196 | 9669815 | 71177216e- | 032614e-09 | | 9423031 | TAAG | CAAG | TTT |
| | | | | | | 2255 | | 09 | | | | | | |
| AAT_F_ | N_F_N | TTC | 99 | 65.793 | 1 | 27.5603 | 28.2226833 | 1.52264471 | 1.081299109 | 162 | 1.51338926 | 1.5:AATTT | 1.5:AATTT | TTC |
| AAT | | TTT | 63 | 96.207 | | 9958168 | 83133337 | 9763884e- | 1735175e-07 | | 16042002 | TAAT | CAAT | TTT |
| | | | | | | 5992 | | 07 | | | | | | |
| AAT_G_ | N_G_L | GGA | 20 | 17.225 | 3 | 28.8505 | 32.7462968 | 2.40729331 | 3.642967937 | 77 | 1.66942416 | 2.2:AATG | 2.5:AATG | GGG |
| CTA | | GGC | 17 | 15.225 | | 9535538 | 94423274 | 35890936e- | 5444093e-07 | | 24050934 | GTCTA | GGCTA | GGA |
| | | GGG | 24 | 9.564 | | 1035 | | 06 | | | | | | GGC |
| | | GGT | 16 | 34.986 | | | | | | | | | | GGT |
| AAT_G_ | N_G_F | GGA | 55 | 36.015 | 3 | 59.6480 | 67.0465901 | 6.98922074 | 1.830142016 | 161 | 1.82749000 | 1.9:AATG | 2.4:AATG | GGA |
| TTT | | GGC | 21 | 31.834 | | 7832695 | 1481192 | 267279e-13 | 4242133e-14 | | 50289415 | GTTTT | GGTTT | GGG |
| | | GGG | 47 | 19.998 | | 4376 | | | | | | | | GGT |
| | | GGT | 38 | 73.153 | | | | | | | | | | GGC |
| AAT_I_ | N_I_K | ATA | 53 | 52.948 | 2 | 43.2681 | 45.7537837 | 4.02192203 | 1.160625439 | 190 | 1.50888026 | 1.8:AATAT | 1.8:AATAT | ATC |
| AAG | | ATC | 87 | 49.057 | | 8204909 | 9709355 | 04350116e- | 0283679e-10 | | 64623646 | TAAG | CAAG | ATA |
| | | ATT | 50 | 87.995 | | 483 | | 10 | | | | | | ATT |
| AAT_L_ | N_L_K | CTA | 39 | 58.342 | 5 | 69.9296 | 68.1488268 | 1.05992793 | 2.486857338 | 412 | 1.40124735 | 2.6:AATCT | 1.6:AATTT | TTG |
| AAA | | CTC | 28 | 23.791 | | 4330235 | 073247 | 70547842e- | 657898e-13 | | 50677285 | TAAA | GAAA | TTA |
| | | CTG | 46 | 46.604 | | 566 | | 13 | | | | | | CTG |
| | | CTT | 20 | 52.918 | | | | | | | | | | CTA |
| | | TTA | 96 | 113.701 | | | | | | | | | | CTC |
| | | TTG | 183 | 116.642 | | | | | | | | | | CTT |
| AAT_L_ | N_L_L | CTA | 29 | 37.951 | 5 | 41.0912 | 45.8368942 | 8.99284628 | 9.803677621 | 268 | 1.44323809 | 1.5:AATCT | 1.9:AATCT | TTA |
| TTA | | CTC | 11 | 15.476 | | 4682727 | 5280767 | 9250306e- | 41165e-09 | | 04792665 | GTTA | TTTA | CTT |
| | | CTG | 20 | 30.316 | | 4674 | | 08 | | | | | | TTG |
| | | CTT | 66 | 34.423 | | | | | | | | | | CTA |
| | | TTA | 89 | 73.961 | | | | | | | | | | CTG |
| | | TTG | 53 | 75.874 | | | | | | | | | | CTC |
| AAT_S_ | N_S_N | AGC | 71 | 39.323 | 5 | 49.6893 | 50.8110442 | 1.60418672 | 9.454936193 | 349 | 1.34996866 | 1.9:AATTC | 1.8:AATA | TCA |
| AAC | | AGT | 70 | 56.957 | | 5506568 | 80036946 | 71308293e- | 051422e-10 | | 48024654 | TAAC | GCAAC | AGC |
| | | TCA | 81 | 73.020 | | 9256 | | 09 | | | | | | AGT |
| | | TCC | 44 | 54.799 | | | | | | | | | | TCT |
| | | TCG | 34 | 34.011 | | | | | | | | | | TCC |
| | | TCT | 49 | 90.890 | | | | | | | | | | TCG |
| AAT_S_ | N_S_N | AGC | 101 | 62.083 | 5 | 59.5046 | 62.0048558 | 1.53840370 | 4.678560388 | 551 | 1.32783576 | 1.6:AATTC | 1.6:AATA | AGT |
| AAT | | AGT | 127 | 89.924 | | 9962126 | 2723733 | 92040555e- | 174597e-12 | | 5620404 | TAAT | GCAAT | TCA |
| | | TCA | 110 | 115.283 | | 79 | | 11 | | | | | | AGC |
| | | TCC | 75 | 86.517 | | | | | | | | | | TCT |
| | | TCG | 48 | 53.696 | | | | | | | | | | TCC |
| | | TCT | 90 | 143.496 | | | | | | | | | | TCG |
| AAT_S_ | N_S_R | AGC | 17 | 22.535 | 5 | 40.6015 | 46.3343745 | 1.12925900 | 7.764196636 | 200 | 1.51730967 | 1.7:AATTC | 1.9:AATTC | TCA |
| AGA | | AGT | 29 | 32.640 | | 6172397 | 3894892 | 49092499e- | 632157e-09 | | 18438794 | TAGA | AAGA | TCT |
| | | TCA | 80 | 41.845 | | 5706 | | 07 | | | | | | AGT |
| | | TCC | 27 | 31.404 | | | | | | | | | | TCC |
| | | TCG | 16 | 19.490 | | | | | | | | | | AGC |
| | | TCT | 31 | 52.086 | | | | | | | | | | TCG |
| AAT_S_ | N_S_S | AGC | 52 | 27.718 | 5 | 41.4273 | 42.4759048 | 7.69061995 | 4.718511863 | 246 | 1.35596341 | 2.0:AATTC | 1.9:AATA | AGT |
| AGT | | AGT | 53 | 40.148 | | 8983964 | 8005072 | 756558e-08 | 8997255e-08 | | 41429218 | TAGT | GCAGT | AGC |
| | | TCA | 51 | 51.469 | | 321 | | | | | | | | TCA |
| | | TCC | 39 | 38.627 | | | | | | | | | | TCC |
| | | TCG | 19 | 23.973 | | | | | | | | | | TCT |
| | | TCT | 32 | 64.066 | | | | | | | | | | TCG |
| AAT_T_ | N_T_N | ACA | 63 | 46.194 | 3 | 38.7053 | 33.0212603 | 2.00388222 | 3.187586829 | 152 | 1.46283312 | 2.8:AATAC | 1.4:AATA | ACA |
| AAC | | ACC | 45 | 32.203 | | 8896307 | 808999 | 57781667e- | 9917215e-07 | | 01773737 | TAAC | CCAAC | ACC |
| | | ACG | 25 | 21.320 | | 939 | | 08 | | | | | | ACG |
| | | ACT | 19 | 52.282 | | | | | | | | | | ACT |
| ACA_A_ | T_A_N | GCA | 59 | 27.702 | 3 | 45.5733 | 50.7296829 | 6.98895124 | 5.585750375 | 94 | 2.02723490 | 2.2:ACAG | 2.1:ACAG | GCA |
| AAT | | GCC | 14 | 21.000 | | 0466628 | 8426413 | 9461336e- | 4320096e-11 | | 73272697 | CTAAT | CAAAT | GCT |
| | | GCG | 5 | 10.747 | | 635 | | 10 | | | | | | GCC |
| | | GCT | 16 | 34.551 | | | | | | | | | | GCG |
| ACA_E_ | T_E_R | GAA | 2 | 15.375 | 1 | 36.0392 | 38.6412811 | 1.93379568 | 5.093025713 | 22 | 3.28680194 | 7.7:ACAG | 3.0:ACAG | GAG |
| CGG | | GAG | 20 | 6.625 | | 8160486 | 58339915 | 43386035e- | 173779e-10 | | 6453628 | AACGG | AGCGG | GAA |
| | | | | | | 363 | | 09 | | | | | | |
| ACA_G_ | T_G_R | GGA | 30 | 4.250 | 3 | 35.9315 | 55.7704975 | 7.74211271 | 4.702409280 | 19 | 4.01227547 | 7.5:ACAG | 5.5:ACAG | GGG |
| CGA | | GGC | 13 | 3.757 | | 4964858 | 8315956 | 4309382e- | 752227e-12 | | 0041511 | GCCGA | GGCGA | GGT |
| | | GGG | 3 | 2.360 | | 126 | | 08 | | | | | | GGA |
| | | GGT | | 8.633 | | | | | | | | | | GGC |
| ACA_R_ | T_R_M | AGA | 30 | 35.561 | 5 | 54.4982 | 101.081877 | 1.65549006 | 3.126096085 | 75 | 1.96272521 | 5.1:ACAC | 6.4:ACAC | AGA |
| ATG | | AGG | 18 | 16.268 | | 5903132 | 84950827 | 84403214e- | 2353453e-20 | | 00912302 | GAATG | GGATG | CGG |
| | | CGA | 1 | 5.122 | | 8 | | 10 | | | | | | AGG |
| | | CGC | 3 | 4.439 | | | | | | | | | | CGT |
| | | CGG | 20 | 3.131 | | | | | | | | | | CGC |
| | | CGT | 3 | 10.480 | | | | | | | | | | CGA |
| ACA_S_ | T_S_E | AGC | 8 | 8.901 | 5 | 34.2844 | 39.2770716 | 2.08983888 | 2.088578751 | 79 | 1.74772689 | 3.8:ACATC | 2.5:ACAA | AGT |
| GAG | | AGT | 32 | 12.893 | | 4640813 | 04700815 | 36705515e- | 957347e-07 | | 41853585 | GGAG | GTGAG | TCA |
| | | TCA | 18 | 16.529 | | 731 | | 06 | | | | | | TCT |
| | | TCC | 5 | 12.404 | | | | | | | | | | AGC |
| | | TCG | 2 | 7.699 | | | | | | | | | | TCC |
| | | TCT | 14 | 20.574 | | | | | | | | | | TCG |
| ACA_T_ | T_T_E | ACA | 61 | 36.469 | 3 | 36.7180 | 36.4955978 | 5.27868019 | 5.882843950 | 120 | 1.70715658 | 2.0:ACAA | 1.7:ACAA | ACA |
| GAA | | ACC | 13 | 25.424 | | 9668494 | 2599087 | 0782374e- | 9600935e-08 | | 88248719 | CTGAA | CAGAA | ACG |
| | | ACG | 25 | 16.832 | | 225 | | 08 | | | | | | ACT |
| | | ACT | 21 | 41.275 | | | | | | | | | | ACC |
| ACC_D_ | T_D_S | GAC | 34 | 16.946 | 1 | 24.5695 | 26.2365253 | 7.16755110 | 3.020550574 | 49 | 2.04548175 | 2.1:ACCG | 2.0:ACCG | GAC |
| TCC | | GAT | 15 | 32.054 | | 4809405 | 41693493 | 4214243e- | 8253525e-07 | | 98652464 | ATTCC | ACTCC | GAT |
| | | | | | | 0493 | | 07 | | | | | | |
| ACC_F_ | T_F_K | TTC | 30 | 15.027 | 1 | 25.4672 | 25.1233204 | 4.49960411 | 5.377854680 | 37 | 2.17490588 | 3.1:ACCTT | 2.0:ACCTT | TTC |
| AAG | | TTT | 7 | 21.973 | | 3291451 | 63304932 | 78521907e- | 767835e-07 | | 68706332 | TAAG | CAAG | TTT |
| | | | | | | 3108 | | 07 | | | | | | |
| ACC_G_ | T_G_T | GGA | 3 | 12.527 | 3 | 44.4054 | 37.5265490 | 1.23765950 | 3.560123656 | 56 | 1.99688310 | 13.9:ACCG | 1.9:ACCG | GGT |
| ACC | | GGC | 5 | 11.073 | | 7570490 | 7479136 | 3956639e- | 383707e-08 | | 22349933 | GGACC | GTACC | GGC |
| | | GGG | 0 | 6.956 | | 31 | | 09 | | | | | | GGA |
| | | GGT | 48 | 25.444 | | | | | | | | | | GGG |
| ACC_I_ | T_I_R | ATA | 26 | 11.147 | 2 | 26.4252 | 28.7841630 | 1.82734409 | 5.618215641 | 40 | 2.16124380 | 3.1:ACCAT | 2.3:ACCAT | ATA |
| AGG | | ATC | 8 | 10.328 | | 9391897 | 78027397 | 50116063e- | 307599e-07 | | 6580611 | TAGG | AAGG | ATC |
| | | ATT | 6 | 18.525 | | 7584 | | 06 | | | | | | ATT |
| ACC_K | T_K_T | AAA | 15 | 34.176 | 1 | 25.6650 | 25.5724812 | 4.06125050 | 4.260727395 | 59 | 1.88931572 | 2.3:ACCA | 1.8:ACCA | AAG |
| ACT | | AAG | 44 | 24.824 | | 0622015 | 3371452 | 34925973e- | 3064813e-07 | | 12916105 | AAACT | AGACT | AAA |
| | | | | | | 4947 | | 07 | | | | | | |
| ACC_L_ | T_L_L | CTA | 5 | 10.479 | 5 | 41.7555 | 51.0195815 | 6.60084709 | 8.569210657 | 74 | 1.80792506 | 8.4:ACCCT | 3.1:ACCCT | CTT |
| TTG | | CTC | 2 | 4.273 | | 5832776 | 51116474 | 0689098e- | 883697e-10 | | 46409788 | GTTG | TTTG | TTG |
| | | CTG | 1 | 8.371 | | 3765 | | 08 | | | | | | TTA |
| | | CTT | 29 | 9.505 | | | | | | | | | | CTA |
| | | TTA | 18 | 20.422 | | | | | | | | | | CTC |
| | | TTG | 19 | 20.950 | | | | | | | | | | CTG |
| ACC_N_ | T_N_V | AAC | 45 | 22.603 | 1 | 37.5383 | 37.2114378 | 8.96346134 | 1.059902865 | 56 | 2.16291013 | 3.0:ACCA | 2.0:ACCA | AAC |
| GTC | | AAT | 11 | 33.397 | | 1896121 | 6201459 | 0184517e- | 8385972e-09 | | 161724 | ATGTC | ACGTC | AAT |
| | | | | | | 581 | | 10 | | | | | | |
| ACC_N_ | T_N_S | AAC | 129 | 62.563 | 1 | 120.740 | 118.301115 | 4.35552271 | 1.489664016 | 155 | 2.25922855 | 3.6:ACCA | 2.1:ACCA | AAC |
| TCC | | AAT | 26 | 92.437 | | 4156005 | 10856642 | 6773161e- | 1954955e-27 | | 5146455 | ATTCC | ACTCC | AAT |
| | | | | | | 7512 | | 28 | | | | | | |
| ACC_S_ | T_S_S | AGC | 33 | 18.253 | 5 | 43.4374 | 43.8320571 | 3.01247917 | 2.505349754 | 162 | 1.63193954 | 3.2:ACCTC | 1.8:ACCA | TCT |
| TCT | | AGT | 16 | 26.439 | | 7151241 | 5978319 | 42189885e- | 3710104e-08 | | 72964637 | GTCT | GCTCT | AGC |
| | | TCA | 25 | 33.894 | | 478 | | 08 | | | | | | TCA |
| | | TCC | 16 | 25.437 | | | | | | | | | | TCC |
| | | TCG | 5 | 15.787 | | | | | | | | | | AGT |
| | | TCT | 67 | 42.189 | | | | | | | | | | TCG |
| ACC_T_ | T_T_T | ACA | 22 | 44.675 | 3 | 44.6563 | 48.5835088 | 1.09471637 | 1.599881426 | 147 | 1.58083273 | 2.0:ACCAC | 2.0:ACCA | ACC |
| ACT | | ACC | 63 | 31.144 | | 2503271 | 0044422 | 0326009e- | 7851008e-10 | | 6356557 | AACT | CCACT | ACT |
| | | ACG | 11 | 20.619 | | 298 | | 09 | | | | | | ACA |
| | | ACT | 51 | 50.562 | | | | | | | | | | ACG |
| ACC_T_ | T_T_E | ACA | 53 | 79.928 | 3 | 41.3690 | 42.5467029 | 5.46053849 | 3.071451177 | 263 | 1.45556312 | 1.8:ACCAC | 1.5:ACCA | ACT |
| GAA | | ACC | 50 | 55.720 | | 9117484 | 6813991 | 50283775e- | 215228e-09 | | 2342773 | GGAA | CTGAA | ACA |
| | | ACG | 21 | 36.889 | | 197 | | 09 | | | | | | ACC |
| | | ACT | 139 | 90.462 | | | | | | | | | | ACG |
| ACC_T_ | T_T_A | ACA | 17 | 36.165 | 3 | 104.922 | 106.531332 | 1.35787538 | 6.119482458 | 119 | 2.39315540 | 8.3:ACCAC | 2.3:ACCA | ACT |
| GCC | | ACC | 6 | 25.212 | | 3356851 | 01322677 | 32695867e- | 516558e-23 | | 9988261 | GGCC | CTGCC | ACA |
| | | ACG | 2 | 16.691 | | 0586 | | 22 | | | | | | ACC |
| | | ACT | 94 | 40.932 | | | | | | | | | | ACG |
| ACC_T_ | T_T_A | ACA | 17 | 34.342 | 3 | 44.3410 | 41.0569061 | 1.27728670 | 6.359972751 | 113 | 1.64238470 | 5.3:ACCAC | 1.7:ACCA | ACT |
| GCT | | ACC | 25 | 23.941 | | 5333280 | 6632813 | 74627807e- | 636909e-09 | | 50125624 | GGCT | CTGCT | ACC |
| | | ACG | 3 | 15.850 | | 036 | | 09 | | | | | | ACA |
| | | ACT | 68 | 38.868 | | | | | | | | | | ACG |
| ACC_V_ | T_V_T | GTA | 2 | 9.037 | 3 | 58.2536 | 76.0273323 | 1.38754458 | 2.182243290 | 42 | 3.39800821 | 4.5:ACCGT | 3.7:ACCGT | GTC |
| ACC | | GTC | 31 | 8.462 | | 8476198 | 6410411 | 94641212e- | 7069344e-16 | | 33133886 | AACC | CACC | GTG |
| | | GTG | 5 | 8.239 | | 709 | | 12 | | | | | | GTT |
| | | GTT | 4 | 16.262 | | | | | | | | | | GTA |
| ACG_E_ | T_E_V | GAA | 4 | 19.569 | 1 | 37.5118 | 41.1345625 | 9.08577575 | 1.421011781 | 28 | 3.07549460 | 4.9:ACGG | 2.8:ACGG | GAG |
| GTC | | GAG | 24 | 8.431 | | 8286533 | 3343488 | 922209e-10 | 693655e-10 | | 20006058 | AAGTC | AGGTC | GAA |
| | | | | | | 327 | | | | | | | | |
| ACG_G_ | T_G_L | GGA | 26 | 8.724 | 3 | 34.8563 | 44.0899704 | 1.30639649 | 1.444207379 | 39 | 2.74317087 | 2.6:ACGG | 3.0:ACGG | GGA |
| CTG | | GGC | 3 | 7.711 | | 9095502 | 9289287 | 37279983e- | 8357619e-09 | | 5231673 | GCCTG | GACTG | GGT |
| | | GGG | 2 | 4.844 | | 211 | | 07 | | | | | | GGC |
| | | GGT | 8 | 17.720 | | | | | | | | | | GGG |
| ACG_R_ | T_R_K | AGA | 16 | 27.026 | 5 | 35.8569 | 58.7869632 | 1.01448900 | 2.164123441 | 57 | 2.12628601 | 2.0:ACGC | 4.6:ACGC | CGA |
| AAA | | AGG | 15 | 12.364 | | 4542756 | 86128604 | 66489718e- | 4580158e-11 | | 5265877 | GTAAA | GAAAA | AGA |
| | | CGA | 18 | 3.892 | | 3595 | | 06 | | | | | | AGG |
| | | CGC | 2 | 3.374 | | | | | | | | | | CGT |
| | | CGG | 2 | 2.379 | | | | | | | | | | CGG |
| | | CGT | 4 | 7.965 | | | | | | | | | | CGC |
| ACG_R_ | T_R_V | AGA | 6 | 13.750 | 5 | 32.4128 | 38.7786759 | 4.92129826 | 2.631307727 | 29 | 2.81977659 | 4.0:ACGC | 3.2:ACGA | AGG |
| GTA | | AGG | 20 | 6.290 | | 4572261 | 8015095 | 4876971e- | 7969864e-07 | | 09681274 | GAGTA | GGGTA | AGA |
| | | CGA | 0 | 1.980 | | 707 | | 06 | | | | | | CGT |
| | | CGC | 1 | 1.716 | | | | | | | | | | CGC |
| | | CGG | 0 | 1.210 | | | | | | | | | | CGG |
| | | CGT | 2 | 4.052 | | | | | | | | | | CGA |
| ACG_S_ | T_S_A | AGC | 21 | 6.535 | 5 | 27.9760 | 38.7223650 | 3.67925693 | 2.700848814 | 58 | 1.93696602 | 1.9:ACGTC | 3.2:ACGA | AGC |
| GCT | | AGT | 11 | 9.466 | | 9132117 | 3526328 | 40171934e- | 860369e-07 | | 36471191 | TGCT | GCGCT | AGT |
| | | TCA | 9 | 12.135 | | 1886 | | 05 | | | | | | TCA |
| | | TCC | 6 | 9.107 | | | | | | | | | | TCT |
| | | TCG | 3 | 5.652 | | | | | | | | | | TCC |
| | | TCT | 8 | 15.105 | | | | | | | | | | TCG |
| ACT_A_ | T_A_S | GCA | 16 | 45.678 | 3 | 107.082 | 116.607074 | 4.65822960 | 4.150263919 | 155 | 2.04134200 | 8.9:ACTGC | 2.5:ACTGC | GCC |
| AGC | | GCC | 88 | 34.628 | | 0786840 | 2811993 | 4714081e- | 883996e-25 | | 43364225 | GAGC | CAGC | GCT |
| | | GCG | 2 | 17.722 | | 884 | | 23 | | | | | | GCA |
| | | GCT | 49 | 56.972 | | | | | | | | | | GCG |
| ACT_E_ | T_E_S | GAA | 115 | 88.758 | 1 | 31.7574 | 25.7660397 | 1.74676074 | 3.854106191 | 127 | 1.41066036 | 3.2:ACTGA | 1.3:ACTG | GAA |
| AGT | | GAG | 12 | 38.242 | | 7067003 | 47334972 | 58692434e- | 010979e-07 | | 81331351 | GAGT | AAAGT | GAG |
| | | | | | | 7176 | | 08 | | | | | | |
| ACT_F_ | T_F_K | TTC | 63 | 38.582 | 1 | 25.4923 | 26.0218077 | 4.44129885 | 3.375824898 | 95 | 1.67562048 | 1.8:ACTTT | 1.6:ACTTT | TTC |
| AAG | | TTT | 32 | 56.418 | | 9601191 | 94633816 | 9283826e- | 791731e-07 | | 18609424 | TAAG | CAAG | TTT |
| | | | | | | 0337 | | 07 | | | | | | |
| ACT_F_ | T_F_T | TTC | 59 | 34.927 | 1 | 27.4441 | 27.9388225 | 1.61693528 | 1.252121581 | 86 | 1.75032197 | 1.9:ACTTT | 1.7:ACTTT | TTC |
| ACT | | TTT | 27 | 51.073 | | 9458637 | 09731494 | 06023055e- | 0923775e-07 | | 19808407 | TACT | CACT | TTT |
| | | | | | | 053 | | 07 | | | | | | |
| ACT_L_ | T_L_K | CTA | 26 | 44.748 | 5 | 108.734 | 102.691226 | 7.58523602 | 1.430957878 | 316 | 1.55548735 | 5.8:ACTCT | 1.8:ACTTT | TTG |
| AAA | | CTC | 12 | 18.248 | | 1699529 | 2614471 | 7145656e- | 8863083e-20 | | 62397284 | TAAA | GAAA | TTA |
| | | CTG | 21 | 35.745 | | 485 | | 22 | | | | | | CTA |
| | | CTT | 7 | 40.588 | | | | | | | | | | CTG |
| | | TTA | 88 | 87.207 | | | | | | | | | | CTC |
| | | TTG | 162 | 89.463 | | | | | | | | | | CTT |
| ACT_L_ | T_L_K | CTA | 18 | 29.738 | 5 | 57.6088 | 51.2946709 | 3.78752473 | 7.526183676 | 210 | 1.46137415 | 5.4:ACTCT | 1.6:ACTTT | TTG |
| AAG | | CTC | 11 | 12.127 | | 8864535 | 14760246 | 80966353e- | 212241e-10 | | 3610331 | TAAG | GAAG | TTA |
| | | CTG | 15 | 23.755 | | 207 | | 11 | | | | | | CTA |
| | | CTT | 5 | 26.973 | | | | | | | | | | CTG |
| | | TTA | 63 | 57.954 | | | | | | | | | | CTC |
| | | TTG | 98 | 59.454 | | | | | | | | | | CTT |
| ACT_L_ | T_L_I | CTA | 6 | 22.799 | 5 | 51.6543 | 46.2980862 | 6.35124745 | 7.897462281 | 161 | 1.58630770 | 3.8:ACTCT | 1.6:ACTTT | TTG |
| ATT | | CTC | 6 | 9.297 | | 0696764 | 8799013 | 2696689e- | 259745e-09 | | 4252867 | AATT | GATT | TTA |
| | | CTG | 10 | 18.212 | | 513 | | 10 | | | | | | CTG |
| | | CTT | 8 | 20.679 | | | | | | | | | | CTT |
| | | TTA | 57 | 44.432 | | | | | | | | | | CTC |
| | | TTG | 74 | 45.581 | | | | | | | | | | CTA |
| ACT_L_ | T_L_E | CTA | 27 | 43.615 | 5 | 41.4136 | 39.9400423 | 7.73978660 | 1.535524806 | 308 | 1.39182234 | 1.8:ACTCT | 1.4:ACTTT | TTG |
| GAA | | CTC | 10 | 17.786 | | 9949735 | 87993934 | 0835489e- | 6391574e-07 | | 94693511 | TGAA | GGAA | TTA |
| | | CTG | 24 | 34.840 | | 466 | | 08 | | | | | | CTA |
| | | CTT | 22 | 39.560 | | | | | | | | | | CTG |
| | | TTA | 100 | 85.000 | | | | | | | | | | CTT |
| | | TTG | 125 | 87.199 | | | | | | | | | | CTC |
| ACT_N_ | T_N_A | AAC | 20 | 48.032 | 1 | 30.8673 | 27.4326561 | 2.76276313 | 1.626611395 | 119 | 1.52836050 | 2.4:ACTAA | 1.4:ACTA | AAT |
| GCT | | AAT | 99 | 70.968 | | 8302368 | 16099004 | 99577274e- | 0537263e-07 | | 44550574 | CGCT | ATGCT | AAC |
| | | | | | | 093 | | 08 | | | | | | |
| ACT_R_ | T_R_N | AGA | 26 | 32.716 | 5 | 48.9532 | 106.757385 | 2.26856973 | 1.983763944 | 69 | 2.15695556 | 2.0:ACTCG | 6.9:ACTCG | AGA |
| AAT | | AGG | 10 | 14.966 | | 9451547 | 42473353 | 9250614e- | 749101e-21 | | 71693394 | CAAT | GAAT | CGG |
| | | CGA | 3 | 4.712 | | 632 | | 09 | | | | | | AGG |
| | | CGC | 2 | 4.084 | | | | | | | | | | CGT |
| | | CGG | 20 | 2.880 | | | | | | | | | | CGA |
| | | CGT | 8 | 9.642 | | | | | | | | | | CGC |
| ACT_S_ | T_S_A | AGC | 0 | 8.112 | 5 | 93.9551 | 122.968532 | 9.90597922 | 7.375367258 | 72 | 2.97835643 | 16.2:ACTA | 3.9:ACTA | AGT |
| GCG | | AGT | 46 | 11.751 | | 1435784 | 96712145 | 9137595e- | 327657e-25 | | 80803966 | GCGCG | GTGCG | TCT |
| | | TCA | 3 | 15.064 | | 631 | | 19 | | | | | | TCC |
| | | TCC | 7 | 11.305 | | | | | | | | | | TCG |
| | | TCG | 4 | 7.017 | | | | | | | | | | TCA |
| | | TCT | 12 | 18.751 | | | | | | | | | | AGC |
| ACT_S_ | T_S_A | AGC | 8 | 23.211 | 5 | 164.410 | 216.773672 | 1.13560294 | 7.294912415 | 206 | 2.37946323 | 2.9:ACTTC | 3.3:ACTA | AGT |
| GCT | | AGT | 110 | 33.620 | | 9706466 | 43512785 | 5545834e- | 516473e-45 | | 43943394 | CGCT | GTGCT | TCT |
| | | TCA | 21 | 43.100 | | 447 | | 33 | | | | | | TCA |
| | | TCC | 11 | 32.346 | | | | | | | | | | TCC |
| | | TCG | 8 | 20.075 | | | | | | | | | | TCG |
| | | TCT | 48 | 53.648 | | | | | | | | | | AGC |
| ACT_T_ | T_T_T | ACA | 20 | 32.822 | 3 | 58.7895 | 72.9361421 | 1.06611012 | 1.003150265 | 108 | 2.14415398 | 2.5:ACTAC | 2.6:ACTAC | ACC |
| ACC | | ACC | 59 | 22.881 | | 8223555 | 0534403 | 53367532e- | 8222284e-15 | | 35199386 | GACC | CACC | ACT |
| | | ACG | 6 | 15.148 | | 0824 | | 12 | | | | | | ACA |
| | | ACT | 23 | 37.148 | | | | | | | | | | ACG |
| ACT_T_ | T_T_T | ACA | 46 | 106.064 | 3 | 288.975 | 368.152882 | 2.41881210 | 1.748824690 | 349 | 2.61343393 | 3.1:ACTAC | 3.0:ACTAC | ACC |
| ACT | | ACC | 220 | 73.941 | | 6435470 | 931379 | 9969673e- | 766946e-79 | | 3015399 | GACT | CACT | ACT |
| | | ACG | 16 | 48.952 | | 89 | | 62 | | | | | | ACA |
| | | ACT | 67 | 120.043 | | | | | | | | | | ACG |
| AGA_A_ | R_A_K | GCA | 28 | 37.132 | 3 | 30.4064 | 35.6694054 | 1.13340336 | 8.795803043 | 126 | 1.63353050 | 1.5:AGAG | 2.0:AGAG | GCC |
| AAG | | GCC | 56 | 28.149 | | 4367822 | 7188571 | 01032045e- | 386687e-08 | | 50178277 | CTAAG | CCAAG | GCT |
| | | GCG | 11 | 14.406 | | 574 | | 06 | | | | | | GCA |
| | | GCT | 31 | 46.313 | | | | | | | | | | GCG |
| AGA_F_ | R_F_K | TTC | 51 | 28.023 | 1 | 31.4631 | 31.7241504 | 2.03258434 | 1.776987036 | 69 | 1.92938208 | 2.3:AGATT | 1.8:AGATT | TTC |
| AAG | | TTT | 18 | 40.977 | | 7795519 | 49054836 | 59487828e- | 0762586e-08 | | 6641928 | TAAG | CAAG | TTT |
| | | | | | | 2805 | | 08 | | | | | | |
| AGA_G_ | R_G_G | GGA | 12 | 29.081 | 3 | 36.7116 | 33.0094909 | 5.29525457 | 3.205860485 | 130 | 1.52215527 | 3.2:AGAG | 1.5:AGAG | GGT |
| GGT | | GGC | 24 | 25.705 | | 6032068 | 7882536 | 2487711e- | 6748036e-07 | | 8487091 | GGGGT | GTGGT | GGC |
| | | GGG | 5 | 16.147 | | 3265 | | 08 | | | | | | GGA |
| | | GGT | 89 | 59.067 | | | | | | | | | | GGG |
| AGA_K_ | R_K_K | AAA | 99 | 143.076 | 1 | 31.7362 | 32.2708643 | 1.76596152 | 1.341092567 | 247 | 1.43253053 | 1.4:AGAA | 1.4:AGAA | AAG |
| AAA | | AAG | 148 | 103.924 | | 3828661 | 2938946 | 17612218e- | 2773355e-08 | | 258474 | AAAAA | AGAAA | AAA |
| | | | | | | 0742 | | 08 | | | | | | |
| AGA_K_ | R_K_K | AAA | 77 | 111.217 | 1 | 24.6052 | 25.0198731 | 7.03609705 | 5.674244103 | 192 | 1.43187098 | 1.4:AGAA | 1.4:AGAA | AAG |
| AAG | | AAG | 115 | 80.783 | | 1752278 | 53291087 | 0934571e- | 339412e-07 | | 75653788 | AAAAG | AGAAG | AAA |
| | | | | | | 7186 | | 07 | | | | | | |
| AGA_L_ | R_L_A | CTA | 12 | 17.701 | 5 | 43.2193 | 48.0506981 | 3.33550752 | 3.468280445 | 125 | 1.80591468 | 2.4:AGACT | 2.0:AGATT | TTG |
| GCT | | CTC | 3 | 7.218 | | 2455154 | 64129095 | 3901354e- | 400312e-09 | | 44518095 | CGCT | GGCT | TTA |
| | | CTG | 7 | 14.140 | | | | 08 | | | | | | CTA |
| | | CTT | 9 | 16.055 | | | | | | | | | | CTT |
| | | TTA | 24 | 34.497 | | | | | | | | | | CTG |
| | | TTG | 70 | 35.389 | | | | | | | | | | CTC |
| AGA_R_ | R_R_R | AGA | 156 | 100.518 | 5 | 68.4317 | 62.3242974 | 2.17190492 | 4.017797113 | 212 | 1.62488603 | 4.4:AGAC | 1.6:AGAA | AGA |
| AGA | | AGG | 33 | 45.984 | | 2655711 | 97701934 | 04353275e- | 754561e-12 | | 6688506 | GGAGA | GAAGA | AGG |
| | | CGA | 4 | 14.477 | | 712 | | 13 | | | | | | CGT |
| | | CGC | 4 | 12.548 | | | | | | | | | | CGC |
| | | CGG | 2 | 8.849 | | | | | | | | | | CGA |
| | | CGT | 13 | 29.625 | | | | | | | | | | CGG |
| AGA_V_ | R_V_T | GTA | 21 | 6.670 | 3 | 30.5622 | 39.2942031 | 1.05096892 | 1.503608890 | 31 | 2.88879412 | 3.0:AGAG | 3.1:AGAG | GTA |
| ACG | | GTC | 3 | 6.246 | | 7398709 | 6931282 | 97751764e- | 8464834e-08 | | 81736196 | TGACG | TAACG | GTT |
| | | GTG | 2 | 6.081 | | 9214 | | 06 | | | | | | GTC |
| | | GTT | 5 | 12.003 | | | | | | | | | | GTG |
| AGC_I_ | S_I_N | ATA | 11 | 26.753 | 2 | 85.0129 | 101.576585 | 3.46481099 | 8.768488151 | 96 | 2.68301102 | 2.6:AGCAT | 2.7:AGCA | ATC |
| AAC | | ATC | 68 | 24.786 | | 1736558 | 47208345 | 44310744e- | 082336e-23 | | 55275206 | TAAC | TCAAC | ATT |
| | | ATT | 17 | 44.461 | | 898 | | 19 | | | | | | ATA |
| AGC_R_ | S_R_H | AGA | 6 | 18.017 | 5 | 35.3668 | 41.1190537 | 1.27113629 | 8.877256546 | 38 | 2.53490808 | 3.2:AGCC | 2.9:AGCA | AGG |
| CAT | | AGG | 24 | 8.242 | | 5590204 | 614329 | 47708582e- | 113084e-08 | | 088771 | GGCAT | GGCAT | AGA |
| | | CGA | 2 | 2.595 | | 5934 | | 06 | | | | | | CGT |
| | | CGC | 3 | 2.249 | | | | | | | | | | CGC |
| | | CGG | 0 | 1.586 | | | | | | | | | | CGA |
| | | CGT | 3 | 5.310 | | | | | | | | | | CGG |
| AGC_R_ | S_R_C | AGA | 2 | 6.638 | 5 | 38.5890 | 92.0439628 | 2.87291970 | 2.499169785 | 14 | 7.21013990 | 3.3:AGCA | 10.5:AGCC | CGA |
| TGC | | AGG | 1 | 3.037 | | 1094258 | 2321252 | 24764107e- | 132985e-18 | | 6059033 | GATGC | GATGC | AGA |
| | | CGA | 10 | 0.956 | | 019 | | 07 | | | | | | CGT |
| | | CGC | 0 | 0.829 | | | | | | | | | | AGG |
| | | CGG | 0 | 0.584 | | | | | | | | | | CGG |
| | | CGT | 1 | 1.956 | | | | | | | | | | CGC |
| AGC_S_ | S_S_N | AGC | 36 | 19.042 | 5 | 35.4108 | 39.0091548 | 1.24569595 | 2.364781652 | 169 | 1.56052741 | 1.6:AGCTC | 1.9:AGCA | AGT |
| AAT | | AGT | 46 | 27.581 | | 1159083 | 5236385 | 26226377e- | 7335897e-07 | | 7375245 | TAAT | GCAAT | AGC |
| | | TCA | 31 | 35.359 | | 713 | | 06 | | | | | | TCA |
| | | TCC | 17 | 26.536 | | | | | | | | | | TCT |
| | | TCG | 11 | 16.469 | | | | | | | | | | TCC |
| | | TCT | 28 | 44.012 | | | | | | | | | | TCG |
| AGC_S_ | S_S_S | AGC | 38 | 13.521 | 5 | 51.3944 | 63.5505465 | 7.17985569 | 2.238760081 | 120 | 1.87771977 | 2.2:AGCTC | 2.8:AGCA | AGC |
| AGC | | AGT | 29 | 19.584 | | 9879936 | 4050935 | 9512534e- | 5967057e-12 | | 41991691 | TAGC | GCAGC | AGT |
| | | TCA | 19 | 25.107 | | 663 | | 10 | | | | | | TCA |
| | | TCC | 13 | 18.842 | | | | | | | | | | TCT |
| | | TCG | 7 | 11.694 | | | | | | | | | | TCC |
| | | TCT | 14 | 31.251 | | | | | | | | | | TCG |
| AGC_S_ | S_S_S | AGC | 22 | 13.295 | 5 | 34.7052 | 37.7464435 | 1.72279152 | 4.242637609 | 118 | 1.66059679 | 2.2:AGCTC | 2.0:AGCA | AGT |
| AGT | | AGT | 39 | 19.258 | | 7143808 | 0257922 | 5419458e- | 4830547e-07 | | 24106848 | TAGT | GTAGT | AGC |
| | | TCA | 18 | 24.689 | | 663 | | 06 | | | | | | TCA |
| | | TCC | 16 | 18.528 | | | | | | | | | | TCC |
| | | TCG | 9 | 11.499 | | | | | | | | | | TCT |
| | | TCT | 14 | 30.731 | | | | | | | | | | TCG |
| AGC_S_ | S_S_D | AGC | 6 | 8.000 | 5 | 41.7011 | 56.7789451 | 6.77026470 | 5.616253365 | 71 | 2.21709083 | 1.9:AGCTC | 3.0:AGCA | AGT |
| GAC | | AGT | 35 | 11.587 | | 4468338 | 53780725 | 425616e-08 | 4040063e-11 | | 14616153 | AGAC | GTGAC | TCT |
| | | TCA | 8 | 14.855 | | 04 | | | | | | | | TCA |
| | | TCC | 7 | 11.148 | | | | | | | | | | TCC |
| | | TCG | 4 | 6.919 | | | | | | | | | | AGC |
| | | TCT | 11 | 18.490 | | | | | | | | | | TCG |
| AGC_S_ | S_S_D | AGC | 21 | 14.760 | 5 | 38.7391 | 44.9504811 | 2.67995579 | 1.484900730 | 131 | 1.69963983 | 2.1:AGCTC | 2.2:AGCA | AGT |
| GAT | | AGT | 47 | 21.379 | | 2999189 | 58048774 | 6856398e- | 200249e-08 | | 0894411 | CGAT | GTGAT | TCT |
| | | TCA | 19 | 27.409 | | 459 | | 07 | | | | | | AGC |
| | | TCC | 10 | 20.569 | | | | | | | | | | TCA |
| | | TCG | 10 | 12.766 | | | | | | | | | | TCG |
| | | TCT | 24 | 34.116 | | | | | | | | | | TCC |
| AGC_S_ | S_S_G | AGC | 19 | 7.436 | 5 | 43.9882 | 48.5845635 | 2.32896054 | 2.698318805 | 66 | 2.20231532 | 4.6:AGCTC | 2.6:AGCA | AGT |
| GGT | | AGT | 24 | 10.771 | | 6786352 | 2734624 | 96707673e- | 7796217e-09 | | 14900293 | AGGT | GCGGT | AGC |
| | | TCA | 3 | 13.809 | | 262 | | 08 | | | | | | TCT |
| | | TCC | 6 | 10.363 | | | | | | | | | | TCC |
| | | TCG | 3 | 6.432 | | | | | | | | | | TCG |
| | | TCT | 11 | 17.188 | | | | | | | | | | TCA |
| AGC_T_ | S_T_N | ACA | 13 | 34.342 | 3 | 106.875 | 138.304132 | 5.16021834 | 8.772101174 | 113 | 2.83745301 | 2.6:AGCA | 3.1:AGCA | ACC |
| AAC | | ACC | 75 | 23.941 | | 4958864 | 48352178 | 67441914e- | 04915e-30 | | 3406579 | CAAAC | CCAAC | ACT |
| | | ACG | 7 | 15.850 | | 5629 | | 23 | | | | | | ACA |
| | | ACT | 18 | 38.868 | | | | | | | | | | ACG |
| AGC_T_ | S_T_N | ACA | 28 | 55.919 | 3 | 82.9865 | 88.8092756 | 7.01919529 | 3.947209671 | 184 | 1.97584980 | 2.2:AGCA | 2.0:AGCA | ACT |
| AAT | | ACC | 20 | 38.983 | | 4098347 | 7685285 | 8200426e- | 651049e-19 | | 00426776 | CGAAT | CTAAT | ACA |
| | | ACG | 12 | 25.808 | | 772 | | 18 | | | | | | ACC |
| | | ACT | 124 | 63.289 | | | | | | | | | | ACG |
| AGC_V_ | S_V_K | GTA | 37 | 16.353 | 3 | 27.7697 | 33.5240639 | 4.05951321 | 2.496776584 | 76 | 1.84467687 | 1.9:AGCGT | 2.3:AGCG | GTA |
| AAA | | GTC | 10 | 15.312 | | 4010847 | 3580913 | 00829975e- | 293233e-07 | | 39783992 | GAAA | TAAAA | GTT |
| | | GTG | 8 | 14.908 | | 936 | | 06 | | | | | | GTC |
| | | GTT | 21 | 29.427 | | | | | | | | | | GTG |
| AGG_I_ | R_I_A | ATA | 27 | 11.704 | 2 | 27.8978 | 29.3414688 | 8.75094931 | 4.251879847 | 42 | 2.10134322 | 5.4:AGGA | 2.3:AGGA | ATA |
| GCT | | ATC | 2 | 10.844 | | 6692758 | 9750594 | 0965376e- | 515117e-07 | | 0033768 | TCGCT | TAGCT | ATT |
| | | ATT | 13 | 19.452 | | 389 | | 07 | | | | | | ATC |
| AGG_P_ | R_P_R | CCA | 0 | 7.741 | 3 | 47.0388 | 66.0614117 | 3.41012292 | 2.973662177 | 19 | 4.73028204 | 15.5:AGGC | 5.2:AGGC | CCC |
| CGA | | CCC | 16 | 3.049 | | 3436744 | 9365878 | 84205885e- | 267539e-14 | | 7984273 | CACGA | CCCGA | CCT |
| | | CCG | 1 | 2.335 | | 266 | | 10 | | | | | | CCG |
| | | CCT | 2 | 5.874 | | | | | | | | | | CCA |
| AGG_R_ | R_R_Q | AGA | 4 | 9.009 | 5 | 26.9359 | 43.1425772 | 5.87034804 | 3.457179068 | 19 | 2.85963485 | 5.3:AGGC | 7.6:AGGC | AGG |
| CAG | | AGG | 7 | 4.121 | | 7874662 | 22928395 | 3455035e- | 4076195e-08 | | 06372733 | GTCAG | GGCAG | CGG |
| | | CGA | 2 | 1.297 | | 9655 | | 05 | | | | | | AGA |
| | | CGC | 0 | 1.125 | | | | | | | | | | CGA |
| | | CGG | 6 | 0.793 | | | | | | | | | | CGT |
| | | CGT | 0 | 2.655 | | | | | | | | | | CGC |
| AGG_S_ | R_S_R | AGC | 26 | 9.465 | 5 | 39.6330 | 46.7619806 | 1.77066708 | 6.352960082 | 84 | 1.94581759 | 2.3:AGGA | 2.7:AGGA | TCA |
| AGA | | AGT | 6 | 13.709 | | 5931340 | 0109398 | 11790053e- | 998853e-09 | | 760475 | GTAGA | GCAGA | AGC |
| | | TCA | 27 | 17.575 | | 3635 | | 07 | | | | | | TCT |
| | | TCC | 7 | 13.190 | | | | | | | | | | TCG |
| | | TCG | 7 | 8.186 | | | | | | | | | | TCC |
| | | TCT | 11 | 21.876 | | | | | | | | | | AGT |
| AGG_S_ | R_S_L | AGC | 2 | 4.282 | 5 | 34.7106 | 48.6170712 | 1.71853941 | 2.657372753 | 38 | 2.74404873 | 3.0:AGGTC | 3.5:AGGA | AGT |
| CTT | | AGT | 22 | 6.202 | | 5302616 | 8965332 | 23053822e- | 6109394e-09 | | 99549283 | CCTT | GTCTT | TCT |
| | | TCA | 4 | 7.951 | | 634 | | 06 | | | | | | TCA |
| | | TCC | 2 | 5.967 | | | | | | | | | | TCG |
| | | TCG | 3 | 3.703 | | | | | | | | | | TCC |
| | | TCT | 5 | 9.896 | | | | | | | | | | AGC |
| AGG_T_ | R_T_N | ACA | 9 | 12.764 | 3 | 28.3326 | 33.6771469 | 3.09247357 | 2.317785465 | 42 | 2.21807514 | 5.9:AGGA | 2.7:AGGA | ACC |
| AAC | | ACC | 24 | 8.898 | | 9561587 | 5709748 | 91500324e- | 9969035e-07 | | 73714237 | CGAAC | CCAAC | ACA |
| | | ACG | 1 | 5.891 | | 192 | | 06 | | | | | | ACT |
| | | ACT | 8 | 14.446 | | | | | | | | | | ACG |
| AGG_T_ | R_T_S | ACA | 6 | 28.568 | 3 | 110.496 | 112.066617 | 8.58152242 | 3.940453576 | 94 | 2.70469668 | 13.2:AGGA | 2.5:AGGA | ACT |
| AGT | | ACC | 6 | 19.915 | | 1201380 | 14833174 | 4283024e- | 527536e-24 | | 2426026 | CGAGT | CTAGT | ACC |
| | | ACG | 1 | 13.185 | | 6117 | | 24 | | | | | | ACA |
| | | ACT | 81 | 32.332 | | | | | | | | | | ACG |
| AGG_V_ | R_V_P | GTA | 17 | 4.519 | 3 | 35.0298 | 44.2678246 | 1.20069296 | 1.323872257 | 21 | 3.71055814 | 8.1:AGGG | 3.8:AGGG | GTA |
| CCG | | GTC | 2 | 4.231 | | 3983774 | 74091436 | 74608235e- | 3372965e-09 | | 0819095 | TTCCG | TACCG | GTC |
| | | GTG | 1 | 4.119 | | 737 | | 07 | | | | | | GTT |
| | | GTT | 1 | 8.131 | | | | | | | | | | GTG |
| AGG_V_ | R_V_V | GTA | 0 | 6.670 | 3 | 48.8401 | 59.8888078 | 1.41075943 | 6.208714772 | 31 | 3.27499571 | 13.3:AGGG | 3.8:AGGG | GTG |
| GTA | | GTC | 4 | 6.246 | | 7655174 | 8642871 | 2303148e- | 206756e-13 | | 63025862 | TAGTA | TGGTA | GTT |
| | | GTG | 23 | 6.081 | | 661 | | 10 | | | | | | GTC |
| | | GTT | 4 | 12.003 | | | | | | | | | | GTA |
| AGT_A_ | S_A_K | GCA | 15 | 26.228 | 3 | 30.3272 | 34.6754584 | 1.17773605 | 1.426572665 | 89 | 1.78580954 | 1.7:AGTGC | 2.1:AGTG | GCC |
| AAG | | GCC | 42 | 19.883 | | 5461131 | 5236999 | 36485024e- | 6753824e-07 | | 21306424 | AAAG | CCAAG | GCT |
| | | GCG | 12 | 10.176 | | 6476 | | 06 | | | | | | GCA |
| | | GCT | 20 | 32.713 | | | | | | | | | | GCG |
| AGT_A_ | S_A_T | GCA | 20 | 58.645 | 3 | 94.1742 | 88.0074160 | 2.77829150 | 5.867936381 | 199 | 1.85354578 | 2.9:AGTGC | 1.7:AGTG | GCT |
| ACT | | GCC | 17 | 44.458 | | 0385183 | 013717 | 08407295e- | 43899e-19 | | 92295262 | AACT | CGACT | GCG |
| | | GCG | 39 | 22.753 | | 229 | | 20 | | | | | | GCA |
| | | GCT | 123 | 73.145 | | | | | | | | | | GCC |
| AGT_L_ | S_L_Q | CTA | 4 | 8.921 | 5 | 40.7477 | 47.5296554 | 1.05507142 | 4.430424558 | 63 | 2.11817858 | 3.6:AGTCT | 3.0:AGTCT | TTA |
| CAG | | CTC | 1 | 3.638 | | 3283072 | 26012326 | 94215785e- | 6384385e-09 | | 8453407 | CCAG | TCAG | CTT |
| | | CTG | 2 | 7.126 | | 825 | | 07 | | | | | | TTG |
| | | CTT | 24 | 8.092 | | | | | | | | | | CTA |
| | | TTA | 24 | 17.386 | | | | | | | | | | CTG |
| | | TTG | 8 | 17.836 | | | | | | | | | | CTC |
| AGT_T_ | S_T_N | ACA | 35 | 47.106 | 3 | 142.179 | 170.762801 | 1.28067622 | 8.709372706 | 155 | 2.51913242 | 4.4:AGTAC | 3.0:AGTA | ACC |
| AAC | | ACC | 98 | 32.839 | | 7597791 | 07239617 | 90891345e- | 546823e-37 | | 9155739 | TAAC | CCAAC | ACA |
| | | ACG | 10 | 21.741 | | 3196 | | 30 | | | | | | ACT |
| | | ACT | 12 | 53.314 | | | | | | | | | | ACG |
| AGT_T_ | S_T_D | ACA | 10 | 20.058 | 3 | 41.5547 | 52.4852899 | 4.98711946 | 2.360331933 | 66 | 2.30805740 | 2.0:AGTAC | 2.7AGTA | ACC |
| GAC | | ACC | 38 | 13.983 | | 4493678 | 2471594 | 8416364e- | 2586817e-11 | | 3004325 | AGAC | CCGAC | ACT |
| | | ACG | 6 | 9.257 | | 018 | | 09 | | | | | | ACA |
| | | ACT | 12 | 22.702 | | | | | | | | | | ACG |
| ATA_G_ | I_G_L | GGA | 4 | 8.053 | 3 | 57.5116 | 89.8977045 | 1.99841428 | 2.304245251 | 36 | 3.64704118 | 5.5:ATAG | 5.1:ATAG | GGG |
| CTG | | GGC | 6 | 7.118 | | 4991092 | 29137 | 1671969e- | 7434286e-19 | | 32907885 | GTCTG | GGCTG | GGC |
| | | GGG | 23 | 4.472 | | 4156 | | 12 | | | | | | GGA |
| | | GGT | 3 | 16.357 | | | | | | | | | | GGT |
| ATA_H_ | I_H_G | CAC | 18 | 6.778 | 1 | 30.1537 | 28.8827664 | 3.99105490 | 7.689402405 | 19 | 2.87777859 | 12.2:ATAC | 2.7:ATAC | CAC |
| GGG | | CAT | 1 | 12.222 | | 9478567 | 49346057 | 6322672e- | 169052e-08 | | 7270655 | ATGGG | ACGGG | CAT |
| | | | | | | 9146 | | 08 | | | | | | |
| ATA_L_ | I_L_S | CTA | 6 | 14.302 | 5 | 40.3562 | 40.9290556 | 1.26562126 | 9.697561606 | 101 | 1.73424695 | 2.6:ATATT | 2.2:ATACT | TTA |
| TCA | | CTC | 3 | 5.832 | | 5303958 | 4701175 | 94515565e- | 745253e-08 | | 47634436 | GTCA | TTCA | CTT |
| | | CTG | 15 | 11.425 | | 162 | | 07 | | | | | | CTG |
| | | CTT | 29 | 12.973 | | | | | | | | | | TTG |
| | | TTA | 37 | 27.873 | | | | | | | | | | CTA |
| | | TTG | 11 | 28.594 | | | | | | | | | | CTC |
| ATA_P_ | I_P_Q | CCA | 27 | 32.185 | 3 | 32.1169 | 44.6021183 | 4.94474353 | 1.124138391 | 79 | 1.82207226 | 1.8:ATACC | 3.0:ATACC | CCG |
| CAA | | CCC | 7 | 12.679 | | 3641812 | 98115806 | 1901096e- | 0204194e-09 | | 24802086 | CCAA | GCAA | CCA |
| | | CCG | 29 | 9.711 | | 914 | | 07 | | | | | | CCT |
| | | CCT | 16 | 24.425 | | | | | | | | | | CCC |
| ATA_R_ | I_R_K | AGA | 72 | 93.880 | 5 | 42.9888 | 44.0047604 | 3.71433345 | 2.311075727 | 198 | 1.54478026 | 2.5:ATACG | 1.7:ATAC | AGA |
| AAA | | AGG | 71 | 42.947 | | 6176081 | 5952405 | 02195066e- | 021717e-08 | | 931686 | TAAA | GAAAA | AGG |
| | | CGA | 23 | 13.521 | | 956 | | 08 | | | | | | CGA |
| | | CGC | 8 | 11.719 | | | | | | | | | | CGG |
| | | CGG | 13 | 8.265 | | | | | | | | | | CGT |
| | | CGT | 11 | 27.668 | | | | | | | | | | CGC |
| ATA_R_ | I_R_R | AGA | 6 | 5.690 | 5 | 24.0436 | 45.4596307 | 0.00021295 | 1.169922984 | 12 | 2.72323526 | 5.2:ATAA | 10.0:ATAC | AGA |
| CGG | | AGG | 0 | 2.603 | | 9462554 | 4753869 | 5224833228 | 1256966e-08 | | 56701986 | GGCGG | GGCGG | CGG |
| | | CGA | 1 | 0.819 | | 237 | | 4 | | | | | | CGA |
| | | CGC | 0 | 0.710 | | | | | | | | | | CGT |
| | | CGG | 5 | 0.501 | | | | | | | | | | CGC |
| | | CGT | 0 | 1.677 | | | | | | | | | | AGG |
| ATA_S_ | I_S_M | AGC | 10 | 12.056 | 5 | 49.8501 | 46.1301417 | 1.48717941 | 8.544545900 | 107 | 1.77695045 | 3.5:ATAA | 1.8:ATATC | TCA |
| ATG | | AGT | 5 | 17.463 | | 3655823 | 8329489 | 5974709e- | 80785e-09 | | 67192838 | GTATG | CATG | TCC |
| | | TCA | 38 | 22.387 | | 8106 | | 09 | | | | | | TCG |
| | | TCC | 31 | 16.801 | | | | | | | | | | AGC |
| | | TCG | 14 | 10.427 | | | | | | | | | | TCT |
| | | TCT | 9 | 27.866 | | | | | | | | | | AGT |
| ATA_S_ | I_S_L | AGC | 2 | 8.563 | 5 | 42.4296 | 47.2962982 | 4.82144179 | 4.943609855 | 76 | 1.64171260 | 6.2:ATAA | 3.1:ATATC | TCG |
| CTA | | AGT | 2 | 12.403 | | 2562214 | 3976672 | 08492964e- | 0027635e-09 | | 353734 | GTCTA | GCTA | TCT |
| | | TCA | 19 | 15.901 | | 601 | | 08 | | | | | | TCA |
| | | TCC | 11 | 11.933 | | | | | | | | | | TCC |
| | | TCG | 23 | 7.406 | | | | | | | | | | AGT |
| | | TCT | 19 | 19.793 | | | | | | | | | | AGC |
| ATC_E_ | I_E_P | GAA | 14 | 32.847 | 1 | 31.9976 | 35.9143672 | 1.54358239 | 2.061827396 | 47 | 2.33605161 | 2.3:ATCGA | 2.3:ATCG | GAG |
| CCA | | GAG | 33 | 14.153 | | 6204872 | 3861179 | 97502272e- | 810233e-09 | | 0416876 | ACCA | AGCCA | GAA |
| | | | | | | 7882 | | 08 | | | | | | |
| ATC_F_ | I_F_K | TTC | 41 | 21.525 | 1 | 29.6951 | 29.6711673 | 5.05611644 | 5.119057918 | 53 | 2.04787925 | 2.6:ATCTT | 1.9:ATCTT | TTC |
| AAG | | TTT | 12 | 31.475 | | 4990819 | 7088419 | 2225633e- | 19207e-08 | | 67867696 | TAAG | CAAG | TTT |
| | | | | | | 1418 | | 08 | | | | | | |
| ATC_G_ | I_G_T | GGA | 20 | 6.935 | 3 | 26.1502 | 32.2272517 | 8.87105263 | 4.686959591 | 31 | 2.57265044 | 3.1:ATCGG | 2.9:ATCG | GGA |
| ACG | | GGC | 2 | 6.130 | | 1942700 | 8650895 | 6336523e- | 8703924e-07 | | 48217726 | CACG | GAACG | GGT |
| | | GGG | 3 | 3.851 | | 8366 | | 06 | | | | | | GGG |
| | | GGT | 6 | 14.085 | | | | | | | | | | GGC |
| ATC_G_ | I_G_F | GGA | 21 | 13.646 | 3 | 41.3786 | 45.7973310 | 5.43520244 | 6.263056140 | 61 | 2.06791511 | 3.5:ATCGG | 2.9:ATCG | GGG |
| TTT | | GGC | 10 | 12.061 | | 1218973 | 82977095 | 332384e-09 | 892518e-10 | | 6350244 | TTTT | GGTTT | GGA |
| | | GGG | 22 | 7.577 | | 58 | | | | | | | | GGC |
| | | GGT | 8 | 27.716 | | | | | | | | | | GGT |
| ATC_L_ | I_L_V | CTA | 4 | 7.930 | 5 | 36.2910 | 58.8748006 | 8.30624863 | 2.075623334 | 56 | 1.98720457 | 2.6:ATCTT | 4.9:ATCCT | CTC |
| GTC | | CTC | 16 | 3.234 | | 2687605 | 45641024 | 4776105e- | 832669e-11 | | 74245408 | AGTC | CGTC | TTG |
| | | CTG | 8 | 6.335 | | 6854 | | 07 | | | | | | CTT |
| | | CTT | 8 | 7.193 | | | | | | | | | | CTG |
| | | TTA | 6 | 15.454 | | | | | | | | | | TTA |
| | | TTG | 14 | 15.854 | | | | | | | | | | CTA |
| ATC_N_ | I_N_V | AAC | 70 | 39.152 | 1 | 40.1982 | 40.7543236 | 2.29450055 | 1.726210882 | 97 | 1.88024266 | 2.1:ATCAA | 1.8:ATCA | AAC |
| GTC | | AAT | 27 | 57.848 | | 8100967 | 3789456 | 8890776e- | 716682e-10 | | 52590514 | TGTC | ACGTC | AAT |
| | | | | | | 079 | | 10 | | | | | | |
| ATC_P_ | I_P_T | CCA | 8 | 13.852 | 3 | 32.4938 | 46.4034387 | 4.11807319 | 4.654898489 | 34 | 2.85835567 | 2.6:ATCCC | 3.7:ATCCC | CCC |
| ACG | | CCC | 20 | 5.457 | | 1941814 | 74517625 | 1284136e- | 2937e-10 | | 6863594 | TACG | CACG | CCA |
| | | CCG | 2 | 4.179 | | 6025 | | 07 | | | | | | CCT |
| | | CCT | 4 | 10.512 | | | | | | | | | | CCG |
| ATC_S_ | I_S_T | AGC | 22 | 8.000 | 5 | 43.3126 | 48.9601965 | 3.19322019 | 2.261213417 | 71 | 2.10256718 | 3.9:ATCAG | 2.8:ATCA | TCC |
| ACC | | AGT | 3 | 11.587 | | 9683161 | 1808599 | 04215274e- | 533984e-09 | | 42774303 | TACC | GCACC | AGC |
| | | TCA | 11 | 14.855 | | 933 | | 08 | | | | | | TCT |
| | | TCC | 22 | 11.148 | | | | | | | | | | TCA |
| | | TCG | 2 | 6.919 | | | | | | | | | | AGT |
| | | TCT | 11 | 18.490 | | | | | | | | | | TCG |
| ATG_D_ | M_D_R | GAC | 53 | 30.433 | 1 | 23.9719 | 25.5795312 | 9.77494088 | 4.245188275 | 88 | 1.70236363 | 1.6:ATGG | 1.7:ATGG | GAC |
| AGA | | GAT | 35 | 57.567 | | 5129019 | 748482 | 2099125e- | 94111e-07 | | 77153024 | ATAGA | ACAGA | GAT |
| | | | | | | 9492 | | 07 | | | | | | |
| ATG_G_ | M_G_F | GGA | 39 | 19.685 | 3 | 31.4059 | 32.6658034 | 6.98148367 | 3.788173615 | 88 | 1.70820834 | 2.2:ATGG | 2.0:ATGG | GGA |
| TTT | | GGC | 16 | 17.400 | | 7013056 | 42851356 | 6906133e- | 9042094e-07 | | 88597917 | GTTTT | GATTT | GGT |
| | | GGG | 15 | 10.930 | | 3116 | | 07 | | | | | | GGC |
| | | GGT | 18 | 39.984 | | | | | | | | | | GGG |
| ATG_L_ | M_L_I | CTA | 18 | 14.161 | 5 | 35.4377 | 42.9611674 | 1.23035694 | 3.762650534 | 100 | 1.68954621 | 2.1:ATGTT | 2.7:ATGCT | CTG |
| ATA | | CTC | 7 | 5.775 | | 4794973 | 40359844 | 14360205e- | 0095945e-08 | | 56383148 | AATA | GATA | TTG |
| | | CTG | 30 | 11.312 | | 3435 | | 06 | | | | | | CTA |
| | | CTT | 13 | 12.844 | | | | | | | | | | TTA |
| | | TTA | 13 | 27.597 | | | | | | | | | | CTT |
| | | TTG | 19 | 28.311 | | | | | | | | | | CTC |
| ATG_L_ | M_L_R | CTA | 7 | 6.939 | 5 | 64.6006 | 105.106370 | 1.35618833 | 4.426290126 | 49 | 3.30163245 | 3.5:ATGTT | 5.1:ATGCT | CTG |
| CGA | | CTC | 1 | 2.830 | | 9355803 | 32643044 | 56616748e- | 920669e-21 | | 00679346 | GCGA | GCGA | CTA |
| | | CTG | 28 | 5.543 | | 995 | | 12 | | | | | | TTA |
| | | CTT | 3 | 6.294 | | | | | | | | | | TTG |
| | | TTA | 6 | 13.523 | | | | | | | | | | CTT |
| | | TTG | 4 | 13.873 | | | | | | | | | | CTC |
| ATG_L_ | M_L_D | CTA | 27 | 28.605 | 5 | 64.5176 | 74.5296110 | 1.41102792 | 1.166202405 | 202 | 1.69511043 | 1.9:ATGTT | 2.4:ATGCT | CTT |
| GAT | | CTC | 15 | 11.665 | | 7244618 | 9815364 | 99638154e- | 9670409e-14 | | 8348459 | AGAT | TGAT | TTG |
| | | CTG | 34 | 22.850 | | 054 | | 12 | | | | | | CTG |
| | | CTT | 61 | 25.945 | | | | | | | | | | TTA |
| | | TTA | 29 | 55.747 | | | | | | | | | | CTA |
| | | TTG | 36 | 57.189 | | | | | | | | | | CTC |
| ATG_L_ | M_L_V | CTA | 13 | 12.461 | 5 | 45.5653 | 56.7312920 | 1.11338669 | 5.744687040 | 88 | 1.94585504 | 2.4:ATGTT | 2.9:ATGCT | CTT |
| GTT | | CTC | 4 | 5.082 | | 6403557 | 9646335 | 59304876e- | 834783e-11 | | 25458956 | AGTT | TGTT | TTG |
| | | CTG | 14 | 9.954 | | 004 | | 08 | | | | | | CTG |
| | | CTT | 33 | 11.303 | | | | | | | | | | CTA |
| | | TTA | 10 | 24.286 | | | | | | | | | | TTA |
| | | TTG | 14 | 24.914 | | | | | | | | | | CTC |
| ATG_L_ | M_L_L | CTA | 28 | 21.949 | 5 | 37.3113 | 41.2048714 | 5.18744728 | 8.529774979 | 155 | 1.55406692 | 1.9:ATGTT | 2.1:ATGCT | CTT |
| TTA | | CTC | 13 | 8.951 | | 5040624 | 7606481 | 7021409e- | 18915e-08 | | 40778813 | GTTA | TTTA | TTA |
| | | CTG | 18 | 17.533 | | 663 | | 07 | | | | | | CTA |
| | | CTT | 42 | 19.909 | | | | | | | | | | TTG |
| | | TTA | 31 | 42.776 | | | | | | | | | | CTG |
| | | TTG | 23 | 43.882 | | | | | | | | | | CTC |
| ATG_L_ | M_L_L | CTA | 26 | 19.400 | 5 | 52.2818 | 61.3295142 | 4.72236991 | 6.454372249 | 137 | 1.72395076 | 2.4:ATGTT | 2.6:ATGCT | CTT |
| TTG | | CTC | 6 | 7.911 | | 4882446 | 4292614 | 1055467e- | 9726e-12 | | 03919462 | GTTG | TTTG | TTA |
| | | CTG | 16 | 15.497 | | 587 | | 10 | | | | | | CTA |
| | | CTT | 45 | 17.597 | | | | | | | | | | TTG |
| | | TTA | 28 | 37.808 | | | | | | | | | | CTG |
| | | TTG | 16 | 38.786 | | | | | | | | | | CTC |
| ATG_R_ | M_R_T | AGA | 14 | 20.388 | 5 | 37.0202 | 50.7403242 | 5.93385970 | 9.775589200 | 43 | 2.45354653 | 5.1:ATGCG | 3.7:ATGC | CGT |
| ACT | | AGG | 5 | 9.327 | | 1189873 | 2434477 | 7390055e- | 346798e-10 | | 4318094 | CACT | GTACT | AGA |
| | | CGA | 1 | 2.936 | | 795 | | 07 | | | | | | AGG |
| | | CGC | 0 | 2.545 | | | | | | | | | | CGG |
| | | CGG | 1 | 1.795 | | | | | | | | | | CGA |
| | | CGT | 22 | 6.009 | | | | | | | | | | CGC |
| ATG_R_ | M_R_V | AGA | 12 | 20.388 | 5 | 24.0442 | 39.4487303 | 0.00021290 | 1.928750586 | 43 | 1.99708617 | 1.7:ATGA | 4.4:ATGC | CGA |
| GTT | | AGG | 7 | 9.327 | | 1021134 | 6230333 | 6637773007 | 1450428e-07 | | 86968288 | GAGTT | GAGTT | AGA |
| | | CGA | 13 | 2.936 | | 523 | | 76 | | | | | | AGG |
| | | CGC | 2 | 2.545 | | | | | | | | | | CGT |
| | | CGG | 3 | 1.795 | | | | | | | | | | CGG |
| | | CGT | 6 | 6.009 | | | | | | | | | | CGC |
| ATG_T_ | M_T_T | ACA | 11 | 21.578 | 3 | 30.9445 | 37.6462284 | 8.73208356 | 3.358414686 | 71 | 1.94841906 | 2.0:ATGAC | 2.4:ATGA | ACC |
| ACC | | ACC | 36 | 15.042 | | 5290994 | 0682933 | 8435169e- | 432946e-08 | | 50635995 | AACC | CCACC | ACT |
| | | ACG | 6 | 9.959 | | 807 | | 07 | | | | | | ACA |
| | | ACT | 18 | 24.421 | | | | | | | | | | ACG |
| ATG_T_ | M_T_S | ACA | 9 | 15.195 | 3 | 23.5050 | 32.6269014 | 3.16869132 | 3.860407419 | 50 | 1.98616519 | 1.7:ATGAC | 3.0:ATGA | ACG |
| AGC | | ACC | 8 | 10.593 | | 6964781 | 34574066 | 6314747e- | 069301e-07 | | 38281764 | AAGC | CGAGC | ACT |
| | | ACG | 21 | 7.013 | | 943 | | 05 | | | | | | ACA |
| | | ACT | 12 | 17.198 | | | | | | | | | | ACC |
| ATG_V_ | M_V_K | GTA | 38 | 36.580 | 3 | 40.2571 | 45.8806636 | 9.39809745 | 6.012698753 | 170 | 1.56401791 | 1.6:ATGGT | 2.0:ATGGT | GTC |
| AAA | | GTC | 68 | 34.251 | | 7188104 | 1213508 | 1208549e- | 471637e-10 | | 03555985 | TAAA | CAAA | GTT |
| | | GTG | 23 | 33.347 | | 6446 | | 09 | | | | | | GTA |
| | | GTT | 41 | 65.822 | | | | | | | | | | GTG |
| ATG_V_ | M_V_A | GTA | 21 | 7.316 | 3 | 30.1226 | 34.6210700 | 1.30045046 | 1.464810605 | 34 | 2.36219762 | 6.9:ATGGT | 2.9:ATGGT | GTA |
| GCG | | GTC | 1 | 6.850 | | 6092553 | 7896383 | 48767009e- | 6706556e-07 | | 36157733 | CGCG | AGCG | GTT |
| | | GTG | 2 | 6.669 | | 9773 | | 06 | | | | | | GTG |
| | | GTT | 10 | 13.164 | | | | | | | | | | GTC |
| ATT_A_ | I_A_K | GCA | 48 | 62.181 | 3 | 33.5394 | 38.1018573 | 2.47822574 | 2.689552236 | 211 | 1.46302634 | 1.4:ATTGC | 1.8:ATTGC | GCC |
| AAG | | GCC | 84 | 47.139 | | 1182946 | 388055 | 70998066e- | 449402e-08 | | 5416694 | TAAG | CAAG | GCT |
| | | GCG | 23 | 24.125 | | 071 | | 07 | | | | | | GCA |
| | | GCT | 56 | 77.556 | | | | | | | | | | GCG |
| ATT_A_ | I_A_I | GCA | 17 | 31.827 | 3 | 34.0681 | 38.4689326 | 1.91661677 | 2.248803468 | 108 | 1.67691274 | 2.1:ATTGC | 2.1:ATTGC | GCC |
| ATC | | GCC | 50 | 24.128 | | 6430051 | 8268412 | 03091065e- | 841912e-08 | | 1624707 | GATC | CATC | GCT |
| | | GCG | 6 | 12.348 | | 729 | | 07 | | | | | | GCA |
| | | GCT | 35 | 39.697 | | | | | | | | | | GCG |
| ATT_A_ | I_A_A | GCA | 23 | 52.456 | 3 | 35.0766 | 31.8488469 | 1.17366649 | 5.631742966 | 178 | 1.44837887 | 2.3:ATTGC | 1.4:ATTGC | GCT |
| GCT | | GCC | 48 | 39.766 | | 3704423 | 8707871 | 3518817e- | 926274e-07 | | 87438858 | AGCT | TGCT | GCC |
| | | GCG | 14 | 20.351 | | 153 | | 07 | | | | | | GCA |
| | | GCT | 93 | 65.426 | | | | | | | | | | GCG |
| ATT_D_ | I_D_K | GAC | 88 | 54.988 | 1 | 28.5402 | 30.2969749 | 9.17686178 | 3.707036057 | 159 | 1.53841761 | 1.5:ATTGA | 1.6:ATTGA | GAC |
| AAG | | GAT | 71 | 104.012 | | 9558259 | 1966013 | 6485416e- | 7531034e-08 | | 35940264 | TAAG | CAAG | GAT |
| | | | | | | 6443 | | 08 | | | | | | |
| ATT_E_ | I_E_L | GAA | 13 | 27.955 | 1 | 23.6823 | 26.5698966 | 1.13619749 | 2.541734863 | 40 | 2.21157881 | 2.2:ATTGA | 2.2:ATTGA | GAG |
| CTC | | GAG | 27 | 12.045 | | 5088020 | 6339867 | 45606382e- | 2555785e-07 | | 52947897 | ACTC | GCTC | GAA |
| | | | | | | 6233 | | 06 | | | | | | |
| ATT_F_ | I_F_K | TTC | 122 | 79.195 | 1 | 38.0631 | 38.9584933 | 6.84908664 | 4.329127073 | 195 | 1.55751804 | 1.6:ATTTT | 1.5:ATTTT | TTC |
| AAA | | TTT | 73 | 115.805 | | 6918927 | 6658557 | 9249811e- | 2949576e-10 | | 2098327 | TAAA | CAAA | TTT |
| | | | | | | 2685 | | 10 | | | | | | |
| ATT_F_ | I_F_N | TTC | 80 | 48.735 | 1 | 33.0979 | 33.7731969 | 8.76294464 | 6.192620661 | 120 | 1.68695118 | 1.8:ATTTT | 1.6:ATTTT | TTC |
| AAC | | TTT | 40 | 71.265 | | 8590964 | 2580683 | 7055646e- | 00098e-09 | | 49119811 | TAAC | CAAC | TTT |
| | | | | | | 119 | | 09 | | | | | | |
| ATT_F_ | I_F_K | TTC | 86 | 49.141 | 1 | 45.9043 | 46.5517994 | 1.24171127 | 8.922827521 | 121 | 1.83279370 | 2.1:ATTTT | 1.8:ATTTT | TTC |
| AAG | | TTT | 35 | 71.859 | | 3109941 | 9083492 | 41168346e- | 90336e-12 | | 38236362 | TAAG | CAAG | TTT |
| | | | | | | 034 | | 11 | | | | | | |
| ATT_F_ | I_F_N | TTC | 97 | 60.919 | 1 | 35.2010 | 35.9838632 | 2.97366394 | 1.989584058 | 150 | 1.62299877 | 1.7:ATTTT | 1.6:ATTTT | TTC |
| AAT | | TTT | 53 | 89.081 | | 2892595 | 5587737 | 8740741e- | 935774e-09 | | 87214508 | TAAT | CAAT | TTT |
| | | | | | | 362 | | 09 | | | | | | |
| ATT_G_ | I_G_L | GGA | 16 | 19.909 | 3 | 48.0192 | 70.0287204 | 2.10944578 | 4.208319525 | 89 | 2.08482630 | 1.6:ATTGG | 3.3:ATTGG | GGG |
| CTT | | GGC | 11 | 17.598 | | 9208588 | 2091923 | 7838356e- | 76807e-15 | | 68270394 | TCTT | GCTT | GGT |
| | | GGG | 37 | 11.055 | | 137 | | 10 | | | | | | GGA |
| | | GGT | 25 | 40.438 | | | | | | | | | | GGC |
| ATT_G_ | I_G_F | GGA | 15 | 20.133 | 3 | 32.1378 | 35.8499087 | 4.89477745 | 8.055993321 | 90 | 1.51846533 | 3.6:ATTGG | 2.5:ATTGG | GGT |
| TTC | | GGC | 5 | 17.795 | | 6316747 | 7316597 | 4191303e- | 999106e-08 | | 44060792 | CTTC | GTTC | GGG |
| | | GGG | 28 | 11.179 | | 404 | | 07 | | | | | | GGA |
| | | GGT | 42 | 40.893 | | | | | | | | | | GGC |
| ATT_G_ | I_G_F | GGA | 47 | 33.555 | 3 | 35.1842 | 39.4671601 | 1.11381166 | 1.381930945 | 150 | 1.61287083 | 1.6:ATTGG | 2.1:ATTGG | GGA |
| TTT | | GGC | 21 | 29.659 | | 2768522 | 9167873 | 52085155e- | 526583e-08 | | 03574275 | TTTT | GTTT | GGT |
| | | GGG | 39 | 18.632 | | 9374 | | 07 | | | | | | GGG |
| | | GGT | 43 | 68.155 | | | | | | | | | | GGC |
| ATT_I_ | I_I_K | ATA | 30 | 39.851 | 2 | 39.8157 | 45.3724796 | 2.26001309 | 1.404401329 | 143 | 1.70459433 | 1.6:ATTAT | 2.0:ATTAT | ATC |
| AAG | | ATC | 72 | 36.921 | | 9046320 | 3658716 | 008817e-09 | 033136e-10 | | 86135336 | TAAG | CAAG | ATT |
| | | ATT | 41 | 66.228 | | 916 | | | | | | | | ATA |
| ATT_L_ | I_L_K | CTA | 54 | 61.033 | 5 | 66.9511 | 59.1317076 | 4.41065639 | 1.836977799 | 431 | 1.28057267 | 3.5:ATTCT | 1.5:ATTTT | TTG |
| AAA | | CTC | 17 | 24.889 | | 7931046 | 573359 | 49486627e- | 8443952e-11 | | 2986552 | TAAA | GAAA | TTA |
| | | CTG | 45 | 48.754 | | 98 | | 13 | | | | | | CTA |
| | | CTT | 16 | 55.359 | | | | | | | | | | CTG |
| | | TTA | 119 | 118.944 | | | | | | | | | | CTC |
| | | TTG | 180 | 122.021 | | | | | | | | | | CTT |
| ATT_L_ | I_L_K | CTA | 26 | 44.323 | 5 | 42.7418 | 39.1891217 | 4.16812416 | 2.175509543 | 313 | 1.35695967 | 2.4:ATTCT | 1.4:ATTTT | TTG |
| AAG | | CTC | 15 | 18.075 | | 2557347 | 8885377 | 1644604e- | 9238677e-07 | | 03973232 | TAAG | GAAG | TTA |
| | | CTG | 29 | 35.406 | | 6184 | | 08 | | | | | | CTG |
| | | CTT | 17 | 40.203 | | | | | | | | | | CTA |
| | | TTA | 103 | 86.380 | | | | | | | | | | CTT |
| | | TTG | 123 | 88.614 | | | | | | | | | | CTC |
| ATT_L_ | I_L_G | CTA | 22 | 31.295 | 5 | 38.5130 | 41.6963571 | 2.97577357 | 6.785376375 | 221 | 1.45952455 | 1.8:ATTCT | 1.9:ATTCT | TTG |
| GGT | | CTC | 21 | 12.762 | | 4736932 | 94772325 | 2804654e- | 79173e-08 | | 54944543 | GGGT | TGGT | CTT |
| | | CTG | 14 | 24.999 | | 8945 | | 07 | | | | | | TTA |
| | | CTT | 53 | 28.386 | | | | | | | | | | CTA |
| | | TTA | 41 | 60.990 | | | | | | | | | | CTC |
| | | TTG | 70 | 62.568 | | | | | | | | | | CTG |
| ATT_L_ | I_L_Y | CTA | 8 | 15.435 | 5 | 33.3849 | 38.0699041 | 3.15582386 | 3.653178714 | 109 | 1.69975234 | 1.9:ATTCT | 2.2:ATTCT | CTG |
| TAC | | CTC | 4 | 6.294 | | 2561832 | 5531715 | 0494381e- | 122812e-07 | | 8654816 | ATAC | GTAC | TTA |
| | | CTG | 27 | 12.330 | | 271 | | 06 | | | | | | CTT |
| | | CTT | 26 | 14.000 | | | | | | | | | | TTG |
| | | TTA | 26 | 30.081 | | | | | | | | | | CTA |
| | | TTG | 18 | 30.859 | | | | | | | | | | CTC |
| ATT_L_ | I_L_Y | CTA | 15 | 19.117 | 5 | 45.3839 | 56.6296502 | 1.21212397 | 6.028508592 | 135 | 1.65627897 | 2.0:ATTTT | 2.6:ATTCT | CTT |
| TAT | | CTC | 10 | 7.796 | | 7635939 | 65029014 | 86348256e- | 524408e-11 | | 39578838 | GTAT | TTAT | TTA |
| | | CTG | 11 | 15.271 | | 7245 | | 08 | | | | | | TTG |
| | | CTT | 45 | 17.340 | | | | | | | | | | CTA |
| | | TTA | 35 | 37.256 | | | | | | | | | | CTG |
| | | TTG | 19 | 38.220 | | | | | | | | | | CTC |
| ATT_L_ | I_L_F | CTA | 14 | 21.241 | 5 | 67.0778 | 87.3201731 | 4.15142535 | 2.454649663 | 150 | 1.83700776 | 2.2:ATTTT | 2.9:ATTCT | CTT |
| TTC | | CTC | 11 | 8.662 | | 2581608 | 6960165 | 93672057e- | 5576763e-17 | | 10979787 | GTTC | TTTC | TTA |
| | | CTG | 15 | 16.968 | | 694 | | 13 | | | | | | TTG |
| | | CTT | 56 | 19.266 | | | | | | | | | | CTG |
| | | TTA | 35 | 41.396 | | | | | | | | | | CTA |
| | | TTG | 19 | 42.467 | | | | | | | | | | CTC |
| ATT_L_ | I_L_L | CTA | 30 | 28.180 | 5 | 34.7566 | 40.1848778 | 1.68260072 | 1.370500472 | 199 | 1.37824229 | 1.6:ATTTT | 2.1:ATTCT | TTA |
| TTG | | CTC | 11 | 11.491 | | 7432032 | 8940624 | 2047318e- | 2238915e-07 | | 6220989 | GTTG | TTTG | CTT |
| | | CTG | 15 | 22.510 | | 823 | | 06 | | | | | | TTG |
| | | CTT | 53 | 25.560 | | | | | | | | | | CTA |
| | | TTA | 55 | 54.919 | | | | | | | | | | CTG |
| | | TTG | 35 | 56.339 | | | | | | | | | | CTC |
| ATT_S_ | I_S_I | AGC | 29 | 43.379 | 5 | 47.6980 | 49.6524140 | 4.09343230 | 1.632342004 | 385 | 1.31905009 | 1.8:ATTAG | 1.7:ATTTC | TCC |
| AAA | | AGT | 34 | 62.833 | | 5302529 | 97937886 | 09376095e- | 7440272e-09 | | 90428895 | TAAA | CAAA | TCT |
| | | TCA | 88 | 80.552 | | 998 | | 09 | | | | | | TCA |
| | | TCC | 103 | 60.452 | | | | | | | | | | TCG |
| | | TCG | 40 | 37.519 | | | | | | | | | | AGT |
| | | TCT | 91 | 100.265 | | | | | | | | | | AGC |
| ATT_S_ | I_S_N | AGC | 13 | 22.873 | 5 | 39.6779 | 40.1690772 | 1.73416800 | 1.380595538 | 203 | 1.43328332 | 2.4:ATTAG | 1.8:ATTTC | TCC |
| AAC | | AGT | 14 | 33.130 | | 4553457 | 9817986 | 65217274e- | 4328538e-07 | | 07200095 | TAAC | CAAC | TCA |
| | | TCA | 53 | 42.473 | | 752 | | 07 | | | | | | TCT |
| | | TCC | 58 | 31.875 | | | | | | | | | | TCG |
| | | TCG | 17 | 19.783 | | | | | | | | | | AGT |
| | | TCT | 48 | 52.867 | | | | | | | | | | AGC |
| ATT_S_ | I_S_K | AGC | 16 | 25.464 | 5 | 45.7679 | 46.1501944 | 1.01254349 | 8.464586078 | 226 | 1.46236118 | 2.5:ATTAG | 1.8:ATTTC | TCC |
| AAG | | AGT | 15 | 36.884 | | 8751524 | 83972584 | 62068408e- | 68611e-09 | | 66548325 | TAAG | CAAG | TCA |
| | | TCA | 53 | 47.285 | | 821 | | 08 | | | | | | TCT |
| | | TCC | 64 | 35.486 | | | | | | | | | | TCG |
| | | TCG | 31 | 22.024 | | | | | | | | | | AGC |
| | | TCT | 47 | 58.857 | | | | | | | | | | AGT |
| ATT_S_ | I_S_R | AGC | 13 | 21.521 | 5 | 38.2652 | 39.7046142 | 3.33743238 | 1.712838299 | 191 | 1.46776535 | 2.2:ATTAG | 1.8:ATTTC | TCA |
| AGA | | AGT | 14 | 31.172 | | 6960231 | 81210695 | 8803442e- | 6379525e-07 | | 14224065 | TAGA | AAGA | TCT |
| | | TCA | 71 | 39.962 | | 792 | | 07 | | | | | | TCC |
| | | TCC | 35 | 29.991 | | | | | | | | | | TCG |
| | | TCG | 18 | 18.613 | | | | | | | | | | AGT |
| | | TCT | 40 | 49.742 | | | | | | | | | | AGC |
| ATT_S_ | I_S_I | AGC | 8 | 12.169 | 5 | 40.5031 | 50.5128523 | 1.18211613 | 1.088238398 | 108 | 1.73916873 | 2.1:ATTTC | 2.5:ATTTC | TCC |
| ATC | | AGT | 11 | 17.626 | | 4145165 | 6777753 | 68092106e- | 042794e-09 | | 51530194 | AATC | CATC | TCT |
| | | TCA | 11 | 22.596 | | 969 | | 07 | | | | | | TCA |
| | | TCC | 43 | 16.958 | | | | | | | | | | AGT |
| | | TCG | 8 | 10.525 | | | | | | | | | | TCG |
| | | TCT | 27 | 28.126 | | | | | | | | | | AGC |
| ATT_S_ | I_S_I | AGC | 16 | 23.774 | 5 | 34.7636 | 39.4546917 | 1.67722671 | 1.923424517 | 211 | 1.41443878 | 1.9:ATTTC | 1.9:ATTTC | TCC |
| ATT | | AGT | 28 | 34.436 | | 3992111 | 76463974 | 84067661e- | 0183666e-07 | | 0786557 | GATT | CATT | TCT |
| | | TCA | 34 | 44.147 | | 6194 | | 06 | | | | | | TCA |
| | | TCC | 64 | 33.131 | | | | | | | | | | AGT |
| | | TCG | 11 | 20.562 | | | | | | | | | | AGC |
| | | TCT | 58 | 54.950 | | | | | | | | | | TCG |
| ATT_S_ | I_S_S | AGC | 13 | 23.211 | 5 | 41.4046 | 40.5275701 | 7.77258654 | 1.168771020 | 206 | 1.41563335 | 2.4:ATTAG | 1.6:ATTTC | TCT |
| TCT | | AGT | 14 | 33.620 | | 1452453 | 02021386 | 8611012e- | 8499561e-07 | | 98254205 | TTCT | TTCT | TCA |
| | | TCA | 45 | 43.100 | | 95 | | 08 | | | | | | TCC |
| | | TCC | 33 | 32.346 | | | | | | | | | | AGT |
| | | TCG | 13 | 20.075 | | | | | | | | | | TCG |
| | | TCT | 88 | 53.648 | | | | | | | | | | AGC |
| ATT_V_ | I_V_K | GTA | 30 | 38.732 | 3 | 34.6006 | 39.5731879 | 1.47940064 | 1.312253780 | 180 | 1.48740284 | 1.5:ATTGT | 1.9:ATTGT | GTC |
| AAG | | GTC | 69 | 36.266 | | 9049244 | 28039296 | 17068548e- | 7642233e-08 | | 50291856 | TAAG | CAAG | GTT |
| | | GTG | 35 | 35.308 | | 6056 | | 07 | | | | | | GTG |
| | | GTT | 46 | 69.694 | | | | | | | | | | GTA |
| ATT_V_ | I_V_I | GTA | 17 | 23.884 | 3 | 37.4002 | 44.3773577 | 3.78603491 | 1.254801935 | 111 | 1.74433380 | 2.2:ATTGT | 2.2:ATTGT | GTC |
| ATC | | GTC | 50 | 22.364 | | 9804363 | 8971802 | 40473504e- | 7579798e-09 | | 88290152 | GATC | CATC | GTT |
| | | GTG | 10 | 21.773 | | 613 | | 08 | | | | | | GTA |
| | | GTT | 34 | 42.978 | | | | | | | | | | GTG |
| ATT_V_ | I_V_I | GTA | 15 | 35.074 | 3 | 63.9347 | 69.4418184 | 8.47591191 | 5.620508081 | 163 | 1.64440146 | 2.5:ATTGT | 2.2:ATTGT | GTC |
| ATT | | GTC | 72 | 32.841 | | 8596507 | 9745802 | 8078541e- | 8876785e-15 | | 60425665 | GATT | CATT | GTT |
| | | GTG | 13 | 31.974 | | 867 | | 14 | | | | | | GTA |
| | | GTT | 63 | 63.112 | | | | | | | | | | GTG |
| CAA_G_ | Q_G_S | GGA | 12 | 10.066 | 3 | 40.3687 | 54.0103045 | 8.89972731 | 1.116396438 | 45 | 2.41226149 | 3.4:CAAG | 3.8:CAAG | GGG |
| AGC | | GGC | 6 | 8.898 | | 8573393 | 3483521 | 1037527e- | 4684065e-11 | | 0693765 | GTAGC | GGAGC | GGA |
| | | GGG | 21 | 5.589 | | 371 | | 09 | | | | | | GGT |
| | | GGT | 6 | 20.446 | | | | | | | | | | GGC |
| CAA_I_ | Q_I_I | ATA | 19 | 25.360 | 2 | 35.9470 | 41.1228804 | 1.56383936 | 1.175655173 | 91 | 1.88211276 | 1.9:CAAAT | 2.1:CAAA | ATC |
| ATC | | ATC | 50 | 23.495 | | 7362830 | 9987413 | 61797665e- | 9034503e-09 | | 62797839 | TATC | TCATC | ATT |
| | | ATT | 22 | 42.145 | | 032 | | 08 | | | | | | ATA |
| CAA_I_ | Q_I_S | ATA | 52 | 27.868 | 2 | 29.4884 | 31.2763371 | 3.95055890 | 1.615956558 | 100 | 1.65084680 | 2.3:CAAAT | 1.9:CAAA | ATA |
| TCA | | ATC | 11 | 25.819 | | 7717652 | 46827963 | 0480532e- | 375736e-07 | | 89668151 | CTCA | TATCA | ATT |
| | | ATT | 37 | 46.313 | | 4103 | | 07 | | | | | | ATC |
| CAA_K_ | Q_K_K | AAA | 113 | 156.398 | 1 | 28.1399 | 28.6216963 | 1.12851972 | 8.799074272 | 270 | 1.38287410 | 1.4:CAAA | 1.4:CAAA | AAG |
| AAA | | AAG | 157 | 113.602 | | 5310815 | 4020832 | 58966307e- | 879636e-08 | | 0187737 | AAAAA | AGAAA | AAA |
| | | | | | | 1076 | | 07 | | | | | | |
| CAA_K_ | Q_K_K | AAA | 81 | 119.326 | 1 | 28.7798 | 29.2573898 | 8.10905127 | 6.337407004 | 206 | 1.45428991 | 1.5:CAAA | 1.4:CAAA | AAG |
| AAG | | AAG | 125 | 86.674 | | 4746183 | 4044208 | 8956942e- | 646516e-08 | | 70356012 | AAAAG | AGAAG | AAA |
| | | | | | | 4663 | | 08 | | | | | | |
| CAA_L_ | Q_L_K | CTA | 54 | 50.129 | 5 | 61.6481 | 54.5201800 | 5.54549349 | 1.638396630 | 354 | 1.37905839 | 3.5:CAACT | 1.4:CAACT | TTG |
| AAA | | CTC | 15 | 20.442 | | 0543039 | 3148646 | 76464595e- | 0452317e-10 | | 31958 | TAAA | GAAA | TTA |
| | | CTG | 58 | 40.044 | | 798 | | 12 | | | | | | CTG |
| | | CTT | 13 | 45.469 | | | | | | | | | | CTA |
| | | TTA | 74 | 97.694 | | | | | | | | | | CTC |
| | | TTG | 140 | 100.222 | | | | | | | | | | CTT |
| CAA_Q_ | Q_Q_Q | CAA | 483 | 565.546 | 1 | 36.2827 | 38.0103405 | 1.70669341 | 7.037068149 | 828 | 1.22872956 | 1.2:CAAC | 1.3:CAAC | CAA |
| CAA | | CAG | 345 | 262.454 | | 3230552 | 0080947 | 28487725e- | 90735e-10 | | 7601233 | AACAA | AGCAA | CAG |
| | | | | | | 6196 | | 09 | | | | | | |
| CAA_Q_ | Q_Q_Q | CAA | 256 | 325.804 | 1 | 44.3237 | 47.1821872 | 2.78314523 | 6.468563303 | 477 | 1.35699223 | 1.3:CAAC | 1.5:CAAC | CAA |
| CAG | | CAG | 221 | 151.196 | | 8198167 | 8087599 | 25338187e- | 914926e-12 | | 49900103 | AACAG | AGCAG | CAG |
| | | | | | | 4 | | 11 | | | | | | |
| CAA_S_ | Q_S_D | AGC | 24 | 18.028 | 5 | 41.1370 | 38.9665694 | 8.80338914 | 2.411916681 | 160 | 1.47947775 | 2.8:CAATC | 1.8:CAAA | TCT |
| GAT | | AGT | 47 | 26.112 | | 1328675 | 68202024 | 3943951e- | 0656804e-07 | | 18345372 | CGAT | GTGAT | AGT |
| | | TCA | 16 | 33.476 | | 576 | | 08 | | | | | | AGC |
| | | TCC | 9 | 25.123 | | | | | | | | | | TCG |
| | | TCG | 17 | 15.592 | | | | | | | | | | TCA |
| | | TCT | 47 | 41.669 | | | | | | | | | | TCC |
| CAA_V_ | Q_V_T | GTA | 41 | 19.151 | 3 | 30.8689 | 34.9136428 | 9.05807231 | 1.270516815 | 89 | 1.69806269 | 2.0:CAAGT | 2.1:CAAG | GTA |
| ACA | | GTC | 18 | 17.931 | | 4675309 | 9626172 | 1688947e- | 3248366e-07 | | 35738778 | TACA | TAACA | GTC |
| | | GTG | 13 | 17.458 | | 442 | | 07 | | | | | | GTT |
| | | GTT | 17 | 34.460 | | | | | | | | | | GTG |
| CAC_G_ | H_G_K | GGA | 4 | 9.619 | 3 | 31.0177 | 46.4393931 | 8.42749277 | 4.573663339 | 43 | 2.38101470 | 2.4:CACG | 3.7:CACG | GGG |
| AAG | | GGC | 6 | 8.502 | | 8655560 | 3202548 | 8357571e- | 052711e-10 | | 830117 | GAAAG | GGAAG | GGT |
| | | GGG | 20 | 5.341 | | 2072 | | 07 | | | | | | GGC |
| | | GGT | 13 | 19.538 | | | | | | | | | | GGA |
| CAC_L_ | H_L_R | CTA | 2 | 3.823 | 5 | 38.4625 | 38.5714125 | 3.04617457 | 2.896428132 | 27 | 2.74681067 | 6.9:CACCT | 2.9:CACTT | TTG |
| CGA | | CTC | 0 | 1.559 | | 4469296 | 399401 | 0352341e- | 280687e-07 | | 97973433 | TCGA | GCGA | TTA |
| | | CTG | 0 | 3.054 | | 661 | | 07 | | | | | | CTA |
| | | CTT | 0 | 3.468 | | | | | | | | | | CTT |
| | | TTA | 3 | 7.451 | | | | | | | | | | CTG |
| | | TTG | 22 | 7.644 | | | | | | | | | | CTC |
| CAG_A_ | Q_A_T | GCA | 8 | 12.672 | 3 | 35.9704 | 54.1208901 | 7.59686291 | 1.057386241 | 43 | 2.42784926 | 2.6:CAGG | 4.1:CAGG | GCG |
| ACA | | GCC | 9 | 9.607 | | 5455978 | 2195521 | 5604791e- | 651209e-11 | | 9283763 | CTACA | CGACA | GCC |
| | | GCG | 20 | 4.916 | | 321 | | 08 | | | | | | GCA |
| | | GCT | 6 | 15.805 | | | | | | | | | | GCT |
| CAG_F_ | Q_F_K | TTC | 60 | 32.896 | 1 | 37.3069 | 37.6024419 | 1.00924849 | 8.673582701 | 81 | 1.93490209 | 2.3:CAGTT | 1.8:CAGTT | TTC |
| AAA | | TTT | 21 | 48.104 | | 3932056 | 52051095 | 39453898e- | 581865e-10 | | 45784507 | TAAA | CAAA | TTT |
| | | | | | | 055 | | 09 | | | | | | |
| CAG_G_ | Q_G_G | GGA | 2 | 5.369 | 3 | 39.7969 | 53.5200658 | 1.17648100 | 1.420241757 | 24 | 3.92504087 | 5.5:CAGG | 4.0:CAGG | GGC |
| GGA | | GGC | 19 | 4.745 | | 9948265 | 1322712 | 60686548e- | 9522068e-11 | | 7723925 | GTGGA | GCGGA | GGT |
| | | GGG | 1 | 2.981 | | 218 | | 08 | | | | | | GGA |
| | | GGT | 2 | 10.905 | | | | | | | | | | GGG |
| CAG_I_ | Q_I_K | ATA | 22 | 27.032 | 2 | 36.1226 | 40.6477212 | 1.43244174 | 1.490937625 | 97 | 1.81685613 | 2.0:CAGAT | 2.1:CAGA | ATC |
| AAA | | ATC | 52 | 25.045 | | 0048453 | 7193013 | 43867102e- | 6160466e-09 | | 20452992 | TAAA | TCAAA | ATT |
| | | ATT | 23 | 44.924 | | 466 | | 08 | | | | | | ATA |
| CAG_I_ | Q_I_C | ATA | 32 | 12.262 | 2 | 39.0593 | 44.4473381 | 3.29889485 | 2.230402002 | 44 | 2.58938336 | 3.4:CAGAT | 2.6:CAGA | ATA |
| TGT | | ATC | 6 | 11.360 | | 5663479 | 80857344 | 36497267e- | 7388762e-10 | | 95949855 | TTGT | TATGT | ATT |
| | | ATT | 6 | 20.378 | | 304 | | 09 | | | | | | ATC |
| CAG_L_ | Q_L_A | CTA | 4 | 5.806 | 5 | 35.2459 | 54.1440461 | 1.34382480 | 1.957710334 | 41 | 2.68352122 | 2.4:CAGCT | 4.0:CAGCT | CTT |
| GCG | | CTC | 1 | 2.368 | | 2773993 | 46126946 | 49233848e- | 9338626e-10 | | 07706105 | CGCG | TGCG | TTG |
| | | CTG | 3 | 4.638 | | 925 | | 06 | | | | | | TTA |
| | | CTT | 21 | 5.266 | | | | | | | | | | CTA |
| | | TTA | 6 | 11.315 | | | | | | | | | | CTG |
| | | TTG | 6 | 11.608 | | | | | | | | | | CTC |
| CAG_P_ | Q_P_E | CCA | 8 | 22.815 | 3 | 35.8983 | 42.2409885 | 7.86824078 | 3.566414839 | 56 | 2.17507547 | 2.9:CAGCC | 2.8:CAGC | CCC |
| GAA | | CCC | 25 | 8.988 | | 5317592 | 44000565 | 2108241e- | 8417185e-09 | | 23946097 | AGAA | CCGAA | CCT |
| | | CCG | 11 | 6.883 | | 9714 | | 08 | | | | | | CCG |
| | | CCT | 12 | 17.314 | | | | | | | | | | CCA |
| CAG_Q_ | Q_Q_Q | CAA | 262 | 334.000 | 1 | 45.9869 | 48.9661469 | 1.19042178 | 2.604185695 | 489 | 1.35960750 | 1.3:CAGC | 1.5:CAGC | CAA |
| CAA | | CAG | 227 | 155.000 | | 6902220 | 4171074 | 23837535e- | 3884798e-12 | | 6027204 | AACAA | AGCAA | CAG |
| | | | | | | 6495 | | 11 | | | | | | |
| CAG_Q_ | Q_Q_Q | CAA | 253 | 356.540 | 1 | 87.8712 | 94.8603080 | 6.98563231 | 2.043055601 | 522 | 1.51696159 | 1.4:CAGC | 1.6:CAGC | CAG |
| CAG | | CAG | 269 | 165.460 | | 6069071 | 6369678 | 1173287e- | 9472275e-22 | | 5988054 | AACAG | AGCAG | CAA |
| | | | | | | 63 | | 21 | | | | | | |
| CAG_R_ | Q_R_I | AGA | 13 | 26.552 | 5 | 36.5362 | 50.7460728 | 7.41823540 | 9.749123902 | 56 | 2.21480859 | 2.3:CAGC | 3.3:CAGC | CGT |
| ATT | | AGG | 9 | 12.147 | | 8968830 | 8476864 | 5989118e- | 010704e-10 | | 43830673 | GGATT | GTATT | AGA |
| | | CGA | 4 | 3.824 | | 904 | | 07 | | | | | | AGG |
| | | CGC | 3 | 3.314 | | | | | | | | | | CGA |
| | | CGG | 1 | 2.337 | | | | | | | | | | CGC |
| | | CGT | 26 | 7.825 | | | | | | | | | | CGG |
| CAG_R_ | Q_R_L | AGA | 22 | 37.457 | 5 | 46.2224 | 58.1311085 | 8.18264369 | 2.955534398 | 79 | 2.12770910 | 5.4:CAGC | 2.8:CAGC | CGT |
| TTG | | AGG | 11 | 17.135 | | 4024316 | 8160352 | 569681e-09 | 7062184e-11 | | 3498453 | GATTG | GTTTG | AGA |
| | | CGA | 1 | 5.395 | | 743 | | | | | | | | AGG |
| | | CGC | 5 | 4.676 | | | | | | | | | | CGG |
| | | CGG | 9 | 3.297 | | | | | | | | | | CGC |
| | | CGT | 31 | 11.039 | | | | | | | | | | CGA |
| CAG_S_ | Q_S_Q | AGC | 7 | 10.929 | 5 | 34.5659 | 47.7951099 | 1.83655274 | 3.910947076 | 97 | 1.66121011 | 2.0:CAGA | 3.1:CAGTC | TCG |
| CAA | | AGT | 8 | 15.831 | | 9422530 | 6434827 | 28223278e- | 868426e-09 | | 5346313 | GTCAA | GCAA | TCT |
| | | TCA | 15 | 20.295 | | 8245 | | 06 | | | | | | TCA |
| | | TCC | 12 | 15.231 | | | | | | | | | | TCC |
| | | TCG | 29 | 9.453 | | | | | | | | | | AGT |
| | | TCT | 26 | 25.262 | | | | | | | | | | AGC |
| CAG_V_ | Q_V_R | GTA | 20 | 5.595 | 3 | 39.3036 | 48.1256196 | 1.49670352 | 2.002355027 | 26 | 3.33363914 | 10.1:CAGG | 3.6:CAGG | GTA |
| CGC | | GTC | 2 | 5.238 | | 3808747 | 7032789 | 4726313e- | 3444165e-10 | | 04104457 | TTCGC | TACGC | GTG |
| | | GTG | 3 | 5.100 | | 676 | | 08 | | | | | | GTC |
| | | GTT | 1 | 10.067 | | | | | | | | | | GTT |
| CAT_F_ | H_F_K | TTC | 58 | 34.927 | 1 | 25.1744 | 25.6658630 | 5.23719619 | 4.059447733 | 86 | 1.71214320 | 1.8:CATTT | 1.7:CATTT | TTC |
| AAA | | TTT | 28 | 51.073 | | 3110550 | 27012847 | 43578e-07 | 272024e-07 | | 27028451 | TAAA | CAAA | TTT |
| | | | | | | 923 | | | | | | | | |
| CAT_G_ | H_G_F | GGA | 12 | 13.646 | 3 | 31.1085 | 42.6779900 | 8.06468331 | 2.880544753 | 61 | 1.91987528 | 2.0:CATGG | 3.2:CATG | GGG |
| TTT | | GGC | 11 | 12.061 | | 4597353 | 3806919 | 6276279e- | 0673023e-09 | | 0581611 | TTTT | GGTTT | GGT |
| | | GGG | 24 | 7.577 | | 2134 | | 07 | | | | | | GGA |
| | | GGT | 14 | 27.716 | | | | | | | | | | GGC |
| CAT_L_ | H_L_K | CTA | 17 | 29.596 | 5 | 68.3319 | 63.3783950 | 2.27810785 | 2.430398411 | 209 | 1.53405052 | 6.7:CATCT | 1.8:CATTT | TTG |
| AAA | | CTC | 11 | 12.069 | | 9611216 | 3987388 | 08768886e- | 108009e-12 | | 14747804 | TAAA | GAAA | TTA |
| | | CTG | 22 | 23.642 | | 261 | | 13 | | | | | | CTG |
| | | CTT | 4 | 26.845 | | | | | | | | | | CTA |
| | | TTA | 49 | 57.678 | | | | | | | | | | CTC |
| | | TTG | 106 | 59.170 | | | | | | | | | | CTT |
| CAT_L_ | H_L_P | CTA | 1 | 7.080 | 5 | 65.8928 | 128.140524 | 7.31543794 | 5.903568836 | 50 | 3.27045670 | 7.1:CATCT | 7.3:CATCT | CTC |
| CCC | | CTC | 21 | 2.887 | | 6899130 | 14587172 | 6384157e- | 642293e-26 | | 17767303 | ACCC | CCCC | CTT |
| | | CTG | 3 | 5.656 | | 144 | | 13 | | | | | | TTA |
| | | CTT | 10 | 6.422 | | | | | | | | | | TTG |
| | | TTA | 8 | 13.799 | | | | | | | | | | CTG |
| | | TTG | 7 | 14.156 | | | | | | | | | | CTA |
| CAT_L_ | H_L_F | CTA | 4 | 11.470 | 5 | 75.1235 | 94.1008168 | 8.76662765 | 9.230957120 | 81 | 2.35780555 | 18.3:CATC | 3.7:CATCT | CTT |
| TTC | | CTC | 8 | 4.677 | | 6797374 | 5342436 | 7397516e- | 905817e-19 | | 52226647 | TGTTC | TTTC | TTG |
| | | CTG | 0 | 9.163 | | 375 | | 15 | | | | | | TTA |
| | | CTT | 38 | 10.404 | | | | | | | | | | CTC |
| | | TTA | 15 | 22.354 | | | | | | | | | | CTA |
| | | TTG | 16 | 22.932 | | | | | | | | | | CTG |
| CAT_R_ | H_R_Q | AGA | 9 | 22.285 | 5 | 59.5781 | 99.7692956 | 1.48555114 | 5.911247825 | 47 | 3.20143279 | 3.9:CATCG | 5.9:CATCG | CGA |
| CAG | | AGG | 8 | 10.194 | | 9967248 | 7745887 | 7379553e- | 950523e-20 | | 44733854 | GCAG | ACAG | AGA |
| | | CGA | 19 | 3.209 | | 035 | | 11 | | | | | | CGC |
| | | CGC | 8 | 2.782 | | | | | | | | | | AGG |
| | | CGG | 0 | 1.962 | | | | | | | | | | CGT |
| | | CGT | 3 | 6.568 | | | | | | | | | | CGG |
| CCA_E_ | P_E_S | GAA | 16 | 32.847 | 1 | 25.5966 | 28.6966699 | 4.20763442 | 8.464916745 | 47 | 2.14262517 | 2.1:CCAG | 2.2:CCAG | GAG |
| TCG | | GAG | 31 | 14.153 | | 7669610 | 35422335 | 91090056e- | 316905e-08 | | 1013442 | AATCG | AGTCG | GAA |
| | | | | | | 3053 | | 07 | | | | | | |
| CCA_F_ | P_F_K | TTC | 51 | 28.429 | 1 | 29.8580 | 30.1752757 | 4.64869840 | 3.947093454 | 70 | 1.89327888 | 2.2:CCATT | 1.8:CCATT | TTC |
| AAG | | TTT | 19 | 41.571 | | 2042663 | 44286367 | 8181602e- | 1370847e-08 | | 51163382 | TAAG | CAAG | TTT |
| | | | | | | 798 | | 08 | | | | | | |
| CCA_G_ | P_G_G | GGA | 3 | 14.988 | 3 | 40.7789 | 37.1477725 | 7.28468530 | 4.281756002 | 67 | 1.94797859 | 5.0:CCAG | 1.8:CCAG | GGT |
| GGT | | GGC | 7 | 13.248 | | 2212180 | 20581995 | 7408518e- | 2099955e-08 | | 9668731 | GAGGT | GTGGT | GGC |
| | | GGG | 2 | 8.322 | | 889 | | 09 | | | | | | GGA |
| | | GGT | 55 | 30.442 | | | | | | | | | | GGG |
| CCA_K_ | P_K_G | AAA | 27 | 51.554 | 1 | 27.5972 | 27.7939192 | 1.49389884 | 1.349495967 | 89 | 1.72887655 | 1.9:CCAA | 1.7:CCAA | AAG |
| GGT | | AAG | 62 | 37.446 | | 6456842 | 1646883 | 44814098e- | 713402e-07 | | 99918776 | AAGGT | AGGGT | AAA |
| | | | | | | 3036 | | 07 | | | | | | |
| CCA_P_ | P_P_R | CCA | 55 | 30.148 | 3 | 37.7658 | 36.0488854 | 3.16826584 | 7.312264586 | 74 | 1.89367635 | 3.8:CCACC | 1.8:CCACC | CCA |
| AGA | | CCC | 8 | 11.876 | | 1305493 | 1193638 | 09033286e- | 470681e-08 | | 18419142 | TAGA | AAGA | CCC |
| | | CCG | 5 | 9.096 | | 613 | | 08 | | | | | | CCT |
| | | CCT | 6 | 22.880 | | | | | | | | | | CCG |
| CCA_P_ | P_P_G | CCA | 9 | 16.296 | 3 | 46.9395 | 76.0955329 | 3.58001569 | 2.110004438 | 40 | 3.27895716 | 3.2:CCACC | 4.7:CCACC | CCG |
| GGC | | CCC | 2 | 6.420 | | 6880680 | 7769995 | 0820228e- | 6194562e-16 | | 43108233 | CGGC | GGGC | CCA |
| | | CCG | 23 | 4.917 | | 9326 | | 10 | | | | | | CCT |
| | | CCT | 6 | 12.367 | | | | | | | | | | CCC |
| CCC_R_ | P_R_E | AGA | 12 | 15.647 | 5 | 53.8950 | 105.876493 | 2.20255995 | 3.044221715 | 33 | 3.54446944 | 3.9:CCCCG | 7.5:CCCCG | CGA |
| GAG | | AGG | 2 | 7.158 | | 2954184 | 27985133 | 74905694e- | 314959e-21 | | 62547126 | CGAG | AGAG | AGA |
| | | CGA | 17 | 2.253 | | 165 | | 10 | | | | | | CGT |
| | | CGC | 0 | 1.953 | | | | | | | | | | AGG |
| | | CGG | 0 | 1.377 | | | | | | | | | | CGG |
| | | CGT | 2 | 4.611 | | | | | | | | | | CGC |
| CCC_S_ | P_S_V | AGC | 4 | 7.662 | 5 | 42.8934 | 47.1541390 | 3.88345528 | 5.284864719 | 68 | 2.11994768 | 4.7:CCCTC | 2.4:CCCTC | TCT |
| GTG | | AGT | 7 | 11.098 | | 4647981 | 064562 | 08327425e- | 6526456e-09 | | 85711364 | AGTG | TGTG | TCC |
| | | TCA | 3 | 14.227 | | 75 | | 08 | | | | | | AGT |
| | | TCC | 8 | 10.677 | | | | | | | | | | TCG |
| | | TCG | 4 | 6.627 | | | | | | | | | | AGC |
| | | TCT | 42 | 17.709 | | | | | | | | | | TCA |
| CCG_G_ | P_G_Y | GGA | 3 | 5.816 | 3 | 57.6509 | 70.0037772 | 1.86615342 | 4.260394479 | 26 | 3.86369943 | 23.6:CCGG | 4.3:CCGG | GGC |
| TAT | | GGC | 22 | 5.141 | | 3833110 | 3465179 | 50096314e- | 686388e-15 | | 85161945 | GTTAT | GCTAT | GGA |
| | | GGG | 1 | 3.229 | | 1486 | | 12 | | | | | | GGG |
| | | GGT | 0 | 11.813 | | | | | | | | | | GGT |
| CCG_L_ | P_L_Y | CTA | 6 | 5.806 | 5 | 39.2382 | 59.7434823 | 2.12651162 | 1.373226649 | 41 | 2.60374114 | 2.8:CCGTT | 4.3:CCGCT | CTG |
| TAC | | CTC | 2 | 2.368 | | 5410074 | 6727016 | 10122422e- | 0107279e-11 | | 011348 | ATAC | GTAC | CTA |
| | | CTG | 20 | 4.638 | | 345 | | 07 | | | | | | TTG |
| | | CTT | 4 | 5.266 | | | | | | | | | | TTA |
| | | TTA | 4 | 11.315 | | | | | | | | | | CTT |
| | | TTG | 5 | 11.608 | | | | | | | | | | CTC |
| CCT_R_ | P_R_L | AGA | 1 | 10.905 | 5 | 48.3609 | 75.5818142 | 2.99756151 | 7.033696003 | 23 | 4.24130299 | 10.9:CCTA | 7.0:CCTCG | CGA |
| CTA | | AGG | 2 | 4.989 | | 4167927 | 7831528 | 0510337e- | 922999e-15 | | 2595476 | GACTA | ACTA | CGT |
| | | CGA | 11 | 1.571 | | 687 | | 09 | | | | | | AGG |
| | | CGC | 1 | 1.361 | | | | | | | | | | CGC |
| | | CGG | 0 | 0.960 | | | | | | | | | | AGA |
| | | CGT | 8 | 3.214 | | | | | | | | | | CGG |
| CCT_R_ | P_R_L | AGA | 3 | 10.431 | 5 | 25.9551 | 44.0313004 | 9.10430516 | 2.282582313 | 22 | 2.87582041 | 3.5:CCTAG | 6.0:CCTCG | CGA |
| CTT | | AGG | 3 | 4.772 | | 0118786 | 5694727 | 8484215e- | 0204454e-08 | | 85343414 | ACTT | ACTT | CGT |
| | | CGA | 9 | 1.502 | | 651 | | 05 | | | | | | AGG |
| | | CGC | 2 | 1.302 | | | | | | | | | | AGA |
| | | CGG | 1 | 0.918 | | | | | | | | | | CGC |
| | | CGT | 4 | 3.074 | | | | | | | | | | CGG |
| CGA_E_ | R_E_P | GAA | 0 | 12.580 | 1 | 43.2091 | 41.7773142 | 4.91898303 | 1.022833151 | 18 | 3.32096190 | 25.2:CGAG | 3.3:CGAG | GAG |
| CCC | | GAG | 18 | 5.420 | | 6097284 | 9488207 | 3538821e- | 9071506e-10 | | 5271226 | AACCC | AGCCC | GAA |
| | | | | | | 805 | | 11 | | | | | | |
| CGA_L_ | R_L_G | CTA | 1 | 4.107 | 5 | 36.3814 | 37.9427859 | 7.96728936 | 3.874421694 | 29 | 2.73656109 | 6.6:CGACT | 2.8:CGATT | TTG |
| GGT | | CTC | 0 | 1.675 | | 1858727 | 4111932 | 2781744e- | 411382e-07 | | 8525587 | GGGT | GGGT | TTA |
| | | CTG | 0 | 3.280 | | 622 | | 07 | | | | | | CTT |
| | | CTT | 1 | 3.725 | | | | | | | | | | CTA |
| | | TTA | 4 | 8.003 | | | | | | | | | | CTG |
| | | TTG | 23 | 8.210 | | | | | | | | | | CTC |
| CGA_Q_ | R_Q_L | CAA | 5 | 17.759 | 1 | 26.6108 | 28.9186856 | 2.48837143 | 7.548123987 | 26 | 2.71617253 | 3.6:CGAC | 2.5:CGAC | CAG |
| TTG | | CAG | 21 | 8.241 | | 9048825 | 6097344 | 40774176e- | 44897e-08 | | 65133447 | AATTG | AGTTG | CAA |
| | | | | | | 327 | | 07 | | | | | | |
| CGA_R_ | R_R_Y | AGA | 1 | 4.741 | 5 | 25.0930 | 47.7307338 | 0.00013368 | 4.031057291 | 10 | 5.11839705 | 4.7:CGAA | 8.8:CGAC | CGA |
| TAC | | AGG | 0 | 2.169 | | 3793731 | 011119 | 6795931917 | 183412e-09 | | 4858626 | GATAC | GATAC | CGT |
| | | CGA | 6 | 0.683 | | 7288 | | 16 | | | | | | CGG |
| | | CGC | 1 | 0.592 | | | | | | | | | | CGC |
| | | CGG | 1 | 0.417 | | | | | | | | | | AGA |
| | | CGT | 1 | 1.397 | | | | | | | | | | AGG |
| CGC_E_ | R_E_F | GAA | 3 | 16.074 | 1 | 32.3480 | 35.3157593 | 1.28886739 | 2.803543152 | 23 | 3.13026115 | 5.4:CGCG | 2.9:CGCG | GAG |
| TTC | | GAG | 20 | 6.926 | | 4614441 | 34878805 | 65603598e- | 953539e-09 | | 99692667 | AATTC | AGTTC | GAA |
| | | | | | | 254 | | 08 | | | | | | |
| CGC_R_ | R_R_T | AGA | 0 | 2.845 | 5 | 19.1982 | 41.0163159 | 0.00176532 | 9.311842721 | 6 | 5.40690012 | 5.7:CGCA | 11.3:CGCC | CGC |
| ACC | | AGG | 1 | 1.301 | | 7712153 | 3718349 | 4466073480 | 383708e-08 | | 9760741 | GAACC | GCACC | CGT |
| | | CGA | 0 | 0.410 | | 177 | | 6 | | | | | | AGG |
| | | CGC | 4 | 0.355 | | | | | | | | | | CGG |
| | | CGG | 0 | 0.250 | | | | | | | | | | CGA |
| | | CGT | 1 | 0.838 | | | | | | | | | | AGA |
| CGC_R_ | R_R_Q | AGA | 1 | 4.741 | 5 | 19.7234 | 38.6251853 | 0.00140820 | 2.825191912 | 10 | 4.27291435 | 4.7:CGCA | 9.6:CGCC | CGG |
| CAA | | AGG | 1 | 2.169 | | 2594323 | 64977874 | 2819230015 | 74817e-07 | | 3405356 | GACAA | GGCAA | CGT |
| | | CGA | 0 | 0.683 | | 9487 | | | | | | | | CGC |
| | | CGC | 2 | 0.592 | | | | | | | | | | AGG |
| | | CGG | 4 | 0.417 | | | | | | | | | | AGA |
| | | CGT | 2 | 1.397 | | | | | | | | | | CGA |
| CGG_L_ | R_L_K | CTA | 5 | 5.948 | 5 | 59.9998 | 91.3411723 | 1.21553872 | 3.511715809 | 42 | 3.11682326 | 5.8:CGGTT | 5.1:CGGCT | CTG |
| AAG | | CTC | 0 | 2.425 | | 5865735 | 8844607 | 41910107e- | 385506e-18 | | 6240415 | AAAG | GAAG | TTG |
| | | CTG | 24 | 4.751 | | 51 | | 11 | | | | | | CTA |
| | | CTT | 2 | 5.395 | | | | | | | | | | TTA |
| | | TTA | 2 | 11.591 | | | | | | | | | | CTT |
| | | TTG | 9 | 11.891 | | | | | | | | | | CTC |
| CGG_Q_ | R_Q_S | CAA | 1 | 15.027 | 1 | 40.8818 | 41.3067320 | 1.61712614 | 1.301199907 | 22 | 3.23970174 | 15.0:CGGC | 3.0:CGGC | CAG |
| TCG | | CAG | 21 | 6.973 | | 9185299 | 24118446 | 52767198e- | 105783e-10 | | 11630995 | AATCG | AGTCG | CAA |
| | | | | | | 5146 | | 10 | | | | | | |
| CGG_R_ | R_R_S | AGA | 0 | 0.948 | 5 | 12.7051 | 45.9156053 | 0.02630392 | 9.448594332 | 2 | 23.9578026 | 1.9:CGGA | 24.0:CGGC | CGG |
| TCT | | AGG | 0 | 0.434 | | 7624797 | 98213266 | 9782050435 | 49579e-09 | | 9910663 | GATCT | GGTCT | CGT |
| | | CGA | 0 | 0.137 | | 1712 | | | | | | | | CGC |
| | | CGC | 0 | 0.118 | | | | | | | | | | CGA |
| | | CGG | 2 | 0.083 | | | | | | | | | | AGG |
| | | CGT | 0 | 0.279 | | | | | | | | | | AGA |
| CGG_S_ | R_S_E | AGC | 1 | 3.606 | 5 | 61.1626 | 104.971780 | 6.98842626 | 4.725487440 | 32 | 4.00202300 | 8.3:CGGTC | 6.4:CGGTC | TCG |
| GAA | | AGT | 1 | 5.222 | | 0579664 | 96724384 | 38676816e- | 903565e-21 | | 4381506 | TGAA | GGAA | TCA |
| | | TCA | 7 | 6.695 | | 949 | | 12 | | | | | | TCC |
| | | TCC | 2 | 5.025 | | | | | | | | | | TCT |
| | | TCG | 20 | 3.118 | | | | | | | | | | AGT |
| | | TCT | 1 | 8.334 | | | | | | | | | | AGC |
| CGG_S_ | R_S_D | AGC | 1 | 2.366 | 5 | 35.7395 | 39.1849414 | 1.07084856 | 2.179729939 | 21 | 3.29117537 | 6.6:CGGTC | 3.3:CGGTC | TCT |
| GAC | | AGT | 1 | 3.427 | | 0429405 | 97559045 | 81934712e- | 6338427e-07 | | 85897853 | CGAC | TGAC | TCA |
| | | TCA | 1 | 4.394 | | 9515 | | 06 | | | | | | AGT |
| | | TCC | 0 | 3.297 | | | | | | | | | | AGC |
| | | TCG | 0 | 2.046 | | | | | | | | | | TCG |
| | | TCT | 18 | 5.469 | | | | | | | | | | TCC |
| CGT_L_ | R_L_R | CTA | 1 | 4.390 | 5 | 35.3184 | 37.5377364 | 1.29975786 | 4.672299240 | 31 | 2.68583536 | 7.0:CGTCT | 2.7:CGTTT | TTG |
| CGT | | CTC | 1 | 1.790 | | 3805942 | 60865766 | 1644524e- | 009048e-07 | | 4466964 | GCGT | GCGT | TTA |
| | | CTG | 0 | 3.507 | | 047 | | 06 | | | | | | CTT |
| | | CTT | 1 | 3.982 | | | | | | | | | | CTC |
| | | TTA | 4 | 8.555 | | | | | | | | | | CTA |
| | | TTG | 24 | 8.776 | | | | | | | | | | CTG |
| CGT_R_ | R_R_R | AGA | 0 | 3.319 | 5 | 24.9984 | 45.9675592 | 0.00013942 | 9.221273869 | 7 | 6.71887713 | 6.6:CGTAG | 9.7:CGTCG | CGC |
| CGC | | AGG | 1 | 1.518 | | 5779772 | 8143284 | 9357865386 | 309018e-09 | | 77646805 | ACGC | CCGC | CGG |
| | | CGA | 0 | 0.478 | | 2658 | | 06 | | | | | | AGG |
| | | CGC | 4 | 0.414 | | | | | | | | | | CGT |
| | | CGG | 2 | 0.292 | | | | | | | | | | CGA |
| | | CGT | 0 | 0.978 | | | | | | | | | | AGA |
| CGT_S_ | R_S_M | AGC | 2 | 5.183 | 5 | 34.6346 | 44.1310683 | 1.77956716 | 2.178568572 | 46 | 2.23785422 | 3.8:CGTAG | 3.2:CGTTC | TCC |
| ATG | | AGT | 2 | 7.507 | | 5412517 | 0812757 | 19672613e- | 6589464e-08 | | 8135717 | TATG | CATG | TCT |
| | | TCA | 4 | 9.624 | | 878 | | 06 | | | | | | TCG |
| | | TCC | 23 | 7.223 | | | | | | | | | | TCA |
| | | TCG | 5 | 4.483 | | | | | | | | | | AGT |
| | | TCT | 10 | 11.980 | | | | | | | | | | AGC |
| CTA_G_ | L_G_R | GGA | 12 | 2.684 | 3 | 35.9390 | 41.6437647 | 7.71406075 | 4.774887719 | 12 | 4.47031373 | 10.9:CTAG | 4.5:CTAG | GGA |
| CGG | | GGC | 0 | 2.373 | | 0621325 | 80072516 | 8834998e- | 0802405e-09 | | 167271 | GTCGG | GACGG | GGT |
| | | GGG | 0 | 1.491 | | 807 | | 08 | | | | | | GGG |
| | | GGT | 0 | 5.452 | | | | | | | | | | GGC |
| CTA_I_ | L_I_K | ATA | 27 | 25.360 | 2 | 60.0633 | 60.1725521 | 9.06604788 | 8.584130619 | 91 | 1.92466699 | 3.8:CTAAT | 2.3:CTAAT | ATC |
| AAG | | ATC | 53 | 23.495 | | 0973614 | 6008214 | 0635049e- | 834704e-14 | | 87494047 | TAAG | CAAG | ATA |
| | | ATT | 11 | 42.145 | | 24 | | 14 | | | | | | ATT |
| CTA_R_ | L_R_C | AGA | 2 | 9.009 | 5 | 24.1840 | 42.4098524 | 0.00020012 | 4.866098703 | 19 | 2.90345342 | 4.5:CTAAG | 7.6:CTACG | CGG |
| TGT | | AGG | 3 | 4.121 | | 3392687 | 8073355 | 6702640095 | 709642e-08 | | 439928 | ATGT | GTGT | CGT |
| | | CGA | 2 | 1.297 | | 6218 | | 86 | | | | | | AGG |
| | | CGC | 1 | 1.125 | | | | | | | | | | CGA |
| | | CGG | 6 | 0.793 | | | | | | | | | | AGA |
| | | CGT | 5 | 2.655 | | | | | | | | | | CGC |
| CTA_S_ | L_S_S | AGC | 7 | 5.634 | 5 | 39.8000 | 52.9450147 | 1.63866342 | 3.451919330 | 50 | 2.47976776 | 2.6:CTATC | 3.3:CTATC | TCC |
| AGC | | AGT | 4 | 8.160 | | 0223634 | 3352247 | 1012493e- | 367396e-10 | | 60740236 | TAGC | CAGC | AGC |
| | | TCA | 6 | 10.461 | | 951 | | 07 | | | | | | TCA |
| | | TCC | 26 | 7.851 | | | | | | | | | | TCT |
| | | TCG | 2 | 4.873 | | | | | | | | | | AGT |
| | | TCT | 5 | 13.021 | | | | | | | | | | TCG |
| CTC_P_ | L_P_L | CCA | 4 | 11.000 | 3 | 60.9955 | 86.4493693 | 3.60192827 | 1.267705099 | 27 | 4.56372445 | 16.7:CTCC | 5.1:CTCCC | CCC |
| TTG | | CCC | 22 | 4.333 | | 0984391 | 0992458 | 9681707e- | 2287093e-18 | | 802666 | CTTTG | CTTG | CCA |
| | | CCG | 1 | 3.319 | | 381 | | 13 | | | | | | CCG |
| | | CCT | 0 | 8.348 | | | | | | | | | | CCT |
| CTC_S_ | L_S_R | AGC | 21 | 4.958 | 5 | 40.2843 | 60.6400389 | 1.30861949 | 8.962805977 | 44 | 2.50979298 | 3.8:CTCTC | 4.2:CTCAG | AGC |
| AGA | | AGT | 4 | 7.181 | | 4168174 | 78100854 | 36190197e- | 162752e-12 | | 4346522 | TAGA | CAGA | TCA |
| | | TCA | 7 | 9.206 | | 92 | | 07 | | | | | | TCC |
| | | TCC | 5 | 6.909 | | | | | | | | | | TCG |
| | | TCG | 4 | 4.288 | | | | | | | | | | AGT |
| | | TCT | 3 | 11.459 | | | | | | | | | | TCT |
| CTG_A_ | L_A_K | GCA | 19 | 25.049 | 3 | 55.0160 | 64.0919661 | 6.81227973 | 7.844629907 | 85 | 2.15821713 | 3.1:CTGGC | 2.6:CTGGC | GCC |
| AAG | | GCC | 49 | 18.990 | | 4071693 | 2736466 | 70191835e- | 642043e-14 | | 52138455 | TAAG | CAAG | GCA |
| | | GCG | 7 | 9.718 | | 041 | | 12 | | | | | | GCT |
| | | GCT | 10 | 31.243 | | | | | | | | | | GCG |
| CTG_A_ | L_A_S | GCA | 18 | 20.039 | 3 | 24.5606 | 34.3543835 | 1.90764319 | 1.667640242 | 68 | 1.73866594 | 1.5:CTGGC | 3.0:CTGGC | GCG |
| TCT | | GCC | 10 | 15.192 | | 3182876 | 88543314 | 54237673e- | 8638804e-07 | | 08793919 | CTCT | GTCT | GCA |
| | | GCG | 23 | 7.775 | | 198 | | 05 | | | | | | GCT |
| | | GCT | 17 | 24.994 | | | | | | | | | | GCC |
| CTG_G_ | L_G_V | GGA | 27 | 10.738 | 3 | 26.8235 | 32.3292549 | 6.41081629 | 4.460552890 | 48 | 2.13388562 | 3.0:CTGGG | 2.5:CTGG | GGA |
| GTT | | GGC | 7 | 9.491 | | 2323884 | 7713816 | 2061468e- | 112313e-07 | | 0549679 | GGTT | GAGTT | GGT |
| | | GGG | 2 | 5.962 | | 45 | | 06 | | | | | | GGC |
| | | GGT | 12 | 21.810 | | | | | | | | | | GGG |
| CTG_Q_ | L_Q_F | CAA | 14 | 31.419 | 1 | 27.6718 | 30.4676800 | 1.43735967 | 3.394752959 | 46 | 2.20963705 | 2.2:CTGCA | 2.2:CTGCA | CAG |
| TTT | | CAG | 32 | 14.581 | | 9420815 | 59169965 | 41920386e- | 6823246e-08 | | 51420015 | ATTT | GTTT | CAA |
| | | | | | | 805 | | 07 | | | | | | |
| CTG_R_ | L_R_N | AGA | 22 | 35.561 | 5 | 60.0820 | 104.601410 | 1.16893857 | 5.657343646 | 75 | 2.31262830 | 3.5:CTGCG | 5.3:CTGCG | CGA |
| AAT | | AGG | 14 | 16.268 | | 1161701 | 3010177 | 64289617e- | 198293e-21 | | 43057483 | TAAT | AAAT | AGA |
| | | CGA | 27 | 5.122 | | 252 | | 11 | | | | | | AGG |
| | | CGC | 5 | 4.439 | | | | | | | | | | CGC |
| | | CGG | 4 | 3.131 | | | | | | | | | | CGG |
| | | CGT | 3 | 10.480 | | | | | | | | | | CGT |
| CTG_R_ | L_R_L | AGA | 4 | 9.957 | 5 | 24.4540 | 42.3171185 | 0.00017755 | 5.081096682 | 21 | 3.18822567 | 2.5:CTGAG | 6.8:CTGCG | CGG |
| CTA | | AGG | 2 | 4.555 | | 1125682 | 69294564 | 9556049671 | 2278284e-08 | | 3247993 | ACTA | GCTA | CGA |
| | | CGA | 4 | 1.434 | | 7187 | | 84 | | | | | | AGA |
| | | CGC | 3 | 1.243 | | | | | | | | | | CGC |
| | | CGG | 6 | 0.877 | | | | | | | | | | CGT |
| | | CGT | 2 | 2.935 | | | | | | | | | | AGG |
| CTG_S_ | L_S_M | AGC | 2 | 5.408 | 5 | 51.2175 | 78.1434692 | 7.80501735 | 2.051553651 | 48 | 2.88345499 | 3.9:CTGAG | 4.7:CTGTC | TCG |
| ATG | | AGT | 2 | 7.834 | | 7354820 | 0717493 | 1633154e- | 2413678e-15 | | 95140875 | TATG | GATG | TCC |
| | | TCA | 5 | 10.043 | | 1145 | | 10 | | | | | | TCT |
| | | TCC | 11 | 7.537 | | | | | | | | | | TCA |
| | | TCG | 22 | 4.678 | | | | | | | | | | AGT |
| | | TCT | 6 | 12.501 | | | | | | | | | | AGC |
| CTT_A_ | L_A_L | GCA | 5 | 8.841 | 3 | 41.2353 | 70.1517185 | 5.82926516 | 3.960687975 | 30 | 3.67704616 | 3.4:CTTGC | 5.2:CTTGC | GCG |
| CTG | | GCC | 2 | 6.702 | | 1155347 | 5098241 | 4023173e- | 340497e-15 | | 88987153 | CCTG | GCTG | GCT |
| | | GCG | 18 | 3.430 | | 392 | | 09 | | | | | | GCA |
| | | GCT | 5 | 11.027 | | | | | | | | | | GCC |
| CTT_F_ | L_F_K | TTC | 63 | 30.053 | 1 | 62.7881 | 60.8181580 | 2.30176879 | 6.259786391 | 74 | 2.30717718 | 4.0:CTTTT | 2.1:CTTTT | TTC |
| AAG | | TTT | 11 | 43.947 | | 7304689 | 0988166 | 4279102e- | 0482034e-15 | | 79460603 | TAAG | CAAG | TTT |
| | | | | | | 031 | | 15 | | | | | | |
| CTT_F_ | L_F_N | TTC | 60 | 36.145 | 1 | 26.0010 | 26.5094706 | 3.41224523 | 2.622492687 | 89 | 1.71129054 | 1.8:CTTTT | 1.7:CTTTT | TTC |
| AAT | | TTT | 29 | 52.855 | | 9071897 | 88248435 | 10250743e- | 470553e-07 | | 71119482 | TAAT | CAAT | TTT |
| | | | | | | 308 | | 07 | | | | | | |
| CTT_F_ | L_F_Q | TTC | 36 | 18.682 | 1 | 27.1302 | 27.0326833 | 1.90199904 | 2.000442845 | 46 | 2.07889301 | 2.7:CTTTT | 1.9:CTTTT | TTC |
| CAG | | TTT | 10 | 27.318 | | 3438793 | 2912699 | 82281682e- | 6917865e-07 | | 81751137 | TCAG | CCAG | TTT |
| | | | | | | 582 | | 07 | | | | | | |
| CTT_G_ | L_G_F | GGA | 34 | 15.659 | 3 | 34.3123 | 37.1529249 | 1.70209534 | 4.271020912 | 70 | 2.01097794 | 2.3:CTTGG | 2.2:CTTGG | GGA |
| TTT | | GGC | 8 | 13.841 | | 2343626 | 232303 | 65606973e- | 052423e-08 | | 6866545 | TTTT | ATTT | GGT |
| | | GGG | 14 | 8.695 | | 7434 | | 07 | | | | | | GGG |
| | | GGT | 14 | 31.806 | | | | | | | | | | GGC |
| CTT_L_ | L_L_K | CTA | 23 | 39.225 | 5 | 63.7325 | 67.7374005 | 2.05256173 | 3.028032347 | 277 | 1.56528369 | 2.2:CTTCT | 1.8:CTTTT | TTG |
| AAA | | CTC | 11 | 15.996 | | 2682295 | 4085735 | 21243047e- | 3389883e-13 | | 28485911 | TAAA | GAAA | TTA |
| | | CTG | 24 | 31.334 | | 603 | | 12 | | | | | | CTG |
| | | CTT | 16 | 35.579 | | | | | | | | | | CTA |
| | | TTA | 65 | 76.445 | | | | | | | | | | CTT |
| | | TTG | 138 | 78.422 | | | | | | | | | | CTC |
| CTT_L_ | L_L_L | CTA | 21 | 17.276 | 5 | 50.5096 | 53.5251043 | 1.08990800 | 2.623813752 | 122 | 1.69048293 | 2.9:CTTTT | 2.4:CTTCT | TTA |
| TTA | | CTC | 5 | 7.045 | | 0062584 | 50486316 | 14587936e- | 915273e-10 | | 5127555 | GTTA | TTTA | CTT |
| | | CTG | 6 | 13.800 | | 741 | | 09 | | | | | | CTA |
| | | CTT | 38 | 15.670 | | | | | | | | | | TTG |
| | | TTA | 40 | 33.669 | | | | | | | | | | CTG |
| | | TTG | 12 | 34.540 | | | | | | | | | | CTC |
| CTT_R_ | L_R_K | AGA | 10 | 27.974 | 5 | 58.0348 | 88.0732910 | 3.09383867 | 1.705769769 | 59 | 2.75265452 | 2.8:CTTAG | 4.7:CTTCG | CGA |
| AAA | | AGG | 9 | 12.797 | | 3806338 | 5250818 | 57572567e- | 666273e-17 | | 8454648 | AAAA | AAAA | AGA |
| | | CGA | 19 | 4.029 | | 0185 | | 11 | | | | | | CGG |
| | | CGC | 6 | 3.492 | | | | | | | | | | AGG |
| | | CGG | 9 | 2.463 | | | | | | | | | | CGT |
| | | CGT | 6 | 8.245 | | | | | | | | | | CGC |
| CTT_R_ | L_R_K | AGA | 12 | 16.121 | 5 | 29.5542 | 51.9384116 | 1.80480656 | 5.553982294 | 34 | 2.40700288 | 3.7:CTTAG | 6.3:CTTCG | AGA |
| AAG | | AGG | 2 | 7.375 | | 1111794 | 748259 | 8198472e- | 584428e-10 | | 49232523 | GAAG | GAAG | CGG |
| | | CGA | 6 | 2.322 | | 187 | | 05 | | | | | | CGA |
| | | CGC | 2 | 2.012 | | | | | | | | | | CGT |
| | | CGG | 9 | 1.419 | | | | | | | | | | CGC |
| | | CGT | 3 | 4.751 | | | | | | | | | | AGG |
| CTT_R_ | L_R_R | AGA | 15 | 15.173 | 5 | 33.8136 | 46.0448898 | 2.59325211 | 8.892979252 | 32 | 2.08447579 | 8.9:CTTCG | 5.0:CTTCG | AGA |
| AGA | | AGG | 2 | 6.941 | | 7556282 | 5420287 | 7837411e- | 159358e-09 | | 11823275 | TAGA | AAGA | CGA |
| | | CGA | 11 | 2.185 | | 483 | | 06 | | | | | | CGG |
| | | CGC | 1 | 1.894 | | | | | | | | | | AGG |
| | | CGG | 3 | 1.336 | | | | | | | | | | CGC |
| | | CGT | 0 | 4.472 | | | | | | | | | | CGT |
| CTT_R_ | L_R_E | AGA | 19 | 31.768 | 5 | 31.3879 | 39.8313867 | 7.85171377 | 1.614964885 | 67 | 1.97072947 | 2.1:CTTAG | 3.3:CTTCG | AGA |
| GAA | | AGG | 7 | 14.533 | | 7836378 | 6970263 | 7364288e- | 1682735e-07 | | 50887956 | GGAA | AGAA | CGT |
| | | CGA | 15 | 4.575 | | 6026 | | 06 | | | | | | CGA |
| | | CGC | 7 | 3.966 | | | | | | | | | | CGC |
| | | CGG | 3 | 2.797 | | | | | | | | | | AGG |
| | | CGT | 16 | 9.363 | | | | | | | | | | CGG |
| CTT_R_ | L_R_E | AGA | 4 | 11.379 | 5 | 30.1085 | 44.5652755 | 1.40405918 | 1.778204707 | 24 | 3.04422131 | 5.2:CTTAG | 5.5:CTTCG | CGA |
| GAG | | AGG | 1 | 5.206 | | 2660015 | 7585916 | 81651407e- | 7903607e-08 | | 63946747 | GGAG | AGAG | CGT |
| | | CGA | 9 | 1.639 | | 358 | | 05 | | | | | | AGA |
| | | CGC | 2 | 1.420 | | | | | | | | | | CGG |
| | | CGG | 2 | 1.002 | | | | | | | | | | CGC |
| | | CGT | 6 | 3.354 | | | | | | | | | | AGG |
| CTT_R_ | L_R_G | AGA | 0 | 3.793 | 5 | 27.9976 | 48.8170738 | 3.64373690 | 2.418741552 | 8 | 6.60375197 | 7.6:CTTAG | 9.0:CTTCG | CGA |
| GGC | | AGG | 1 | 1.735 | | 4505976 | 93617405 | 3953551e- | 8102733e-09 | | 7947322 | AGGC | GGGC | CGG |
| | | CGA | 4 | 0.546 | | 1062 | | 05 | | | | | | AGG |
| | | CGC | 0 | 0.473 | | | | | | | | | | CGT |
| | | CGG | 3 | 0.334 | | | | | | | | | | CGC |
| | | CGT | 0 | 1.118 | | | | | | | | | | AGA |
| CTT_R_ | L_R_F | AGA | 8 | 15.647 | 5 | 27.9226 | 38.4174889 | 3.76887919 | 3.110380961 | 33 | 2.46666987 | 3.6:CTTAG | 4.0:CTTCG | CGA |
| TTT | | AGG | 2 | 7.158 | | 1224762 | 7419877 | 1744005e- | 2390825e-07 | | 51668026 | GTTT | ATTT | AGA |
| | | CGA | 9 | 2.253 | | 2972 | | 05 | | | | | | CGT |
| | | CGC | 2 | 1.953 | | | | | | | | | | CGG |
| | | CGG | 5 | 1.377 | | | | | | | | | | CGC |
| | | CGT | 7 | 4.611 | | | | | | | | | | AGG |
| CTT_S_ | L_S_K | AGC | 5 | 22.309 | 5 | 46.1811 | 40.1390728 | 8.34248128 | 1.399969817 | 198 | 1.36903752 | 4.5:CTTAG | 1.6:CTTTC | TCT |
| AAA | | AGT | 15 | 32.314 | | 8388474 | 6407812 | 123318e-09 | 2863094e-07 | | 62973886 | CAAA | CAAA | TCC |
| | | TCA | 48 | 41.427 | | 2474 | | | | | | | | TCA |
| | | TCC | 50 | 31.090 | | | | | | | | | | TCG |
| | | TCG | 29 | 19.296 | | | | | | | | | | AGT |
| | | TCT | 51 | 51.565 | | | | | | | | | | AGC |
| CTT_S_ | L_S_N | AGC | 3 | 12.169 | 5 | 58.2129 | 51.2720516 | 2.84281352 | 7.606943191 | 108 | 1.78010990 | 8.8:CTTAG | 2.0:CTTTC | TCC |
| AAC | | AGT | 2 | 17.626 | | 5759176 | 4761694 | 9629753e- | 226615e-10 | | 84291472 | TAAC | CAAC | TCA |
| | | TCA | 34 | 22.596 | | 351 | | 11 | | | | | | TCT |
| | | TCC | 34 | 16.958 | | | | | | | | | | TCG |
| | | TCG | 17 | 10.525 | | | | | | | | | | AGC |
| | | TCT | 18 | 28.126 | | | | | | | | | | AGT |
| CTT_S_ | L_S_K | AGC | 2 | 12.507 | 5 | 48.1971 | 41.8261215 | 3.23746902 | 6.387423514 | 111 | 1.62704418 | 6.3:CTTAG | 1.8:CTTTC | TCA |
| AAG | | AGT | 4 | 18.115 | | 9343215 | 15009596 | 05138357e- | 096102e-08 | | 69037429 | CAAG | CAAG | TCC |
| | | TCA | 37 | 23.224 | | 6354 | | 09 | | | | | | TCT |
| | | TCC | 32 | 17.429 | | | | | | | | | | TCG |
| | | TCG | 13 | 10.817 | | | | | | | | | | AGT |
| | | TCT | 23 | 28.908 | | | | | | | | | | AGC |
| CTT_S_ | L_S_N | AGC | 2 | 17.577 | 5 | 58.5004 | 48.4423247 | 2.47986547 | 2.885008234 | 156 | 1.43574631 | 8.8:CTTAG | 1.9:CTTTC | TCT |
| AAT | | AGT | 7 | 25.459 | | 1782422 | 60302725 | 42114117e- | 988034e-09 | | 523567 | CAAT | CAAT | TCC |
| | | TCA | 40 | 32.639 | | 76 | | 11 | | | | | | TCA |
| | | TCC | 46 | 24.495 | | | | | | | | | | TCG |
| | | TCG | 15 | 15.203 | | | | | | | | | | AGT |
| | | TCT | 46 | 40.627 | | | | | | | | | | AGC |
| CTT_T_ | L_T_G | ACA | 8 | 10.029 | 3 | 55.4305 | 70.9453720 | 5.55716792 | 2.677980269 | 33 | 2.95891863 | 22.7:CTTA | 4.5:CTTAC | ACG |
| GGA | | ACC | 4 | 6.992 | | 6537996 | 0385508 | 9313938e- | 981928e-15 | | 07817372 | CTGGA | GGGA | ACA |
| | | ACG | 21 | 4.629 | | 783 | | 12 | | | | | | ACC |
| | | ACT | 0 | 11.351 | | | | | | | | | | ACT |
| GAA_A_ | E_A_L | GCA | 55 | 30.648 | 3 | 31.4714 | 32.6684545 | 6.76328477 | 3.783300315 | 104 | 1.70605991 | 2.1:GAAG | 1.8:GAAG | GCA |
| CTA | | GCC | 11 | 23.234 | | 3672721 | 9416085 | 0048341e- | 7808927e-07 | | 7995285 | CCCTA | CACTA | GCT |
| | | GCG | 15 | 11.891 | | 389 | | 07 | | | | | | GCG |
| | | GCT | 23 | 38.226 | | | | | | | | | | GCC |
| GAA_A_ | E_A_L | GCA | 9 | 15.914 | 3 | 33.8910 | 51.9511664 | 2.08896296 | 3.067721406 | 54 | 2.24005097 | 1.8:GAAG | 3.7:GAAG | GCG |
| CTC | | GCC | 8 | 12.064 | | 2133672 | 7606496 | 54422323e- | 2126626e-11 | | 48710573 | CACTC | CGCTC | GCT |
| | | GCG | 23 | 6.174 | | 005 | | 07 | | | | | | GCA |
| | | GCT | 14 | 19.848 | | | | | | | | | | GCC |
| GAA_A_ | E_A_E | GCA | 96 | 105.502 | 3 | 35.1688 | 34.9056204 | 1.12217775 | 1.275484574 | 358 | 1.32399694 | 1.7:GAAG | 1.4:GAAG | GCT |
| GAA | | GCC | 48 | 79.980 | | 4710539 | 32914915 | 04411289e- | 2957839e-07 | | 5015043 | CCGAA | CTGAA | GCA |
| | | GCG | 32 | 40.932 | | 044 | | 07 | | | | | | GCC |
| | | GCT | 182 | 131.587 | | | | | | | | | | GCG |
| GAA_F_ | E_F_K | TTC | 127 | 86.505 | 1 | 31.1648 | 31.9201467 | 2.37023961 | 1.606423299 | 213 | 1.46923057 | 1.5:GAATT | 1.5:GAATT | TTC |
| AAA | | TTT | 86 | 126.495 | | 1107284 | 8597876 | 82007058e- | 229625e-08 | | 6554999 | TAAA | CAAA | TTT |
| | | | | | | 305 | | 08 | | | | | | |
| GAA_F_ | E_F_D | TTC | 55 | 91.785 | 1 | 26.5064 | 24.8240375 | 2.62664432 | 6.280931531 | 226 | 1.36056658 | 1.7:GAATT | 1.3:GAATT | TTT |
| GAT | | TTT | 171 | 134.215 | | 1489139 | 77611563 | 98690824e- | 100653e-07 | | 6645506 | CGAT | TGAT | TTC |
| | | | | | | 398 | | 07 | | | | | | |
| GAA_G_ | E_G_V | GGA | 14 | 36.463 | 3 | 43.8688 | 39.8229842 | 1.60918779 | 1.161655888 | 163 | 1.51669160 | 2.6:GAAG | 1.5:GAAG | GGT |
| GTT | | GGC | 30 | 32.230 | | 1851502 | 6170389 | 80949446e- | 6937229e-08 | | 78165464 | GAGTT | GTGTT | GGC |
| | | GGG | 8 | 20.246 | | 119 | | 09 | | | | | | GGA |
| | | GGT | 111 | 74.061 | | | | | | | | | | GGG |
| GAA_G_ | E_G_F | GGA | 55 | 36.910 | 3 | 37.2897 | 40.6320203 | 3.99564142 | 7.826406215 | 165 | 1.61143333 | 1.6:GAAG | 2.0:GAAG | GGA |
| TTT | | GGC | 20 | 32.625 | | 1433144 | 147132 | 04473314e- | 09287e-09 | | 36298383 | GCTTT | GGTTT | GGT |
| | | GGG | 40 | 20.495 | | 258 | | 08 | | | | | | GGG |
| | | GGT | 50 | 74.970 | | | | | | | | | | GGC |
| GAA_I_ | E_I_K | ATA | 69 | 69.948 | 2 | 34.2552 | 36.2999728 | 3.64394059 | 1.310874297 | 251 | 1.38226257 | 1.5:GAAA | 1.6:GAAA | ATC |
| AAG | | ATC | 104 | 64.806 | | 3013490 | 5337337 | 27157784e- | 4934256e-08 | | 1596577 | TTAAG | TCAAG | ATT |
| | | ATT | 78 | 116.246 | | 979 | | 08 | | | | | | ATA |
| GAA_I_ | E_I_E | ATA | 114 | 140.453 | 2 | 33.0346 | 32.8948892 | 6.70826284 | 7.193919261 | 504 | 1.28549536 | 1.4:GAAA | 1.3:GAAA | ATT |
| GAA | | ATC | 93 | 130.129 | | 8143394 | 4352821 | 4845118e- | 578336e-08 | | 46297118 | TCGAA | TTGAA | ATA |
| | | ATT | 297 | 233.419 | | 356 | | 08 | | | | | | ATC |
| GAA_I_ | E_I_V | ATA | 47 | 58.801 | 2 | 35.7369 | 33.8578261 | 1.73711556 | 4.444945770 | 211 | 1.44307516 | 2.1:GAAA | 1.4:GAAA | ATT |
| GTT | | ATC | 26 | 54.479 | | 0945873 | 5039289 | 18065218e- | 397595e-08 | | 040918 | TCGTT | TTGTT | ATA |
| | | ATT | 138 | 97.721 | | 791 | | 08 | | | | | | ATC |
| GAA_K_ | E_K_K | AAA | 195 | 311.638 | 1 | 102.250 | 103.755590 | 4.89348489 | 2.288783219 | 538 | 1.54480286 | 1.6:GAAA | 1.5:GAAA | AAG |
| AAA | | AAG | 343 | 226.362 | | 1666951 | 75673644 | 08515474e- | 7377122e-24 | | 32158135 | AAAAA | AGAAA | AAA |
| | | | | | | 668 | | 24 | | | | | | |
| GAA_K_ | E_K_K | AAA | 154 | 232.860 | 1 | 62.4667 | 63.4742048 | 2.70987679 | 1.624806026 | 402 | 1.48363313 | 1.5:GAAA | 1.5:GAAA | AAG |
| AAG | | AAG | 248 | 169.140 | | 0332163 | 2049384 | 1201589e- | 5888797e-15 | | 66726083 | AAAAG | AGAAG | AAA |
| | | | | | | 41 | | 15 | | | | | | |
| GAA_K_ | E_K_Q | AAA | 107 | 147.130 | 1 | 25.5770 | 26.0149993 | 4.25065422 | 3.387750935 | 254 | 1.37531515 | 1.4:GAAA | 1.4:GAAA | AAG |
| CAA | | AAG | 147 | 106.870 | | 4844934 | 06051983 | 36084143e- | 408177e-07 | | 88652779 | AACAA | AGCAA | AAA |
| | | | | | | 6833 | | 07 | | | | | | |
| GAA_K_ | E__E | AAA | 313 | 389.838 | 1 | 35.4361 | 35.9950986 | 2.63549778 | 1.978144932 | 673 | 1.25925849 | 1.2:GAAA | 1.3:GAAA | AAG |
| GAA | | AAG | 360 | 283.162 | | 4979283 | 0272166 | 3525921e- | 1012027e-09 | | 40902833 | AAGAA | AGGAA | AAA |
| | | | | | | 842 | | 09 | | | | | | |
| GAA_L_ | E_L_K | CTA | 102 | 86.806 | 5 | 65.8496 | 61.7754400 | 7.46827658 | 5.219046778 | 613 | 1.32957420 | 2.1:GAACT | 1.4:GAAC | TTG |
| AAA | | CTC | 29 | 35.398 | | 0266106 | 13173366 | 9836579e- | 573212e-12 | | 5455067 | TAAA | TGAAA | TTA |
| | | CTG | 98 | 69.341 | | 316 | | 13 | | | | | | CTA |
| | | CTT | 37 | 78.736 | | | | | | | | | | CTG |
| | | TTA | 126 | 169.171 | | | | | | | | | | CTT |
| | | TTG | 221 | 173.548 | | | | | | | | | | CTC |
| GAA_L_ | E_L_K | CTA | 58 | 51.687 | 5 | 50.0241 | 48.6236152 | 1.37012336 | 2.649205410 | 365 | 1.37484454 | 2.1:GAACT | 1.5:GAATT | TTG |
| AAG | | CTC | 18 | 21.077 | | 4119704 | 12595766 | 55207048e- | 558978e-09 | | 34599583 | TAAG | GAAG | TTA |
| | | CTG | 45 | 41.288 | | 2795 | | 09 | | | | | | CTA |
| | | CTT | 22 | 46.882 | | | | | | | | | | CTG |
| | | TTA | 69 | 100.730 | | | | | | | | | | CTT |
| | | TTG | 153 | 103.336 | | | | | | | | | | CTC |
| GAA_L_ | E_L_I | CTA | 46 | 33.136 | 5 | 36.6358 | 39.2338843 | 7.08527610 | 2.130824543 | 234 | 1.39916543 | 2.0:GAACT | 1.9:GAAC | TTG |
| ATA | | CTC | 15 | 13.513 | | 6618268 | 2911362 | 9061001e- | 840872e-07 | | 38009272 | TATA | TGATA | CTG |
| | | CTG | 50 | 26.470 | | 731 | | 07 | | | | | | TTA |
| | | CTT | 15 | 30.056 | | | | | | | | | | CTA |
| | | TTA | 46 | 64.578 | | | | | | | | | | CTT |
| | | TTG | 62 | 66.248 | | | | | | | | | | CTC |
| GAA_L_ | E_L_S | CTA | 35 | 33.986 | 5 | 41.4424 | 39.3025538 | 7.63694713 | 2.064044375 | 240 | 1.39385634 | 2.1:GAATT | 1.7:GAAC | TTA |
| TCT | | CTC | 19 | 13.859 | | 3483256 | 8079535 | 6818593e- | 4065687e-07 | | 1771762 | GTCT | TTTCT | CTT |
| | | CTG | 21 | 27.148 | | 4686 | | 08 | | | | | | CTA |
| | | CTT | 51 | 30.826 | | | | | | | | | | TTG |
| | | TTA | 82 | 66.234 | | | | | | | | | | CTG |
| | | TTG | 32 | 67.947 | | | | | | | | | | CTC |
| GAA_S_ | E_S_T | AGC | 18 | 24.675 | 5 | 232.033 | 321.829210 | 3.92054764 | 2.022898173 | 219 | 2.90460599 | 4.2:GAATC | 3.7:GAAA | AGT |
| ACC | | AGT | 133 | 35.741 | | 3443715 | 0251212 | 5771847e- | 3187055e-67 | | 5063836 | AACC | GTACC | TCT |
| | | TCA | 11 | 45.820 | | 0374 | | 48 | | | | | | TCC |
| | | TCC | 19 | 34.387 | | | | | | | | | | AGC |
| | | TCG | 6 | 21.342 | | | | | | | | | | TCA |
| | | TCT | 32 | 57.034 | | | | | | | | | | TCG |
| GAA_S_ | E_S_I | AGC | 43 | 34.591 | 5 | 39.5325 | 40.2386851 | 1.85521939 | 1.336671126 | 307 | 1.42574727 | 1.5:GAATC | 1.5:GAAA | AGT |
| ATT | | AGT | 74 | 50.103 | | 2093281 | 7188826 | 96332943e- | 6956296e-07 | | 22057476 | AATT | GTATT | TCC |
| | | TCA | 42 | 64.232 | | 293 | | 07 | | | | | | TCT |
| | | TCC | 70 | 48.205 | | | | | | | | | | AGC |
| | | TCG | 21 | 29.918 | | | | | | | | | | TCA |
| | | TCT | 57 | 79.952 | | | | | | | | | | TCG |
| GAA_S_ | E_S_E | AGC | 72 | 56.900 | 5 | 151.982 | 178.615116 | 5.05150119 | 1.057280205 | 505 | 1.67568893 | 2.0:GAATC | 2.3:GAAA | AGT |
| GAA | | AGT | 186 | 82.417 | | 7451919 | 52477031 | 7679998e- | 3017363e-36 | | 64666352 | CGAA | GTGAA | TCT |
| | | TCA | 78 | 105.659 | | 4516 | | 31 | | | | | | TCA |
| | | TCC | 39 | 79.294 | | | | | | | | | | AGC |
| | | TCG | 26 | 49.213 | | | | | | | | | | TCC |
| | | TCT | 104 | 131.517 | | | | | | | | | | TCG |
| GAA_S_ | E_S_D | AGC | 60 | 47.548 | 5 | 74.4200 | 80.9236678 | 1.22922762 | 5.377486790 | 422 | 1.43569640 | 2.2:GAATC | 1.9:GAAA | AGT |
| GAT | | AGT | 129 | 68.871 | | 4780472 | 6247729 | 85992985e- | 198703e-16 | | 10346014 | GGAT | GTGAT | TCT |
| | | TCA | 68 | 88.293 | | 514 | | 14 | | | | | | TCA |
| | | TCC | 43 | 66.262 | | | | | | | | | | AGC |
| | | TCG | 19 | 41.125 | | | | | | | | | | TCC |
| | | TCT | 103 | 109.901 | | | | | | | | | | TCG |
| GAA_S_ | E_S_G | AGC | 23 | 22.760 | 5 | 65.9198 | 64.9264812 | 7.22165619 | 1.160789718 | 202 | 1.46944698 | 3.9:GAATC | 2.1:GAAA | AGT |
| GGT | | AGT | 68 | 32.967 | | 6635430 | 878808 | 900754e-13 | 9058918e-12 | | 77827803 | GGGT | GTGGT | TCT |
| | | TCA | 16 | 42.263 | | 078 | | | | | | | | TCC |
| | | TCC | 33 | 31.718 | | | | | | | | | | AGC |
| | | TCG | 5 | 19.685 | | | | | | | | | | TCA |
| | | TCT | 57 | 52.607 | | | | | | | | | | TCG |
| GAA_S_ | E_S_S | AGC | 78 | 34.703 | 5 | 50.0009 | 63.3117847 | 1.38516444 | 2.508868889 | 308 | 1.38961125 | 1.6:GAATC | 2.2:GAAA | AGC |
| TCT | | AGT | 44 | 50.266 | | 6949638 | 43544194 | 5552955e- | 455102e-12 | | 18514785 | GTCT | GCTCT | TCT |
| | | TCA | 51 | 64.441 | | 423 | | 09 | | | | | | TCA |
| | | TCC | 40 | 48.362 | | | | | | | | | | AGT |
| | | TCG | 19 | 30.015 | | | | | | | | | | TCC |
| | | TCT | 76 | 80.212 | | | | | | | | | | TCG |
| GAA_V_ | E_V_R | GTA | 3 | 4.949 | 3 | 33.0586 | 43.5403623 | 3.13026258 | 1.889592410 | 23 | 3.46368406 | 4.6:GAAG | 3.8:GAAG | GTG |
| CGG | | GTC | 1 | 4.634 | | 2279305 | 45217865 | 0375659e- | 0289535e-09 | | 6362724 | TCCGG | TGCGG | GTA |
| | | GTG | 17 | 4.512 | | 583 | | 07 | | | | | | GTT |
| | | GTT | 2 | 8.905 | | | | | | | | | | GTC |
| GAC_D_ | D_D_K | GAC | 49 | 26.975 | 1 | 25.7257 | 27.4902060 | 3.93531751 | 1.578920509 | 78 | 1.79508199 | 1.8:GACG | 1.8:GACG | GAC |
| AAG | | GAT | 29 | 51.025 | | 9513740 | 41042825 | 5400039e- | 4588936e-07 | | 04156843 | ATAAG | ACAAG | GAT |
| | | | | | | 5925 | | 07 | | | | | | |
| GAC_G_ | D_G_L | GGA | 16 | 4.698 | 3 | 32.6973 | 37.2912657 | 3.73054160 | 3.992622113 | 21 | 3.09231728 | 9.5:GACG | 3.4:GACG | GGA |
| CTC | | GGC | 1 | 4.152 | | 7637871 | 3616635 | 5209547e- | 917608e-08 | | 13025204 | GTCTC | GACTC | GGG |
| | | GGG | 3 | 2.608 | | 7706 | | 07 | | | | | | GGT |
| | | GGT | 1 | 9.542 | | | | | | | | | | GGC |
| GAC_L_ | D_L_K | CTA | 27 | 31.862 | 5 | 53.2986 | 52.3090228 | 2.92039520 | 4.662137376 | 225 | 1.49564660 | 3.2:GACCT | 1.7:GACTT | TTG |
| AAA | | CTC | 6 | 12.993 | | 7306461 | 06624455 | 98345084e- | 809013e-10 | | 23932524 | TAAA | GAAA | TTA |
| | | CTG | 27 | 25.451 | | 475 | | 10 | | | | | | CTG |
| | | CTT | 9 | 28.900 | | | | | | | | | | CTA |
| | | TTA | 48 | 62.094 | | | | | | | | | | CTT |
| | | TTG | 108 | 63.700 | | | | | | | | | | CTC |
| GAC_L_ | D_L_K | CTA | 18 | 30.021 | 5 | 41.5773 | 41.0731352 | 7.17216557 | 9.068938979 | 212 | 1.43705513 | 2.7:GACCT | 1.6:GACTT | TTG |
| AAG | | CTC | 13 | 12.242 | | 0509900 | 2412589 | 9533535e- | 285104e-08 | | 33196482 | TAAG | GAAG | TTA |
| | | CTG | 23 | 23.981 | | 661 | | 08 | | | | | | CTG |
| | | CTT | 10 | 27.230 | | | | | | | | | | CTA |
| | | TTA | 50 | 58.506 | | | | | | | | | | CTC |
| | | TTG | 98 | 60.020 | | | | | | | | | | CTT |
| GAC_R_ | D_R_V | AGA | 6 | 11.854 | 5 | 27.8284 | 38.5408756 | 3.93195552 | 2.937675623 | 25 | 2.84733177 | 3.4:GACC | 4.0:GACC | CGT |
| GTC | | AGG | 2 | 5.423 | | 5984842 | 30740615 | 0670206e- | 750734e-07 | | 8867363 | GAGTC | GTGTC | AGA |
| | | CGA | 0 | 1.707 | | 0223 | | 05 | | | | | | CGC |
| | | CGC | 2 | 1.480 | | | | | | | | | | AGG |
| | | CGG | 1 | 1.044 | | | | | | | | | | CGG |
| | | CGT | 14 | 3.493 | | | | | | | | | | CGA |
| GAC_R_ | D_R_L | AGA | 43 | 49.311 | 5 | 29.2664 | 44.2568037 | 2.05572002 | 2.054185539 | 104 | 1.52396033 | 2.2:GACC | 3.4:GACC | AGA |
| TTG | | AGG | 19 | 22.558 | | 3392270 | 3595955 | 2754678e- | 4875484e-08 | | 86597116 | GGTTG | GATTG | CGA |
| | | CGA | 24 | 7.102 | | 364 | | 05 | | | | | | AGG |
| | | CGC | 6 | 6.155 | | | | | | | | | | CGT |
| | | CGG | 2 | 4.341 | | | | | | | | | | CGC |
| | | CGT | 10 | 14.533 | | | | | | | | | | CGG |
| GAC_S_ | D_S_D | AGC | 28 | 13.633 | 5 | 37.2828 | 41.0898713 | 5.25630245 | 8.998602833 | 121 | 1.70972365 | 1.9:GACTC | 2.1:GACA | AGT |
| GAC | | AGT | 36 | 19.747 | | 0149256 | 2455584 | 466145e-07 | 064653e-08 | | 34728628 | CGAC | GCGAC | AGC |
| | | TCA | 19 | 25.316 | | 313 | | | | | | | | TCA |
| | | TCC | 10 | 18.999 | | | | | | | | | | TCT |
| | | TCG | 11 | 11.792 | | | | | | | | | | TCG |
| | | TCT | 17 | 31.512 | | | | | | | | | | TCC |
| GAC_S_ | D_S_D | AGC | 37 | 24.112 | 5 | 36.5893 | 39.8651572 | 7.23906589 | 1.589845906 | 214 | 1.48458685 | 1.7:GACTC | 1.8:GACA | AGT |
| GAT | | AGT | 62 | 34.925 | | 0682283 | 1969571 | 987142e-07 | 8954704e-07 | | 33801976 | GGAT | GTGAT | TCT |
| | | TCA | 35 | 44.774 | | 777 | | | | | | | | AGC |
| | | TCC | 21 | 33.602 | | | | | | | | | | TCA |
| | | TCG | 12 | 20.855 | | | | | | | | | | TCC |
| | | TCT | 47 | 55.732 | | | | | | | | | | TCG |
| GAG_A_ | E_A_A | GCA | 8 | 10.314 | 3 | 40.5213 | 64.7265873 | 8.26097599 | 5.739079359 | 35 | 3.04153798 | 3.9:GAGG | 4.7:GAGG | GCG |
| GCG | | GCC | 2 | 7.819 | | 3942782 | 7675347 | 4964996e- | 844873e-14 | | 62379934 | CCGCG | CGGCG | GCA |
| | | GCG | 19 | 4.002 | | 3904 | | 09 | | | | | | GCT |
| | | GCT | 6 | 12.865 | | | | | | | | | | GCC |
| GAG_C_ | E_C_T | TGC | 30 | 12.046 | 1 | 45.5464 | 42.9136824 | 1.49060598 | 5.720913731 | 32 | 2.71610375 | 10.0:GAGT | 2.5:GAGT | TGC |
| ACC | | TGT | 2 | 19.954 | | 6316346 | 01950396 | 1265762e- | 861019e-11 | | 80822924 | GTACC | GCACC | TGT |
| | | | | | | 138 | | 11 | | | | | | |
| GAG_F_ | E_F_K | TTC | 53 | 30.866 | 1 | 26.2977 | 26.7278006 | 2.92630202 | 2.342272193 | 76 | 1.78791272 | 2.0:GAGTT | 1.7:GAGTT | TTC |
| AAG | | TTT | 23 | 45.134 | | 4467020 | 2000927 | 00478625e- | 062238e-07 | | 48014608 | TAAG | CAAG | TTT |
| | | | | | | 812 | | 07 | | | | | | |
| GAG_G_ | E_G_N | GGA | 37 | 18.343 | 3 | 52.6248 | 46.6839541 | 2.20410453 | 4.057447387 | 82 | 1.86090510 | 4.7:GAGG | 2.0:GAGG | GGA |
| AAT | | GGC | 21 | 16.214 | | 1087345 | 4904819 | 54368606e- | 4683053e-10 | | 40304536 | GTAAT | GAAAT | GGC |
| | | GGG | 16 | 10.185 | | 7754 | | 11 | | | | | | GGG |
| | | GGT | 8 | 37.258 | | | | | | | | | | GGT |
| GAG_L_ | E_L_K | CTA | 38 | 41.208 | 5 | 52.0319 | 56.8365589 | 5.31400540 | 5.464794878 | 291 | 1.40237960 | 2.3:GAGCT | 2.3:GAGC | TTG |
| AAA | | CTC | 39 | 16.804 | | 4454183 | 6107976 | 6116372e- | 678549e-11 | | 5515432 | TAAA | TCAAA | TTA |
| | | CTG | 48 | 32.917 | | 273 | | 10 | | | | | | CTG |
| | | CTT | 16 | 37.377 | | | | | | | | | | CTC |
| | | TTA | 57 | 80.308 | | | | | | | | | | CTA |
| | | TTG | 93 | 82.386 | | | | | | | | | | CTT |
| GAG_L_ | E_L_I | CTA | 21 | 27.897 | 5 | 28.0921 | 38.6297578 | 3.49189833 | 2.819215528 | 197 | 1.30235737 | 1.3:GAGCT | 2.7:GAGC | TTG |
| ATT | | CTC | 31 | 11.376 | | 9406116 | 6867777 | 80375875e- | 7625367e-07 | | 24634725 | AATT | TCATT | TTA |
| | | CTG | 23 | 22.284 | | 713 | | 05 | | | | | | CTC |
| | | CTT | 23 | 25.303 | | | | | | | | | | CTT |
| | | TTA | 42 | 54.367 | | | | | | | | | | CTG |
| | | TTG | 57 | 55.773 | | | | | | | | | | CTA |
| GAG_L_ | E_L_D | CTA | 31 | 26.056 | 5 | 33.1162 | 39.0913020 | 3.56852966 | 2.276434327 | 184 | 1.45383203 | 1.5:GAGTT | 2.2:GAGC | CTG |
| GAT | | CTC | 13 | 10.625 | | 9109112 | 3874892 | 56747936e- | 7830406e-07 | | 98781115 | GGAT | TGGAT | TTA |
| | | CTG | 45 | 20.814 | | 552 | | 06 | | | | | | TTG |
| | | CTT | 23 | 23.633 | | | | | | | | | | CTA |
| | | TTA | 38 | 50.779 | | | | | | | | | | CTT |
| | | TTG | 34 | 52.093 | | | | | | | | | | CTC |
| GAG_L_ | E_L_S | CTA | 28 | 17.418 | 5 | 46.0180 | 47.4141868 | 9.00568694 | 4.677348915 | 123 | 1.73462788 | 2.5:GAGTT | 2.0:GAGC | CTT |
| TCT | | CTC | 14 | 7.103 | | 2504267 | 4364153 | 5033207e- | 776447e-09 | | 09054805 | GTCT | TTTCT | TTA |
| | | CTG | 6 | 13.913 | | 857 | | 09 | | | | | | CTA |
| | | CTT | 32 | 15.798 | | | | | | | | | | TTG |
| | | TTA | 29 | 33.945 | | | | | | | | | | CTC |
| | | TTG | 14 | 34.823 | | | | | | | | | | CTG |
| GAG_L_ | E_L_C | CTA | 12 | 8.496 | 5 | 47.6188 | 81.1087905 | 4.24869506 | 4.918500768 | 60 | 2.29112915 | 3.4:GAGCT | 5.5:GAGC | CTC |
| TGT | | CTC | 19 | 3.465 | | 1360587 | 4656228 | 0927305e- | 555583e-16 | | 74071124 | GTGT | TCTGT | CTA |
| | | CTG | 2 | 6.787 | | 48 | | 09 | | | | | | TTA |
| | | CTT | 8 | 7.707 | | | | | | | | | | TTG |
| | | TTA | 11 | 16.558 | | | | | | | | | | CTT |
| | | TTG | 8 | 16.987 | | | | | | | | | | CTG |
| GAG_R_ | E_R_L | AGA | 8 | 21.336 | 5 | 83.4197 | 227.371767 | 1.61393619 | 3.912813396 | 45 | 4.45922393 | 3.1:GAGC | 11.7:GAGC | CGG |
| CTG | | AGG | 8 | 9.761 | | 0197450 | 38538028 | 38107453e- | 1766e-47 | | 6683471 | GACTG | GGCTG | AGG |
| | | CGA | 1 | 3.073 | | 13 | | 16 | | | | | | AGA |
| | | CGC | 3 | 2.663 | | | | | | | | | | CGT |
| | | CGG | 22 | 1.878 | | | | | | | | | | CGC |
| | | CGT | 3 | 6.288 | | | | | | | | | | CGA |
| GAG_R_ | E_R_W | AGA | 6 | 18.017 | 5 | 57.7995 | 121.397095 | 3.45962078 | 1.587721550 | 38 | 3.71231093 | 3.0:GAGA | 8.0:GAGC | CGC |
| TGG | | AGG | 4 | 8.242 | | 7221063 | 8844032 | 62551156e- | 0420795e-24 | | 3766096 | GATGG | GCTGG | CGT |
| | | CGA | 2 | 2.595 | | 796 | | 11 | | | | | | AGA |
| | | CGC | 18 | 2.249 | | | | | | | | | | AGG |
| | | CGG | 1 | 1.586 | | | | | | | | | | CGA |
| | | CGT | 7 | 5.310 | | | | | | | | | | CGG |
| GAG_S_ | E_S_N | AGC | 47 | 18.253 | 5 | 45.4763 | 57.9859693 | 1.16079765 | 3.166497683 | 162 | 1.64667747 | 1.6:GAGTC | 2.6:GAGA | AGC |
| AAT | | AGT | 33 | 26.439 | | 4736172 | 1343502 | 63085564e- | 238478e-11 | | 36356822 | TAAT | GCAAT | AGT |
| | | TCA | 25 | 33.894 | | 9374 | | 08 | | | | | | TCT |
| | | TCC | 19 | 25.437 | | | | | | | | | | TCA |
| | | TCG | 12 | 15.787 | | | | | | | | | | TCC |
| | | TCT | 26 | 42.189 | | | | | | | | | | TCG |
| GAG_S_ | E_S_A | AGC | 8 | 7.211 | 5 | 43.6379 | 50.2261270 | 2.74313593 | 1.245723108 | 64 | 1.81423673 | 5.0:GAGTC | 3.4:GAGT | TCG |
| GCT | | AGT | 10 | 10.445 | | 9250577 | 9183408 | 02360783e- | 8709542e-09 | | 40545333 | CGCT | CGGCT | TCT |
| | | TCA | 3 | 13.390 | | 493 | | 08 | | | | | | AGT |
| | | TCC | 2 | 10.049 | | | | | | | | | | AGC |
| | | TCG | 21 | 6.237 | | | | | | | | | | TCA |
| | | TCT | 20 | 16.667 | | | | | | | | | | TCC |
| GAG_S_ | E_S_Y | AGC | 6 | 7.887 | 5 | 31.1662 | 44.9812794 | 8.68523721 | 1.463647405 | 70 | 1.86650526 | 1.8:GAGTC | 3.4:GAGT | TCG |
| TAT | | AGT | 13 | 11.424 | | 3718444 | 43410884 | 8279651e- | 3462986e-08 | | 6643758 | CTAT | CGTAT | TCT |
| | | TCA | 9 | 14.646 | | 8313 | | 06 | | | | | | AGT |
| | | TCC | 6 | 10.991 | | | | | | | | | | TCA |
| | | TCG | 23 | 6.822 | | | | | | | | | | TCC |
| | | TCT | 13 | 18.230 | | | | | | | | | | AGC |
| GAG_V_ | E_V_W | GTA | 4 | 7.101 | 3 | 32.6230 | 41.3657647 | 3.86759527 | 5.469418190 | 33 | 2.72787978 | 6.6:GAGG | 3.2:GAGG | GTG |
| TGG | | GTC | 1 | 6.649 | | 7019258 | 20759366 | 26544793e- | 292964e-09 | | 2348297 | TCTGG | TGTGG | GTT |
| | | GTG | 21 | 6.473 | | 5516 | | 07 | | | | | | GTA |
| | | GTT | 7 | 12.777 | | | | | | | | | | GTC |
| GAT_F_ | D_F_K | TTC | 109 | 64.574 | 1 | 50.5509 | 51.4655749 | 1.16112285 | 7.286362570 | 159 | 1.74863171 | 1.9:GATTT | 1.7:GATTT | TTC |
| AAG | | TTT | 50 | 94.426 | | 2779760 | 2166983 | 06657451e- | 840747e-13 | | 93406067 | TAAG | CAAG | TTT |
| | | | | | | 554 | | 12 | | | | | | |
| GAT_G_ | D_G_G | GGA | 4 | 13.422 | 3 | 28.6411 | 35.8892190 | 2.66400918 | 7.903303976 | 60 | 1.74218878 | 3.4:GATG | 3.0:GATG | GGT |
| GGC | | GGC | 9 | 11.864 | | 1021556 | 2793636 | 6601589e- | 678835e-08 | | 75014093 | GAGGC | GGGGC | GGG |
| | | GGG | 22 | 7.453 | | 929 | | 06 | | | | | | GGC |
| | | GGT | 25 | 27.262 | | | | | | | | | | GGA |
| GAT_G_ | D_G_F | GGA | 48 | 36.910 | 3 | 44.0976 | 49.9753958 | 1.43876595 | 8.086154735 | 165 | 1.60000998 | 1.8:GATG | 2.2:GATG | GGA |
| TTT | | GGC | 29 | 32.625 | | 8781924 | 475915 | 35210326e- | 467642e-11 | | 4322213 | GTTTT | GGTTT | GGG |
| | | GGG | 46 | 20.495 | | 773 | | 09 | | | | | | GGT |
| | | GGT | 42 | 74.970 | | | | | | | | | | GGC |
| GAT_I_ | D_I_K | ATA | 81 | 86.111 | 2 | 48.1825 | 51.9752852 | 3.44583399 | 5.172615571 | 309 | 1.43616389 | 1.5:GATAT | 1.7:GATAT | ATC |
| AAA | | ATC | 133 | 79.781 | | 4012022 | 84295836 | 1963921e- | 198624e-12 | | 29312315 | TAAA | CAAA | ATT |
| | | ATT | 95 | 143.108 | | 912 | | 11 | | | | | | ATA |
| GAT_I_ | D_I_N | ATA | 48 | 46.539 | 2 | 27.9916 | 29.2078678 | 8.35027268 | 4.545609159 | 167 | 1.43052192 | 1.6:GATAT | 1.6:GATAT | ATC |
| AAC | | ATC | 71 | 43.118 | | 0291095 | 07847705 | 7233924e- | 014489e-07 | | 25523156 | TAAC | CAAC | ATT |
| | | ATT | 48 | 77.343 | | 005 | | 07 | | | | | | ATA |
| GAT_I_ | D_I_K | ATA | 47 | 59.637 | 2 | 34.5697 | 38.6855828 | 3.11362150 | 3.976787821 | 214 | 1.49242444 | 1.4:GATAT | 1.7:GATAT | ATC |
| AAG | | ATC | 95 | 55.253 | | 8845098 | 472755 | 08899247e- | 109751e-09 | | 56880957 | TAAG | CAAG | ATT |
| | | ATT | 72 | 99.110 | | 141 | | 08 | | | | | | ATA |
| GAT_L_ | D_L_K | CTA | 47 | 76.185 | 5 | 87.9864 | 87.9210432 | 1.77891778 | 1.836025994 | 538 | 1.40595628 | 2.3:GATCT | 1.6:GATTT | TTG |
| AAA | | CTC | 34 | 31.067 | | 1959078 | 7826965 | 4450194e- | 3540603e-17 | | 66419515 | TAAA | GAAA | TTA |
| | | CTG | 52 | 60.857 | | 412 | | 17 | | | | | | CTG |
| | | CTT | 30 | 69.102 | | | | | | | | | | CTA |
| | | TTA | 134 | 148.473 | | | | | | | | | | CTC |
| | | TTG | 241 | 152.314 | | | | | | | | | | CTT |
| GAT_L_ | D_L_N | CTA | 24 | 36.252 | 5 | 47.1110 | 47.7818978 | 5.39290227 | 3.935303094 | 256 | 1.46666346 | 2.1:GATCT | 1.6:GATTT | TTG |
| AAC | | CTC | 19 | 14.783 | | 3595664 | 1751615 | 5503992e- | 3219055e-09 | | 3060866 | GAAC | GAAC | TTA |
| | | CTG | 14 | 28.958 | | 494 | | 09 | | | | | | CTA |
| | | CTT | 18 | 32.881 | | | | | | | | | | CTC |
| | | TTA | 64 | 70.649 | | | | | | | | | | CTT |
| | | TTG | 117 | 72.477 | | | | | | | | | | CTG |
| GAT_L_ | D_L_K | CTA | 32 | 50.271 | 5 | 67.3537 | 68.3826091 | 3.63809249 | 2.223577315 | 355 | 1.44190305 | 2.3:GATCT | 1.6:GATTT | TTG |
| AAG | | CTC | 20 | 20.500 | | 6533502 | 8055431 | 69811595e- | 1379489e-13 | | 02945913 | TAAG | GAAG | TTA |
| | | CTG | 25 | 40.157 | | 019 | | 13 | | | | | | CTA |
| | | CTT | 20 | 45.597 | | | | | | | | | | CTG |
| | | TTA | 93 | 97.970 | | | | | | | | | | CTT |
| | | TTG | 165 | 100.505 | | | | | | | | | | CTC |
| GAT_L_ | D_L_S | CTA | 12 | 14.586 | 5 | 49.2589 | 78.0393061 | 1.96458728 | 2.157022227 | 103 | 1.79468524 | 2.6:GATCT | 4.4:GATCT | TTG |
| AGC | | CTC | 26 | 5.948 | | 5873845 | 773889 | 5425801e- | 116615e-15 | | 71770545 | TAGC | CAGC | CTC |
| | | CTG | 7 | 11.651 | | 429 | | 09 | | | | | | TTA |
| | | CTT | 5 | 13.230 | | | | | | | | | | CTA |
| | | TTA | 20 | 28.425 | | | | | | | | | | CTG |
| | | TTG | 33 | 29.161 | | | | | | | | | | CTT |
| GAT_L_ | D_L_C | CTA | 30 | 10.621 | 5 | 32.2927 | 42.7094507 | 5.19849283 | 4.231551848 | 75 | 1.85046461 | 2.1:GATCT | 2.8:GATCT | CTA |
| TGC | | CTC | 4 | 4.331 | | 8550614 | 3682768 | 0061062e- | 6371756e-08 | | 4780177 | GTGC | ATGC | TTA |
| | | CTG | 4 | 8.484 | | 9286 | | 06 | | | | | | TTG |
| | | CTT | 8 | 9.633 | | | | | | | | | | CTT |
| | | TTA | 17 | 20.698 | | | | | | | | | | CTG |
| | | TTG | 12 | 21.233 | | | | | | | | | | CTC |
| GAT_L_ | D_L_F | CTA | 17 | 29.454 | 5 | 57.8875 | 73.8774612 | 3.31810964 | 1.595193262 | 208 | 1.57329067 | 1.8:GATCT | 2.5:GATCT | CTT |
| TTC | | CTC | 12 | 12.011 | | 0544023 | 0398918 | 37616376e- | 2098474e-14 | | 23397868 | GTTC | TTTC | TTA |
| | | CTG | 13 | 23.528 | | 824 | | 11 | | | | | | TTG |
| | | CTT | 67 | 26.716 | | | | | | | | | | CTA |
| | | TTA | 53 | 57.402 | | | | | | | | | | CTG |
| | | TTG | 46 | 58.887 | | | | | | | | | | CTC |
| GAT_P_ | D_P_Q | CCA | 21 | 37.889 | 3 | 37.0556 | 36.1447602 | 4.47825556 | 6.978789776 | 93 | 1.78426781 | 3.7:GATCC | 1.8:GATCC | CCT |
| CAA | | CCC | 4 | 14.926 | | 7350309 | 666282 | 6425187e- | 394897e-08 | | 8048624 | CCAA | TCAA | CCA |
| | | CCG | 16 | 11.431 | | 8404 | | 08 | | | | | | CCG |
| | | CCT | 52 | 28.754 | | | | | | | | | | CCC |
| GAT_P_ | D_P_H | CCA | 9 | 24.037 | 3 | 32.9249 | 35.3540397 | 3.34016324 | 1.025482539 | 59 | 2.06580244 | 2.7:GATCC | 2.1:GATCC | CCT |
| CAT | | CCC | 5 | 9.469 | | 9251227 | 74819306 | 13744955e- | 7431961e-07 | | 84631325 | ACAT | TCAT | CCA |
| | | CCG | 6 | 7.252 | | 561 | | 07 | | | | | | CCG |
| | | CCT | 39 | 18.242 | | | | | | | | | | CCC |
| GAT_V_ | D_V_K | GTA | 29 | 42.174 | 3 | 42.3095 | 49.2461056 | 3.44883064 | 1.156204739 | 196 | 1.54315704 | 1.5:GATGT | 2.0:GATGT | GTC |
| AAG | | GTC | 78 | 39.489 | | 9071015 | 5149896 | 2473037e- | 3165924e-10 | | 84650834 | TAAG | CAAG | GTT |
| | | GTG | 37 | 38.447 | | 517 | | 09 | | | | | | GTG |
| | | GTT | 52 | 75.889 | | | | | | | | | | GTA |
| GAT_V_ | D_V_V | GTA | 15 | 20.011 | 3 | 28.9759 | 35.5417573 | 2.26568318 | 9.359569566 | 93 | 1.74759867 | 1.6:GATGT | 2.2:GATGT | GTG |
| GTG | | GTC | 14 | 18.737 | | 1028500 | 4960228 | 3493441e- | 191827e-08 | | 56182127 | TGTG | GGTG | GTT |
| | | GTG | 41 | 18.243 | | 755 | | 06 | | | | | | GTA |
| | | GTT | 23 | 36.009 | | | | | | | | | | GTC |
| GCA_G_ | A_G_Q | GGA | 4 | 6.935 | 3 | 42.5211 | 52.8903531 | 3.11003818 | 1.934746734 | 31 | 3.08946675 | 7.0:GCAG | 3.6:GCAG | GGC |
| CAG | | GGC | 22 | 6.130 | | 5912553 | 5493904 | 42646685e- | 7329835e-11 | | 13887186 | GTCAG | GCCAG | GGA |
| | | GGG | 3 | 3.851 | | 496 | | 09 | | | | | | GGG |
| | | GGT | 2 | 14.085 | | | | | | | | | | GGT |
| GCA_L_ | A_L_A | CTA | 6 | 6.514 | 5 | 37.2539 | 55.9737064 | 5.32679170 | 8.228509312 | 46 | 2.38440857 | 3.0:GCACT | 4.0:GCACT | CTG |
| GCG | | CTC | 1 | 2.656 | | 5812484 | 3250873 | 0264022e- | 897271e-11 | | 78948303 | TGCG | GGCG | TTA |
| | | CTG | 21 | 5.203 | | 6704 | | 07 | | | | | | TTG |
| | | CTT | 2 | 5.908 | | | | | | | | | | CTA |
| | | TTA | 10 | 12.695 | | | | | | | | | | CTT |
| | | TTG | 6 | 13.023 | | | | | | | | | | CTC |
| GCA_S_ | A_S_T | AGC | 39 | 14.422 | 5 | 36.8265 | 49.3560547 | 6.48857281 | 1.876804729 | 128 | 1.61082685 | 1.6:GCATC | 2.7:GCAA | AGC |
| ACA | | AGT | 17 | 20.890 | | 7087140 | 4771468 | 6900233e- | 8787636e-09 | | 92638415 | GACA | GCACA | TCT |
| | | TCA | 21 | 26.781 | | 598 | | 07 | | | | | | TCC |
| | | TCC | 21 | 20.098 | | | | | | | | | | TCA |
| | | TCG | 8 | 12.474 | | | | | | | | | | AGT |
| | | TCT | 22 | 33.335 | | | | | | | | | | TCG |
| GCA_V_ | A_V_Q | GTA | 25 | 8.177 | 3 | 37.2347 | 45.5296485 | 4.10413645 | 7.139902509 | 38 | 2.66601351 | 3.7:GCAGT | 3.1:GCAG | GTA |
| CAG | | GTC | 3 | 7.656 | | 2971577 | 9206956 | 51645335e- | 555135e-10 | | 4081875 | TCAG | TACAG | GTG |
| | | GTG | 6 | 7.454 | | 8535 | | 08 | | | | | | GTT |
| | | GTT | 4 | 14.713 | | | | | | | | | | GTC |
| GCA_V_ | A_V_S | GTA | 20 | 7.101 | 3 | 28.0324 | 31.9264899 | 3.57556554 | 5.423519293 | 33 | 2.29435060 | 6.6:GCAGT | 2.8:GCAG | GTA |
| TCC | | GTC | 1 | 6.649 | | 2271439 | 95346575 | 9937698e- | 502566e-07 | | 7664937 | CTCC | TATCC | GTT |
| | | GTG | 2 | 6.473 | | 171 | | 06 | | | | | | GTG |
| | | GTT | 10 | 12.777 | | | | | | | | | | GTC |
| GCC_A_ | A_A_I | GCA | 4 | 15.030 | 3 | 34.2474 | 38.2193937 | 1.75665751 | 2.539764934 | 51 | 2.06824126 | 3.8:GCCGC | 2.5:GCCG | GCC |
| ATC | | GCC | 29 | 11.394 | | 2649222 | 8809799 | 01418362e- | 5000957e-08 | | 45591532 | AATC | CCATC | GCT |
| | | GCG | 2 | 5.831 | | 9295 | | 07 | | | | | | GCA |
| | | GCT | 16 | 18.746 | | | | | | | | | | GCG |
| GCC_A_ | A_A_A | GCA | 12 | 25.639 | 3 | 40.0251 | 37.0859693 | 1.05250316 | 4.412642962 | 87 | 1.75740041 | 9.9:GCCGC | 1.8:GCCG | GCT |
| GCT | | GCC | 16 | 19.436 | | 6228151 | 97448466 | 1054687e- | 192927e-08 | | 986658 | GGCT | CTGCT | GCC |
| | | GCG | 1 | 9.947 | | 3744 | | 08 | | | | | | GCA |
| | | GCT | 58 | 31.978 | | | | | | | | | | GCG |
| GCC_G_ | A_G_G | GGA | 4 | 14.093 | 3 | 41.7494 | 34.1377814 | 4.53467718 | 1.852837975 | 63 | 1.82892825 | 15.7:GCCG | 1.8:GCCG | GGT |
| GGT | | GGC | 8 | 12.457 | | 1817543 | 0638699 | 3522818e- | 6591188e-07 | | 23512247 | GGGGT | GTGGT | GGC |
| | | GGG | 0 | 7.825 | | 96 | | 09 | | | | | | GGA |
| | | GGT | 51 | 28.625 | | | | | | | | | | GGG |
| GCC_K_ | A_K_K | AAA | 69 | 101.949 | 1 | 24.8968 | 25.3088543 | 6.04802120 | 4.884613461 | 176 | 1.45762644 | 1.5:GCCA | 1.4:GCCA | AAG |
| AAG | | AAG | 107 | 74.051 | | 8048870 | 1143259 | 068721e-07 | 727177e-07 | | 56706434 | AAAAG | AGAAG | AAA |
| | | | | | | 9762 | | | | | | | | |
| GCC_L_ | A_L_F | CTA | 6 | 7.222 | 5 | 43.1393 | 57.4648153 | 3.46232392 | 4.055692812 | 51 | 2.22371991 | 4.8:GCCTT | 3.7:GCCCT | CTT |
| TTC | | CTC | 2 | 2.945 | | 9159587 | 1827524 | 3190074e- | 296156e-11 | | 73479016 | GTTC | TTTC | TTA |
| | | CTG | 3 | 5.769 | | 761 | | 08 | | | | | | CTA |
| | | CTT | 24 | 6.551 | | | | | | | | | | TTG |
| | | TTA | 13 | 14.075 | | | | | | | | | | CTG |
| | | TTG | 3 | 14.439 | | | | | | | | | | CTC |
| GCC_L_ | A_L_F | CTA | 12 | 14.444 | 5 | 43.3252 | 65.7222488 | 3.17451183 | 7.936911516 | 102 | 1.70835407 | 2.9:GCCCT | 4.1:GCCCT | TTA |
| TTT | | CTC | 24 | 5.890 | | 8108985 | 639211 | 61560456e- | 119991e-13 | | 5942903 | GTTT | CTTT | CTC |
| | | CTG | 4 | 11.538 | | 214 | | 08 | | | | | | TTG |
| | | CTT | 17 | 13.101 | | | | | | | | | | CTT |
| | | TTA | 26 | 28.149 | | | | | | | | | | CTA |
| | | TTG | 19 | 28.877 | | | | | | | | | | CTG |
| GCC_S_ | A_S_T | AGC | 36 | 11.155 | 5 | 80.1517 | 89.9549385 | 7.80029538 | 6.867381488 | 99 | 2.21529620 | 5.2:GCCTC | 3.2:GCCA | AGC |
| ACC | | AGT | 25 | 16.157 | | 3747076 | 5365255 | 4790536e- | 711143e-18 | | 95771844 | TACC | GCACC | AGT |
| | | TCA | 8 | 20.713 | | 7 | | 16 | | | | | | TCC |
| | | TCC | 21 | 15.545 | | | | | | | | | | TCA |
| | | TCG | 4 | 9.648 | | | | | | | | | | TCT |
| | | TCT | 5 | 25.782 | | | | | | | | | | TCG |
| GCC_S_ | A_S_I | AGC | 47 | 9.915 | 5 | 103.000 | 160.902464 | 1.23167151 | 6.356360885 | 88 | 2.93568669 | 4.6:GCCTC | 4.7:GCCA | AGC |
| ATC | | AGT | 7 | 14.362 | | 0036103 | 3257372 | 01867901e- | 492482e-33 | | 65684993 | AATC | GCATC | TCT |
| | | TCA | 4 | 18.412 | | 0034 | | 20 | | | | | | TCC |
| | | TCC | 11 | 13.818 | | | | | | | | | | AGT |
| | | TCG | 2 | 8.576 | | | | | | | | | | TCA |
| | | TCT | 17 | 22.918 | | | | | | | | | | TCG |
| GCG_A_ | A_A_S | GCA | 7 | 8.841 | 3 | 38.5199 | 61.9573827 | 2.19354008 | 2.243670690 | 30 | 3.22865567 | 3.7:GCGG | 5.0:GCGG | GCG |
| AGT | | GCC | 3 | 6.702 | | 5730046 | 84344645 | 25032887e- | 0378534e-13 | | 8046206 | CTAGT | CGAGT | GCA |
| | | GCG | 17 | 3.430 | | 461 | | 08 | | | | | | GCT |
| | | GCT | 3 | 11.027 | | | | | | | | | | GCC |
| GCG_L_ | A_L_E | CTA | 6 | 7.080 | 5 | 54.9068 | 109.965794 | 1.36421747 | 4.166099967 | 50 | 2.71626077 | 6.4:GCGCT | 6.9:GCGCT | CTC |
| GAG | | CTC | 20 | 2.887 | | 9823321 | 28782584 | 04512548e- | 70383e-22 | | 47061003 | TGAG | CGAG | TTA |
| | | CTG | 5 | 5.656 | | 529 | | 10 | | | | | | TTG |
| | | CTT | 1 | 6.422 | | | | | | | | | | CTA |
| | | TTA | 10 | 13.799 | | | | | | | | | | CTG |
| | | TTG | 8 | 14.156 | | | | | | | | | | CTT |
| GCG_L_ | A_L_G | CTA | 2 | 6.797 | 5 | 41.6827 | 65.1094993 | 6.82852837 | 1.063628399 | 48 | 2.58383296 | 3.4:GCGCT | 4.2:GCGCT | CTG |
| GGA | | CTC | 2 | 2.772 | | 4458061 | 0225205 | 6685487e- | 562051e-12 | | 47944582 | AGGA | GGGA | TTG |
| | | CTG | 23 | 5.430 | | 6216 | | 08 | | | | | | TTA |
| | | CTT | 3 | 6.165 | | | | | | | | | | CTT |
| | | TTA | 8 | 13.247 | | | | | | | | | | CTC |
| | | TTG | 10 | 13.589 | | | | | | | | | | CTA |
| GCG_S_ | A_S_A | AGC | 2 | 2.817 | 5 | 38.7385 | 50.3752370 | 2.68071765 | 1.161177606 | 25 | 3.20942622 | 10.5:GCGT | 4.2:GCGA | AGT |
| GCC | | AGT | 17 | 4.080 | | 1638125 | 0255552 | 26179934e- | 5862873e-09 | | 45673977 | CAGCC | GTGCC | TCT |
| | | TCA | 0 | 5.231 | | 535 | | 07 | | | | | | AGC |
| | | TCC | 1 | 3.925 | | | | | | | | | | TCG |
| | | TCG | 1 | 2.436 | | | | | | | | | | TCC |
| | | TCT | 4 | 6.511 | | | | | | | | | | TCA |
| GCG_T_ | A_T_T | ACA | 2 | 24.313 | 3 | 92.3348 | 91.7560090 | 6.90244531 | 9.190755335 | 80 | 2.61405847 | 12.2:GCGA | 2.5:GCGA | ACT |
| ACC | | ACC | 6 | 16.949 | | 0966500 | 5038786 | 206069e-20 | 486977e-20 | | 9740813 | CAACC | CTACC | ACC |
| | | ACG | 4 | 11.221 | | 07 | | | | | | | | ACG |
| | | ACT | 68 | 27.517 | | | | | | | | | | ACA |
| GCT_A_ | A_A_A | GCA | 18 | 37.427 | 3 | 36.9034 | 37.3727583 | 4.82294344 | 3.837185254 | 127 | 1.66405908 | 2.1:GCTGC | 1.7:GCTGC | GCT |
| GCC | | GCC | 23 | 28.373 | | 6213276 | 50513124 | 83484256e- | 585222e-08 | | 85853912 | AGCC | TGCC | GCC |
| | | GCG | 7 | 14.520 | | 699 | | 08 | | | | | | GCA |
| | | GCT | 79 | 46.680 | | | | | | | | | | GCG |
| GCT_A_ | A_A_A | GCA | 32 | 76.621 | 3 | 82.5251 | 81.1259822 | 8.81663674 | 1.759926450 | 260 | 1.64705933 | 2.4:GCTGC | 1.7:GCTGC | GCT |
| GCT | | GCC | 52 | 58.086 | | 1149334 | 7476815 | 5395354e- | 202247e-17 | | 42201214 | AGCT | TGCT | GCC |
| | | GCG | 14 | 29.727 | | 325 | | 18 | | | | | | GCA |
| | | GCT | 162 | 95.566 | | | | | | | | | | GCG |
| GCT_A_ | A_A_G | GCA | 41 | 70.727 | 3 | 38.7372 | 36.4493892 | 1.97297902 | 6.016726269 | 240 | 1.41613471 | 2.1:GCTGC | 1.4:GCTGC | GCT |
| GGT | | GCC | 61 | 53.618 | | 5604293 | 4308222 | 7263867e- | 5278e-08 | | 08134836 | GGGT | TGGT | GCC |
| | | GCG | 13 | 27.440 | | 931 | | 08 | | | | | | GCA |
| | | GCT | 125 | 88.215 | | | | | | | | | | GCG |
| GCT_A_ | A_A_W | GCA | 10 | 16.503 | 3 | 29.5115 | 43.5345620 | 1.74826127 | 1.894959623 | 56 | 2.02653381 | 1.7:GCTGC | 3.4:GCTGC | GCG |
| TGG | | GCC | 11 | 12.511 | | 9775376 | 9257341 | 60462643e- | 6140917e-09 | | 0318722 | ATGG | GTGG | GCT |
| | | GCG | 22 | 6.403 | | 719 | | 06 | | | | | | GCC |
| | | GCT | 13 | 20.583 | | | | | | | | | | GCA |
| GCT_A_ | A_A_L | GCA | 32 | 56.877 | 3 | 54.8185 | 52.4542883 | 7.50632775 | 2.396521475 | 193 | 1.64161877 | 2.4:GCTGC | 1.6:GCTGC | GCT |
| TTG | | GCC | 18 | 43.118 | | 2944935 | 2340451 | 2445391e- | 6037353e-11 | | 87436765 | CTTG | GTTG | GCG |
| | | GCG | 35 | 22.066 | | 7776 | | 12 | | | | | | GCA |
| | | GCT | 108 | 70.939 | | | | | | | | | | GCC |
| GCT_G_ | A_G_Q | GGA | 12 | 12.080 | 3 | 32.8400 | 38.3763518 | 3.48080666 | 2.352649706 | 54 | 2.00514955 | 2.7:GCTGG | 2.6:GCTG | GGC |
| CAG | | GGC | 28 | 10.677 | | 6535756 | 4356957 | 3748456e- | 033494e-08 | | 42226365 | TCAG | GCCAG | GGA |
| | | GGG | 5 | 6.707 | | 193 | | 07 | | | | | | GGT |
| | | GGT | 9 | 24.536 | | | | | | | | | | GGG |
| GCT_G_ | A_G_G | GGA | 17 | 34.226 | 3 | 52.2933 | 48.0984677 | 2.59351958 | 2.029174112 | 153 | 1.66211261 | 4.8:GCTGG | 1.6:GCTG | GGT |
| GGT | | GGC | 21 | 30.252 | | 3108821 | 0098589 | 3092138e- | 247e-10 | | 32823854 | GGGT | GTGGT | GGC |
| | | GGG | 4 | 19.004 | | 7805 | | 11 | | | | | | GGA |
| | | GGT | 111 | 69.518 | | | | | | | | | | GGG |
| GCT_G_ | A_G_V | GGA | 2 | 16.106 | 3 | 47.9477 | 42.7403753 | 2.18474921 | 2.794030370 | 72 | 1.96977495 | 8.1:GCTGG | 1.8:GCTG | GGT |
| GTC | | GGC | 7 | 14.236 | | 0723954 | 0343509 | 9079959e- | 052986e-09 | | 41476555 | AGTC | GTGTC | GGC |
| | | GGG | 3 | 8.943 | | 9786 | | 10 | | | | | | GGG |
| | | GGT | 60 | 32.714 | | | | | | | | | | GGA |
| GCT_G_ | A_G_V | GGA | 9 | 26.844 | 3 | 68.3509 | 63.9792394 | 9.62319278 | 8.292393294 | 120 | 1.95987061 | 5.0:GCTGG | 1.8:GCTG | GGT |
| GTT | | GGC | 10 | 23.727 | | 1696581 | 88393695 | 847116e-15 | 113863e-14 | | 14431523 | GGTT | GTGTT | GGC |
| | | GGG | 3 | 14.905 | | 435 | | | | | | | | GGA |
| | | GGT | 98 | 54.524 | | | | | | | | | | GGG |
| GCT_L_ | A_L_K | CTA | 27 | 43.332 | 5 | 60.8416 | 65.9615214 | 8.14234397 | 7.079307148 | 306 | 1.54912620 | 1.6:GCTCT | 1.7:GCTTT | TTG |
| AAA | | CTC | 19 | 17.670 | | 8658649 | 4656762 | 775908e-12 | 335749e-13 | | 90132325 | TAAA | GAAA | TTA |
| | | CTG | 23 | 34.614 | | 578 | | | | | | | | CTA |
| | | CTT | 24 | 39.304 | | | | | | | | | | CTT |
| | | TTA | 64 | 84.448 | | | | | | | | | | CTG |
| | | TTG | 149 | 86.632 | | | | | | | | | | CTC |
| GCT_L_ | A_L_N | CTA | 21 | 21.666 | 5 | 35.8980 | 37.8124029 | 9.95473643 | 4.115208714 | 153 | 1.55460084 | 2.2:GCTCT | 1.8:GCTTT | TTG |
| AAC | | CTC | 6 | 8.835 | | 3908248 | 5492329 | 4933137e- | 9168235e-07 | | 00774964 | GAAC | GAAC | TTA |
| | | CTG | 8 | 17.307 | | 1084 | | 07 | | | | | | CTA |
| | | CTT | 10 | 19.652 | | | | | | | | | | CTT |
| | | TTA | 32 | 42.224 | | | | | | | | | | CTG |
| | | TTG | 76 | 43.316 | | | | | | | | | | CTC |
| GCT_L_ | A_L_K | CTA | 14 | 33.844 | 5 | 85.2233 | 88.5289643 | 6.75825024 | 1.368543760 | 239 | 1.70244626 | 3.1:GCTCT | 1.9:GCTTT | TTG |
| AAG | | CTC | 11 | 13.801 | | 6599174 | 9220335 | 0710555e- | 9979315e-17 | | 66381404 | TAAG | GAAG | TTA |
| | | CTG | 17 | 27.035 | | 686 | | 17 | | | | | | CTG |
| | | CTT | 10 | 30.698 | | | | | | | | | | CTA |
| | | TTA | 57 | 65.958 | | | | | | | | | | CTC |
| | | TTG | 130 | 67.664 | | | | | | | | | | CTT |
| GCT_L_ | A_L_I | CTA | 12 | 22.516 | 5 | 35.3273 | 37.3891997 | 1.29445771 | 5.004216436 | 159 | 1.59313446 | 1.9:GCTCT | 1.7:GCTTT | TTG |
| ATT | | CTC | 13 | 9.182 | | 2353839 | 9254596 | 33907067e- | 828705e-07 | | 81854367 | AATT | GATT | TTA |
| | | CTG | 12 | 17.986 | | 528 | | 06 | | | | | | CTC |
| | | CTT | 12 | 20.422 | | | | | | | | | | CTT |
| | | TTA | 33 | 43.880 | | | | | | | | | | CTG |
| | | TTG | 77 | 45.015 | | | | | | | | | | CTA |
| GCT_R_ | A_R_R | AGA | 8 | 14.698 | 5 | 29.3219 | 39.0603973 | 2.00472688 | 2.309279145 | 31 | 2.56794163 | 4.2:GCTCG | 3.7:GCTCG | CGT |
| CGT | | AGG | 3 | 6.724 | | 8543451 | 28023186 | 75147163e- | 7032612e-07 | | 55428595 | ACGT | TCGT | AGA |
| | | CGA | 0 | 2.117 | | 657 | | 05 | | | | | | AGG |
| | | CGC | 2 | 1.835 | | | | | | | | | | CGG |
| | | CGG | 2 | 1.294 | | | | | | | | | | CGC |
| | | CGT | 16 | 4.332 | | | | | | | | | | CGA |
| GCT_S_ | A_S_K | AGC | 13 | 20.845 | 5 | 37.4844 | 44.9927219 | 4.78883414 | 1.455828638 | 185 | 1.52590133 | 1.6:GCTAG | 2.1:GCTTC | TCC |
| AAG | | AGT | 22 | 30.192 | | 2207485 | 58286765 | 4392263e- | 019195e-08 | | 31081934 | CAAG | CAAG | TCT |
| | | TCA | 29 | 38.707 | | 826 | | 07 | | | | | | TCA |
| | | TCC | 61 | 29.048 | | | | | | | | | | AGT |
| | | TCG | 21 | 18.029 | | | | | | | | | | TCG |
| | | TCT | 39 | 48.179 | | | | | | | | | | AGC |
| GCT_S_ | A_S_T | AGC | 41 | 13.070 | 5 | 65.7414 | 81.5450458 | 7.86446267 | 3.985756440 | 116 | 1.93728105 | 3.5:GCTTC | 3.1:GCTA | AGC |
| ACC | | AGT | 10 | 18.931 | | 3846350 | 0092268 | 1304969e- | 8663274e-16 | | 94209233 | AACC | GCACC | TCT |
| | | TCA | 7 | 24.270 | | 843 | | 13 | | | | | | TCC |
| | | TCC | 25 | 18.214 | | | | | | | | | | AGT |
| | | TCG | 6 | 11.304 | | | | | | | | | | TCA |
| | | TCT | 27 | 30.210 | | | | | | | | | | TCG |
| GCT_S_ | A_S_T | AGC | 24 | 18.253 | 5 | 35.4691 | 38.9733370 | 1.21269567 | 2.404363997 | 162 | 1.50623398 | 2.6:GCTTC | 2.0:GCTTC | TCC |
| ACT | | AGT | 17 | 26.439 | | 7929707 | 7837965 | 95822978e- | 039414e-07 | | 79182252 | GACT | CACT | TCT |
| | | TCA | 30 | 33.894 | | 765 | | 06 | | | | | | TCA |
| | | TCC | 51 | 25.437 | | | | | | | | | | AGC |
| | | TCG | 6 | 15.787 | | | | | | | | | | AGT |
| | | TCT | 34 | 42.189 | | | | | | | | | | TCG |
| GCT_S_ | A_S_M | AGC | 5 | 12.845 | 5 | 43.3526 | 53.0356698 | 3.13421760 | 3.307108006 | 114 | 1.72137555 | 2.6:GCTAG | 2.5:GCTTC | TCC |
| ATG | | AGT | 11 | 18.605 | | 3811886 | 3366308 | 41337134e- | 200493e-10 | | 0484876 | CATG | CATG | TCT |
| | | TCA | 16 | 23.852 | | 5954 | | 08 | | | | | | TCA |
| | | TCC | 45 | 17.900 | | | | | | | | | | AGT |
| | | TCG | 7 | 11.110 | | | | | | | | | | TCG |
| | | TCT | 30 | 29.689 | | | | | | | | | | AGC |
| GCT_S_ | A_S_I | AGC | 5 | 14.648 | 5 | 41.0359 | 42.2224020 | 9.22735303 | 5.310464274 | 130 | 1.54911101 | 3.2:GCTTC | 2.1:GCTTC | TCC |
| ATT | | AGT | 21 | 21.216 | | 1025214 | 9879181 | 9377363e- | 027886e-08 | | 53909318 | GATT | CATT | TCT |
| | | TCA | 17 | 27.199 | | 307 | | 08 | | | | | | AGT |
| | | TCC | 43 | 20.412 | | | | | | | | | | TCA |
| | | TCG | 4 | 12.669 | | | | | | | | | | AGC |
| | | TCT | 40 | 33.856 | | | | | | | | | | TCG |
| GCT_T_ | A_T_T | ACA | 8 | 70.811 | 3 | 242.314 | 231.464108 | 3.00593536 | 6.671963484 | 233 | 2.35354733 | 8.9:GCTAC | 2.3:GCTAC | ACT |
| ACC | | ACC | 33 | 49.364 | | 5292188 | 37042345 | 3544477e- | 339983e-50 | | 1654003 | AACC | TACC | ACC |
| | | ACG | 4 | 32.681 | | 6844 | | 52 | | | | | | ACA |
| | | ACT | 188 | 80.143 | | | | | | | | | | ACG |
| GCT_T_ | A_T_L | ACA | 22 | 36.773 | 3 | 29.7333 | 31.6858691 | 1.57025620 | 6.095133790 | 121 | 1.65523667 | 1.7:GCTAC | 1.7:GCTAC | ACT |
| TTG | | ACC | 17 | 25.636 | | 9038516 | 6833709 | 55190318e- | 999681e-07 | | 73884144 | ATTG | TTTG | ACA |
| | | ACG | 11 | 16.972 | | 893 | | 06 | | | | | | ACC |
| | | ACT | 71 | 41.619 | | | | | | | | | | ACG |
| GCT_V_ | A_V_F | GTA | 5 | 18.935 | 3 | 41.7675 | 39.0520304 | 4.49462884 | 1.692167659 | 88 | 1.76838613 | 3.8:GCTGT | 1.8:GCTGT | GTT |
| TTC | | GTC | 16 | 17.730 | | 7512672 | 224692 | 46770786e- | 557919e-08 | | 14048395 | ATTC | TTTC | GTC |
| | | GTG | 6 | 17.262 | | 246 | | 09 | | | | | | GTG |
| | | GTT | 61 | 34.073 | | | | | | | | | | GTA |
| GGA_E_ | G_E_* | GAA | 0 | 11.881 | 1 | 40.8086 | 39.4563523 | 1.67888138 | 3.354746405 | 17 | 3.32096190 | 23.8:GGAG | 3.3:GGAG | GAG |
| TGA | | GAG | 17 | 5.119 | | 5202991 | 8961085 | 99211265e- | 774033e-10 | | 5271227 | AATGA | AGTGA | GAA |
| | | | | | | 2054 | | 10 | | | | | | |
| GGA_L_ | G_L_T | CTA | 7 | 9.063 | 5 | 24.6030 | 38.9165859 | 0.00016620 | 2.468434997 | 64 | 1.60817243 | 2.1:GGACT | 4.1:GGAC | TTA |
| ACT | | CTC | 15 | 3.696 | | 0150459 | 6775969 | 3378377925 | 46359e-07 | | 14160122 | TACT | TCACT | TTG |
| | | CTG | 4 | 7.240 | | 7632 | | | | | | | | CTC |
| | | CTT | 4 | 8.220 | | | | | | | | | | CTA |
| | | TTA | 18 | 17.662 | | | | | | | | | | CTT |
| | | TTG | 16 | 18.119 | | | | | | | | | | CTG |
| GGA_L_ | G_L_Q | CTA | 8 | 6.514 | 5 | 35.9081 | 51.3869914 | 9.90841751 | 7.205338473 | 46 | 2.38175484 | 3.0:GGACT | 3.8:GGAC | CTG |
| CAG | | CTC | 3 | 2.656 | | 6725305 | 122624 | 8450867e- | 839713e-10 | | 4142991 | TCAG | TGCAG | CTA |
| | | CTG | 20 | 5.203 | | 2895 | | 07 | | | | | | TTA |
| | | CTT | 2 | 5.908 | | | | | | | | | | TTG |
| | | TTA | 7 | 12.695 | | | | | | | | | | CTC |
| | | TTG | 6 | 13.023 | | | | | | | | | | CTT |
| GGA_L_ | G_L_A | CTA | 8 | 8.355 | 5 | 32.5435 | 46.8552249 | 4.63625207 | 6.080956046 | 59 | 1.99146223 | 1.9:GGATT | 3.4:GGAC | CTG |
| GCC | | CTC | 3 | 3.407 | | 3033637 | 7218369 | 4148128e- | 6681e-09 | | 31544347 | GGCC | TGGCC | TTG |
| | | CTG | 23 | 6.674 | | 212 | | 06 | | | | | | TTA |
| | | CTT | 7 | 7.578 | | | | | | | | | | CTA |
| | | TTA | 9 | 16.282 | | | | | | | | | | CTT |
| | | TTG | 9 | 16.704 | | | | | | | | | | CTC |
| GGA_L_ | G_L_C | CTA | 5 | 4.956 | 5 | 41.9373 | 41.3236954 | 6.06478659 | 8.070911163 | 35 | 2.39781787 | 7.9:GGACT | 2.7:GGATT | TTA |
| TGC | | CTC | 0 | 2.021 | | 8352681 | 7046006 | 8671358e- | 438775e-08 | | 16926877 | GTGC | ATGC | CTA |
| | | CTG | 0 | 3.959 | | 5225 | | 08 | | | | | | TTG |
| | | CTT | 2 | 4.496 | | | | | | | | | | CTT |
| | | TTA | 26 | 9.659 | | | | | | | | | | CTG |
| | | TTG | 2 | 9.909 | | | | | | | | | | CTC |
| GGA_R_ | G_R_H | AGA | 4 | 8.535 | 5 | 49.8929 | 146.762480 | 1.45747071 | 6.524097268 | 18 | 6.64793089 | 5.0:GGAC | 14.6:GGAC | CGG |
| CAC | | AGG | 2 | 3.904 | | 6955093 | 90984762 | 01792221e- | 29795e-30 | | 0805834 | GTCAC | GGCAC | AGA |
| | | CGA | 1 | 1.229 | | 134 | | 09 | | | | | | AGG |
| | | CGC | 0 | 1.065 | | | | | | | | | | CGA |
| | | CGG | 11 | 0.751 | | | | | | | | | | CGT |
| | | CGT | 0 | 2.515 | | | | | | | | | | CGC |
| GGA_R_ | G_R_Q | AGA | 2 | 14.224 | 5 | 37.9453 | 43.7395107 | 3.86984082 | 2.616068032 | 30 | 2.78353562 | 7.1:GGAA | 3.2:GGAA | AGG |
| CAG | | AGG | 21 | 6.507 | | 4389192 | 65850525 | 6181235e- | 6896903e-08 | | 07294066 | GACAG | GGCAG | CGT |
| | | CGA | 1 | 2.049 | | 7976 | | 07 | | | | | | CGC |
| | | CGC | 2 | 1.776 | | | | | | | | | | AGA |
| | | CGG | 1 | 1.252 | | | | | | | | | | CGG |
| | | CGT | 3 | 4.192 | | | | | | | | | | CGA |
| GGA_T_ | G_T_K | ACA | 28 | 27.352 | 3 | 30.8913 | 36.2780622 | 8.96003425 | 6.540221625 | 90 | 1.58702194 | 2.2:GGAA | 2.5:GGAA | ACG |
| AAA | | ACC | 17 | 19.068 | | 9521465 | 02430476 | 4185827e- | 271933e-08 | | 23358451 | CTAAA | CGAAA | ACA |
| | | ACG | 31 | 12.624 | | 401 | | 07 | | | | | | ACC |
| | | ACT | 14 | 30.957 | | | | | | | | | | ACT |
| GGC_G_ | G_G_P | GGA | 21 | 7.382 | 3 | 26.5092 | 32.7036855 | 7.46049702 | 3.719130361 | 33 | 2.53184190 | 4.1:GGCG | 2.8:GGCG | GGA |
| CCA | | GGC | 4 | 6.525 | | 6520549 | 9540804 | 4720439e- | 0155597e-07 | | 3168642 | GGCCA | GACCA | GGT |
| | | GGG | 1 | 4.099 | | 372 | | 06 | | | | | | GGC |
| | | GGT | 7 | 14.994 | | | | | | | | | | GGG |
| GGC_L_ | G_L_C | CTA | 1 | 5.239 | 5 | 67.9293 | 159.896118 | 2.76229401 | 1.041590829 | 37 | 4.48117924 | 5.2:GGCCT | 9.4:GGCCT | CTC |
| TGT | | CTC | 20 | 2.137 | | 4611343 | 95204274 | 825302e-13 | 3242084e-32 | | 5210491 | ATGT | CTGT | TTA |
| | | CTG | 3 | 4.185 | | 928 | | | | | | | | TTG |
| | | CTT | 4 | 4.752 | | | | | | | | | | CTT |
| | | TTA | 5 | 10.211 | | | | | | | | | | CTG |
| | | TTG | 4 | 10.475 | | | | | | | | | | CTA |
| GGC_Q_ | G_Q_K | CAA | 30 | 49.861 | 1 | 22.8098 | 24.9583842 | 1.78843465 | 5.858121895 | 73 | 1.77499875 | 1.7:GGCC | 1.9:GGCC | CAG |
| AAA | | CAG | 43 | 23.139 | | 8503402 | 74937547 | 82571686e- | 717873e-07 | | 16763859 | AAAAA | AGAAA | CAA |
| | | | | | | 4642 | | 06 | | | | | | |
| GGC_R_ | G_R_R | AGA | 1 | 6.638 | 5 | 40.1222 | 60.9640246 | 1.41096109 | 7.681577436 | 14 | 5.49225367 | 6.6:GGCA | 6.1:GGCC | CGT |
| CGT | | AGG | 0 | 3.037 | | 3431708 | 5321832 | 45252585e- | 644162e-12 | | 0566476 | GACGT | GTCGT | CGC |
| | | CGA | 0 | 0.956 | | 1 | | 07 | | | | | | AGA |
| | | CGC | 1 | 0.829 | | | | | | | | | | CGG |
| | | CGG | 0 | 0.584 | | | | | | | | | | CGA |
| | | CGT | 12 | 1.956 | | | | | | | | | | AGG |
| GGC_R_ | G_R_G | AGA | 15 | 18.966 | 5 | 31.3852 | 39.7739657 | 7.86149742 | 1.658586252 | 40 | 2.15986353 | 5.5:GGCC | 3.4:GGCC | CGT |
| GGT | | AGG | 3 | 8.676 | | 4239589 | 7734867 | 0716145e- | 7251132e-07 | | 59364717 | GAGGT | GTGGT | AGA |
| | | CGA | 0 | 2.731 | | 3123 | | 06 | | | | | | AGG |
| | | CGC | 2 | 2.367 | | | | | | | | | | CGC |
| | | CGG | 1 | 1.670 | | | | | | | | | | CGG |
| | | CGT | 19 | 5.590 | | | | | | | | | | CGA |
| GGC_R_ | G_R_C | AGA | 1 | 5.690 | 5 | 25.9705 | 40.8797391 | 9.04165909 | 9.922554367 | 12 | 3.94277114 | 5.7:GGCA | 7.3:GGCC | CGA |
| TGC | | AGG | 0 | 2.603 | | 6561026 | 3392494 | 708182e-05 | 212886e-08 | | 5527697 | GATGC | GATGC | CGT |
| | | CGA | 6 | 0.819 | | 0614 | | | | | | | | CGG |
| | | CGC | 1 | 0.710 | | | | | | | | | | CGC |
| | | CGG | 1 | 0.501 | | | | | | | | | | AGA |
| | | CGT | 3 | 1.677 | | | | | | | | | | AGG |
| GGC_S_ | G_S_R | AGC | 2 | 4.056 | 5 | 32.9003 | 44.7348406 | 3.93879391 | 1.642581493 | 36 | 2.57810648 | 3.1:GGCTC | 3.5:GGCTC | TCC |
| AGG | | AGT | 2 | 5.875 | | 7600208 | 16807126 | 1557698e- | 7878728e-08 | | 17991814 | TAGG | CAGG | TCA |
| | | TCA | 6 | 7.532 | | 587 | | 06 | | | | | | TCT |
| | | TCC | 20 | 5.653 | | | | | | | | | | TCG |
| | | TCG | 3 | 3.508 | | | | | | | | | | AGT |
| | | TCT | 3 | 9.375 | | | | | | | | | | AGC |
| GGC_V_ | G_V_G | GTA | 16 | 4.734 | 3 | 26.6554 | 34.2875682 | 6.95249611 | 1.722705767 | 22 | 3.20787882 | 4.4:GGCGT | 3.4:GGCG | GTA |
| GGG | | GTC | 1 | 4.432 | | 3320217 | 5696101 | 1072808e- | 5268895e-07 | | 7319401 | CGGG | TAGGG | GTT |
| | | GTG | 2 | 4.315 | | 6606 | | 06 | | | | | | GTG |
| | | GTT | 3 | 8.518 | | | | | | | | | | GTC |
| GGG_G_ | G_G_I | GGA | 2 | 6.264 | 3 | 39.6780 | 53.8389115 | 1.24679898 | 1.214423175 | 28 | 3.63729163 | 3.5:GGGG | 3.8:GGGG | GGC |
| ATA | | GGC | 21 | 5.536 | | 4295157 | 93949184 | 83886008e- | 0197565e-11 | | 92497987 | GGATA | GCATA | GGT |
| | | GGG | 1 | 3.478 | | 6664 | | 08 | | | | | | GGA |
| | | GGT | 4 | 12.722 | | | | | | | | | | GGG |
| GGG_G_ | G_G_L | GGA | 0 | 4.027 | 3 | 48.9630 | 63.3227290 | 1.32831638 | 1.145689314 | 18 | 4.92136446 | 8.2:GGGG | 4.8:GGGG | GGC |
| CTC | | GGC | 17 | 3.559 | | 2354275 | 5302022 | 3349689e- | 498539e-13 | | 0835655 | GTCTC | GCCTC | GGT |
| | | GGG | 0 | 2.236 | | 809 | | 10 | | | | | | GGG |
| | | GGT | 1 | 8.179 | | | | | | | | | | GGA |
| GGG_I_ | G_I_Y | ATA | 7 | 11.147 | 2 | 27.8711 | 32.4959545 | 8.86884379 | 8.781993771 | 40 | 2.34400286 | 2.6:GGGA | 2.5:GGGA | ATC |
| TAC | | ATC | 26 | 10.328 | | 0242613 | 62124346 | 6088281e- | 374553e-08 | | 43315447 | TTTAC | TCTAC | ATT |
| | | ATT | 7 | 18.525 | | 36 | | 07 | | | | | | ATA |
| GGG_L_ | G_L_T | CTA | 2 | 4.956 | 5 | 49.5906 | 79.1566592 | 1.68048837 | 1.259659022 | 35 | 3.62502271 | 4.0:GGGCT | 4.9:GGGC | CTT |
| ACG | | CTC | 2 | 2.021 | | 9246446 | 669063 | 3599869e- | 0257256e-15 | | 52083014 | GACG | TTACG | TTG |
| | | CTG | 1 | 3.959 | | 2215 | | 09 | | | | | | TTA |
| | | CTT | 22 | 4.496 | | | | | | | | | | CTC |
| | | TTA | 3 | 9.659 | | | | | | | | | | CTA |
| | | TTG | 5 | 9.909 | | | | | | | | | | CTG |
| GGG_L_ | G_L_F | CTA | 3 | 7.788 | 5 | 50.6285 | 75.4863456 | 1.03046547 | 7.363978073 | 55 | 2.64079176 | 2.6:GGGCT | 4.2:GGGC | CTG |
| TTC | | CTC | 3 | 3.176 | | 4927650 | 1313683 | 9283889e- | 040801e-15 | | 3595358 | ATTC | TGTTC | CTT |
| | | CTG | 26 | 6.221 | | 825 | | 09 | | | | | | TTG |
| | | CTT | 9 | 7.064 | | | | | | | | | | TTA |
| | | TTA | 7 | 15.179 | | | | | | | | | | CTC |
| | | TTG | 7 | 15.571 | | | | | | | | | | CTA |
| GGG_R_ | G_R_N | AGA | 10 | 18.492 | 5 | 34.2591 | 58.1190740 | 2.11419429 | 2.972480118 | 39 | 2.64202402 | 2.1:GGGA | 5.3:GGGC | CGA |
| AAT | | AGG | 4 | 8.459 | | 8666471 | 7308829 | 2146368e- | 4047836e-11 | | 03204757 | GGAAT | GAAAT | AGA |
| | | CGA | 14 | 2.663 | | 076 | | 06 | | | | | | CGC |
| | | CGC | 5 | 2.308 | | | | | | | | | | CGT |
| | | CGG | 2 | 1.628 | | | | | | | | | | AGG |
| | | CGT | 4 | 5.450 | | | | | | | | | | CGG |
| GGG_S_ | G_S_Q | AGC | 23 | 6.986 | 5 | 51.5291 | 60.1177555 | 6.73766903 | 1.149224218 | 62 | 2.35072007 | 9.7:GGGTC | 3.3:GGGA | AGC |
| CAA | | AGT | 19 | 10.119 | | 8183963 | 2774759 | 9660678e- | 7225931e-11 | | 097688 | CCAA | GCCAA | AGT |
| | | TCA | 8 | 12.972 | | 311 | | 10 | | | | | | TCT |
| | | TCC | 1 | 9.735 | | | | | | | | | | TCA |
| | | TCG | 2 | 6.042 | | | | | | | | | | TCG |
| | | TCT | 9 | 16.147 | | | | | | | | | | TCC |
| GGG_T_ | G_T_T | ACA | 7 | 7.902 | 3 | 30.6859 | 43.0718474 | 9.89846730 | 2.376058984 | 26 | 2.83386616 | 4.5:GGGA | 4.1:GGGA | ACG |
| ACC | | ACC | 2 | 5.508 | | 0554894 | 9725026 | 6244563e- | 7990184e-09 | | 338933 | CTACC | CGACC | ACA |
| | | ACG | 15 | 3.647 | | 0916 | | 07 | | | | | | ACT |
| | | ACT | 2 | 8.943 | | | | | | | | | | ACC |
| GGT_A_ | G_A_N | GCA | 16 | 27.996 | 3 | 31.2060 | 35.9428382 | 7.69235317 | 7.699684036 | 95 | 1.72935443 | 2.2:GGTGC | 2.1:GGTG | GCC |
| AAC | | GCC | 45 | 21.224 | | 2627311 | 287277 | 2048897e- | 240479e-08 | | 39840388 | GAAC | CCAAC | GCT |
| | | GCG | 5 | 10.862 | | 093 | | 07 | | | | | | GCA |
| | | GCT | 29 | 34.918 | | | | | | | | | | GCG |
| GGT_A_ | G_A_I | GCA | 33 | 50.099 | 3 | 32.9087 | 36.0766515 | 3.36654944 | 7.214083639 | 170 | 1.47386485 | 1.9:GGTGC | 1.8:GGTG | GCC |
| ATT | | GCC | 69 | 37.979 | | 9052006 | 19474964 | 03860107e- | 374375e-08 | | 0548011 | GATT | CCATT | GCT |
| | | GCG | 10 | 19.437 | | 9815 | | 07 | | | | | | GCA |
| | | GCT | 58 | 62.485 | | | | | | | | | | GCG |
| GGT_A_ | G_A_A | GCA | 35 | 55.698 | 3 | 43.0833 | 40.4547160 | 2.36277967 | 8.534097196 | 189 | 1.47743399 | 3.6:GGTGC | 1.6:GGTG | GCT |
| GCT | | GCC | 40 | 42.224 | | 1098099 | 7590445 | 7590284e- | 031388e-09 | | 6213505 | GGCT | CTGCT | GCC |
| | | GCG | 6 | 21.609 | | 374 | | 09 | | | | | | GCA |
| | | GCT | 108 | 69.469 | | | | | | | | | | GCG |
| GGT_E_ | G_E_A | GAA | 8 | 20.966 | 1 | 23.7486 | 26.6306778 | 1.09772311 | 2.463016639 | 30 | 2.48349908 | 2.6:GGTG | 2.4:GGTG | GAG |
| GCG | | GAG | 22 | 9.034 | | 5057754 | 71702467 | 90352535e- | 949158e-07 | | 47055796 | AAGCG | AGGCG | GAA |
| | | | | | | 6222 | | 06 | | | | | | |
| GGT_F_ | G_F_K | TTC | 48 | 23.555 | 1 | 43.6019 | 42.7152318 | 4.02446014 | 6.331732983 | 58 | 2.23077731 | 3.4:GGTTT | 2.0:GGTTT | TTC |
| AAG | | TTT | 10 | 34.445 | | 1026570 | 3380463 | 58823923e- | 988645e-11 | | 07355127 | TAAG | CAAG | TTT |
| | | | | | | 0006 | | 11 | | | | | | |
| GGT_F_ | G_F_L | TTC | 42 | 23.149 | 1 | 25.6220 | 25.8479673 | 4.15266391 | 3.693934900 | 57 | 1.92154371 | 2.3:GGTTT | 1.8:GGTTT | TTC |
| TTG | | TTT | 15 | 33.851 | | 5244421 | 33799232 | 3050388e- | 549426e-07 | | 48824102 | TTTG | CTTG | TTT |
| | | | | | | 3615 | | 07 | | | | | | |
| GGT_G_ | G_G_I | GGA | 21 | 39.818 | 3 | 46.9063 | 46.2231835 | 3.63868218 | 5.084418732 | 178 | 1.64199120 | 2.0:GGTG | 1.6:GGTG | GGT |
| ATT | | GGC | 18 | 35.195 | | 8111249 | 6323652 | 4816721e- | 052398e-10 | | 32808117 | GCATT | GTATT | GGA |
| | | GGG | 13 | 22.109 | | 7414 | | 10 | | | | | | GGC |
| | | GGT | 126 | 80.877 | | | | | | | | | | GGG |
| GGT_G_ | G_G_G | GGA | 21 | 50.332 | 3 | 98.0764 | 90.4234499 | 4.02762466 | 1.776656879 | 225 | 1.78149997 | 5.6:GGTG | 1.7:GGTG | GGT |
| GGT | | GGC | 27 | 44.489 | | 7429732 | 9997599 | 9913965e- | 6921948e-19 | | 38840842 | GGGGT | GTGGT | GGC |
| | | GGG | 5 | 27.947 | | 68 | | 21 | | | | | | GGA |
| | | GGT | 172 | 102.232 | | | | | | | | | | GGG |
| GGT_G_ | G_G_V | GGA | 15 | 34.226 | 3 | 45.0807 | 43.8253709 | 8.89402378 | 1.643748146 | 153 | 1.66950979 | 2.3:GGTG | 1.6:GGTG | GGT |
| GTT | | GGC | 19 | 30.252 | | 9107352 | 8039373 | 8013594e- | 5639313e-09 | | 5096565 | GAGTT | GTGTT | GGC |
| | | GGG | 9 | 19.004 | | 131 | | 10 | | | | | | GGA |
| | | GGT | 110 | 69.518 | | | | | | | | | | GGG |
| GGT_G_ | G_G_L | GGA | 25 | 32.213 | 3 | 26.8603 | 32.0335612 | 6.29801181 | 5.148921773 | 144 | 1.38955053 | 1.8:GGTG | 2.2:GGTG | GGT |
| TTA | | GGC | 16 | 28.473 | | 0661820 | 5281672 | 7329132e- | 451713e-07 | | 57542064 | GCTTA | GGTTA | GGG |
| | | GGG | 39 | 17.886 | | 6934 | | 06 | | | | | | GGA |
| | | GGT | 64 | 65.429 | | | | | | | | | | GGC |
| GGT_G_ | G_G_F | GGA | 51 | 29.752 | 3 | 44.7333 | 44.7325859 | 1.05424573 | 1.054621724 | 133 | 1.67247494 | 2.2:GGTG | 1.8:GGTG | GGA |
| TTT | | GGC | 25 | 26.298 | | 1472369 | 5731221 | 53896801e- | 6376175e-09 | | 99893978 | GTTTT | GGTTT | GGG |
| | | GGG | 30 | 16.520 | | 729 | | 09 | | | | | | GGT |
| | | GGT | 27 | 60.430 | | | | | | | | | | GGC |
| GGT_I_ | G_I_K | ATA | 32 | 37.621 | 2 | 28.4913 | 31.6780142 | 6.50406818 | 1.321924342 | 135 | 1.56350018 | 1.6:GGTAT | 1.8:GGTAT | ATC |
| AAG | | ATC | 63 | 34.856 | | 3558947 | 81013652 | 812336e-07 | 8120703e-07 | | 2991456 | TAAG | CAAG | ATT |
| | | ATT | 40 | 62.523 | | 792 | | | | | | | | ATA |
| GGT_I_ | G_I_A | ATA | 13 | 26.474 | 2 | 29.3607 | 28.6242086 | 4.21100388 | 6.086001581 | 95 | 1.69857840 | 2.0:GGTAT | 1.6:GGTAT | ATT |
| GCC | | ATC | 12 | 24.528 | | 8915619 | 7924871 | 96165684e- | 503203e-07 | | 22083468 | CGCC | TGCC | ATA |
| | | ATT | 70 | 43.998 | | 7868 | | 07 | | | | | | ATC |
| GGT_I_ | G_I_G | ATA | 14 | 44.309 | 2 | 38.1915 | 31.7490318 | 5.09120035 | 1.275808052 | 159 | 1.38125401 | 3.2:GGTAT | 1.4:GGTAT | ATT |
| GGT | | ATC | 43 | 41.053 | | 0441565 | 13555803 | 6158559e- | 051736e-07 | | 76926957 | AGGT | TGGT | ATC |
| | | ATT | 102 | 73.638 | | 873 | | 09 | | | | | | ATA |
| GGT_K_ | G_K_K | AAA | 69 | 112.375 | 1 | 39.2374 | 39.7916211 | 3.75275457 | 2.825546551 | 194 | 1.56529459 | 1.6:GGTA | 1.5:GGTA | AAG |
| AAG | | AAG | 125 | 81.625 | | 3592151 | 764002 | 48975857e- | 8163467e-10 | | 66461997 | AAAAG | AGAAG | AAA |
| | | | | | | 233 | | 10 | | | | | | |
| GGT_L_ | G_L_K | CTA | 11 | 24.781 | 5 | 43.8545 | 42.2696165 | 2.47918163 | 5.194867351 | 175 | 1.47358171 | 2.8:GGTCT | 1.7:GGTTT | TTG |
| AAA | | CTC | 12 | 10.106 | | 2579784 | 1417214 | 6842857e- | 9988685e-08 | | 82630029 | TAAA | GAAA | TTA |
| | | CTG | 13 | 19.796 | | 1426 | | 08 | | | | | | CTG |
| | | CTT | 8 | 22.478 | | | | | | | | | | CTC |
| | | TTA | 48 | 48.295 | | | | | | | | | | CTA |
| | | TTG | 83 | 49.545 | | | | | | | | | | CTT |
| GGT_L_ | G_L_F | CTA | 13 | 19.117 | 5 | 41.7823 | 49.6636017 | 6.51909970 | 1.623763467 | 135 | 1.60382275 | 2.0:GGTTT | 2.4:GGTCT | CTT |
| TTC | | CTC | 12 | 7.796 | | 1372480 | 61917725 | 1062626e- | 3119465e-09 | | 34205767 | GTTC | TTTC | TTA |
| | | CTG | 12 | 15.271 | | 993 | | 08 | | | | | | TTG |
| | | CTT | 42 | 17.340 | | | | | | | | | | CTA |
| | | TTA | 37 | 37.256 | | | | | | | | | | CTG |
| | | TTG | 19 | 38.220 | | | | | | | | | | CTC |
| GGT_P_ | G_P_Q | CCA | 6 | 15.889 | 3 | 44.8246 | 54.8252359 | 1.00815800 | 7.481641044 | 39 | 2.39725361 | 12.5:GGTC | 4.0:GGTCC | CCG |
| CAG | | CCC | 0 | 6.259 | | 7066600 | 5842684 | 0785647e- | 559504e-12 | | 83228117 | CCCAG | GCAG | CCT |
| | | CCG | 19 | 4.794 | | 1154 | | 09 | | | | | | CCA |
| | | CCT | 14 | 12.058 | | | | | | | | | | CCC |
| GGT_P_ | G_P_R | CCA | 3 | 11.407 | 3 | 33.3219 | 39.1130765 | 2.75443416 | 1.642519076 | 28 | 2.41390218 | 6.9:GGTCC | 3.6:GGTCC | CCC |
| CGT | | CCC | 16 | 4.494 | | 2605286 | 33205856 | 1138596e- | 4045587e-08 | | 69379547 | GCGT | CCGT | CCT |
| | | CCG | 0 | 3.442 | | 8726 | | 07 | | | | | | CCA |
| | | CCT | 9 | 8.657 | | | | | | | | | | CCG |
| GGT_R_ | G_R_R | AGA | 6 | 14.224 | 5 | 45.8251 | 69.9232605 | 9.85792269 | 1.063174518 | 30 | 3.74191833 | 4.1:GGTCG | 4.8:GGTC | CGT |
| CGT | | AGG | 2 | 6.507 | | 2066963 | 0329065 | 4518181e- | 4158417e-13 | | 36065043 | ACGT | GTCGT | AGA |
| | | CGA | 0 | 2.049 | | 896 | | 09 | | | | | | AGG |
| | | CGC | 1 | 1.776 | | | | | | | | | | CGG |
| | | CGG | 1 | 1.252 | | | | | | | | | | CGC |
| | | CGT | 20 | 4.192 | | | | | | | | | | CGA |
| GGT_R_ | G_R_G | AGA | 18 | 19.914 | 5 | 32.9450 | 38.3977256 | 3.85924127 | 3.138968083 | 42 | 1.95553548 | 5.7:GGTCG | 3.2:GGTC | CGT |
| GGC | | AGG | 2 | 9.110 | | 1223316 | 40242726 | 419016e-06 | 2689716e-07 | | 2389202 | AGGC | GTGGC | AGA |
| | | CGA | 0 | 2.868 | | 229 | | | | | | | | CGC |
| | | CGC | 2 | 2.486 | | | | | | | | | | AGG |
| | | CGG | 1 | 1.753 | | | | | | | | | | CGG |
| | | CGT | 19 | 5.869 | | | | | | | | | | CGA |
| GGT_S_ | G_S_S | AGC | 5 | 8.451 | 5 | 36.6976 | 37.5051375 | 6.88628965 | 4.743214367 | 75 | 1.77513141 | 5.9:GGTTC | 2.2:GGTTC | TCA |
| TCG | | AGT | 6 | 12.240 | | 2432896 | 30102616 | 7594723e- | 7387296e-07 | | 96490464 | CTCG | ATCG | TCT |
| | | TCA | 35 | 15.692 | | 867 | | 07 | | | | | | AGT |
| | | TCC | 2 | 11.776 | | | | | | | | | | TCG |
| | | TCG | 5 | 7.309 | | | | | | | | | | AGC |
| | | TCT | 22 | 19.532 | | | | | | | | | | TCC |
| GGT_S_ | G_S_L | AGC | 8 | 18.253 | 5 | 46.8892 | 42.3524658 | 5.98458691 | 4.998051964 | 162 | 1.51829355 | 2.9:GGTA | 1.6:GGTTC | TCT |
| TTG | | AGT | 9 | 26.439 | | 6181632 | 0955737 | 3129825e- | 764922e-08 | | 91056144 | GTTTG | TTTG | TCA |
| | | TCA | 39 | 33.894 | | 002 | | 09 | | | | | | TCC |
| | | TCC | 31 | 25.437 | | | | | | | | | | AGT |
| | | TCG | 6 | 15.787 | | | | | | | | | | AGC |
| | | TCT | 69 | 42.189 | | | | | | | | | | TCG |
| GGT_T_ | G_T_N | ACA | 26 | 35.254 | 3 | 37.1986 | 44.9273627 | 4.17685514 | 9.587500782 | 116 | 1.77282224 | 1.6:GGTAC | 2.2:GGTA | ACC |
| AAC | | ACC | 54 | 24.576 | | 8292982 | 6597085 | 4736773e- | 7044e-10 | | 45910586 | TAAC | CCAAC | ACA |
| | | ACG | 11 | 16.271 | | 0016 | | 08 | | | | | | ACT |
| | | ACT | 25 | 39.900 | | | | | | | | | | ACG |
| GGT_T_ | G_T_F | ACA | 17 | 29.175 | 3 | 40.3619 | 38.7142873 | 8.92949341 | 1.995204674 | 96 | 1.72920887 | 4.1:GGTAC | 1.8:GGTA | ACT |
| TTC | | ACC | 5 | 20.339 | | 4606442 | 37105446 | 5091553e- | 522823e-08 | | 9310669 | CTTC | CTTTC | ACA |
| | | ACG | 14 | 13.465 | | 874 | | 09 | | | | | | ACG |
| | | ACT | 60 | 33.020 | | | | | | | | | | ACC |
| GGT_V_ | G_V_I | GTA | 11 | 26.036 | 3 | 34.0082 | 36.7801449 | 1.97328347 | 5.121531978 | 121 | 1.52941191 | 2.4:GGTGT | 2.0:GGTGT | GTC |
| ATC | | GTC | 49 | 24.379 | | 2394735 | 4936028 | 55320343e- | 830536e-08 | | 7367953 | AATC | CATC | GTT |
| | | GTG | 15 | 23.735 | | 8076 | | 07 | | | | | | GTG |
| | | GTT | 46 | 46.850 | | | | | | | | | | GTA |
| GGT_V_ | G_V_I | GTA | 14 | 39.377 | 3 | 55.1142 | 57.6889057 | 6.49134158 | 1.831643991 | 183 | 1.51347888 | 2.8:GGTGT | 2.0:GGTGT | GTC |
| ATT | | GTC | 74 | 36.870 | | 7843000 | 6563937 | 7394169e- | 0916626e-12 | | 0524835 | AATT | CATT | GTT |
| | | GTG | 24 | 35.897 | | 775 | | 12 | | | | | | GTG |
| | | GTT | 71 | 70.856 | | | | | | | | | | GTA |
| GGT_V_ | G_V_G | GTA | 20 | 37.656 | 3 | 45.8278 | 41.7677262 | 6.17023760 | 4.494297042 | 175 | 1.52151782 | 2.9:GGTGT | 1.5:GGTGT | GTT |
| GGT | | GTC | 40 | 35.258 | | 3136635 | 3204043 | 1472323e- | 11671e-09 | | 94396792 | GGGT | TGGT | GTC |
| | | GTG | 12 | 34.328 | | 807 | | 10 | | | | | | GTA |
| | | GTT | 103 | 67.758 | | | | | | | | | | GTG |
| GTA_A_ | V_A_Q | GCA | 10 | 9.725 | 3 | 28.2978 | 40.4270995 | 3.14500184 | 8.649937876 | 33 | 2.32297020 | 3.7:GTAGC | 4.0:GTAG | GCG |
| CAG | | GCC | 2 | 7.372 | | 5773915 | 6145659 | 18283243e- | 831974e-09 | | 05716586 | CCAG | CGCAG | GCA |
| | | GCG | 15 | 3.773 | | 4573 | | 06 | | | | | | GCT |
| | | GCT | 6 | 12.130 | | | | | | | | | | GCC |
| GTA_A_ | V_A_V | GCA | 7 | 12.672 | 3 | 29.9982 | 46.0820311 | 1.38121770 | 5.448226036 | 43 | 2.32997505 | 1.8:GTAGC | 3.9:GTAG | GCG |
| GTT | | GCC | 8 | 9.607 | | 6429470 | 22135234 | 8980713e- | 933693e-10 | | 5118617 | AGTT | CGGTT | GCT |
| | | GCG | 19 | 4.916 | | 4605 | | 06 | | | | | | GCC |
| | | GCT | 9 | 15.805 | | | | | | | | | | GCA |
| GTA_G_ | V_G_R | GGA | 8 | 10.290 | 3 | 42.6657 | 62.1855771 | 2.89788882 | 2.005308021 | 46 | 2.53166335 | 3.0:GTAG | 4.0:GTAG | GGG |
| AGA | | GGC | 8 | 9.095 | | 0874917 | 0854549 | 37201227e- | 9722085e-13 | | 79372385 | GTAGA | GGAGA | GGC |
| | | GGG | 23 | 5.714 | | 193 | | 09 | | | | | | GGA |
| | | GGT | 7 | 20.901 | | | | | | | | | | GGT |
| GTA_P_ | V_P_L | CCA | 9 | 13.037 | 3 | 38.4601 | 58.7410429 | 2.25841735 | 1.091864352 | 32 | 3.03445642 | 4.9:GTACC | 4.6:GTACC | CCG |
| CTG | | CCC | 3 | 5.136 | | 8423323 | 7776867 | 80393925e- | 314595e-12 | | 88485922 | TCTG | GCTG | CCA |
| | | CCG | 18 | 3.933 | | 919 | | 08 | | | | | | CCC |
| | | CCT | 2 | 9.894 | | | | | | | | | | CCT |
| GTA_P_ | V_P_Y | CCA | 12 | 17.111 | 3 | 28.7460 | 42.8226963 | 2.53218730 | 2.683829235 | 42 | 2.30870874 | 2.2:GTACC | 3.7:GTACC | CCG |
| TAC | | CCC | 5 | 6.741 | | 3274085 | 8708583 | 85794825e- | 712093e-09 | | 09652733 | TTAC | GTAC | CCA |
| | | CCG | 19 | 5.163 | | 0254 | | 06 | | | | | | CCT |
| | | CCT | 6 | 12.986 | | | | | | | | | | CCC |
| GTA_R_ | V_R_N | AGA | 41 | 42.199 | 5 | 32.6318 | 48.2337672 | 4.45303644 | 3.182275778 | 89 | 1.58299412 | 2.6:GTACG | 3.6:GTAC | AGA |
| AAT | | AGG | 13 | 19.304 | | 1557145 | 7279858 | 8802433e- | 8086243e-09 | | 37039036 | CAAT | GAAAT | CGA |
| | | CGA | 22 | 6.078 | | 576 | | 06 | | | | | | AGG |
| | | CGC | 2 | 5.268 | | | | | | | | | | CGT |
| | | CGG | 4 | 3.715 | | | | | | | | | | CGG |
| | | CGT | 7 | 12.437 | | | | | | | | | | CGC |
| GTA_R_ | V_R_E | AGA | 24 | 29.397 | 5 | 36.3850 | 62.6470145 | 7.95401810 | 3.444843306 | 62 | 2.00863237 | 2.6:GTACG | 4.9:GTAC | AGA |
| GAG | | AGG | 9 | 13.448 | | 3509012 | 5329906 | 2438732e- | 4780977e-12 | | 41310355 | GGAG | GCGAG | CGC |
| | | CGA | 6 | 4.234 | | 8255 | | 07 | | | | | | AGG |
| | | CGC | 18 | 3.670 | | | | | | | | | | CGA |
| | | CGG | 1 | 2.588 | | | | | | | | | | CGT |
| | | CGT | 4 | 8.664 | | | | | | | | | | CGG |
| GTA_S_ | V_S_Y | AGC | 3 | 8.000 | 5 | 33.5145 | 48.7050910 | 2.97401551 | 2.549589123 | 71 | 1.83095877 | 2.7:GTAA | 3.5:GTATC | TCG |
| TAT | | AGT | 7 | 11.587 | | 5151702 | 3495874 | 1021691e- | 9288088e-09 | | 72363738 | GCTAT | GTAT | TCT |
| | | TCA | 14 | 14.855 | | 111 | | 06 | | | | | | TCA |
| | | TCC | 8 | 11.148 | | | | | | | | | | TCC |
| | | TCG | 24 | 6.919 | | | | | | | | | | AGT |
| | | TCT | 15 | 18.490 | | | | | | | | | | AGC |
| GTA_T_ | V_T_R | ACA | 2 | 6.686 | 3 | 47.6390 | 73.4426899 | 2.54142782 | 7.813289785 | 22 | 4.73255904 | 9.3:GTAAC | 5.5:GTAA | ACG |
| CGA | | ACC | 0 | 4.661 | | 5377189 | 9451039 | 82059887e- | 217982e-16 | | 126049 | CCGA | CGCGA | ACT |
| | | ACG | 17 | 3.086 | | 679 | | 10 | | | | | | ACA |
| | | ACT | 3 | 7.567 | | | | | | | | | | ACC |
| GTC_G_ | V_G_K | GGA | 14 | 15.659 | 3 | 32.2908 | 45.1187547 | 4.54445500 | 8.730311804 | 70 | 1.90308159 | 2.0:GTCGG | 3.1:GTCG | GGG |
| AAA | | GGC | 7 | 13.841 | | 6410393 | 4909798 | 3354668e- | 938842e-10 | | 69154518 | CAAA | GGAAA | GGT |
| | | GGG | 27 | 8.695 | | 377 | | 07 | | | | | | GGA |
| | | GGT | 22 | 31.806 | | | | | | | | | | GGC |
| GTC_I_ | V_I_N | ATA | 16 | 23.687 | 2 | 25.9548 | 29.8991681 | 2.31191861 | 3.217200547 | 85 | 1.76378876 | 1.6:GTCAT | 2.0:GTCAT | ATC |
| AAC | | ATC | 44 | 21.946 | | 6561482 | 29674948 | 84355857e- | 1591265e-07 | | 25572934 | TAAC | CAAC | ATT |
| | | ATT | 25 | 39.366 | | 4506 | | 06 | | | | | | ATA |
| GTC_I_ | V_I_K | ATA | 18 | 25.360 | 2 | 41.0242 | 47.3260941 | 1.23511521 | 5.287729449 | 91 | 1.97885534 | 2.0:GTCAT | 2.2:GTCAT | ATC |
| AAG | | ATC | 52 | 23.495 | | 0316673 | 69581 | 04937968e- | 545677e-11 | | 26601203 | TAAG | CAAG | ATT |
| | | ATT | 21 | 42.145 | | 657 | | 09 | | | | | | ATA |
| GTC_I_ | V_I_R | ATA | 14 | 24.802 | 2 | 25.4127 | 28.8608210 | 3.03170792 | 5.406949869 | 89 | 1.71062785 | 1.8:GTCAT | 2.0:GTCAT | ATC |
| AGA | | ATC | 45 | 22.979 | | 6885254 | 2387919 | 30560085e- | 874547e-07 | | 17540223 | AAGA | CAGA | ATT |
| | | ATT | 30 | 41.219 | | 0456 | | 06 | | | | | | ATA |
| GTC_L_ | V_L_F | CTA | 6 | 6.231 | 5 | 29.1286 | 39.6808555 | 2.18780313 | 1.731827775 | 44 | 2.22260132 | 2.5:GTCCT | 3.4:GTCCT | CTT |
| TTC | | CTC | 4 | 2.541 | | 6918381 | 4474282 | 35317333e- | 922706e-07 | | 34675475 | GTTC | TTTC | TTA |
| | | CTG | 2 | 4.977 | | 0743 | | 05 | | | | | | TTG |
| | | CTT | 19 | 5.651 | | | | | | | | | | CTA |
| | | TTA | 7 | 12.143 | | | | | | | | | | CTC |
| | | TTG | 6 | 12.457 | | | | | | | | | | CTG |
| GTC_R_ | V_R_T | AGA | 14 | 48.837 | 5 | 179.848 | 225.266256 | 5.76554297 | 1.105828392 | 103 | 3.79000345 | 14.1:GTCC | 3.8:GTCA | AGG |
| ACT | | AGG | 85 | 22.341 | | 2997860 | 52607208 | 9995669e- | 0983666e-46 | | 6617659 | GAACT | GGACT | AGA |
| | | CGA | 0 | 7.034 | | 3145 | | 37 | | | | | | CGT |
| | | CGC | 0 | 6.096 | | | | | | | | | | CGG |
| | | CGG | 1 | 4.299 | | | | | | | | | | CGC |
| | | CGT | 3 | 14.393 | | | | | | | | | | CGA |
| GTC_S_ | V_S_G | AGC | 5 | 3.268 | 5 | 34.8973 | 42.6049281 | 1.57730558 | 4.442968395 | 29 | 2.84101122 | 5.7:GTCTC | 3.6:GTCA | AGT |
| GGG | | AGT | 17 | 4.733 | | 6386022 | 26391556 | 68416065e- | 778805e-08 | | 345028 | GGGG | GTGGG | AGC |
| | | TCA | 3 | 6.068 | | 044 | | 06 | | | | | | TCA |
| | | TCC | 2 | 4.554 | | | | | | | | | | TCT |
| | | TCG | 0 | 2.826 | | | | | | | | | | TCC |
| | | TCT | 2 | 7.552 | | | | | | | | | | TCG |
| GTC_T_ | V_T_K | ACA | 24 | 28.264 | 3 | 40.3356 | 47.1088750 | 9.04466568 | 3.295118661 | 93 | 1.86241586 | 2.3:GTCAC | 2.3:GTCAC | ACC |
| AAG | | ACC | 46 | 19.703 | | 9454232 | 61056764 | 6691584e- | 753927e-10 | | 77186735 | TAAG | CAAG | ACA |
| | | ACG | 9 | 13.044 | | 7886 | | 09 | | | | | | ACT |
| | | ACT | 14 | 31.989 | | | | | | | | | | ACG |
| GTC_T_ | V_T_G | ACA | 19 | 32.518 | 3 | 38.7266 | 44.6034655 | 1.98324703 | 1.123397714 | 107 | 1.74767918 | 2.5:GTCAC | 2.2:GTCAC | ACC |
| GGT | | ACC | 50 | 22.670 | | 1281058 | 32230494 | 0371239e- | 2634421e-09 | | 50594606 | GGGT | CGGT | ACT |
| | | ACG | 6 | 15.008 | | 3376 | | 08 | | | | | | ACA |
| | | ACT | 32 | 36.804 | | | | | | | | | | ACG |
| GTG_E_ | V_E_A | GAA | 29 | 48.223 | 1 | 22.9069 | 25.4476639 | 1.70032008 | 4.545477156 | 69 | 1.81023988 | 1.7:GTGG | 1.9:GTGG | GAG |
| GCA | | GAG | 40 | 20.777 | | 9447924 | 56885332 | 14812525e- | 5454876e-07 | | 32589104 | AAGCA | AGGCA | GAA |
| | | | | | | 4435 | | 06 | | | | | | |
| GTG_G_ | V_G_I | GGA | 5 | 8.948 | 3 | 33.3510 | 51.9658542 | 2.71579838 | 3.045689745 | 40 | 2.65314762 | 1.8:GTGG | 4.0:GTGG | GGG |
| ATC | | GGC | 5 | 7.909 | | 0223523 | 1875498 | 89329995e- | 275462e-11 | | 0053344 | GTATC | GGATC | GGT |
| | | GGG | 20 | 4.968 | | 9775 | | 07 | | | | | | GGC |
| | | GGT | 10 | 18.175 | | | | | | | | | | GGA |
| GTG_L_ | V_L_Q | CTA | 7 | 7.222 | 5 | 43.5874 | 66.7665558 | 2.80873428 | 4.817789113 | 51 | 2.49520616 | 3.3:GTGCT | 4.2:GTGCT | CTG |
| CAG | | CTC | 1 | 2.945 | | 0400570 | 6878958 | 6234008e- | 652135e-13 | | 45279535 | TCAG | GCAG | TTA |
| | | CTG | 24 | 5.769 | | 255 | | 08 | | | | | | TTG |
| | | CTT | 2 | 6.551 | | | | | | | | | | CTA |
| | | TTA | 9 | 14.075 | | | | | | | | | | CTT |
| | | TTG | 8 | 14.439 | | | | | | | | | | CTC |
| GTG_P_ | V_P_H | CCA | 7 | 14.667 | 3 | 39.2459 | 62.8292040 | 1.53943149 | 1.460786987 | 36 | 3.09864287 | 2.9:GTGCC | 4.5:GTGCC | CCG |
| CAT | | CCC | 2 | 5.778 | | 4240462 | 6190534 | 49425013e- | 8983721e-13 | | 46408206 | CCAT | GCAT | CCT |
| | | CCG | 20 | 4.425 | | 841 | | 08 | | | | | | CCA |
| | | CCT | 7 | 11.131 | | | | | | | | | | CCC |
| GTG_R_ | V_R_S | AGA | 9 | 18.966 | 5 | 64.1545 | 168.669441 | 1.67808801 | 1.402719508 | 40 | 3.89359455 | 2.7:GTGCG | 10.8:GTGC | CGG |
| TCA | | AGG | 4 | 8.676 | | 8628445 | 709613 | 66908373e- | 1221756e-34 | | 41677146 | ATCA | GGTCA | AGA |
| | | CGA | 1 | 2.731 | | 384 | | 12 | | | | | | CGT |
| | | CGC | 2 | 2.367 | | | | | | | | | | AGG |
| | | CGG | 18 | 1.670 | | | | | | | | | | CGC |
| | | CGT | 6 | 5.590 | | | | | | | | | | CGA |
| GTG_S_ | V_S_F | AGC | 4 | 7.887 | 5 | 44.3876 | 64.3336380 | 1.93224199 | 1.540615074 | 70 | 2.02787582 | 2.6:GTGTC | 3.8:GTGTC | TCG |
| TTT | | AGT | 11 | 11.424 | | 6817335 | 1943922 | 96687728e- | 6335866e-12 | | 44358137 | TTTT | GTTT | TCC |
| | | TCA | 10 | 14.646 | | 372 | | 08 | | | | | | AGT |
| | | TCC | 12 | 10.991 | | | | | | | | | | TCA |
| | | TCG | 26 | 6.822 | | | | | | | | | | TCT |
| | | TCT | 7 | 18.230 | | | | | | | | | | AGC |
| GTG_T_ | V_T_R | ACA | 31 | 20.058 | 3 | 39.1961 | 33.6605048 | 1.57724949 | 2.336606403 | 66 | 1.82341745 | 5.7:GTGAC | 1.9:GTGA | ACA |
| AGA | | ACC | 26 | 13.983 | | 9561250 | 3633267 | 47201336e- | 335857e-07 | | 85862784 | TAGA | CCAGA | ACC |
| | | ACG | 5 | 9.257 | | 052 | | 08 | | | | | | ACG |
| | | ACT | 4 | 22.702 | | | | | | | | | | ACT |
| GTG_V_ | V_V_W | GTA | 21 | 6.670 | 3 | 31.4704 | 39.7172441 | 6.76653714 | 1.223174368 | 31 | 2.84678768 | 3.1:GTGGT | 3.1:GTGGT | GTA |
| TGG | | GTC | 2 | 6.246 | | 4552276 | 7059597 | 1519455e- | 595867e-08 | | 45524756 | CTGG | ATGG | GTT |
| | | GTG | 4 | 6.081 | | 393 | | 07 | | | | | | GTG |
| | | GTT | 4 | 12.003 | | | | | | | | | | GTC |
| GTT_E_ | V_E_C | GAA | 7 | 24.461 | 1 | 37.2018 | 41.3927448 | 1.06509989 | 1.245186695 | 35 | 2.80645400 | 3.5:GTTGA | 2.7:GTTGA | GAG |
| TGC | | GAG | 28 | 10.539 | | 9935782 | 593211 | 25469339e- | 4359976e-10 | | 6709004 | ATGC | GTGC | GAA |
| | | | | | | 011 | | 09 | | | | | | |
| GTT_F_ | V_F_K | TTC | 82 | 47.111 | 1 | 42.8742 | 43.5079988 | 5.83753720 | 4.222299737 | 116 | 1.81983585 | 2.0:GTTTT | 1.7:GTTTT | TTC |
| AAA | | TTT | 34 | 68.889 | | 0324737 | 59288854 | 7119363e- | 600827e-11 | | 63168981 | TAAA | CAAA | TTT |
| | | | | | | 028 | | 11 | | | | | | |
| GTT_F_ | V_F_N | TTC | 77 | 45.080 | 1 | 37.4246 | 38.0577826 | 9.50152718 | 6.868021383 | 111 | 1.77566938 | 1.9:GTTTT | 1.7:GTTTT | TTC |
| AAC | | TTT | 34 | 65.920 | | 1666274 | 0500118 | 2055685e- | 989614e-10 | | 44384408 | TAAC | CAAC | TTT |
| | | | | | | 081 | | 10 | | | | | | |
| GTT_G_ | V_G_G | GGA | 22 | 30.199 | 3 | 36.1618 | 33.0148816 | 6.92088907 | 3.197477833 | 135 | 1.55188550 | 3.3:GTTGG | 1.5:GTTGG | GGT |
| GGT | | GGC | 8 | 26.693 | | 7189174 | 0603794 | 7702467e- | 213983e-07 | | 77789075 | CGGT | TGGT | GGA |
| | | GGG | 12 | 16.768 | | 322 | | 08 | | | | | | GGG |
| | | GGT | 93 | 61.339 | | | | | | | | | | GGC |
| GTT_G_ | V_G_C | GGA | 24 | 9.172 | 3 | 29.1809 | 33.3289164 | 2.05171669 | 2.745095664 | 41 | 2.16043419 | 5.1:GTTGG | 2.6:GTTGG | GGA |
| TGC | | GGC | 8 | 8.107 | | 1835383 | 5461682 | 71065374e- | 76394e-07 | | 38852327 | GTGC | ATGC | GGT |
| | | GGG | 1 | 5.093 | | 7228 | | 06 | | | | | | GGC |
| | | GGT | 8 | 18.629 | | | | | | | | | | GGG |
| GTT_G_ | V_G_F | GGA | 47 | 23.041 | 3 | 30.4452 | 34.6688403 | 1.11227676 | 1.431171729 | 103 | 1.69433481 | 1.9:GTTGG | 2.0:GTTGG | GGA |
| TTT | | GGC | 11 | 20.366 | | 7507883 | 97210836 | 56261378e- | 4535943e-07 | | 74447868 | CTTT | ATTT | GGT |
| | | GGG | 14 | 12.794 | | 5728 | | 06 | | | | | | GGG |
| | | GGT | 31 | 46.800 | | | | | | | | | | GGC |
| GTT_I_ | V_I_N | ATA | 19 | 27.032 | 2 | 36.7589 | 42.4269497 | 1.04208948 | 6.124983329 | 97 | 1.87706018 | 1.8:GTTAT | 2.1:GTTAT | ATC |
| AAC | | ATC | 53 | 25.045 | | 0585422 | 9043945 | 40373302e- | 548578e-10 | | 9921175 | TAAC | CAAC | ATT |
| | | ATT | 25 | 44.924 | | 658 | | 08 | | | | | | ATA |
| GTT_I_ | V_I_A | ATA | 6 | 23.130 | 2 | 34.1356 | 30.9544255 | 3.86837510 | 1.898155944 | 83 | 1.72348345 | 3.9:GTTAT | 1.6:GTTAT | ATT |
| GCT | | ATC | 14 | 21.430 | | 9239167 | 83502207 | 01643324e- | 2249997e-07 | | 82166836 | AGCT | TGCT | ATC |
| | | ATT | 63 | 38.440 | | 931 | | 08 | | | | | | ATA |
| GTT_L_ | V_L_N | CTA | 12 | 33.278 | 5 | 41.1849 | 39.6224527 | 8.60910759 | 1.779402914 | 235 | 1.40507128 | 2.8:GTTCT | 1.8:GTTCT | TTG |
| AAT | | CTC | 24 | 13.570 | | 7574586 | 7242126 | 5420283e- | 709821e-07 | | 92423767 | AAAT | CAAT | TTA |
| | | CTG | 26 | 26.583 | | 1856 | | 08 | | | | | | CTG |
| | | CTT | 22 | 30.184 | | | | | | | | | | CTC |
| | | TTA | 54 | 64.854 | | | | | | | | | | CTT |
| | | TTG | 97 | 66.531 | | | | | | | | | | CTA |
| GTT_L_ | V_L_R | CTA | 9 | 20.108 | 5 | 40.1106 | 38.3825168 | 1.41855213 | 3.161145225 | 142 | 1.52076624 | 2.7:GTTCT | 1.7:GTTTT | TTG |
| AGA | | CTC | 11 | 8.200 | | 8101443 | 1841166 | 3881703e- | 863303e-07 | | 43454717 | GAGA | GAGA | TTA |
| | | CTG | 6 | 16.063 | | 105 | | 07 | | | | | | CTC |
| | | CTT | 8 | 18.239 | | | | | | | | | | CTA |
| | | TTA | 40 | 39.188 | | | | | | | | | | CTT |
| | | TTG | 68 | 40.202 | | | | | | | | | | CTG |
| GTT_L_ | V_L_I | CTA | 8 | 16.427 | 5 | 35.0491 | 51.9019819 | 1.47104603 | 5.650348924 | 116 | 1.54598932 | 2.1:GTTCT | 3.6:GTTCT | TTG |
| ATC | | CTC | 24 | 6.699 | | 5093756 | 7952069 | 41632884e- | 468496e-10 | | 64901032 | AATC | CATC | TTA |
| | | CTG | 9 | 13.122 | | 3915 | | 06 | | | | | | CTC |
| | | CTT | 18 | 14.899 | | | | | | | | | | CTT |
| | | TTA | 28 | 32.013 | | | | | | | | | | CTG |
| | | TTG | 29 | 32.841 | | | | | | | | | | CTA |
| GTT_R_ | V_R_H | AGA | 6 | 11.379 | 5 | 27.2828 | 40.6367589 | 5.02442162 | 1.110933337 | 24 | 2.78959598 | 5.2:GTTAG | 5.6:GTTCG | CGC |
| CAT | | AGG | 1 | 5.206 | | 5330283 | 463662 | 1843276e- | 2656627e-07 | | 5771993 | GCAT | CCAT | CGT |
| | | CGA | 3 | 1.639 | | 3573 | | 05 | | | | | | AGA |
| | | CGC | 8 | 1.420 | | | | | | | | | | CGA |
| | | CGG | 0 | 1.002 | | | | | | | | | | AGG |
| | | CGT | 6 | 3.354 | | | | | | | | | | CGG |
| GTT_R_ | V_R_R | AGA | 3 | 11.854 | 5 | 41.7005 | 62.5115695 | 6.77212276 | 3.674644484 | 25 | 3.92023212 | 4.0:GTTAG | 4.9:GTTCG | CGT |
| CGT | | AGG | 2 | 5.423 | | 5546283 | 51266476 | 3320859e- | 258823e-12 | | 20664906 | ACGT | TCGT | AGA |
| | | CGA | 1 | 1.707 | | 818 | | 08 | | | | | | CGC |
| | | CGC | 2 | 1.480 | | | | | | | | | | AGG |
| | | CGG | 0 | 1.044 | | | | | | | | | | CGA |
| | | CGT | 17 | 3.493 | | | | | | | | | | CGG |
| GTT_S_ | V_S_I | AGC | 5 | 10.253 | 5 | 30.1471 | 37.4042407 | 1.37969759 | 4.969565102 | 91 | 1.68791591 | 2.2:GTTTC | 2.4:GTTTC | TCC |
| ATC | | AGT | 13 | 14.851 | | 3080379 | 2095747 | 4109997e- | 256235e-07 | | 78857213 | GATC | CATC | TCT |
| | | TCA | 15 | 19.039 | | 5164 | | 05 | | | | | | TCA |
| | | TCC | 35 | 14.289 | | | | | | | | | | AGT |
| | | TCG | 4 | 8.868 | | | | | | | | | | AGC |
| | | TCT | 19 | 23.699 | | | | | | | | | | TCG |
| GTT_S_ | V_S_Q | AGC | 8 | 19.831 | 5 | 47.7823 | 43.2931049 | 3.93439009 | 3.222565407 | 176 | 1.40721580 | 3.6:GTTAG | 1.7:GTTTC | TCT |
| CAA | | AGT | 8 | 28.724 | | 9160868 | 1250396 | 6586785e- | 435378e-08 | | 64731262 | TCAA | TCAA | TCA |
| | | TCA | 38 | 36.824 | | 709 | | 09 | | | | | | TCC |
| | | TCC | 27 | 27.635 | | | | | | | | | | TCG |
| | | TCG | 18 | 17.152 | | | | | | | | | | AGT |
| | | TCT | 77 | 45.835 | | | | | | | | | | AGC |
| GTT_V_ | V_V_V | GTA | 10 | 30.985 | 3 | 41.4987 | 36.5356807 | 5.12545505 | 5.769121821 | 144 | 1.54906244 | 3.1:GTTGT | 1.5:GTTGT | GTT |
| GTT | | GTC | 38 | 29.013 | | 3520874 | 0599182 | 5341179e- | 0815245e-08 | | 78088684 | AGTT | TGTT | GTC |
| | | GTG | 14 | 28.247 | | 134 | | 09 | | | | | | GTG |
| | | GTT | 82 | 55.755 | | | | | | | | | | GTA |
| TAC_L_ | Y_L_N | CTA | 15 | 17.135 | 5 | 33.6315 | 40.9261924 | 2.81892863 | 9.710483875 | 121 | 1.52964890 | 3.1:TACCT | 3.0:TACCT | TTG |
| AAC | | CTC | 21 | 6.987 | | 1240578 | 8605032 | 5794649e- | 301592e-08 | | 0518691 | TAAC | CAAC | TTA |
| | | CTG | 11 | 13.687 | | 756 | | 06 | | | | | | CTC |
| | | CTT | 5 | 15.542 | | | | | | | | | | CTA |
| | | TTA | 25 | 33.393 | | | | | | | | | | CTG |
| | | TTG | 44 | 34.257 | | | | | | | | | | CTT |
| TAC_L_ | Y_L_I | CTA | 10 | 16.285 | 5 | 35.0567 | 55.6486309 | 1.46588562 | 9.599518678 | 115 | 1.52797719 | 1.6:TACCT | 3.8:TACCT | TTG |
| ATT | | CTC | 25 | 6.641 | | 9747331 | 67602884 | 15437001e- | 454947e-11 | | 11311428 | AATT | CATT | TTA |
| | | CTG | 13 | 13.009 | | 85 | | 06 | | | | | | CTC |
| | | CTT | 11 | 14.771 | | | | | | | | | | CTG |
| | | TTA | 25 | 31.737 | | | | | | | | | | CTT |
| | | TTG | 31 | 32.558 | | | | | | | | | | CTA |
| TAC_L_ | Y_L_A | CTA | 4 | 8.780 | 5 | 32.0972 | 47.1103197 | 5.68345665 | 5.394716003 | 62 | 2.00090523 | 2.2:TACCT | 3.4:TACCT | CTG |
| GCC | | CTC | 3 | 3.580 | | 8017628 | 17476806 | 8500531e- | 67274e-09 | | 08831943 | AGCC | GGCC | TTG |
| | | CTG | 24 | 7.013 | | 348 | | 06 | | | | | | TTA |
| | | CTT | 5 | 7.963 | | | | | | | | | | CTT |
| | | TTA | 13 | 17.110 | | | | | | | | | | CTA |
| | | TTG | 13 | 17.553 | | | | | | | | | | CTC |
| TAC_R_ | Y_R_Q | AGA | 12 | 20.388 | 5 | 70.1913 | 193.016690 | 9.35013645 | 8.849153921 | 43 | 4.16389469 | 2.9:TACCG | 11.1:TACC | CGG |
| CAG | | AGG | 5 | 9.327 | | 2352012 | 5092189 | 6009449e- | 666536e-40 | | 5873348 | ACAG | GGCAG | AGA |
| | | CGA | 1 | 2.936 | | 554 | | 14 | | | | | | AGG |
| | | CGC | 2 | 2.545 | | | | | | | | | | CGT |
| | | CGG | 20 | 1.795 | | | | | | | | | | CGC |
| | | CGT | 3 | 6.009 | | | | | | | | | | CGA |
| TAC_V_ | Y_V_K | GTA | 14 | 20.011 | 3 | 40.9187 | 50.4935922 | 6.80393068 | 6.271520952 | 93 | 1.95125712 | 1.9:TACGT | 2.5:TACGT | GTC |
| AAG | | GTC | 46 | 18.737 | | 3593037 | 26428305 | 860455e-09 | 670566e-11 | | 53893651 | TAAG | CAAG | GTT |
| | | GTG | 14 | 18.243 | | 35 | | | | | | | | GTG |
| | | GTT | 19 | 36.009 | | | | | | | | | | GTA |
| TAC_V_ | Y_V_G | GTA | 1 | 7.531 | 3 | 47.3802 | 60.8935405 | 2.88500115 | 3.787263014 | 35 | 3.21998737 | 7.5:TACGT | 3.6:TACGT | GTG |
| GGG | | GTC | 5 | 7.052 | | 2335708 | 47705456 | 7653471e- | 2059145e-13 | | 3685222 | AGGG | GGGG | GTC |
| | | GTG | 25 | 6.866 | | 0254 | | 10 | | | | | | GTT |
| | | GTT | 4 | 13.552 | | | | | | | | | | GTA |
| TAT_G_ | Y_G_F | GGA | 28 | 19.462 | 3 | 47.1818 | 56.5589479 | 3.17940819 | 3.191948204 | 87 | 2.04216001 | 2.3:TATGG | 2.9:TATGG | GGG |
| TTT | | GGC | 11 | 17.202 | | 5542076 | 88501004 | 54786863e- | 8620995e-12 | | 97448178 | TTTT | GTTT | GGA |
| | | GGG | 31 | 10.806 | | 27 | | 10 | | | | | | GGT |
| | | GGT | 17 | 39.530 | | | | | | | | | | GGC |
| TAT_L_ | Y_L_K | CTA | 21 | 35.968 | 5 | 70.7429 | 77.6953845 | 7.17777493 | 2.545258500 | 254 | 1.68221815 | 1.8:TATCT | 1.9:TATTT | TTG |
| AAA | | CTC | 15 | 14.668 | | 5451005 | 8864996 | 2180903e- | 4839572e-15 | | 06113123 | TAAA | GAAA | TTA |
| | | CTG | 22 | 28.732 | | 89 | | 14 | | | | | | CTG |
| | | CTT | 18 | 32.624 | | | | | | | | | | CTA |
| | | TTA | 44 | 70.097 | | | | | | | | | | CTT |
| | | TTG | 134 | 71.911 | | | | | | | | | | CTC |
| TAT_L_ | Y_L_L | CTA | 32 | 25.914 | 5 | 39.9613 | 47.2024715 | 1.52044148 | 5.166284444 | 183 | 1.51564269 | 2.1:TATCT | 2.2:TATCT | CTT |
| TTA | | CTC | 5 | 10.568 | | 0412709 | 5831482 | 40524406e- | 295324e-09 | | 3748388 | CTTA | TTTA | TTA |
| | | CTG | 18 | 20.701 | | 6966 | | 07 | | | | | | TTG |
| | | CTT | 52 | 23.505 | | | | | | | | | | CTA |
| | | TTA | 43 | 50.503 | | | | | | | | | | CTG |
| | | TTG | 33 | 51.810 | | | | | | | | | | CTC |
| TAT_L_ | Y_L_L | CTA | 17 | 22.374 | 5 | 51.7233 | 67.5096070 | 6.14744520 | 3.376717529 | 158 | 1.65744236 | 2.0:TATCT | 2.7:TATCT | CTT |
| TTG | | CTC | 9 | 9.124 | | 9407645 | 0542092 | 35903e-10 | 082173e-13 | | 55628092 | GTTG | TTTG | TTA |
| | | CTG | 9 | 17.873 | | 0376 | | | | | | | | TTG |
| | | CTT | 54 | 20.294 | | | | | | | | | | CTA |
| | | TTA | 40 | 43.604 | | | | | | | | | | CTG |
| | | TTG | 29 | 44.732 | | | | | | | | | | CTC |
| TAT_R_ | Y_R_R | AGA | 9 | 16.121 | 5 | 62.4966 | 178.604979 | 3.70087062 | 1.062562989 | 34 | 4.77826953 | 2.4:TATCG | 12.0:TATC | CGG |
| AGG | | AGG | 4 | 7.375 | | 5270561 | 88719342 | 0526198e- | 0951137e-36 | | 6436087 | TAGG | GGAGG | AGA |
| | | CGA | 1 | 2.322 | | 325 | | 12 | | | | | | AGG |
| | | CGC | 1 | 2.012 | | | | | | | | | | CGT |
| | | CGG | 17 | 1.419 | | | | | | | | | | CGC |
| | | CGT | 2 | 4.751 | | | | | | | | | | CGA |
| TAT_R_ | Y_R_G | AGA | 14 | 20.862 | 5 | 36.6155 | 49.5782841 | 7.15197104 | 1.690337264 | 44 | 2.40617410 | 3.7:TATCG | 3.6:TATCG | CGT |
| GGT | | AGG | 4 | 9.544 | | 5219825 | 162998 | 4596863e- | 399712e-09 | | 06968376 | GGGT | TGGT | AGA |
| | | CGA | 1 | 3.005 | | 4825 | | 07 | | | | | | AGG |
| | | CGC | 3 | 2.604 | | | | | | | | | | CGC |
| | | CGG | 0 | 1.837 | | | | | | | | | | CGA |
| | | CGT | 22 | 6.149 | | | | | | | | | | CGG |
| TCA_G_ | S_G_F | GGA | 30 | 17.896 | 3 | 38.7790 | 42.0967495 | 1.93312260 | 3.826865080 | 80 | 1.96243844 | 2.4:TCAGG | 2.4:TCAG | GGA |
| TTT | | GGC | 11 | 15.818 | | 9978368 | 48570216 | 11735558e- | 372529e-09 | | 19829708 | TTTT | GGTTT | GGG |
| | | GGG | 24 | 9.937 | | 406 | | 08 | | | | | | GGT |
| | | GGT | 15 | 36.349 | | | | | | | | | | GGC |
| TCA_Q_ | S_Q_P | CAA | 7 | 20.491 | 1 | 25.5918 | 28.0216462 | 4.21815554 | 1.199659913 | 30 | 2.52884244 | 2.9:TCACA | 2.4:TCACA | CAG |
| CCC | | CAG | 23 | 9.509 | | 5780192 | 85845698 | 24973535e- | 953086e-07 | | 2421435 | ACCC | GCCC | CAA |
| | | | | | | 854 | | 07 | | | | | | |
| TCA_S_ | S_S_E | AGC | 15 | 12.732 | 5 | 35.3161 | 42.2468159 | 1.30111484 | 5.250374298 | 113 | 1.64240123 | 2.5:TCATC | 2.6:TCATC | TCG |
| GAG | | AGT | 24 | 18.442 | | 6893670 | 902739 | 05826479e- | 146105e-08 | | 88111595 | CGAG | GGAG | AGT |
| | | TCA | 17 | 23.642 | | 865 | | 06 | | | | | | TCT |
| | | TCC | 7 | 17.743 | | | | | | | | | | TCA |
| | | TCG | 29 | 11.012 | | | | | | | | | | AGC |
| | | TCT | 21 | 29.428 | | | | | | | | | | TCC |
| TCC_E_ | S_E_R | GAA | 1 | 11.881 | 1 | 31.5182 | 33.0939472 | 1.97572136 | 8.781165129 | 17 | 3.38102034 | 11.9:TCCG | 3.1:TCCGA | GAG |
| CGC | | GAG | 16 | 5.119 | | 7401549 | 7018711 | 0484395e- | 057818e-09 | | 13039535 | AACGC | GCGC | GAA |
| | | | | | | 2725 | | 08 | | | | | | |
| TCC_L_ | S_L_F | CTA | 4 | 9.488 | 5 | 32.3366 | 41.8489276 | 5.09557478 | 6.319926037 | 67 | 1.97079272 | 2.4:TCCCT | 2.9:TCCCT | CTT |
| TTC | | CTC | 7 | 3.869 | | 0245862 | 0121851 | 9706772e- | 540431e-08 | | 24559953 | ATTC | TTTC | TTA |
| | | CTG | 4 | 7.579 | | 461 | | 06 | | | | | | TTG |
| | | CTT | 25 | 8.606 | | | | | | | | | | CTC |
| | | TTA | 15 | 18.490 | | | | | | | | | | CTG |
| | | TTG | 12 | 18.969 | | | | | | | | | | CTA |
| TCC_N_ | S_N_T | AAC | 86 | 46.821 | 1 | 54.4477 | 54.9720272 | 1.59634277 | 1.222574974 | 116 | 1.94807455 | 2.3:TCCAA | 1.8:TCCAA | AAC |
| ACT | | AAT | 30 | 69.179 | | 7578808 | 77662484 | 55078095e- | 7552338e-13 | | 6280247 | TACT | CACT | AAT |
| | | | | | | 6105 | | 13 | | | | | | |
| TCC_S_ | S_S_T | AGC | 97 | 27.154 | 5 | 146.082 | 210.882214 | 9.10272792 | 1.332144082 | 241 | 2.05814356 | 2.3:TCCTC | 3.6:TCCAG | AGC |
| ACT | | AGT | 18 | 39.332 | | 5968322 | 31999523 | 3224158e- | 319934e-43 | | 82482203 | AACT | CACT | TCT |
| | | TCA | 22 | 50.423 | | 254 | | 30 | | | | | | TCC |
| | | TCC | 30 | 37.841 | | | | | | | | | | TCA |
| | | TCG | 18 | 23.486 | | | | | | | | | | TCG |
| | | TCT | 56 | 62.763 | | | | | | | | | | AGT |
| TCC_S_ | S_S_E | AGC | 23 | 18.366 | 5 | 34.0534 | 38.0150109 | 2.32332123 | 3.747130933 | 163 | 1.50275837 | 2.1:TCCTC | 2.0:TCCAG | AGT |
| GAA | | AGT | 53 | 26.602 | | 9753951 | 5243964 | 16805244e- | 309858e-07 | | 22429758 | CGAA | TGAA | TCT |
| | | TCA | 26 | 34.104 | | 111 | | 06 | | | | | | TCA |
| | | TCC | 12 | 25.594 | | | | | | | | | | AGC |
| | | TCG | 13 | 15.885 | | | | | | | | | | TCG |
| | | TCT | 36 | 42.450 | | | | | | | | | | TCC |
| TCG_A_ | S_A_T | GCA | 7 | 6.778 | 3 | 30.8974 | 40.8812377 | 8.93376790 | 6.929669219 | 23 | 2.53951828 | 10.3:TCGG | 4.6:TCGGC | GCG |
| ACG | | GCC | 0 | 5.138 | | 5124799 | 67981865 | 2277777e- | 6948315e-09 | | 7886255 | CCACG | GACG | GCA |
| | | GCG | 12 | 2.630 | | 2162 | | 07 | | | | | | GCT |
| | | GCT | 4 | 8.454 | | | | | | | | | | GCC |
| TCG_S_ | S_S_R | AGC | 23 | 6.310 | 5 | 52.9653 | 63.8064891 | 3.41883439 | 1.981379944 | 56 | 2.26159981 | 7.3:TCGTC | 3.6:TCGA | AGC |
| AGA | | AGT | 3 | 9.139 | | 8762469 | 8303538 | 8338685e- | 7720155e-12 | | 2394971 | TAGA | GCAGA | TCA |
| | | TCA | 12 | 11.717 | | 922 | | 10 | | | | | | TCG |
| | | TCC | 6 | 8.793 | | | | | | | | | | TCC |
| | | TCG | 10 | 5.457 | | | | | | | | | | AGT |
| | | TCT | 2 | 14.584 | | | | | | | | | | TCT |
| TCG_T_ | S_T_Y | ACA | 2 | 8.509 | 3 | 49.2021 | 77.0837532 | 1.18140396 | 1.295464308 | 28 | 4.24934636 | 5.9:TCGAC | 5.1:TCGAC | ACG |
| TAC | | ACC | 1 | 5.932 | | 2510651 | 6896265 | 41251486e- | 8977584e-16 | | 2217466 | CTAC | GTAC | ACT |
| | | ACG | 20 | 3.927 | | 1545 | | 10 | | | | | | ACA |
| | | ACT | 5 | 9.631 | | | | | | | | | | ACC |
| TCT_F_ | S_F_K | TTC | 53 | 31.272 | 1 | 24.9792 | 25.4214856 | 5.79519015 | 4.607577625 | 77 | 1.75781114 | 1.9:TCTTT | 1.7:TCTTT | TTC |
| AAG | | TTT | 24 | 45.728 | | 0815209 | 56680723 | 0287654e- | 955305e-07 | | 45100537 | TAAG | CAAG | TTT |
| | | | | | | 4458 | | 07 | | | | | | |
| TCT_L_ | S_L_K | CTA | 30 | 43.049 | 5 | 75.6152 | 76.0780217 | 6.92147569 | 5.540965637 | 304 | 1.52782126 | 3.3:TCTCT | 1.7:TCTTT | TTG |
| AAA | | CTC | 17 | 17.555 | | 7406230 | 6943991 | 20665014e- | 349504e-15 | | 28931913 | TAAA | GAAA | TTA |
| | | CTG | 22 | 34.388 | | 785 | | 15 | | | | | | CTA |
| | | CTT | 12 | 39.047 | | | | | | | | | | CTG |
| | | TTA | 73 | 83.896 | | | | | | | | | | CTC |
| | | TTG | 150 | 86.066 | | | | | | | | | | CTT |
| TCT_L_ | S_L_K | CTA | 17 | 34.694 | 5 | 60.0825 | 52.5598094 | 1.16862051 | 4.141205080 | 245 | 1.46064057 | 3.9:TCTCT | 1.5:TCTTT | TTG |
| AAG | | CTC | 19 | 14.148 | | 8349875 | 25167145 | 59576313e- | 326172e-10 | | 01309122 | TAAG | GAAG | TTA |
| | | CTG | 17 | 27.714 | | 6314 | | 11 | | | | | | CTC |
| | | CTT | 8 | 31.469 | | | | | | | | | | CTG |
| | | TTA | 79 | 67.613 | | | | | | | | | | CTA |
| | | TTG | 105 | 69.363 | | | | | | | | | | CTT |
| TCT_R_ | S_R_T | AGA | 11 | 22.285 | 5 | 64.8092 | 142.816223 | 1.22764015 | 4.507404819 | 47 | 3.61096856 | 2.2:TCTCG | 7.9:TCTCG | CGC |
| ACA | | AGG | 6 | 10.194 | | 3712072 | 94465973 | 27124713e- | 6408696e-29 | | 7172898 | TACA | CACA | AGA |
| | | CGA | 4 | 3.209 | | 044 | | 12 | | | | | | AGG |
| | | CGC | 22 | 2.782 | | | | | | | | | | CGA |
| | | CGG | 1 | 1.962 | | | | | | | | | | CGT |
| | | CGT | 3 | 6.568 | | | | | | | | | | CGG |
| TCT_R_ | S_R_G | AGA | 14 | 22.285 | 5 | 34.3717 | 46.7680147 | 2.00775683 | 6.334996410 | 47 | 2.42861200 | 3.2:TCTCG | 3.3:TCTCG | CGT |
| GGT | | AGG | 5 | 10.194 | | 8608960 | 3594018 | 11523777e- | 659402e-09 | | 5147134 | AGGT | TGGT | AGA |
| | | CGA | 1 | 3.209 | | 736 | | 06 | | | | | | AGG |
| | | CGC | 1 | 2.782 | | | | | | | | | | CGG |
| | | CGG | 4 | 1.962 | | | | | | | | | | CGC |
| | | CGT | 22 | 6.568 | | | | | | | | | | CGA |
| TCT_S_ | S_S_P | AGC | 4 | 19.943 | 5 | 75.2369 | 64.2424150 | 8.30177517 | 1.609188337 | 177 | 1.65820733 | 5.0:TCTAG | 1.7:TCTTC | TCT |
| CCA | | AGT | 7 | 28.887 | | 3891077 | 3222692 | 8770996e- | 6118733e-12 | | 32926326 | CCCA | ACCA | TCA |
| | | TCA | 62 | 37.033 | | 01 | | 15 | | | | | | TCG |
| | | TCC | 14 | 27.792 | | | | | | | | | | TCC |
| | | TCG | 24 | 17.249 | | | | | | | | | | AGT |
| | | TCT | 66 | 46.096 | | | | | | | | | | AGC |
| TCT_S_ | S_S_A | AGC | 5 | 26.929 | 5 | 78.7935 | 79.4383037 | 1.50033169 | 1.099894043 | 239 | 1.61693720 | 5.4:TCTAG | 1.9:TCTTC | TCT |
| GCT | | AGT | 29 | 39.005 | | 1923904 | 067868 | 37091926e- | 6380759e-15 | | 94205763 | CGCT | TGCT | TCA |
| | | TCA | 37 | 50.005 | | 187 | | 15 | | | | | | TCC |
| | | TCC | 35 | 37.527 | | | | | | | | | | AGT |
| | | TCG | 14 | 23.291 | | | | | | | | | | TCG |
| | | TCT | 119 | 62.243 | | | | | | | | | | AGC |
| TCT_S_ | S_S_S | AGC | 22 | 40.562 | 5 | 50.6429 | 45.8168118 | 1.02348603 | 9.896384239 | 360 | 1.33913152 | 2.3:TCTAG | 1.3:TCTTC | TCT |
| TCA | | AGT | 26 | 58.753 | | 6245551 | 8163625 | 7530684e- | 150334e-09 | | 19063376 | TTCA | TTCA | TCA |
| | | TCA | 100 | 75.321 | | 4024 | | 09 | | | | | | TCC |
| | | TCC | 51 | 56.527 | | | | | | | | | | TCG |
| | | TCG | 36 | 35.083 | | | | | | | | | | AGT |
| | | TCT | 125 | 93.754 | | | | | | | | | | AGC |
| TCT_S_ | S_S_S | AGC | 30 | 54.872 | 5 | 110.804 | 100.865597 | 2.77000196 | 3.472153887 | 487 | 1.43885377 | 3.2:TCTAG | 1.6:TCTTC | TCT |
| TCT | | AGT | 25 | 79.479 | | 4423066 | 6976273 | 06508943e- | 053695e-20 | | 90906994 | TTCT | TTCT | TCC |
| | | TCA | 100 | 101.893 | | 365 | | 22 | | | | | | TCA |
| | | TCC | 101 | 76.468 | | | | | | | | | | TCG |
| | | TCG | 33 | 47.459 | | | | | | | | | | AGC |
| | | TCT | 198 | 126.829 | | | | | | | | | | AGT |
| TGC_A_ | C_A_D | GCA | 1 | 2.358 | 3 | 18.6989 | 32.1225537 | 0.00031551 | 4.931281339 | 8 | 5.22125200 | 3.6:TGCGC | 6.6:TGCGC | GCG |
| GAC | | GCC | 0 | 1.787 | | 4872271 | 95303546 | 5564549174 | 292999e-07 | | 3058486 | CGAC | GGAC | GCT |
| | | GCG | 6 | 0.915 | | 2714 | | 65 | | | | | | GCA |
| | | GCT | 1 | 2.940 | | | | | | | | | | GCC |
| TGC_R_ | C_R_E | AGA | 6 | 16.595 | 5 | 63.2793 | 173.713173 | 2.54795835 | 1.176820046 | 35 | 4.39316281 | 2.8:TGCAG | 11.6:TGCC | CGG |
| GAA | | AGG | 5 | 7.592 | | 7562986 | 08106347 | 6190585e- | 0722038e-35 | | 07751886 | AGAA | GGGAA | AGA |
| | | CGA | 2 | 2.390 | | 283 | | 12 | | | | | | AGG |
| | | CGC | 1 | 2.072 | | | | | | | | | | CGT |
| | | CGG | 17 | 1.461 | | | | | | | | | | CGA |
| | | CGT | 4 | 4.891 | | | | | | | | | | CGC |
| TGC_S_ | C_S_S | AGC | 21 | 4.056 | 5 | 48.9000 | 80.8798891 | 2.32613018 | 5.492143473 | 36 | 3.45265139 | 5.9:TGCAG | 5.2:TGCA | AGC |
| AGT | | AGT | 1 | 5.875 | | 4726534 | 0665548 | 8031796e- | 505034e-16 | | 7340153 | TAGT | GCAGT | TCT |
| | | TCA | 4 | 7.532 | | 075 | | 09 | | | | | | TCA |
| | | TCC | 3 | 5.653 | | | | | | | | | | TCG |
| | | TCG | 3 | 3.508 | | | | | | | | | | TCC |
| | | TCT | 4 | 9.375 | | | | | | | | | | AGT |
| TGG_A_ | W_A_A | GCA | 6 | 10.609 | 3 | 32.6388 | 52.9360075 | 3.83813989 | 1.891868904 | 36 | 2.77920713 | 2.0:TGGGC | 4.4:TGGG | GCG |
| GCA | | GCC | 4 | 8.043 | | 1574338 | 7842901 | 7941649e- | 0902343e-11 | | 46381246 | CGCA | CGGCA | GCT |
| | | GCG | 18 | 4.116 | | 3695 | | 07 | | | | | | GCA |
| | | GCT | 8 | 13.232 | | | | | | | | | | GCC |
| TGG_G_ | W_G_F | GGA | 4 | 10.514 | 3 | 25.3568 | 32.3939531 | 1.30028661 | 4.322641067 | 47 | 1.80890026 | 2.6:TGGG | 3.1:TGGG | GGT |
| TTC | | GGC | 4 | 9.293 | | 0909962 | 0215945 | 28092507e- | 3959026e-07 | | 38868865 | GATTC | GGTTC | GGG |
| | | GGG | 18 | 5.838 | | 3334 | | 05 | | | | | | GGC |
| | | GGT | 21 | 21.355 | | | | | | | | | | GGA |
| TGG_S_ | W_S_N | AGC | 23 | 7.324 | 5 | 31.2160 | 41.6514078 | 8.49046515 | 6.928907808 | 65 | 1.87236374 | 2.4:TGGTC | 3.1:TGGA | AGC |
| AAC | | AGT | 8 | 10.608 | | 9775660 | 8396645 | 0773429e- | 577932e-08 | | 3393983 | TAAC | GCAAC | TCA |
| | | TCA | 12 | 13.600 | | 7642 | | 06 | | | | | | TCG |
| | | TCC | 7 | 10.206 | | | | | | | | | | AGT |
| | | TCG | 8 | 6.334 | | | | | | | | | | TCT |
| | | TCT | 7 | 16.928 | | | | | | | | | | TCC |
| TGG_T_ | W_T_A | ACA | 8 | 12.764 | 3 | 31.0376 | 45.3108236 | 8.34665017 | 7.947062273 | 42 | 2.46351994 | 2.1:TGGAC | 3.6:TGGA | ACG |
| GCA | | ACC | 6 | 8.898 | | 6758718 | 8844089 | 1987353e- | 862598e-10 | | 45135227 | TGCA | CGGCA | ACA |
| | | ACG | 21 | 5.891 | | 3346 | | 07 | | | | | | ACT |
| | | ACT | 7 | 14.446 | | | | | | | | | | ACC |
| TGT_A_ | C_A_L | GCA | 5 | 9.136 | 3 | 24.8570 | 36.3894730 | 1.65400695 | 6.194864626 | 31 | 2.31599703 | 3.5:TGTGC | 3.9:TGTGC | GCG |
| CTT | | GCC | 2 | 6.926 | | 9567496 | 1737206 | 17194634e- | 477173e-08 | | 41988108 | CCTT | GCTT | GCT |
| | | GCG | 14 | 3.544 | | 1563 | | 05 | | | | | | GCA |
| | | GCT | 10 | 11.394 | | | | | | | | | | GCC |
| TGT_G_ | C_G_S | GGA | 3 | 9.172 | 3 | 30.6265 | 39.2950791 | 1.01874076 | 1.502966458 | 41 | 2.45261943 | 3.1:TGTGG | 3.0:TGTGG | GGC |
| TCC | | GGC | 24 | 8.107 | | 3952198 | 0807935 | 03757552e- | 2441697e-08 | | 0527976 | ATCC | CTCC | GGT |
| | | GGG | 3 | 5.093 | | 6846 | | 06 | | | | | | GGG |
| | | GGT | 11 | 18.629 | | | | | | | | | | GGA |
| TGT_N_ | C_N_D | AAC | 40 | 21.796 | 1 | 25.2469 | 25.4937517 | 5.04395006 | 4.438179152 | 54 | 1.94586983 | 2.3:TGTAA | 1.8:TGTAA | AAC |
| GAT | | AAT | 14 | 32.204 | | 4115866 | 17738327 | 4668597e- | 4621746e-07 | | 29389332 | TGAT | CGAT | AAT |
| | | | | | | 0594 | | 07 | | | | | | |
| TGT_P_ | C_P_V | CCA | 6 | 11.407 | 3 | 24.5786 | 35.1338941 | 1.89120704 | 1.141424068 | 28 | 2.75221050 | 2.2:TGTCC | 3.6:TGTCC | CCC |
| GTT | | CCC | 16 | 4.494 | | 1704168 | 0984883 | 5539058e- | 7422168e-07 | | 50617385 | TGTT | CGTT | CCA |
| | | CCG | 2 | 3.442 | | 6204 | | 05 | | | | | | CCT |
| | | CCT | 4 | 8.657 | | | | | | | | | | CCG |
| TGT_P_ | C_P_S | CCA | 5 | 12.222 | 3 | 34.4371 | 49.9896489 | 1.60187264 | 8.029834703 | 30 | 3.20549702 | 3.7:TGTCC | 3.9:TGTCC | CCC |
| TCA | | CCC | 19 | 4.815 | | 3289291 | 594401 | 0677404e- | 862732e-11 | | 19154844 | GTCA | CTCA | CCT |
| | | CCG | 1 | 3.688 | | 87 | | 07 | | | | | | CCA |
| | | CCT | 5 | 9.275 | | | | | | | | | | CCG |
| TTA_A_ | L_A_R | GCA | 61 | 30.354 | 3 | 40.8351 | 44.9589155 | 7.08729146 | 9.440606649 | 103 | 1.87474295 | 2.0:TTAGC | 2.0:TTAGC | GCA |
| AGA | | GCC | 13 | 23.011 | | 7967386 | 03477066 | 77819425e- | 688654e-10 | | 88029303 | TAGA | AAGA | GCT |
| | | GCG | 10 | 11.776 | | 081 | | 09 | | | | | | GCC |
| | | GCT | 19 | 37.859 | | | | | | | | | | GCG |
| TTA_A_ | L_A_F | GCA | 47 | 28.291 | 3 | 36.2851 | 36.5162052 | 6.51764067 | 5.824100243 | 96 | 1.80741571 | 2.4:TTAGC | 1.9:TTAGC | GCA |
| TTT | | GCC | 13 | 21.447 | | 6518844 | 512956 | 4333993e- | 005351e-08 | | 75741081 | TTTT | GTTT | GCG |
| | | GCG | 21 | 10.976 | | 6044 | | 08 | | | | | | GCT |
| | | GCT | 15 | 35.286 | | | | | | | | | | GCC |
| TTA_C_ | L_C_K | TGC | 33 | 15.058 | 1 | 33.9960 | 34.2869220 | 5.52227462 | 4.755668080 | 40 | 2.38615241 | 3.6:TTATG | 2.2:TTATG | TGC |
| AAG | | TGT | 7 | 24.942 | | 9659358 | 4615614 | 45917235e- | 085938e-09 | | 42715838 | TAAG | CAAG | TGT |
| | | | | | | 874 | | 09 | | | | | | |
| TTA_F_ | L_F_K | TTC | 72 | 45.080 | 1 | 26.4838 | 27.0686833 | 2.65755415 | 1.963532405 | 111 | 1.62926625 | 1.7:TTATT | 1.6:TTATT | TTC |
| AAA | | TTT | 39 | 65.920 | | 1491505 | 88094897 | 453881e-07 | 815934e-07 | | 28808823 | TAAA | CAAA | TTT |
| | | | | | | 5326 | | | | | | | | |
| TTA_G_ | L_G_H | GGA | 11 | 14.988 | 3 | 26.1885 | 33.4565434 | 8.70865261 | 2.580048313 | 67 | 1.80881277 | 1.8:TTAGG | 2.8:TTAGG | GGG |
| CAT | | GGC | 16 | 13.248 | | 3552930 | 3673933 | 0755225e- | 3649597e-07 | | 51575809 | TCAT | GCAT | GGT |
| | | GGG | 23 | 8.322 | | 868 | | 06 | | | | | | GGC |
| | | GGT | 17 | 30.442 | | | | | | | | | | GGA |
| TTA_I_ | L_I_N | ATA | 29 | 35.671 | 2 | 33.3706 | 37.5348022 | 5.67090303 | 7.070029989 | 128 | 1.65652332 | 1.6:TTAAT | 1.9:TTAAT | ATC |
| AAC | | ATC | 63 | 33.049 | | 6474554 | 3037545 | 89063374e- | 944562e-09 | | 83083847 | TAAC | CAAC | ATT |
| | | ATT | 36 | 59.281 | | 632 | | 08 | | | | | | ATA |
| TTA_I_ | L_I_K | ATA | 43 | 42.916 | 2 | 36.2301 | 38.2981016 | 1.35744235 | 4.826950982 | 154 | 1.51901420 | 1.8:TTAAT | 1.8:TTAAT | ATC |
| AAG | | ATC | 71 | 39.762 | | 5686998 | 7056091 | 65225935e- | 004029e-09 | | 28988018 | TAAG | CAAG | ATA |
| | | ATT | 40 | 71.322 | | 954 | | 08 | | | | | | ATT |
| TTA_P_ | L_P_R | CCA | 50 | 27.296 | 3 | 34.0965 | 32.7436822 | 1.89033174 | 3.647596119 | 67 | 1.91351802 | 5.4:TTACC | 1.8:TTACC | CCA |
| AGA | | CCC | 2 | 10.753 | | 7108606 | 538446 | 73552026e- | 0134545e-07 | | 95466208 | CAGA | AAGA | CCT |
| | | CCG | 4 | 8.236 | | 025 | | 07 | | | | | | CCG |
| | | CCT | 11 | 20.715 | | | | | | | | | | CCC |
| TTC_E_ | F_E_L | GAA | 5 | 18.171 | 1 | 28.5367 | 31.7044460 | 9.19383870 | 1.795107781 | 26 | 2.84364100 | 3.6:TTCGA | 2.7:TTCGA | GAG |
| CTC | | GAG | 21 | 7.829 | | 1690703 | 12241654 | 8134858e- | 2211043e-08 | | 32265607 | ACTC | GCTC | GAA |
| | | | | | | 8894 | | 08 | | | | | | |
| TTC_F_ | F_F_N | TTC | 67 | 37.770 | 1 | 37.6288 | 38.0911185 | 8.55681413 | 6.751675963 | 93 | 1.86557392 | 2.1:TTCTT | 1.8:TTCTT | TTC |
| AAT | | TTT | 26 | 55.230 | | 8027655 | 3996725 | 3533808e- | 71414e-10 | | 81473795 | TAAT | CAAT | TTT |
| | | | | | | 161 | | 10 | | | | | | |
| TTC_G_ | F_G_A | GGA | 2 | 4.474 | 3 | 33.8350 | 51.6750946 | 2.14655683 | 3.512767454 | 20 | 3.68998155 | 7.9:TTCGG | 5.2:TTCGG | GGG |
| GCG | | GGC | 0 | 3.955 | | 6411344 | 9643036 | 11012522e- | 530547e-11 | | 1622438 | CGCG | GGCG | GGT |
| | | GGG | 13 | 2.484 | | 2726 | | 07 | | | | | | GGA |
| | | GGT | 5 | 9.087 | | | | | | | | | | GGC |
| TTC_G_ | F_G_G | GGA | 7 | 21.251 | 3 | 44.4974 | 40.0992283 | 1.18322660 | 1.015132998 | 95 | 1.77414397 | 5.9:TTCGG | 1.7:TTCGG | GGT |
| GGT | | GGC | 13 | 18.784 | | 1056912 | 2888957 | 05756333e- | 0811976e-08 | | 06281443 | GGGT | TGGT | GGC |
| | | GGG | 2 | 11.800 | | 764 | | 09 | | | | | | GGA |
| | | GGT | 73 | 43.165 | | | | | | | | | | GGG |
| TTC_I_ | F_I_K | ATA | 24 | 35.113 | 2 | 45.2725 | 52.3500789 | 1.47639006 | 4.288691432 | 126 | 1.85176335 | 1.7:TTCAT | 2.1:TTCAT | ATC |
| AAG | | ATC | 68 | 32.532 | | 0192802 | 13783285 | 94067783e- | 571085e-12 | | 02341002 | TAAG | CAAG | ATT |
| | | ATT | 34 | 58.355 | | 147 | | 10 | | | | | | ATA |
| TTC_I_ | F_I_N | ATA | 43 | 50.440 | 2 | 29.5640 | 32.7363473 | 3.80404828 | 7.787400148 | 181 | 1.48221997 | 1.4:TTCAT | 1.7:TTCAT | ATC |
| AAT | | ATC | 80 | 46.733 | | 5962526 | 64005475 | 6929706e- | 03845e-08 | | 69326416 | TAAT | CAAT | ATT |
| | | ATT | 58 | 83.827 | | 1585 | | 07 | | | | | | ATA |
| TTC_L_ | F_L_T | CTA | 1 | 5.381 | 5 | 38.1470 | 54.2848923 | 3.52510708 | 1.831450741 | 38 | 2.66766748 | 5.4:TTCCT | 4.2:TTCCT | CTG |
| ACG | | CTC | 4 | 2.194 | | 4043584 | 94110486 | 561456e-07 | 1245249e-10 | | 4060965 | AACG | GACG | TTG |
| | | CTG | 18 | 4.298 | | 1724 | | | | | | | | TTA |
| | | CTT | 1 | 4.881 | | | | | | | | | | CTC |
| | | TTA | 7 | 10.487 | | | | | | | | | | CTT |
| | | TTG | 7 | 10.758 | | | | | | | | | | CTA |
| TTC_L_ | F_L_Y | CTA | 14 | 13.736 | 5 | 46.5566 | 80.9028881 | 6.99542926 | 5.431607692 | 97 | 1.80118387 | 1.6:TTCTT | 4.6:TTCCT | CTC |
| TAT | | CTC | 26 | 5.601 | | 6018864 | 3768674 | 6936029e- | 898021e-16 | | 15847095 | GTAT | CTAT | TTA |
| | | CTG | 9 | 10.972 | | 842 | | 09 | | | | | | TTG |
| | | CTT | 12 | 12.459 | | | | | | | | | | CTA |
| | | TTA | 19 | 26.769 | | | | | | | | | | CTT |
| | | TTG | 17 | 27.462 | | | | | | | | | | CTG |
| TTC_P_ | F_P_I | CCA | 22 | 35.037 | 3 | 35.4273 | 38.7209459 | 9.89568862 | 1.988735819 | 86 | 1.68456016 | 5.3:TTCCC | 2.4:TTCCC | CCC |
| ATT | | CCC | 33 | 13.802 | | 0010436 | 91366946 | 0106094e- | 8520597e-08 | | 64462353 | GATT | CATT | CCT |
| | | CCG | 2 | 10.571 | | 9706 | | 08 | | | | | | CCA |
| | | CCT | 29 | 26.590 | | | | | | | | | | CCG |
| TTC_R_ | F_R_M | AGA | 19 | 27.026 | 5 | 46.0541 | 88.7003047 | 8.85431711 | 1.259749214 | 57 | 2.38538998 | 2.7:TTCCG | 5.9:TTCCG | CGC |
| ATG | | AGG | 9 | 12.364 | | 8355333 | 5527831 | 8710294e- | 4604077e-17 | | 70262225 | TATG | CATG | AGA |
| | | CGA | 3 | 3.892 | | 002 | | 09 | | | | | | AGG |
| | | CGC | 20 | 3.374 | | | | | | | | | | CGT |
| | | CGG | 3 | 2.379 | | | | | | | | | | CGG |
| | | CGT | 3 | 7.965 | | | | | | | | | | CGA |
| TTC_R_ | F_R_V | AGA | 12 | 16.595 | 5 | 32.5447 | 42.5135036 | 4.63367104 | 4.636502643 | 35 | 2.39960762 | 4.8:TTCCG | 3.7:TTCCG | CGT |
| GTC | | AGG | 4 | 7.592 | | 4978769 | 7635169 | 8849248e- | 488522e-08 | | 8861654 | AGTC | TGTC | AGA |
| | | CGA | 0 | 2.390 | | 161 | | 06 | | | | | | AGG |
| | | CGC | 1 | 2.072 | | | | | | | | | | CGC |
| | | CGG | 0 | 1.461 | | | | | | | | | | CGG |
| | | CGT | 18 | 4.891 | | | | | | | | | | CGA |
| TTC_T_ | F_T_T | ACA | 9 | 20.058 | 3 | 40.7702 | 47.0559330 | 7.31556060 | 3.381682703 | 66 | 2.06014548 | 4.6:TTCAC | 2.6:TTCAC | ACC |
| ACC | | ACC | 36 | 13.983 | | 6056501 | 8426324 | 4357518e- | 925989e-10 | | 13568436 | GACC | CACC | ACT |
| | | ACG | 2 | 9.257 | | 424 | | 09 | | | | | | ACA |
| | | ACT | 19 | 22.702 | | | | | | | | | | ACG |
| TTG_A_ | L_A_K | GCA | 43 | 58.055 | 3 | 39.5191 | 45.4823541 | 1.34731836 | 7.307112300 | 197 | 1.54382854 | 1.4:TTGGC | 1.9:TTGGC | GCC |
| AAG | | GCC | 83 | 44.011 | | 4549730 | 3168187 | 3938697e- | 936298e-10 | | 72102897 | TAAG | CAAG | GCT |
| | | GCG | 21 | 22.524 | | 9935 | | 08 | | | | | | GCA |
| | | GCT | 50 | 72.410 | | | | | | | | | | GCG |
| TTG_A_ | L_A_V | GCA | 14 | 12.377 | 3 | 25.4087 | 33.3523008 | 1.26813842 | 2.714085477 | 42 | 2.00698600 | 3.1:TTGGC | 3.3:TTGGC | GCG |
| GTA | | GCC | 3 | 9.383 | | 8108892 | 649378 | 89543812e- | 293427e-07 | | 12089666 | CGTA | GGTA | GCA |
| | | GCG | 16 | 4.802 | | 3718 | | 05 | | | | | | GCT |
| | | GCT | 9 | 15.438 | | | | | | | | | | GCC |
| TTG_F_ | L_F_K | TTC | 61 | 37.364 | 1 | 24.6651 | 25.1776360 | 6.82076081 | 5.228500103 | 92 | 1.67525177 | 1.8:TTGTT | 1.6:TTGTT | TTC |
| AAA | | TTT | 31 | 54.636 | | 1738418 | 00569198 | 8308639e- | 845871e-07 | | 48721272 | TAAA | CAAA | TTT |
| | | | | | | 7177 | | 07 | | | | | | |
| TTG_G_ | L_G_R | GGA | 1 | 3.355 | 3 | 34.7561 | 42.8155522 | 1.37165580 | 2.693218386 | 15 | 4.06706032 | 13.6:TTGG | 4.4:TTGGG | GGC |
| CGA | | GGC | 13 | 2.966 | | 7224641 | 1577901 | 00802107e- | 940242e-09 | | 4824074 | GTCGA | CCGA | GGG |
| | | GGG | 1 | 1.863 | | 847 | | 07 | | | | | | GGA |
| | | GGT | 0 | 6.815 | | | | | | | | | | GGT |
| TTG_G_ | L_G_G | GGA | 9 | 21.922 | 3 | 42.0478 | 35.4101719 | 3.91938603 | 9.978512812 | 98 | 1.63491382 | 12.2:TTGG | 1.6:TTGGG | GGT |
| GGT | | GGC | 16 | 19.377 | | 6106218 | 65722526 | 1094367e- | 181827e-08 | | 89784637 | GGGGT | TGGT | GGC |
| | | GGG | 1 | 12.173 | | 074 | | 09 | | | | | | GGA |
| | | GGT | 72 | 44.528 | | | | | | | | | | GGG |
| TTG_R_ | L_R_R | AGA | 107 | 67.328 | 5 | 59.4187 | 48.8561150 | 1.60261844 | 2.374716094 | 142 | 1.63054105 | 16.8:TTGC | 1.6:TTGAG | AGA |
| AGA | | AGG | 21 | 30.800 | | 1797865 | 53301195 | 75019526e- | 3118415e-09 | | 56867457 | GCAGA | AAGA | AGG |
| | | CGA | 6 | 9.697 | | 251 | | 11 | | | | | | CGA |
| | | CGC | 0 | 8.405 | | | | | | | | | | CGT |
| | | CGG | 3 | 5.927 | | | | | | | | | | CGG |
| | | CGT | 5 | 19.843 | | | | | | | | | | CGC |
| TTG_R_ | L_R_S | AGA | 18 | 26.552 | 5 | 37.4600 | 40.9191316 | 4.84296128 | 9.742424648 | 56 | 2.07448894 | 4.7:TTGCG | 2.6:TTGAG | AGG |
| AGC | | AGG | 31 | 12.147 | | 9684611 | 8825175 | 2457307e- | 89357e-08 | | 7654107 | GAGC | GAGC | AGA |
| | | CGA | 1 | 3.824 | | 054 | | 07 | | | | | | CGC |
| | | CGC | 4 | 3.314 | | | | | | | | | | CGT |
| | | CGG | 0 | 2.337 | | | | | | | | | | CGA |
| | | CGT | 2 | 7.825 | | | | | | | | | | CGG |
| TTG_R_ | L_R_S | AGA | 10 | 24.181 | 5 | 43.1046 | 60.5801279 | 3.51885382 | 9.222113330 | 51 | 2.50617565 | 3.0:TTGCG | 3.6:TTGCG | CGT |
| TCC | | AGG | 8 | 11.062 | | 9646081 | 9335526 | 26854796e- | 92188e-12 | | 4751704 | CTCC | TTCC | AGA |
| | | CGA | 4 | 3.483 | | 573 | | 08 | | | | | | AGG |
| | | CGC | 1 | 3.019 | | | | | | | | | | CGA |
| | | CGG | 2 | 2.129 | | | | | | | | | | CGG |
| | | CGT | 26 | 7.127 | | | | | | | | | | CGC |
| TTG_S_ | L_S_K | AGC | 54 | 28.957 | 5 | 52.2399 | 56.6307482 | 4.81675865 | 6.025369190 | 257 | 1.53348458 | 2.1:TTGTC | 1.9:TTGAG | AGT |
| AAA | | AGT | 69 | 41.943 | | 5312212 | 023017 | 5050081e- | 305502e-11 | | 85040287 | GAAA | CAAA | AGC |
| | | TCA | 43 | 53.771 | | 2024 | | 10 | | | | | | TCT |
| | | TCC | 35 | 40.354 | | | | | | | | | | TCA |
| | | TCG | 12 | 25.045 | | | | | | | | | | TCC |
| | | TCT | 44 | 66.930 | | | | | | | | | | TCG |
| TTG_S_ | L_S_R | AGC | 16 | 3.042 | 5 | 41.7826 | 64.0050657 | 6.51822453 | 1.802226337 | 27 | 3.29917346 | 8.5:TTGTC | 5.3:TTGAG | AGC |
| CGA | | AGT | 1 | 4.406 | | 0196475 | 7943416 | 8931221e- | 6573123e-12 | | 7478398 | CCGA | CCGA | TCT |
| | | TCA | 4 | 5.649 | | 844 | | 08 | | | | | | TCA |
| | | TCC | 0 | 4.240 | | | | | | | | | | TCG |
| | | TCG | 2 | 2.631 | | | | | | | | | | AGT |
| | | TCT | 4 | 7.032 | | | | | | | | | | TCC |
| TTG_S_ | L_S_E | AGC | 40 | 24.788 | 5 | 56.8841 | 64.9508608 | 5.34280227 | 1.147352212 | 220 | 1.67470787 | 1.6:TTGTC | 2.0:TTGAG | AGT |
| GAA | | AGT | 73 | 35.904 | | 3505020 | 3286067 | 35483194e- | 2496002e-12 | | 31635254 | GGAA | TGAA | AGC |
| | | TCA | 31 | 46.030 | | 3206 | | 11 | | | | | | TCT |
| | | TCC | 24 | 34.544 | | | | | | | | | | TCA |
| | | TCG | 13 | 21.439 | | | | | | | | | | TCC |
| | | TCT | 39 | 57.294 | | | | | | | | | | TCG |
| TTG_S_ | L_S_G | AGC | 26 | 6.986 | 5 | 52.5979 | 66.1650786 | 4.06720562 | 6.422976282 | 62 | 2.18837281 | 12.1:TTGT | 3.7:TTGAG | AGC |
| GGC | | AGT | 13 | 10.119 | | 6669048 | 198016 | 6022017e- | 04882e-13 | | 1854458 | CGGGC | CGGC | AGT |
| | | TCA | 5 | 12.972 | | 5 | | 10 | | | | | | TCT |
| | | TCC | 8 | 9.735 | | | | | | | | | | TCC |
| | | TCG | 0 | 6.042 | | | | | | | | | | TCA |
| | | TCT | 10 | 16.147 | | | | | | | | | | TCG |
| TTG_T_ | L_T_K | ACA | 47 | 55.919 | 3 | 59.2329 | 66.4256951 | 8.57226048 | 2.485124346 | 184 | 1.65170312 | 2.1:TTGAC | 2.1:TTGAC | ACC |
| AAG | | ACC | 82 | 38.983 | | 9421671 | 0126049 | 447061e-13 | 737293e-14 | | 81606915 | TAAG | CAAG | ACA |
| | | ACG | 25 | 25.808 | | 835 | | | | | | | | ACT |
| | | ACT | 30 | 63.289 | | | | | | | | | | ACG |
| TTT_A_ | F_A_K | GCA | 45 | 69.549 | 3 | 46.8332 | 53.6615345 | 3.77141409 | 1.324938388 | 236 | 1.53629944 | 1.5:TTTGC | 1.9:TTTGC | GCC |
| AAA | | GCC | 99 | 52.724 | | 2610160 | 0541012 | 38681947e- | 5999494e-11 | | 5222163 | AAAA | CAAA | GCT |
| | | GCG | 24 | 26.983 | | 495 | | 10 | | | | | | GCA |
| | | GCT | 68 | 86.745 | | | | | | | | | | GCG |
| TTT_A_ | F_A_I | GCA | 34 | 58.350 | 3 | 51.0414 | 57.3367141 | 4.79362463 | 2.177873961 | 198 | 1.59284289 | 1.9:TTTGC | 2.0:TTTGC | GCC |
| ATT | | GCC | 87 | 44.235 | | 4594483 | 7479119 | 78696884e- | 170604e-12 | | 44492286 | GATT | CATT | GCT |
| | | GCG | 12 | 22.638 | | 625 | | 11 | | | | | | GCA |
| | | GCT | 65 | 72.777 | | | | | | | | | | GCG |
| TTT_F_ | F_F_K | TTC | 154 | 80.819 | 1 | 111.679 | 111.579378 | 4.20090512 | 4.417589270 | 199 | 2.04885787 | 2.6:TTTTT | 1.9:TTTTT | TTC |
| AAA | | TTT | 45 | 118.181 | | 0884147 | 06008312 | 1952221e- | 6388993e-26 | | 6167616 | TAAA | CAAA | TTT |
| | | | | | | 7501 | | 26 | | | | | | |
| TTT_F_ | F_F_N | TTC | 76 | 45.892 | 1 | 32.6158 | 33.2597125 | 1.12293363 | 8.063599982 | 113 | 1.70610433 | 1.8:TTTTT | 1.7:TTTTT | TTC |
| AAC | | TTT | 37 | 67.108 | | 8241571 | 8496911 | 12816693e- | 82159e-09 | | 96870728 | TAAC | CAAC | TTT |
| | | | | | | 158 | | 08 | | | | | | |
| TTT_F_ | F_F_K | TTC | 84 | 45.080 | 1 | 56.3584 | 56.5801443 | 6.03931175 | 5.395390256 | 111 | 1.98994334 | 2.4:TTTTT | 1.9:TTTTT | TTC |
| AAG | | TTT | 27 | 65.920 | | 5189592 | 6747516 | 7399961e- | 833026e-14 | | 06363636 | TAAG | CAAG | TTT |
| | | | | | | 602 | | 14 | | | | | | |
| TTT_F_ | F_F_N | TTC | 107 | 65.793 | 1 | 42.5636 | 43.4589421 | 6.84179515 | 4.329486755 | 162 | 1.66704832 | 1.7:TTTTT | 1.6:TTTTT | TTC |
| AAT | | TTT | 55 | 96.207 | | 8371171 | 44057445 | 6551465e- | 932981e-11 | | 88048688 | TAAT | AAT | TTT |
| | | | | | | 351 | | 11 | | | | | | |
| TTT_G_ | F_G_F | GGA | 66 | 38.476 | 3 | 57.4193 | 59.8091548 | 2.09118180 | 6.456815200 | 172 | 1.74713864 | 2.1:TTTGG | 1.9:TTTGG | GGA |
| TTT | | GGC | 27 | 34.009 | | 4613621 | 18803094 | 43250405e- | 415913e-13 | | 78195043 | TTTT | GTTT | GGG |
| | | GGG | 41 | 21.364 | | 085 | | 12 | | | | | | GGT |
| | | GGT | 38 | 78.151 | | | | | | | | | | GGC |
| TTT_L_ | F_L_K | CTA | 37 | 54.802 | 5 | 65.5091 | 64.2194500 | 8.78784571 | 1.626926272 | 387 | 1.39458339 | 2.6:TTTCT | 1.6:TTTTT | TTG |
| AAA | | CTC | 24 | 22.348 | | 0534241 | 8134486 | 7005689e- | 2189815e-12 | | 50251376 | TAAA | GAAA | TTA |
| | | CTG | 34 | 43.777 | | 652 | | 13 | | | | | | CTA |
| | | CTT | 19 | 49.707 | | | | | | | | | | CTG |
| | | TTA | 100 | 106.802 | | | | | | | | | | CTC |
| | | TTG | 173 | 109.564 | | | | | | | | | | CTT |
| TTT_L_ | F_L_N | CTA | 15 | 31.862 | 5 | 60.3844 | 57.7488362 | 1.01222884 | 3.543994433 | 225 | 1.55328312 | 2.8:TTTCT | 1.7:TTTTT | TTG |
| AAC | | CTC | 7 | 12.993 | | 6724548 | 7723571 | 94180918e- | 085979e-11 | | 15052536 | GAAC | GAAC | TTA |
| | | CTG | 9 | 25.451 | | 044 | | 11 | | | | | | CTT |
| | | CTT | 16 | 28.900 | | | | | | | | | | CTA |
| | | TTA | 72 | 62.094 | | | | | | | | | | CTG |
| | | TTG | 106 | 63.700 | | | | | | | | | | CTC |
| TTT_L_ | F_L_K | CTA | 19 | 40.641 | 5 | 79.3121 | 73.9440143 | 1.16881024 | 1.545013964 | 287 | 1.45385673 | 4.1:TTTCT | 1.7:TTTTT | TTG |
| AAG | | CTC | 17 | 16.573 | | 0773194 | 8701377 | 3085348e- | 1702368e-14 | | 80978666 | TAAG | GAAG | TTA |
| | | CTG | 25 | 32.465 | | 37 | | 15 | | | | | | CTG |
| | | CTT | 9 | 36.863 | | | | | | | | | | CTA |
| | | TTA | 79 | 79.204 | | | | | | | | | | CTC |
| | | TTG | 138 | 81.253 | | | | | | | | | | CTT |
| TTT_L_ | F_L_N | CTA | 25 | 46.447 | 5 | 48.0880 | 41.5967310 | 3.40789754 | 7.107584964 | 328 | 1.29976840 | 2.8:TTTCT | 1.4:TTTTT | TTG |
| AAT | | CTC | 22 | 18.941 | | 6187989 | 1307689 | 89537756e- | 09957e-08 | | 14269717 | TAAT | GAAT | TTA |
| | | CTG | 45 | 37.103 | | 671 | | 09 | | | | | | CTG |
| | | CTT | 15 | 42.129 | | | | | | | | | | CTA |
| | | TTA | 95 | 90.519 | | | | | | | | | | CTC |
| | | TTG | 126 | 92.861 | | | | | | | | | | CTT |
| TTT_L_ | F_L_T | CTA | 7 | 20.108 | 5 | 47.2016 | 40.6494683 | 5.16820878 | 1.104389165 | 142 | 1.56264731 | 3.2:TTTCT | 1.7:TTTCT | TTA |
| ACT | | CTC | 14 | 8.200 | | 7841334 | 397195 | 8130768e- | 0053284e-07 | | 91046426 | GACT | CACT | TTG |
| | | CTG | 5 | 16.063 | | 545 | | 09 | | | | | | CTC |
| | | CTT | 6 | 18.239 | | | | | | | | | | CTA |
| | | TTA | 57 | 39.188 | | | | | | | | | | CTT |
| | | TTG | 53 | 40.202 | | | | | | | | | | CTG |
| TTT_L_ | F_L_E | CTA | 27 | 45.031 | 5 | 37.0620 | 37.5276486 | 5.82040747 | 4.694130600 | 318 | 1.33798552 | 1.7:TTTCT | 1.5:TTTTT | TTG |
| GAA | | CTC | 15 | 18.363 | | 3041904 | 3302657 | 5557532e- | 9807075e-07 | | 01935547 | AGAA | GGAA | TTA |
| | | CTG | 26 | 35.971 | | 3836 | | 07 | | | | | | CTA |
| | | CTT | 26 | 40.845 | | | | | | | | | | CTT |
| | | TTA | 90 | 87.759 | | | | | | | | | | CTG |
| | | TTG | 134 | 90.030 | | | | | | | | | | CTC |
| TTT_L_ | F_L_F | CTA | 14 | 18.692 | 5 | 33.8882 | 40.1609868 | 2.50617824 | 1.385793202 | 132 | 1.56266123 | 2.5:TTTCT | 2.3:TTTCT | CTT |
| TTC | | CTC | 12 | 7.623 | | 1069883 | 45061 | 5630436e- | 2876635e-07 | | 04185414 | GTTC | TTTC | TTG |
| | | CTG | 6 | 14.932 | | 835 | | 06 | | | | | | TTA |
| | | CTT | 39 | 16.954 | | | | | | | | | | CTA |
| | | TTA | 28 | 36.428 | | | | | | | | | | CTC |
| | | TTG | 33 | 37.371 | | | | | | | | | | CTG |
| TTT_P_ | F_P_N | CCA | 31 | 46.037 | 3 | 37.6413 | 48.2124915 | 3.36644508 | 1.918904608 | 113 | 1.74587642 | 1.7:TTTCC | 2.5:TTTCC | CCC |
| AAT | | CCC | 45 | 18.136 | | 2810681 | 4783214 | 04376885e- | 1612617e-10 | | 42940095 | GAAT | CAAT | CCA |
| | | CCG | 8 | 13.890 | | 763 | | 08 | | | | | | CCT |
| | | CCT | 29 | 34.938 | | | | | | | | | | CCG |
| TTT_R_ | F_R_Q | AGA | 19 | 32.716 | 5 | 110.323 | 272.669482 | 3.50105553 | 7.475102052 | 69 | 3.70486998 | 9.6:TTTCG | 10.4:TTTC | CGG |
| CAG | | AGG | 8 | 14.966 | | 1850926 | 9282285 | 20780096e- | 475831e-57 | | 4251732 | TCAG | GGCAG | AGA |
| | | CGA | 6 | 4.712 | | 948 | | 22 | | | | | | AGG |
| | | CGC | 5 | 4.084 | | | | | | | | | | CGA |
| | | CGG | 30 | 2.880 | | | | | | | | | | CGC |
| | | CGT | 1 | 9.642 | | | | | | | | | | CGT |
| TTT_R_ | F_R_E | AGA | 16 | 23.233 | 5 | 37.1218 | 69.1461076 | 5.66184019 | 1.542718055 | 49 | 2.26988964 | 2.9:TTTCG | 6.4:TTTCG | AGA |
| GAG | | AGG | 5 | 10.628 | | 5851290 | 0503581 | 2422441e- | 7635178e-13 | | 7081225 | CGAG | GGAG | CGG |
| | | CGA | 7 | 3.346 | | 12 | | 07 | | | | | | CGT |
| | | CGC | 1 | 2.900 | | | | | | | | | | CGA |
| | | CGG | 13 | 2.045 | | | | | | | | | | AGG |
| | | CGT | 7 | 6.847 | | | | | | | | | | CGC |
| TTT_R_ | F_R_V | AGA | 4 | 16.595 | 5 | 50.0010 | 59.6256199 | 1.38508753 | 1.452418074 | 35 | 3.16003406 | 4.9:TTTCG | 3.4:TTTAG | AGG |
| GTG | | AGG | 26 | 7.592 | | 8733584 | 1251108 | 91347145e- | 6969981e-11 | | 1294116 | TGTG | GGTG | AGA |
| | | CGA | 2 | 2.390 | | 164 | | 09 | | | | | | CGG |
| | | CGC | 0 | 2.072 | | | | | | | | | | CGA |
| | | CGG | 2 | 1.461 | | | | | | | | | | CGT |
| | | CGT | 1 | 4.891 | | | | | | | | | | CGC |
| TTT_R_ | F_R_S | AGA | 21 | 29.397 | 5 | 53.8637 | 114.440282 | 2.23535458 | 4.716608291 | 62 | 2.54296681 | 2.1:TTTCG | 7.3:TTTCG | AGA |
| TCT | | AGG | 8 | 13.448 | | 9455115 | 74090633 | 63589472e- | 1280316e-23 | | 7388512 | ATCT | GTCT | CGG |
| | | CGA | 2 | 4.234 | | 356 | | 10 | | | | | | AGG |
| | | CGC | 7 | 3.670 | | | | | | | | | | CGC |
| | | CGG | 19 | 2.588 | | | | | | | | | | CGT |
| | | CGT | 5 | 8.664 | | | | | | | | | | CGA |
| TTT_S_ | F_S_K | AGC | 16 | 22.985 | 5 | 40.6521 | 40.1530385 | 1.10303076 | 1.390918500 | 204 | 1.53347003 | 2.0:TTTAG | 1.6:TTTTC | TCA |
| AAG | | AGT | 17 | 33.293 | | 1588164 | 7628567 | 44402926e- | 993783e-07 | | 40928389 | TAAG | CAAG | TCC |
| | | TCA | 59 | 42.682 | | 31 | | 07 | | | | | | TCT |
| | | TCC | 51 | 32.032 | | | | | | | | | | TCG |
| | | TCG | 29 | 19.880 | | | | | | | | | | AGT |
| | | TCT | 32 | 53.127 | | | | | | | | | | AGC |
| TTT_S_ | F_S_T | AGC | 12 | 15.211 | 5 | 32.8208 | 39.9112384 | 4.08466981 | 1.556196662 | 135 | 1.58728652 | 1.7:TTTAG | 2.2:TTTTC | TCC |
| ACT | | AGT | 13 | 22.032 | | 0681106 | 2309104 | 15059556e- | 0960933e-07 | | 82291564 | TACT | CACT | TCT |
| | | TCA | 20 | 28.245 | | 412 | | 06 | | | | | | TCA |
| | | TCC | 47 | 21.198 | | | | | | | | | | TCG |
| | | TCG | 15 | 13.156 | | | | | | | | | | AGT |
| | | TCT | 28 | 35.158 | | | | | | | | | | AGC |
| TTT_S_ | F_S_R | AGC | 29 | 9.465 | 5 | 36.1833 | 48.8241687 | 8.72891330 | 2.410680844 | 84 | 1.78263472 | 2.3:TTTAG | 3.1:TTTAG | AGC |
| AGG | | AGT | 6 | 13.709 | | 2228613 | 3100737 | 089871e-07 | 7761882e-09 | | 15323453 | TAGG | CAGG | TCA |
| | | TCA | 15 | 17.575 | | 066 | | | | | | | | TCT |
| | | TCC | 12 | 13.190 | | | | | | | | | | TCC |
| | | TCG | 9 | 8.186 | | | | | | | | | | TCG |
| | | TCT | 13 | 21.876 | | | | | | | | | | AGT |
| TTT_S_ | F_S_Q | AGC | 3 | 12.056 | 5 | 45.9655 | 46.7885753 | 9.22983303 | 6.274166715 | 107 | 1.77141171 | 4.0:TTTAG | 2.1:TTTTC | TCA |
| CAG | | AGT | 6 | 17.463 | | 8006460 | 59426716 | 7550147e- | 753652e-09 | | 1284881 | CCAG | ACAG | TCC |
| | | TCA | 47 | 22.387 | | 656 | | 09 | | | | | | TCT |
| | | TCC | 23 | 16.801 | | | | | | | | | | TCG |
| | | TCG | 6 | 10.427 | | | | | | | | | | AGT |
| | | TCT | 22 | 27.866 | | | | | | | | | | AGC |
| TTT_S_ | F_S_V | AGC | 28 | 9.577 | 5 | 31.2352 | 42.0478845 | 8.41692759 | 5.760433672 | 85 | 1.68718146 | 2.3:TTTAG | 2.9:TTTAG | AGC |
| GTA | | AGT | 6 | 13.872 | | 1904605 | 1820383 | 9230485e- | 2795666e-08 | | 8649225 | TGTA | CGTA | TCT |
| | | TCA | 14 | 17.784 | | 2638 | | 06 | | | | | | TCA |
| | | TCC | 12 | 13.347 | | | | | | | | | | TCC |
| | | TCG | 8 | 8.283 | | | | | | | | | | TCG |
| | | TCT | 17 | 22.136 | | | | | | | | | | AGT |
| TTT_V_ | F_V_T | GTA | 16 | 27.758 | 3 | 46.2990 | 56.5370520 | 4.89912544 | 3.226481827 | 129 | 1.81992008 | 1.7:TTTGT | 2.3:TTTGT | GTC |
| ACT | | GTC | 60 | 25.990 | | 0389394 | 514044 | 2420075e- | 0356095e-12 | | 8735407 | AACT | CACT | GTT |
| | | GTG | 15 | 25.304 | | 773 | | 10 | | | | | | GTA |
| | | GTT | 38 | 49.948 | | | | | | | | | | GTG |

The examples and embodiments described herein are for illustrative purposes only and various modifications or changes suggested to persons skilled in the art are to be included within the spirit and purview of this application and scope of the appended claims.

### REFERENCES

1. Engineered dual selection for directed evolution of SpCas9 PAM specificity. Nat Commun. 2021 Jan. 13, which is incorporated by reference herein in its entirety.
2. Superloser: A Plasmid Shuffling Vector for Saccharomyces cerevisiae with Exceedingly Low Background. G3 (Bethesda). 2019 Aug. 8, which is incorporated by reference herein in its entirety.
3. Rapid and Efficient CRISPR/Cas9-Based Mating-Type Switching of Saccharomyces cerevisiae. G3 (Bethesda). 2018 Jan. 4, which is incorporated by reference herein in its entirety.
4, Resetting the Yeast Epigenome with Human Nucleosomes, Cell. 2017 Dec. 14, which is incorporated by reference herein in its entirety.
5. Low escape-rate genome safeguards with minimal molecular perturbation of Saccharomyces cerevisiae. Proc Natl Acad Sci USA. 2017 Feb. 21, which is incorporated by reference herein in its entirety.
6. Circular permutation of a synthetic eukaryotic chromosome with the telomerator. Proc Natl Acad Sci USA. 2014 Dec. 2, which is incorporated by reference herein in its entirety.
7. Multichange isothermal mutagenesis: a new strategy for multiple site-directed mutations in plasmid DNA. ACS Synth Biol. 2013 Aug. 16, which is incorporated by reference herein in its entirety.
8. Pathway Engineering in yeast for synthesizing a complex polyketide: bikaverin, Nature Comms. 2020, which is incorporated by reference herein in its entirety.
9. Emulsion-based directed evolution of enzymes and proteins in yeast. Methods Enzymol. 2020, which is incorporated by reference herein in its entirety.
10. Phylogenetic debugging of a complete human biosynthetic pathway transplanted into yeast. Nucleic Acids Res. 2019, which is incorporated by reference herein in its entirety.
11, A scalable peptide-GPCR language for engineering multicellular communication. Nature Comms. 2018., which is incorporated by reference herein in its entirety.
12. Coupling Yeast Golden Gate and VEGAS for Efficient Assembly of the Violacein Pathway in Saccharomyces cerevisiae. Methods Mol Biol. 2018, which is incorporated by reference herein in its entirety.
13. Yeast Golden Gate (yGG) for the Efficient Assembly of S. cerevisiae Transcription Units. ACS Synth Biol. 2015 Jul. 17, which is incorporated by reference herein in its entirety.
14. Versatile genetic assembly system (VEGAS) to assemble pathways for expression in S. cerevisiae. Nucleic Acids Res. 2015 Jul. 27, which is incorporated by reference herein in its entirety.
15. New Orthogonal Transcriptional Switches Derived from Tet Repressor Homologues for Saccharomyces cerevisiae Regulated by 2,4-Diacetylphloroglucinol and Other Ligands. ACS Synth Biol. 2016, which is incorporated by reference herein in its entirety.
16. Intrinsic biocontainment: multiplex genome safeguards combine transcriptional and recombinational control of essential yeast genes. Proc Natl Acad Sci USA. 2015 Feb. 10, which is incorporated by reference herein in its entirety.
17. Development of a tightly controlled off switch for Saccharomyces cerevisiae regulated by camphor, a low-cost natural product, G3. 2015, which is incorporated by reference herein in its entirety.
18. A versatile platform for locus-scale genome rewriting and verification. Proc Natl Acad Sci USA. 2021 Mar. 9, which is incorporated by reference herein in its entirety.
19. Technological challenges and milestones for writing genomes. Science. 2019 Oct. 18, which is incorporated by reference herein in its entirety.
20. Design of a synthetic yeast genome. Science. 2017 Mar. 10, which is incorporated by reference herein in its entirety.
21. RADOM, an efficient in vivo method for assembling designed DNA fragments up to 10 kb long in Saccharomyces cerevisiae. ACS Synth Biol. 2015 Mar. 20, which is incorporated by reference herein in its entirety.
22. Design of a synthetic yeast genome. Science. 2017 Mar. 10, which is incorporated by reference herein in its entirety.
23. Engineering the ribosomal DNA in a megabase synthetic chromosome. Science. 2017 Mar. 10, which is incorporated by reference herein in its entirety.
24. Synthesis, debugging, and effects of synthetic chromosome consolidation: synVI and beyond. Science. 2017 Mar. 10, which is incorporated by reference herein in its entirety.
25. “Perfect” designer chromosome V and behavior of a ring derivative. Science. 2017 Mar. 10, which is incorporated by reference herein in its entirety.
26. Deep functional analysis of synII, a 770-kilobase synthetic yeast chromosome. Science. 2017 Mar. 10, which is incorporated by reference herein in its entirety.
27. Bug mapping and fitness testing of chemically synthesized chromosome X. Science. 2017 Mar. 10, which is incorporated by reference herein in its entirety.
28. qPCRTag Analysis-A High Throughput, Real Time PCR Assay for Sc2.0 Genotyping. J Vis Exp. 2015 May 25, which is incorporated by reference herein in its entirety.
29. Total synthesis of a functional designer eukaryotic chromosome. Science. 2014 Apr. 4, which is incorporated by reference herein in its entirety.
30. Total synthesis of Escherichia coli with a recoded genome. Nature. 2019 May, which is incorporated by reference herein in its entirety.
31. Custom selenoprotein production enabled by laboratory evolution of recoded bacterial strains. Nat Biotechnol, 2018 August, which is incorporated by reference herein in its entirety.
32. Design, synthesis and testing toward a 57-codon genome. Science. 2016 Aug., which is incorporated by reference herein in its entirety.
33. Defining synonymous codon compression schemes by genome recoding. Nature. 2016 Nov. 3, which is incorporated by reference herein in its entirety.
34. tRNA genes rapidly change in evolution to meet novel translational demands, eLife. 2013, which is incorporated by reference herein in its entirety.
35. Retrotransposon Ty1 integration targets specifically positioned asymmetric nucleosomal DNA segments in tRNA hotspots. Genome Res. 2012, which is incorporated by reference herein in its entirety.
36. TFIIIB Subunit Bdp1p is Required for Periodic Integration of the Ty1 Retrotransposon and Targeting of Isw2p to S. cerevisiae tDNAs, Genes Dev. 2005, which is incorporated by reference herein in its entirety.
37. Local definition of Ty1 target preference by Long Terminal Repeats and clustered tRNA genes. Genome Research, 2004, which is incorporated by reference herein in its entirety.
38. Interactions between tRNA genes, flanking genes and Ty elements: a genomic point of view. Genome Res. 2003, which is incorporated by reference herein in its entirety.
39. The yeast retrotransposon uses the anticodon stem-loop of the initiator methionine tRNA as a primer for reverse transcription. R(NA. 1999, which is incorporated by reference herein in its entirety.
40. Multiple molecular determinants for retrotransposition in a primer tRNA. Mol, Cell.

Biol. 1995, which is incorporated by reference herein in its entirety.
41. Yeast retrotransposons and tRNAs. Trends Genet. 1993, which is incorporated by reference herein in its entirety.
42. A rare tRNA-Arg(CCU) that regulates Ty1 element ribosomal frameshifting is essential for Ty1 retrotransposition in Saccharomyces cerevisiae. Genetics. 1993, which is incorporated by reference herein in its entirety.
43. Hotspots for unselected Ty1 transposition events on yeast chromosome III are near tRNA genes and LTR sequences. Cell. 1993, which is incorporated by reference herein in its entirety.
44. Initiator methionine tRNA is essential for Ty1 transposition in yeast. Proc. Natl. Acad. 1992, which is incorporated by reference herein in its entirety.
45. Host genes that influence transposition in yeast: the abundance of a rare tRNA regulates Ty1 transposition frequency. Proc. Natl. Acad. Sci. 1990, which is incorporated by reference herein in its entirety.
46. Future prospects for noncanonical amino acids in biological therapeutics. Curr Opin Biotechnol. 2019 Dec., which is incorporated by reference herein in its entirety.
47. A Robust and Quantitative Reporter System To Evaluate Noncanonical Amino Acid Incorporation in Yeast. ACS Synth Biol. 2018 Sep. 21, which is incorporated by reference herein in its entirety.
48. Directed Evolution of Heterologous tRNAs Leads to Reduced Dependence on Post-transcriptional Modifications. ACS Synth Biol. 2018 May 18, which is incorporated by reference herein in its entirety.
49. Evolving Orthogonal Suppressor tRNAs To Incorporate Modified Amino Acids. ACS Synth Biol. 2017 Jan. 20, which is incorporated by reference herein in its entirety.
50. Rapid and Inexpensive Evaluation of Nonstandard Amino Acid Incorporation in Escherichia coli. ACS Synth Biol. 2017 Jan. 20, which is incorporated by reference herein in its entirety.
51. Addicting diverse bacteria to a noncanonical amino acid. Nat Chem Biol. 2016 Mar., which is incorporated by reference herein in its entirety.
52. A switchable yeast display/secretion system. Protein Eng Des Sel. 2015 Oct., which is incorporated by reference herein in its entirety.
53. Efficient genetic encoding of phosphoserine and its nonhydrolyzable analog. Nat Chem Biol. 2015 Jul., which is incorporated by reference herein in its entirety.
54. Optimized orthogonal translation of unnatural amino acids enables spontaneous protein double-labelling and FRET, Nat Chem. 2014 May, which is incorporated by reference herein in its entirety.
55, Encoding multiple unnatural amino acids via evolution of a quadruplet-decoding ribosome. Nature. 2010 Mar., which is incorporated by reference herein in its entirety.
56. Evolved orthogonal ribosomes enhance the efficiency of synthetic genetic code expansion, Nat Biotechnol, 2007 Jul., which is incorporated by reference herein in its entirety.
57. Ranked List Loss for Deep Metric Learning, IEEE Trans. Pattern Analysis and Machine Intelligence, 2021 Jan., which is incorporated by reference herein in its entirety.
58. ProSelfLC: Progressive Self Label Correction for Training Robust Deep Neural Networks, CVPR 2021, which is incorporated by reference herein in its entirety.
59. MAMBA: Multi-level Aggregation via Memory Bank for Video Object Detection, AAAI 2020, which is incorporated by reference herein in its entirety,
60. DADA: Differentiable Automatic Data Augmentation, ECCV 2020, which is incorporated by reference herein in its entirety.
61. Deep Metric Learning by Online Soft Mining and Class-Aware Attention, AAAI 2019, which is incorporated by reference herein in its entirety.
62, Ranked List Loss for Deep Metric Learning, CVPR 2019, which is incorporated by reference herein in its entirety.
63. Deep Metric Learning for Proteomics, IEEE Int. Conf. Machine Learning Applications, 2020, Sep., which is incorporated by reference herein in its entirety.
64. Expanding the Vocabulary of a Protein: Application of Subword Algorithms to Protein Sequence Modelling, IEEE Eng. Med. Bio, 2020 Aug., which is incorporated by reference herein in its entirety.
65. Low escape-rate genome safeguards with minimal molecular perturbation of Saccharomyces cerevisiae. Proc Natl Acad Sci USA. 2017, which is incorporated by reference herein in its entirety.
66. Intrinsic biocontainment: Multiplex genome safeguards combine transcriptional and recombinational control of essential yeast genes. Proc Natl Acad Sci. 2015, which is incorporated by reference herein in its entirety.
67. Freedom and Responsibility in Synthetic Genomics: The Sc2.0 Project. Genetics 2015, which is incorporated by reference herein in its entirety.
68. Regulation of the Dot1 historic H3K79 methyltransferase by histone H4K16 acetylation. Science. 2021, which is incorporated by reference herein in its entirety. 69, Genetic interaction mapping informs integrative structure determination of molecular assemblies, Science. 2020, which is incorporated by reference herein in its entirety.
70. Dissecting nucleosome function with a comprehensive histone H2A and H2B mutant library. G3. 2017, which is incorporated by reference herein in its entirety.
71. Construction of comprehensive dosage-matching core histone mutant libraries for Saccharomyces cerevisiae. Genetics. 2017, which is incorporated by reference herein in its entirety.
72. Interplay between histone 1-13 lysine 56 deacetylation and chromatin modifiers in the response to replicative DNA damage, Genetics. 2015, which is incorporated by reference herein in its entirety.
73. A high-resolution view of histone modifications and transcription across distinct metabolic states in budding yeast. Nature Struct Molec Biol. 2014, which is incorporated by reference herein in its entirety.
74. Identification of histone H3 and H4 residues that regulate chromosome segregation in budding yeast, Genetics. 2013, which is incorporated by reference herein in its entirety.
75. Strain construction and screening methods for a yeast histone H3/H4 mutant library. In Randall H Morse (ed.), Chromatin Remodeling: Methods and Protocols, Methods in Molecular Biology. 2012, which is incorporated by reference herein in its entirety.
76, Differential contributions of histone 1-13 and 1-14 residues to heterochromatin structure, Genetics. 2011, which is incorporated by reference herein in its entirety.
77. A “Young” Lysine Residue in Histone H3 Attenuates Transcriptional Output in Saccharomyces cerevisiae. Genes Dev. 2011, which is incorporated by reference herein in its entirety.
78. Yin and yang of histone 1-1213 roles in silencing and longevity: A tale of two arginines. Genetics. 2010, which is incorporated by reference herein in its entirety.
79. Histone H3 Exerts Key Function in Mitotic Checkpoint Control. Mol, Cell Biol. 2009, which is incorporated by reference herein in its entirety.
80. A comprehensive synthetic genetic interaction network governing yeast histone acetylation and deacetylation. Genes Dev. 2008, which is incorporated by reference herein in its entirety.
81. Probing nucleosome function: A highly versatile library of synthetic histone 13 and H4 mutants. Cell. 2008, which is incorporated by reference herein in its entirety.
82, The LRS and SIN domains: Two structurally equivalent but functionally distinct nucleosomal surfaces required for transcriptional silencing. Mol. Cell Biol. 2006, which is incorporated by reference herein in its entirety.
83. The sirtuins Hst3 and Hst4p preserve genome integrity by controlling histone 1-13 lysine 56 deacetylation. Current Biology. 2006, which is incorporated by reference herein in its entirety.
84. Insights into the Role of Histone H3 and Histone H4 Core Modifiable Residues in Saccharomyces cerevisiae. Mol. Cell Biol. 2005, which is incorporated by reference herein in its entirety.
85, Regulated nucleosome mobility and the histone code, Nature Struct. Mol, Biol. 2004, which is incorporated by reference herein in its entirety.
86. SPT10 and SPT21 are required for transcription of particular histone genes in Saccharomyces cerevisiae, Mol. Cell. Biol. 1994, which is incorporated by reference herein in its entirety,
87, Engineered dual selection for directed evolution of SpCas9's PAM specificity. Nature Comms. in press. 2021, which is incorporated by reference herein in its entirety.
88. CRISPR-Cas12a system in fission yeast for multiplex genomic editing and CRISPR interference. Nucleic Acids Res. 2020, which is incorporated by reference herein in its entirety.
89. Construction of Designer Selectable Marker Deletions with a CRISR-Cas9 Toolbox in Schizosaccharomyces pombe and Optimized Design of Common Entry Vectors. G3. 2017, which is incorporated by reference herein in its entirety.
90. Rapid and Efficient CRISPR/Cas9-Based Mating-Type Switching of Saccharomyces cerevisiae. G3 (Bethesda). 2017 Nov. 22, which is incorporated by reference herein in its entirety.
91. Versatile Genetic Assembly System (VEGAS) to assemble pathways for expression in S. cerevisiae. Nucl Acids Res. 2015, which is incorporated by reference herein in its entirety.
92. Yeast Golden Gate (yGG) for efficient assembly of Saccharomyces cerevisiae transcription units, ACS Synth Biol. 2015, which is incorporated by reference herein in its entirety.
93. Circular permutation of a synthetic eukaryotic chromosome with the telomerator. Proc Natl Acad Sci USA. 2014, which is incorporated by reference herein in its entirety.
94, RADOM, an Efficient In Vivo Method for Assembling Designed DNA Fragments up to 10 kb Long in Saccharomyces cerevisiae. ACS Synth Biol. 2014, which is incorporated by reference herein in its entirety.
95. GeneDesign 3.0: an Updated Synthetic Biology Toolkit. Nucl Acids Res. 2010, which is incorporated by reference herein in its entirety.
96, CloneQC: Lightweight sequence verification for synthetic biology. Nucl. Acids Res. 2010, which is incorporated by reference herein in its entirety.
97. Automated Design of Assemblable, Modular, Synthetic Chromosomes. 8th International Conference, PPAM 2009, Wroclaw, Poland, Sep. 13-16, 2009, which is incorporated by reference herein in its entirety.
98. GeneDesign: Rapid, Automated Design of Multikilobase Synthetic Genes. Genome Res. 2006, which is incorporated by reference herein in its entirety.
99. A robust and quantitative report system to evaluate noncanonical amino aid incorporation in yeast. ACS Synth Biol. 2018 Sep. 21; 7(9): 2256-2269, which is incorporated by reference herein in its entirety.

1. A method comprising:
a) analyzing at least a portion of a genome of an organism to identify a first plurality of codons based on at least in part on a first local context of a codon-of-interest in the genome of the organism to be rewritten;
b) rewriting the first plurality of codons in the genome of the organism to a second codon, wherein the first plurality of codons and the second codon encode a first amino acid, and wherein the rewriting of the first plurality of codons modulates an occurrence of the first plurality of codons;
c) synthesizing a nucleic acid construct comprising the portion of the genome of the organism, wherein the first plurality of codons is rewritten to the second codon; and
d) introducing the nucleic acid construct into a cell of the organism to replace the portion of the genome of the organism.

2. (canceled)

3. The method of claim 1, wherein the modulating of the occurrence of the first plurality of codons comprises eliminating the occurrence of the first plurality of codons.

4. The method of claim 1, wherein the analyzing comprises identifying one or more synonymous codons with a least number of occurrences in the genome of the organism, wherein the first plurality of codons comprises the one or more synonymous codons with the least number of occurrences.

5. (canceled)

6. The method of claim 4, wherein the analyzing further comprises determining a number of occurrences of the first local context of the codon-of-interest, wherein the first local context of the codon-of-interest comprises
C₍ₙ₋₁₎−Cₙ−C₍ₙ₊₁₎, wherein
C₍ₙ₋₁₎ denotes a codon downstream of the codon-of-interest;
Cₙ denotes the codon-of-interest; and
C₍ₙ₊₁₎ denotes a codon upstream of the codon-of-interest.

7. (canceled)

8. The method of claim 6, the preceding, wherein the analyzing further comprises determining a relative synonymous codon usage (RSCU) of the codon-of-interest.

9. The method of claim 8, wherein the analyzing further comprises identifying the first plurality of codons based at least in part on a second local context of the codon-of-interest in the genome of the organism, wherein the second local context of the codon-of-interest comprises
C₍ₙ₋₁₎−AAₙ−C₍ₙ₊₁₎, wherein
C₍ₙ₋₁₎ denotes a codon downstream of the codon-of-interest;
AAₙ denotes an amino acid encoded by the codon-of-interest; and
C₍ₙ₊₁₎ denotes a codon upstream of the codon-of-interest.

10. (canceled)

11. The method of claim 9, wherein the analyzing further comprises determining a number of occurrences of the second local context of the codon-of-interest.

12. The method of claim 11, wherein the analyzing further comprises determining an expected number of occurrences of the first local context of the codon-of-interest, wherein the expected number of occurrences of the first local context of the codon-of-interest is determined as a product of: a number of occurrences of the second local context of the codon-of-interest, and the determined RCSU of the codon-of-interest.

13. (canceled)

14. The method of claim 1, wherein the analyzing comprises processing the at least the portion of the genome of the organism using a machine learning-based computer system, wherein the machine learning-based computer system comprises one or more storage units comprising, respectively, one or more storage devices included within respective storage arrays controlled by a respective one or more storage controllers; and one or more computer processing units, wherein the one or more computer processing units communicate with the one or more storage units over a communication interface.

15. (canceled)

16. The method of claim 1, wherein the analyzing further comprises identifying one or more statistically significant evolutionary signals, wherein the one or more statistically significant evolutionary signals comprise a negative evolutionary selection signal, a positive evolutionary selection signal, or a combination thereof;
wherein the negative selection signal comprises a frameshift, a ribosome stall, or a secondary RNA structure interfering with transcription or translation; and
wherein the positive selection signal comprises a regulatory element within an open reading frame (ORF).

17.-19. (canceled)

20. The method of claim 1, wherein the method further comprises reassigning the first plurality of codons to a second amino acid.

21. (canceled)

22. The method of claim 1, wherein the first amino acid comprises arginine, leucine, or serine.

23. (canceled)

24. The method of claim 1, wherein the first plurality of codons comprises CGA, CGG, or a combination thereof.

25. (canceled)

26. The method of claim 1, wherein the first plurality of codons comprises CTA, CTG, or a combination thereof.

27. (canceled)

28. The method of claim 1, wherein the first plurality of codons comprises AGT, AGC, TCG, TCA, or a combination thereof.

29. The method of claim 1, wherein the rewriting further comprises removing a plurality of tRNA molecules with anticodons that recognize the first plurality of codons, wherein the removing comprises deleting one or more genes that encode the plurality of tRNA molecules that recognize the first plurality of codons.

30. (canceled)

31. The method of claim 20, further comprising providing the cell (i) additional tRNA molecules that recognize the first plurality of codons and aminoacyl-tRNA synthetases (aaRSs) for charging the additional tRNA molecules with the second amino acid: (ii) a tRNA pre-charged with the second amino acid: or (iii) both (i) and (ii).

32. (canceled)

33. The method of claim 20, wherein the second amino acid comprises a non-canonical amino acid, wherein the non-canonical amino acid comprises an azide-containing ncAA, an alkene-containing ncAA, an alkyne-containing ncAA, p-azidophenylalanine, 2-aminoisobutyric acid (Aib), N6-[(propargyloxy)carbonyl]-L-lysine, O—4-allyl-L-tyrosine, or a combination thereof.

34. (canceled)

35. The method of claim 1, wherein the rewriting of the first plurality of codons comprises modulating one or more codons in the first plurality of codons, wherein the one or more codons are within 4 codons of each other.

36. The method of claim 1, wherein the rewriting of the first plurality of codons comprises modulating a codon fragment of one or more codons in the first plurality of codons, wherein the codon fragment comprises a trimer, a hexamer, a 9 mer, or a combination thereof.

37. (canceled)

38. A method of producing a polypeptide comprising a non-canonical amino acid (ncAA) or a population of polypeptide molecules comprising the ncAA in an organism, the method comprising:
rewriting a first codon encoding a first amino acid to a second codon encoding the first amino acid in a genome of the organism, wherein the rewriting comprises identifying the first codon based at least in part on a first local context of a codon-of-interest in the genome of the organism;
reassigning the first codon to encode the ncAA in the genome of the organism; and
introducing into the organism an aminoacyl-tRNA synthetase (aaRS)/tRNA pair engineered to recognize the first codon and incorporate the ncAA into an amino acid sequence of the polypeptide or the population of the polypeptide molecules.

39.-67. (canceled)

68. A cell or a population of cells comprising a genome, wherein a first plurality of codons in the genome of the organism is rewritten to a second codon, wherein the first plurality of codons and the second codon encode a first amino acid, and wherein an occurrence of the first plurality of codons is modulated responsive to being rewritten to the second codon, wherein the first plurality of codons is reassigned to a second amino acid.

69.-103. (canceled)
